SEQUENCE LISTING (1 sequence)

SEQ ID NO: 1 - RNA, 1653 nt, unidentified
  source 1..1653: mol_type = mRNA
atggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtga

(19) United States
(12) Patent Application Publication
(10) Pub. No.: US 2025/0276087 A1
(43) Pub. Date: Sep. 4, 2025

(54) POLYMER-BASED NUCLEIC ACID MOLECULE DELIVERY VEHICLE HAVING IONIZATION MOIETY

(71) Applicants: POSTECH RESEARCH AND BUSINESS DEVELOPMENT FOUNDATION, Pohang-si (KR); OMNIAMED CO.LTD, Pohang-si (KR)

(72) Inventors: Won Jong KIM, Pohang-si (KR); Hong Lyun KIM, Pohang-si (KR)

(21) Appl. No.: 19/194,035

(22) Filed: Apr. 30, 2025

Related U.S. Application Data
(63) Continuation of application No. PCT/KR2023/017176, filed on Oct. 31, 2023.

(30) Foreign Application Priority Data
Nov. 1, 2022 (KR) 10-2022-0143417

(51) Int. Cl. A61K 48/00; A61K 39/385; A61K 47/59

(52) U.S. Cl. CPC A61K 48/0041; A61K 39/385; A61K 47/593

(57) ABSTRACT

Provided is a polymer-based nucleic acid molecule (e.g., mRNA) delivery vehicle having an ionizable moiety. Specifically, the present invention provides a novel polymer capable of delivering all negatively charged genetic materials to a desired site according to the monomers and the length of the polymer. The polymer according to the present invention can be effectively used in gene therapeutic agents or vaccines comprising genetic material, such as mRNA, for therapeutic or preventive purposes.

### INCORPORATION OF SEQUENCE LISTING

This application contains a sequence listing submitted in Computer Readable Form (CRF). The CRF file containing the sequence listing entitled “PK6387466-SequenceListing.xml”, which was created on May 14, 2025, and is 3,687 bytes in size. The information in the sequence listing is incorporated herein by reference in its entirety.

### TECHNICAL FIELD

The present invention relates to a polymer-based nucleic acid molecule delivery vehicle having an ionizable moiety, and more specifically, to a polymer based on poly beta-aminoester or poly beta-aminoacrylate. Furthermore, the polymer according to the present invention can provide high mRNA delivery efficiency through pH sensitivity by attaching PEG to both ends to synthesize a triple copolymer.

### BACKGROUND ART

mRNA is a substance that carries genetic information for producing proteins in the human body. Through mRNA delivery, it is possible to induce the production of proteins that are originally present in the human body, as well as the production of proteins that do not exist in the body. Since the outbreak of COVID-19, the development of mRNA delivery vehicles has been actively progressing as mRNA vaccines that activate immune responses through mRNA delivery have been utilized. Several mRNA delivery vehicles based on lipid nanoparticles have been developed, but they have problems due to rapid diffusion to organs other than the target site (off-target effect) and high immunogenicity, which can cause various side effects such as myocarditis and pericarditis. In addition, since vaccines are inoculated up to 2 to 4 times, the possibility of exposure to these side effects increases. Therefore, it is necessary to develop an mRNA delivery vehicle with high mRNA expression efficiency, low off-target effect, relatively low immunogenicity, and low side effects.

In order to improve the disadvantages of the above mRNA delivery vehicle, the present researchers studied mRNA delivery using polymers. As a result, a novel polymer that has pH sensitivity and has high mRNA delivery efficiency in vivo was newly discovered. Based on the above, the present researchers completed the present invention.

### DETAILED DESCRIPTION OF INVENTION

### Technical Problem

An object of the present invention is to provide a nucleic acid molecule delivery vehicle based on a polymer having pH sensitivity for high nucleic acid molecule delivery efficiency and low side effects.

One aspect of the object of the present invention is to provide a mRNA delivery vehicle based on a polymer having a relatively long duration of expression in order to overcome the short-term expression of mRNA in general.

Another object of the present invention is to provide a novel polymer for providing a nucleic acid molecule delivery vehicle based on a polymer having pH sensitivity.

### Solution to Problem

In order to achieve the above object, the present invention provides a polymer suitable for intracellular nucleic acid delivery, comprising a repeating unit of Formula 1 below:

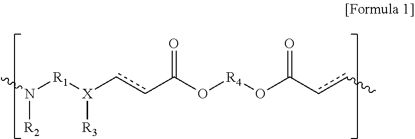

in the formula,
R1 is a bond or a C1-6 alkylene,
R2 and R3 are each independently (i) hydrogen, (ii) C1-6 alkyl which may be substituted with alkylamine or hydroxy, (iii) a 5- or 6-membered carbocycle or heterocycle, or (iv) C1-6 alkyl substituted with a 5- or 6-membered carbocycle or heterocycle,
said R2 and R3 may be linked together to form a C1-6 alkylene,
said 5- or 6-membered carbocycle or heterocycle may be substituted with C1-3 alkyl or C1-3 alkoxyacyl,
X is a nitrogen atom or a bond, and when X is a bond, said R3 is absent,
R4 is a linear or branched C1-15 alkylene, 1 to 5 carbon atoms of said alkylene may be replaced by oxygen or sulfur, and
[symbol] may represent a single bond or a double bond.

The above repeating unit may be n (an integer between 1 and 500), and in this case, Formula 1 may be represented as follows.

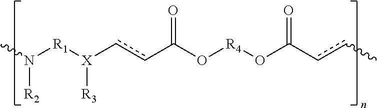

(the definitions of the groups are as defined in relation to Formula 1 above)

The present invention provides a polymer having a repeating unit of Formula 2 below:

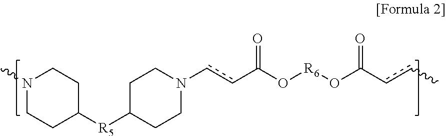

in the formula,
R5 is a C1-6 alkylene,
R6 is a linear or branched C1-15 alkylene, 1 to 5 carbon atoms of said alkylene may be replaced by oxygen or sulfur, and
[symbol] may represent a single bond or a double bond.

The above repeating unit may be n (an integer between 1 and 500), and in this case, Formula 2 may be represented as follows.

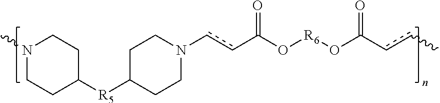

(the definitions of the groups are as defined in relation to Formula 2 above)

In one embodiment, the polymer may be a polymer in which the repeating unit of Formula 1 is formed by a reaction between any one of the monomers of Formulas 3 to 13 below and any one of the monomers of Formulas 14 to 23 below.

| Formula | Structure |
|---|---|
| 3 | 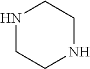 |
| 4 | 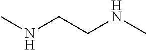 |
| 5 | 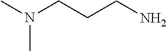 |
| 6 | 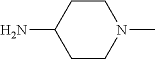 |
| 7 | 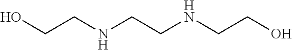 |
| 8 | 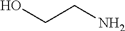 |
| 9 | 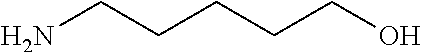 |
| 10 | 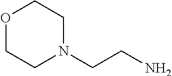 |
| 11 | 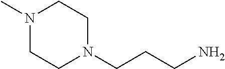 |
| 12 | 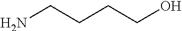 |
| 13 | 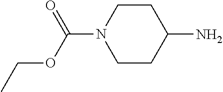 |
| 14 | 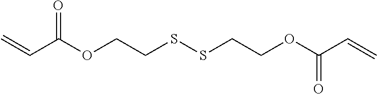 [SMILES: C=CC(=O)OCCSSCCOC(=O)C=C] |
| 15 | 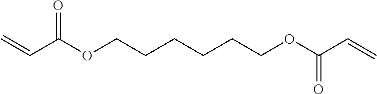 [SMILES: C=CC(=O)OCCCCCCOC(=O)C=C] |
| 16 | 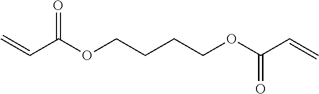 |
| 17 | 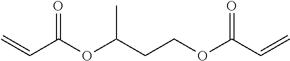 |
| 18 | 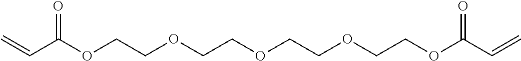 [SMILES: C=CC(=O)OCCOCCOCCOCCOC(=O)C=C] |
| 19 | 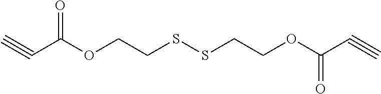 [SMILES: C#CC(=O)OCCSSCCOC(=O)C#C] |
| 20 | 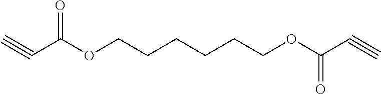 [SMILES: C#CC(=O)OCCCCCCOC(=O)C#C] |
| 21 | 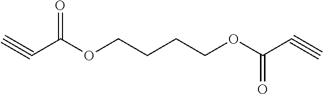 |
| 22 | 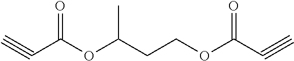 |
| 23 | 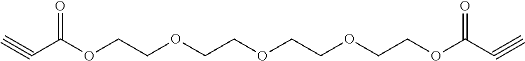 [SMILES: C#CC(=O)OCCOCCOCCOCCOC(=O)C#C] |

In one embodiment, the polymer may be a polymer in which the repeating unit of Formula 2 is formed by a reaction between a monomer of Formula 24 below and any one of the monomers of Formulas 14 to 23 below.

| Formula | Structure |
|---|---|
| 14 | 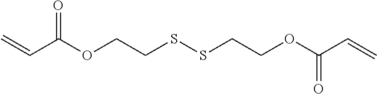 [SMILES: C=CC(=O)OCCSSCCOC(=O)C=C] |
| 15 | 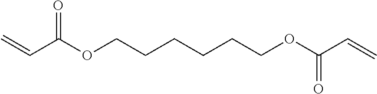 [SMILES: C=CC(=O)OCCCCCCOC(=O)C=C] |
| 16 | 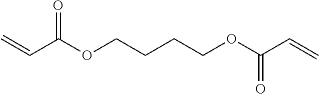 |
| 17 | 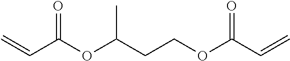 |
| 18 | 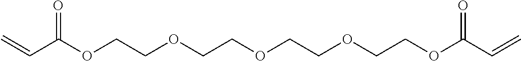 [SMILES: C=CC(=O)OCCOCCOCCOCCOC(=O)C=C] |
| 19 | 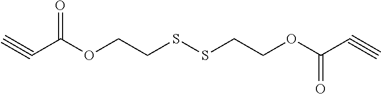 [SMILES: C#CC(=O)OCCSSCCOC(=O)C#C] |
| 20 | 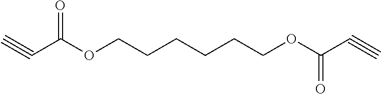 [SMILES: C#CC(=O)OCCCCCCOC(=O)C#C] |
| 21 | 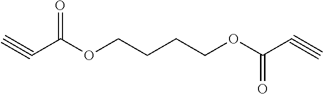 |
| 22 | 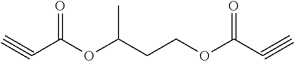 |
| 23 | 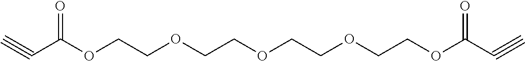 [SMILES: C#CC(=O)OCCOCCOCCOCCOC(=O)C#C] |
| 24 | 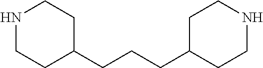 [SMILES: C(CC1CCNCC1)CC1CCNCC1] |

The present invention provides a polymer comprising a repeating unit selected from the group consisting of repeating units set forth in the table below.

| Man- | | |
|---|---|---|
| ufac- | | |
| tur- | | |
| ing | | |
| Ex- | | |
| am- | Code | |
| ple | No. | Chemical structural formula |
| 1 | 1A | 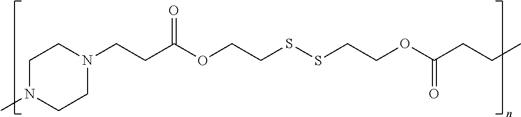 |
| 2 | 1B | 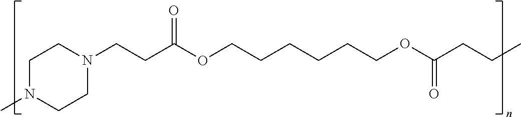 |
| 3 | 1C | 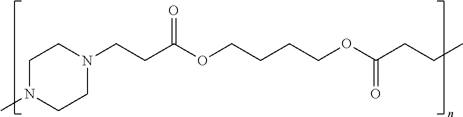 |
| 4 | 1D | 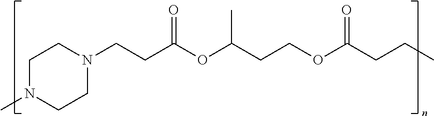 |
| 5 | 1E | 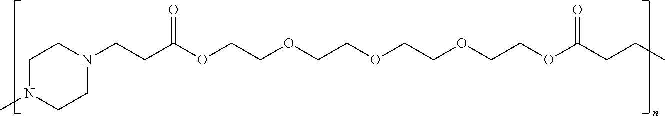 |
| 6 | 2A | 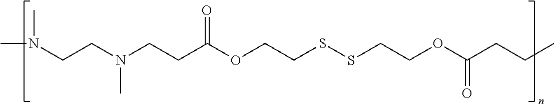 |
| 7 | 2B | 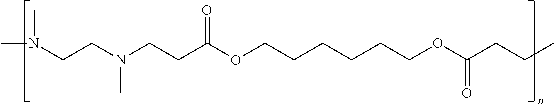 |
| 8 | 2C | 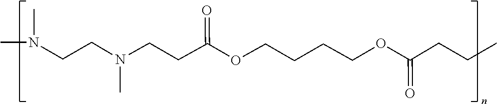 |
| 9 | 2D | 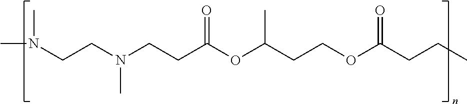 |
| 10 | 2E | 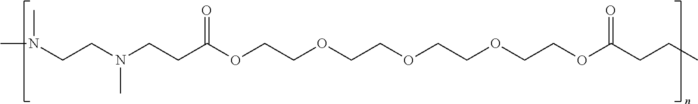 |
| 11 | 3A | 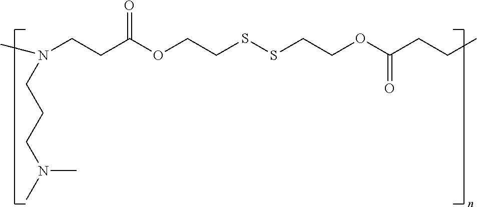 |
| 12 | 3B | 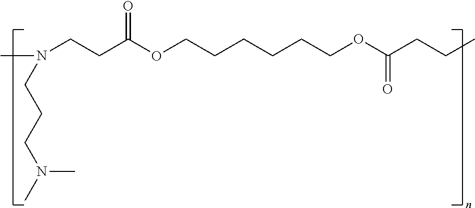 |
| 13 | 3C | 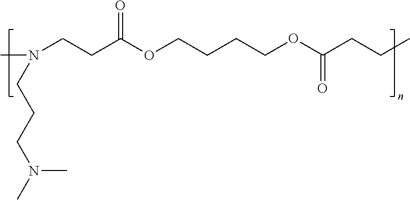 |
| 14 | 3D | 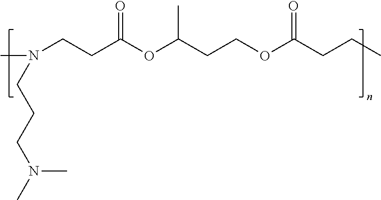 |
| 15 | 3E | 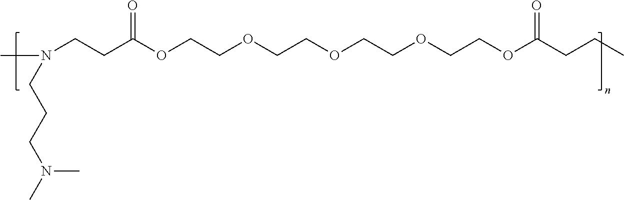 |
| 16 | 4A | 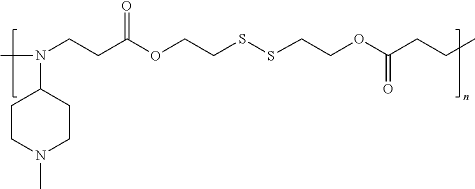 |
| 17 | 4B | 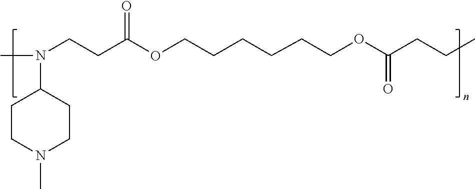 |
| 18 | 4C | 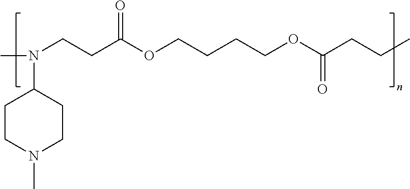 |
| 19 | 4D | 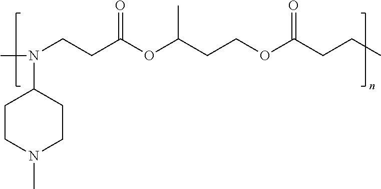 |
| 20 | 4E | 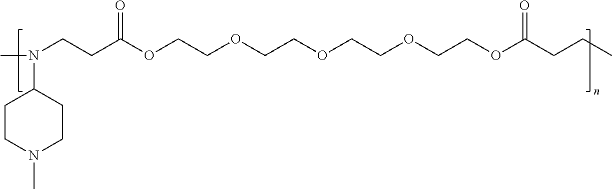 |
| 21 | 5A | 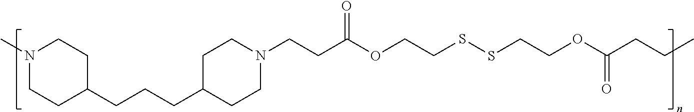 |
| 22 | 5B | 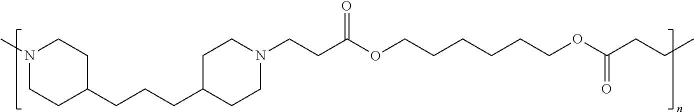 |
| 23 | 5C | 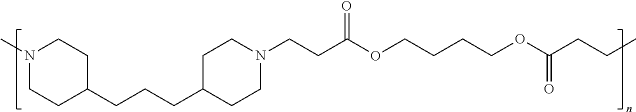 |
| 24 | 5D | 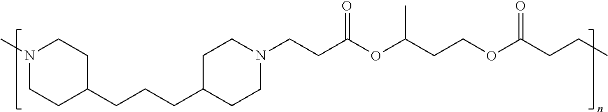 |
| 25 | 5E | 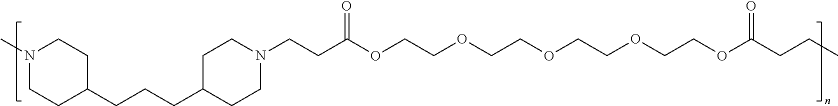 |
| 26 | 6A | 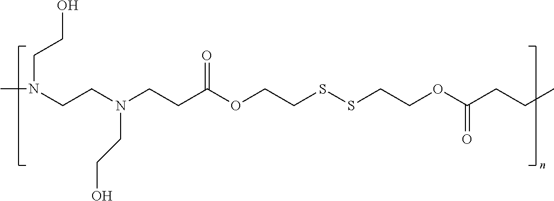 |
| 27 | 6B | 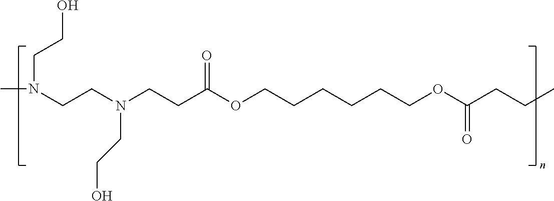 |
| 28 | 6C | 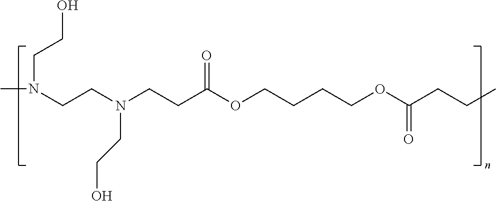 |
| 29 | 6D | 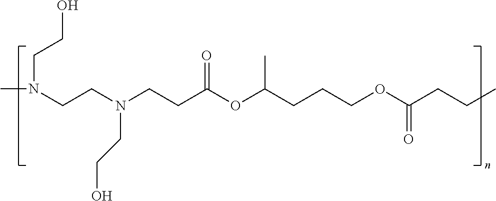 |
| 30 | 6E | 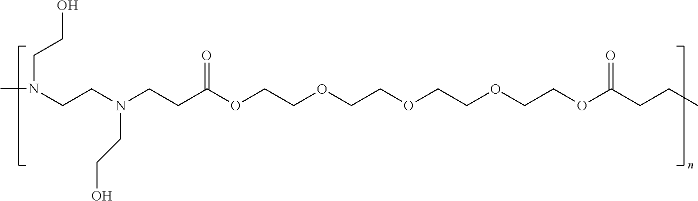 |
| 31 | 7A | 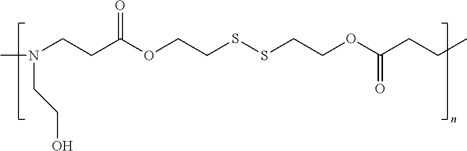 |
| 32 | 7B | 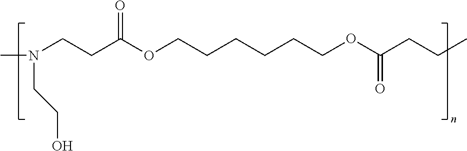 |
| 33 | 7C | 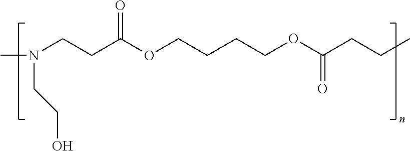 |
| 34 | 7D | 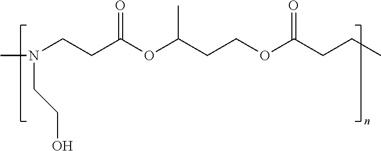 |
| 35 | 7E | 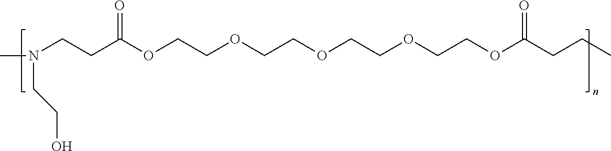 |
| 36 | 8A | 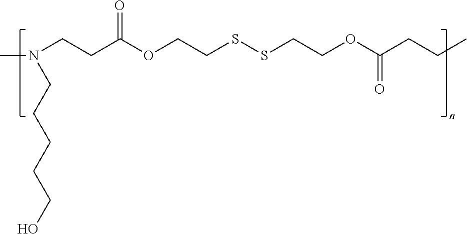 |
| 37 | 8B | 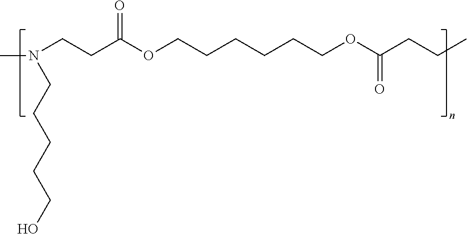 |
| 38 | 8C | 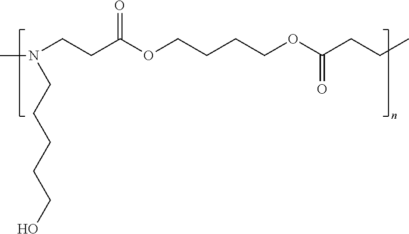 |
| 39 | 8D | 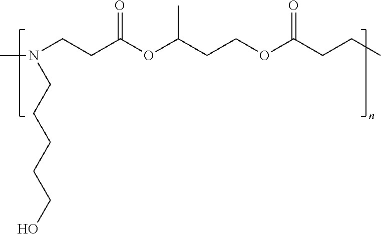 |
| 40 | 8E | 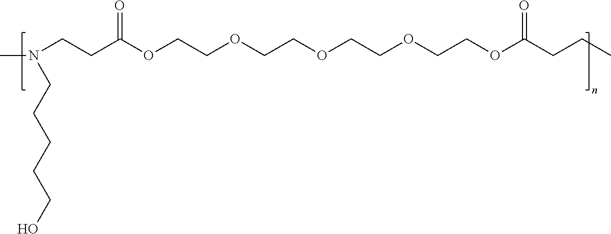 |
| 41 | 9A | 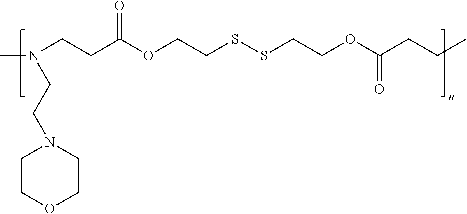 |
| 42 | 9B | 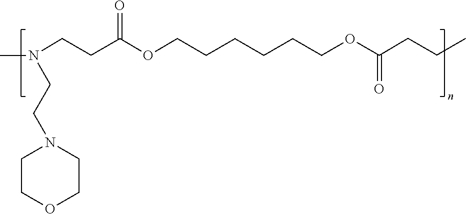 |
| 43 | 9C | 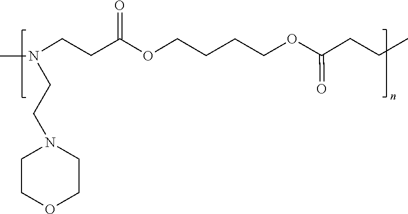 |
| 44 | 9D | 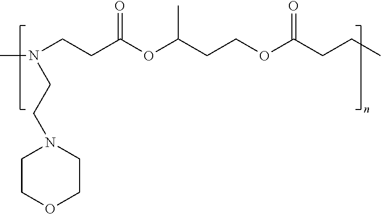 |
| 45 | 9E | 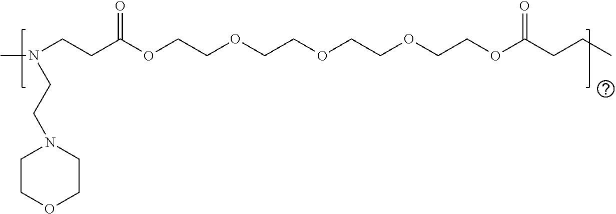 |
| 46 | 10A | 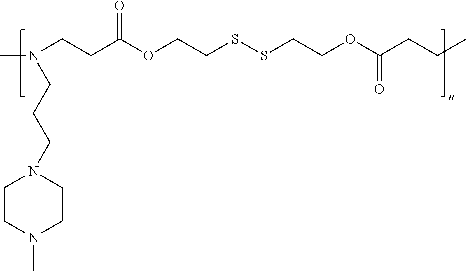 |
| 47 | 10B | 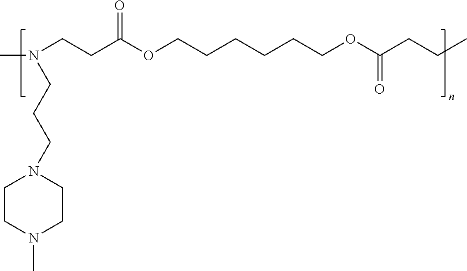 |
| 48 | 100 | 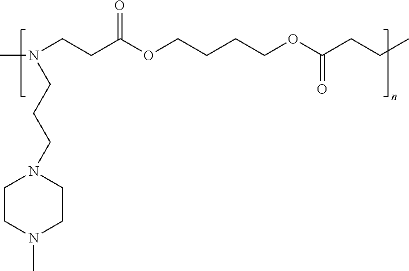 |
| 49 | 10D | 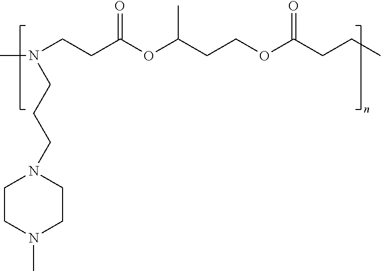 |
| 50 | 10E | 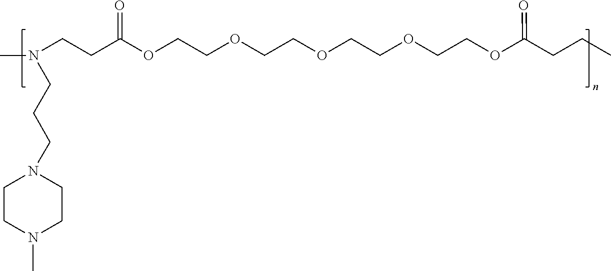 |
| 51 | 11A | 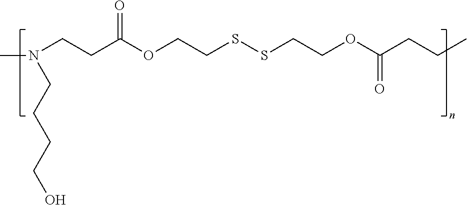 |
| 52 | 11B | 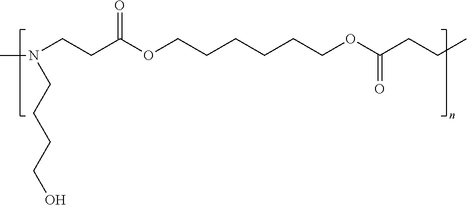 |
| 53 | 11C | 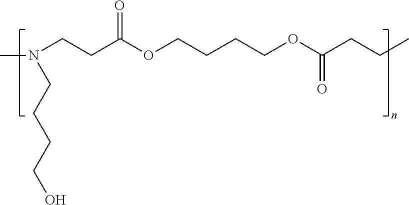 |
| 54 | 11D | 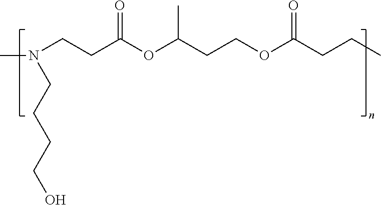 |
| 55 | 11E | 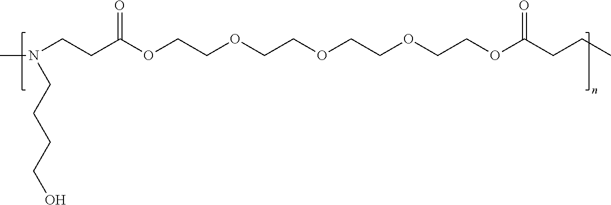 |
| 56 | 1F | 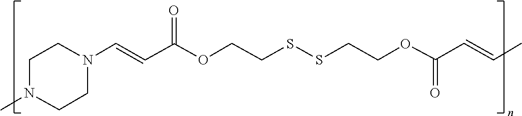 |
| 57 | 1G | 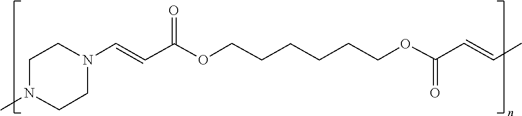 |
| 58 | 1H | 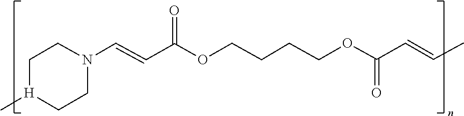 |
| 59 | 11 | 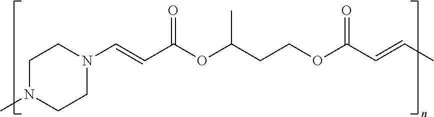 |
| 60 | 1J | 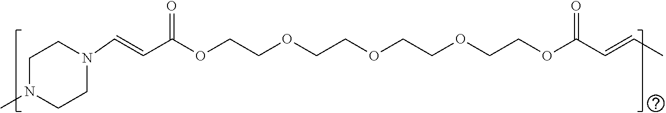 |
| 61 | 2F | 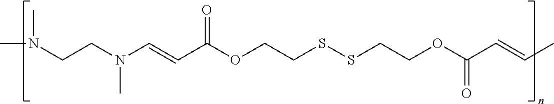 |
| 62 | 2G | 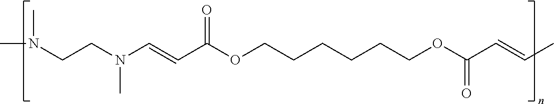 |
| 63 | 2H | 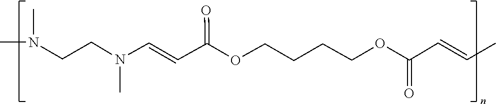 |
| 64 | 21 | 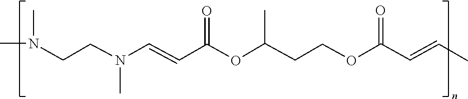 |
| 65 | 2J | 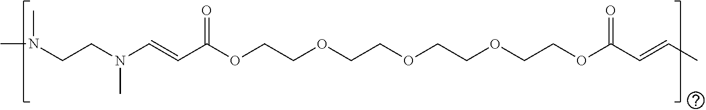 |
| 66 | 3F | 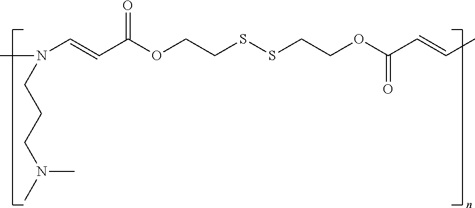 |
| 67 | 3G | 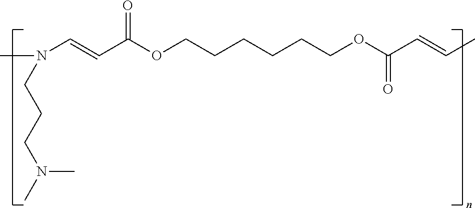 |
| 68 | 3H | 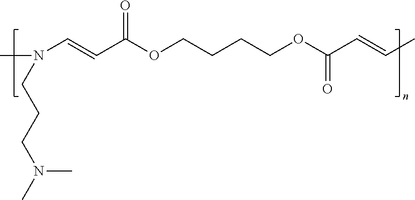 |
| 69 | 3I | 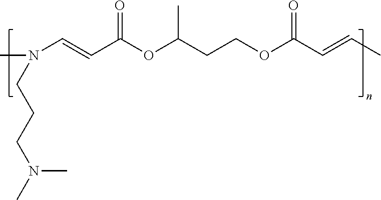 |
| 70 | 3J | 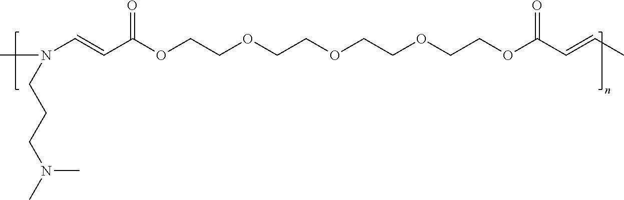 |
| 71 | 4F | 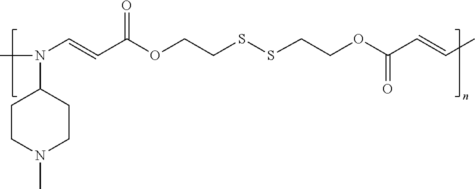 |
| 72 | 4G | 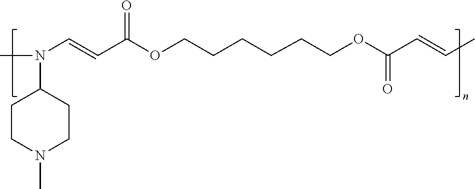 |
| 73 | 4H | 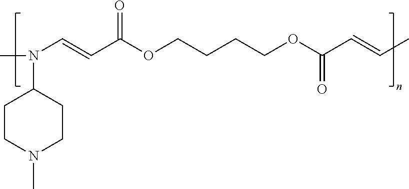 |
| 74 | 4I | 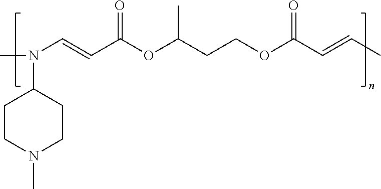 |
| 75 | 4J | 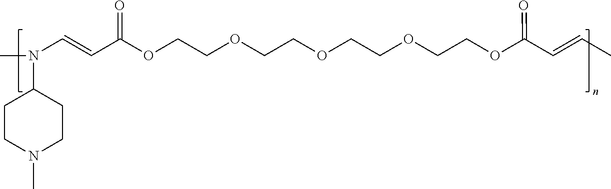 |
| 76 | 5F | 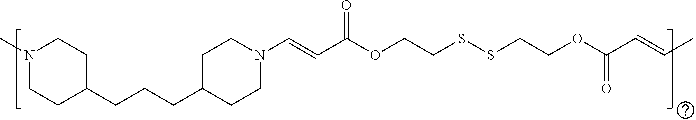 |
| 77 | 5G | 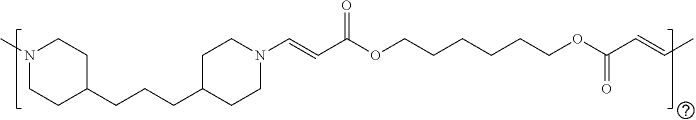 |
| 78 | 5H | 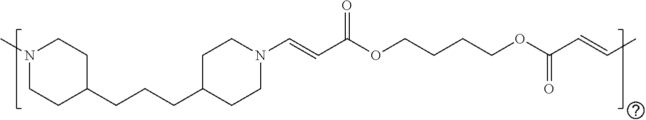 |
| 79 | 5I | 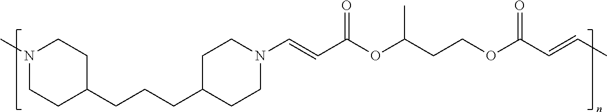 |
| 80 | 5J | 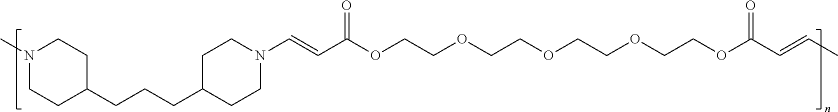 |
| 81 | 6F | 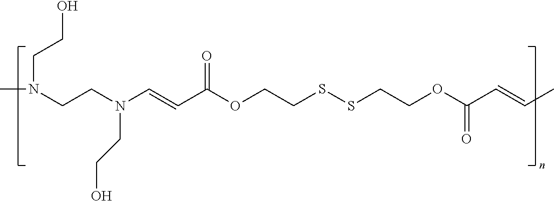 |
| 82 | 6G | 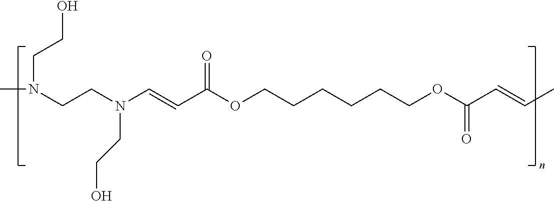 |
| 83 | 6H | 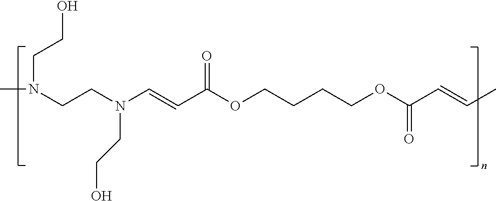 |
| 84 | 6I | 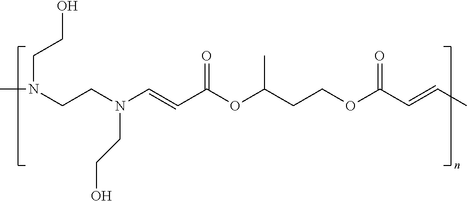 |
| 85 | 6J | 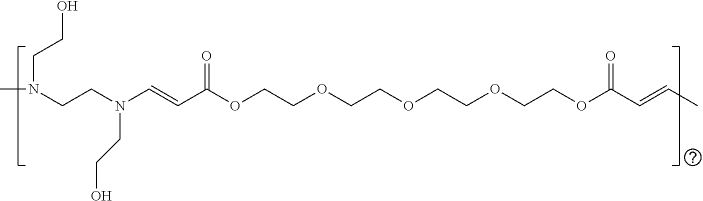 |
| 86 | 7F | 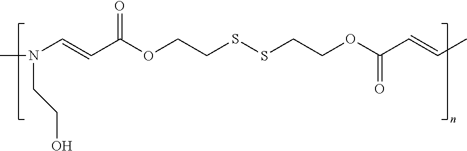 |
| 87 | 7G | 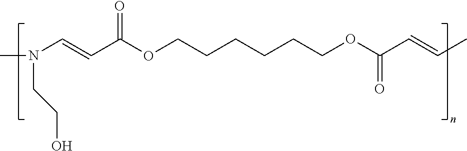 |
| 88 | 7H | 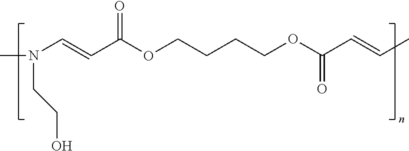 |
| 89 | 7I | 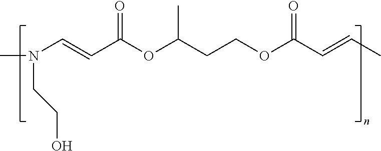 |
| 90 | 7J | 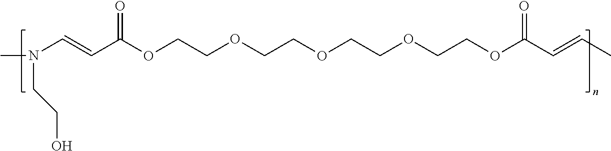 |
| 91 | 8F | 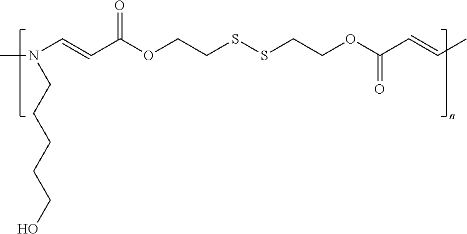 |
| 92 | 8G | 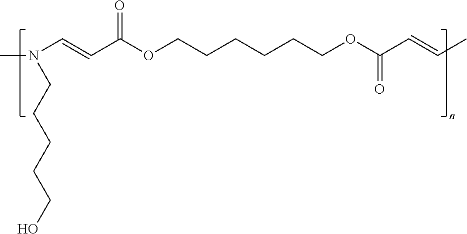 |
| 93 | 8H | 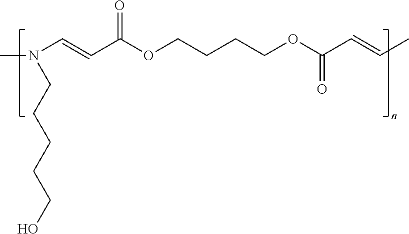 |
| 94 | 8I | 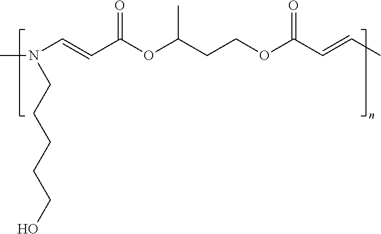 |
| 95 | 8J | 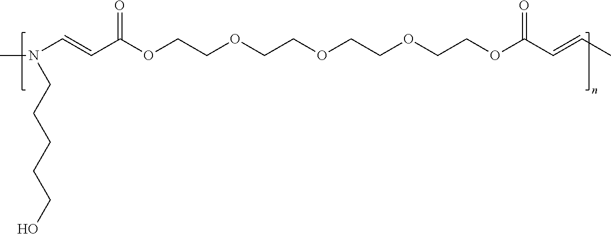 |
| 96 | 9F | 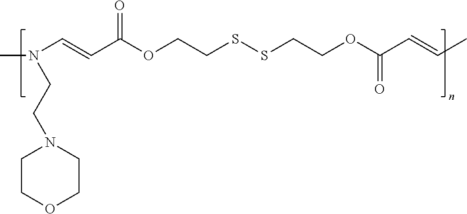 |
| 97 | 9G | 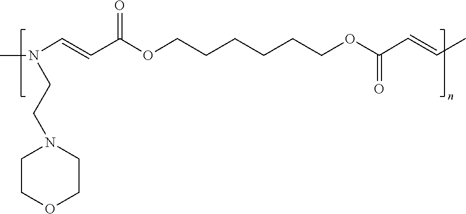 |
| 98 | 9H | 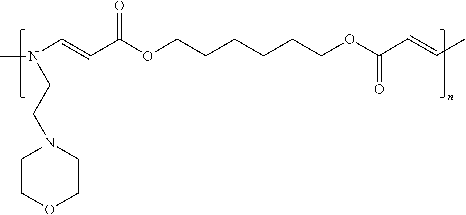 |
| 99 | 9I | 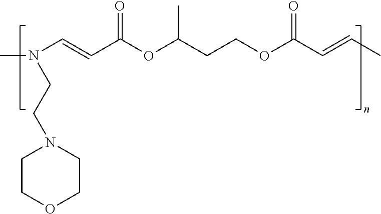 |
| 100 | 9J | 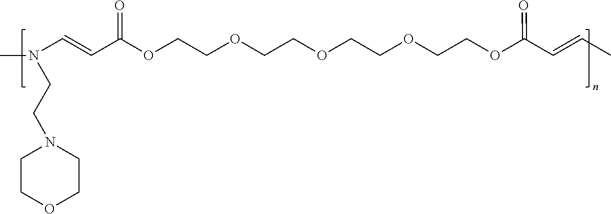 |
| 101 | 10F | 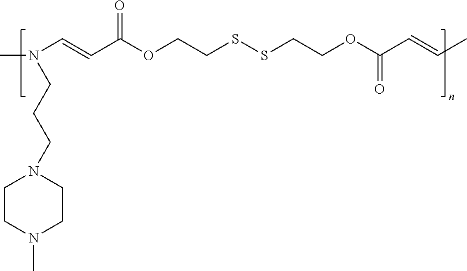 |
| 102 | 10G | 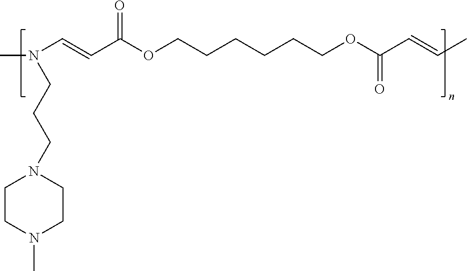 |
| 103 | 10H | 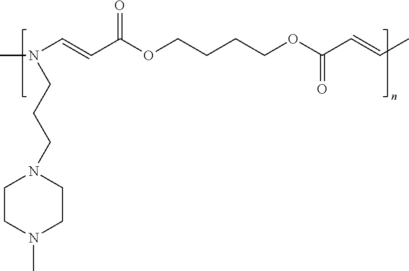 |
| 104 | 10I | 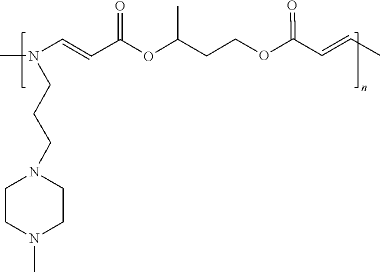 |
| 105 | 10J | 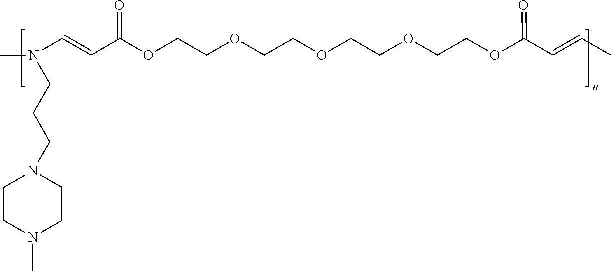 |
| 106 | 11F | 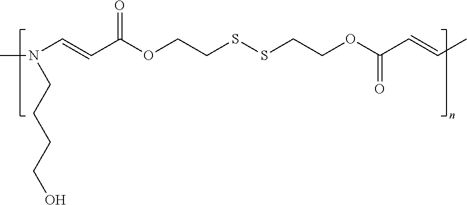 |
| 107 | 11G | 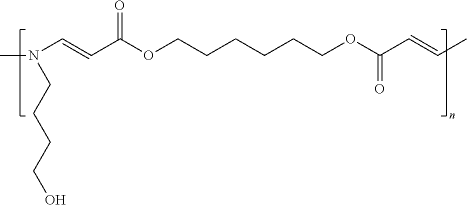 |
| 108 | 11H | 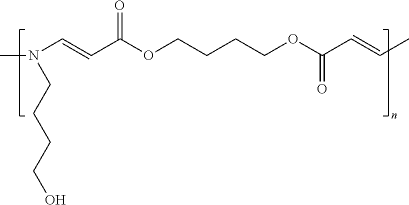 |
| 109 | 11I | 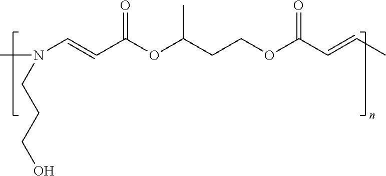 |
| 110 | 11J | 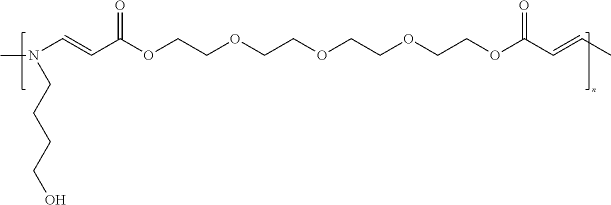 |
| 111 | PEG- 1A | 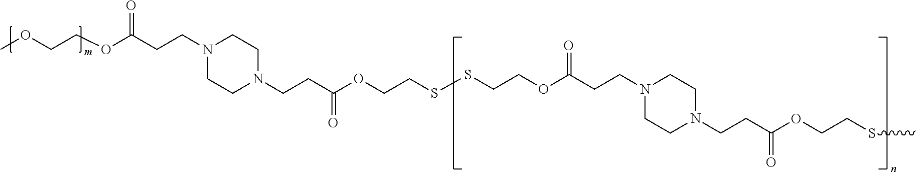 |
| 112 | PEG- 1B | 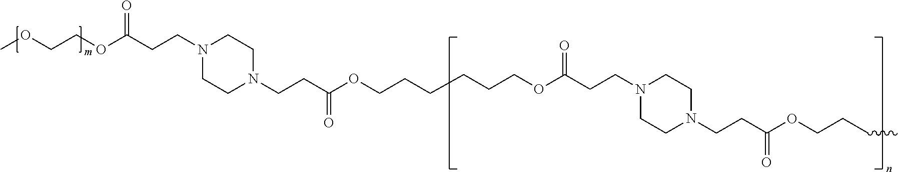 |
| 113 | PEG- 1C | 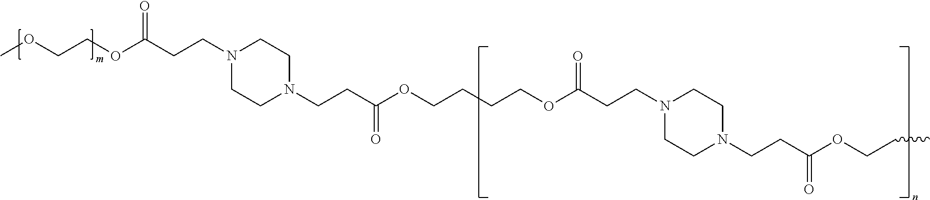 |
| 114 | PEG- 1D | 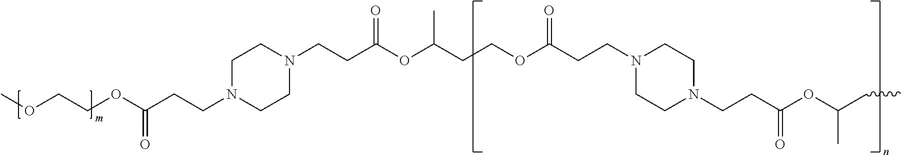 |
| 115 | PEG- 1E | 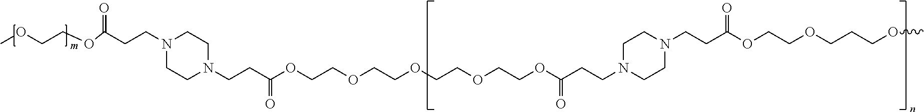 |
| 116 | PEG- 2A | 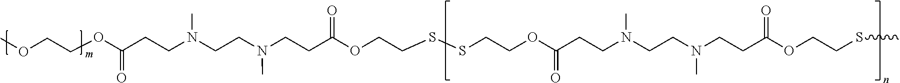 |
| 117 | PEG- 2B | 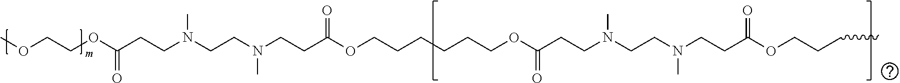 |
| 118 | PEG- 2C | 179 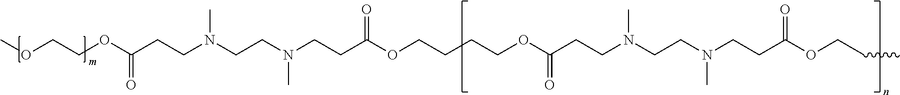 |
| 119 | PEG- 2D | 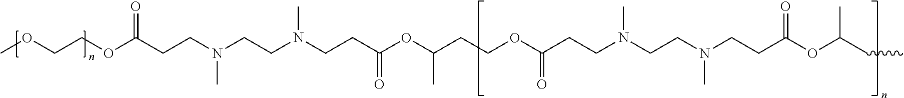 |
| 120 | PEG- 2E | 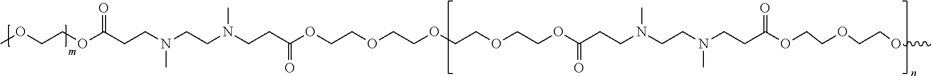 |
| 121 | PEG- 3A | 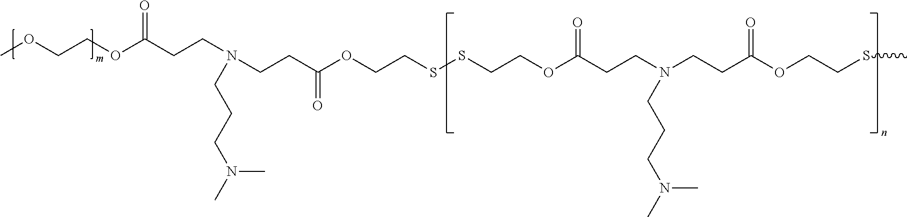 |
| 122 | PEG- 3B | 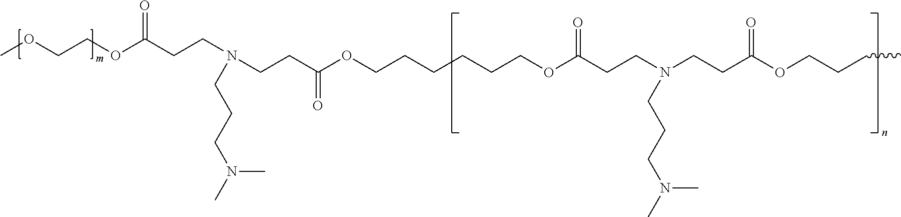 |
| 123 | PEG- 3C | 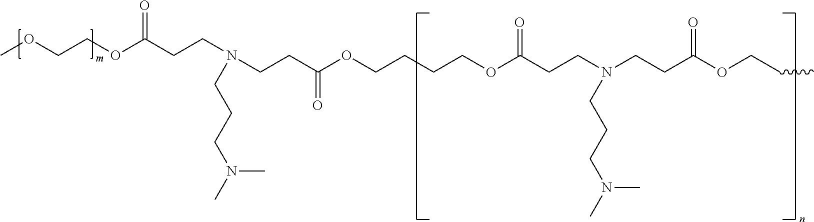 |
| 124 | PEG- 3D | 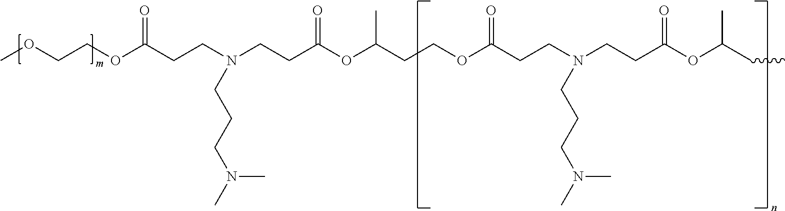 |
| 125 | PEG- 3E | 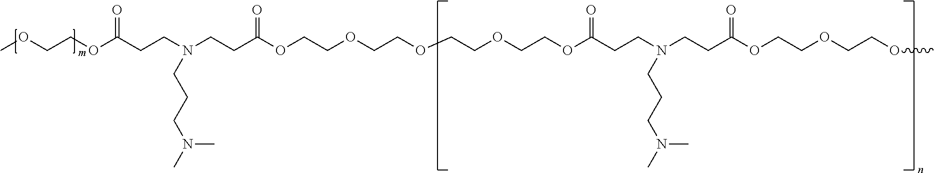 |
| 126 | PEG- 4A | 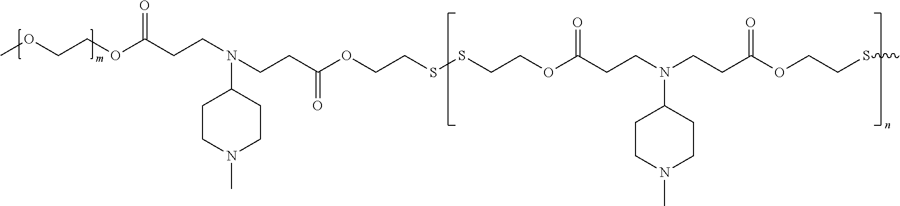 |
| 127 | PEG- 4B | 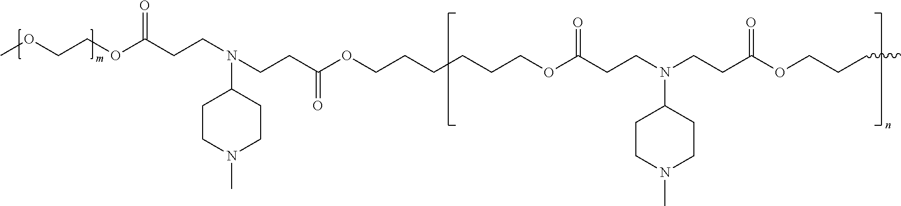 |
| 128 | PEG- 4C | 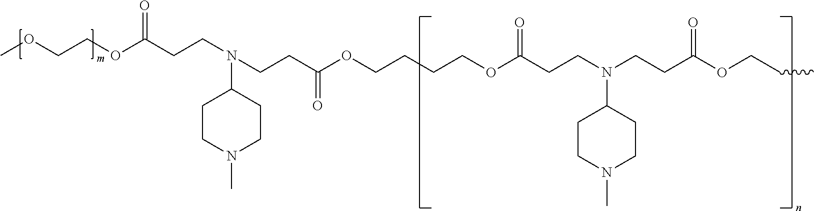 |
| 129 | PEG- 4D | 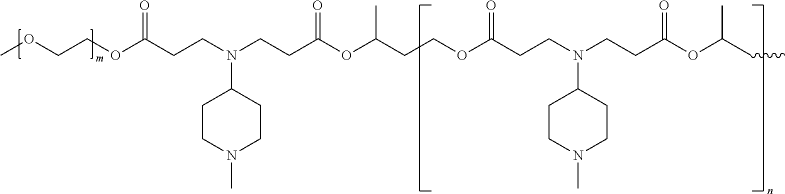 |
| 130 | PEG- 4E | 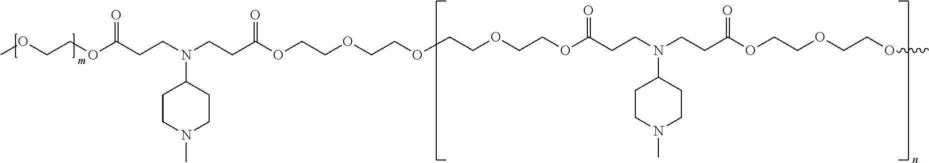 |
| 131 | PEG- 5A | 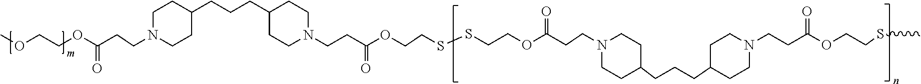 |
| 132 | PEG- 5B | 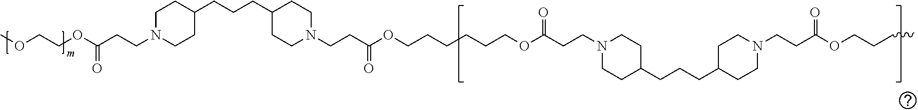 |
| 133 | PEG- 5C | 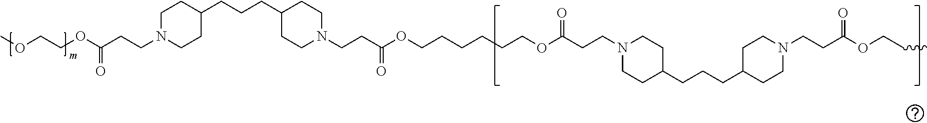 |
| 134 | PEG- 5D | 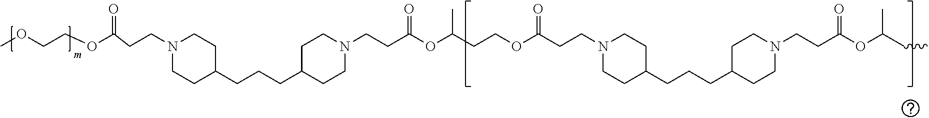 |
| 135 | PEG- 5E | 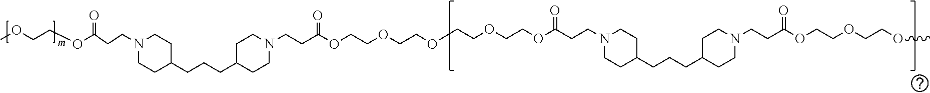 |
| 136 | PEG- 6A | 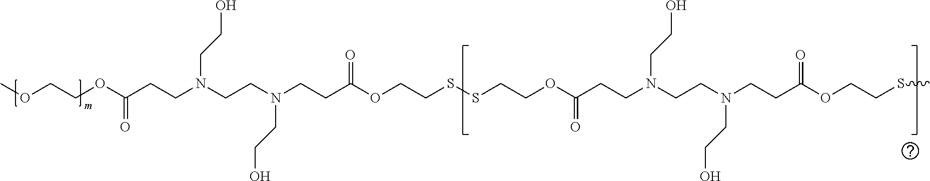 |
| 137 | PEG- 6B | 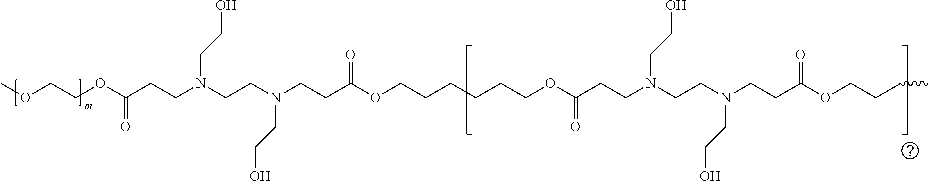 |
| 138 | PEG- 6C | 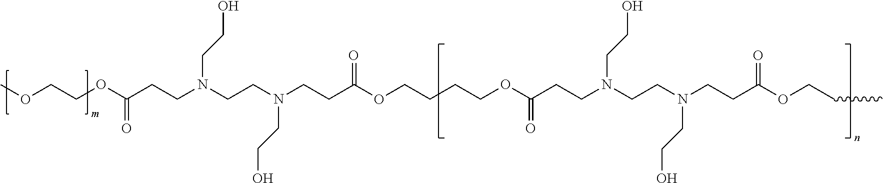 |
| 139 | PEG- 6D | 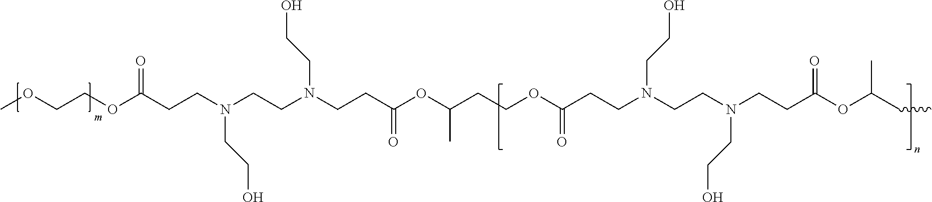 |
| 140 | PEG- 6E | 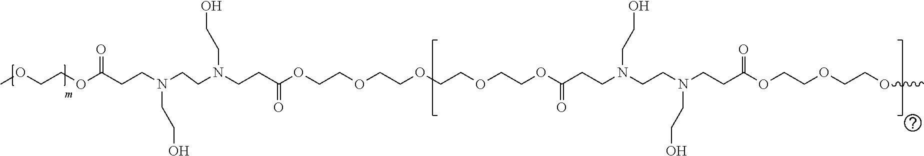 |
| 141 | PEG- 7A | 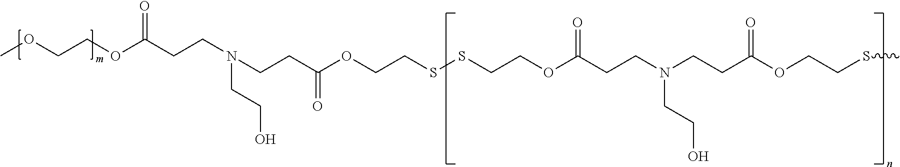 |
| 142 | PEG- 7B | 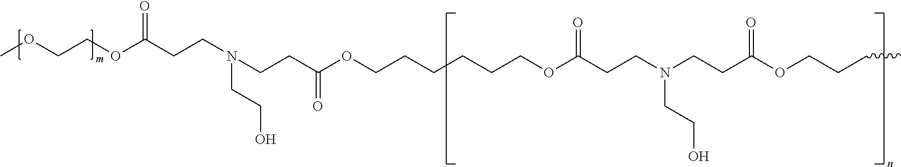 |
| 143 | PEG- 7C | 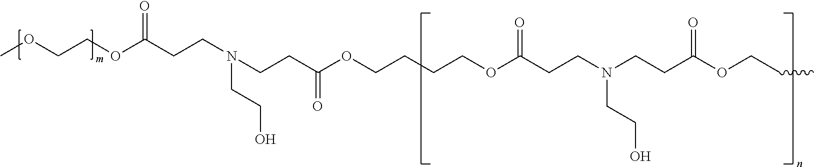 |
| 144 | PEG- 7D | 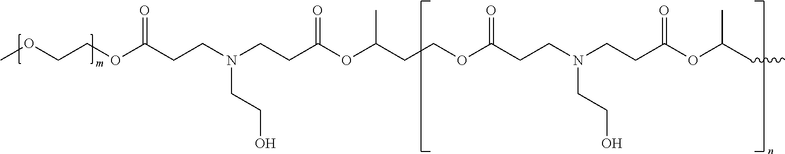 |
| 145 | PEG- 7E | 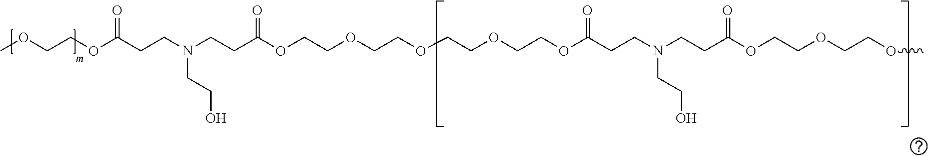 |
| 146 | PEG- 8A | 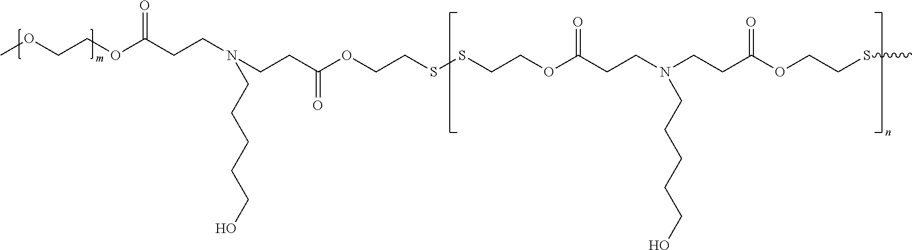 |
| 147 | PEG- 8B | 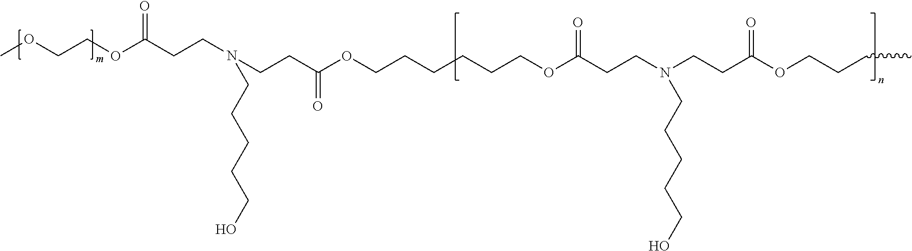 |
| 148 | PEG- 8C | 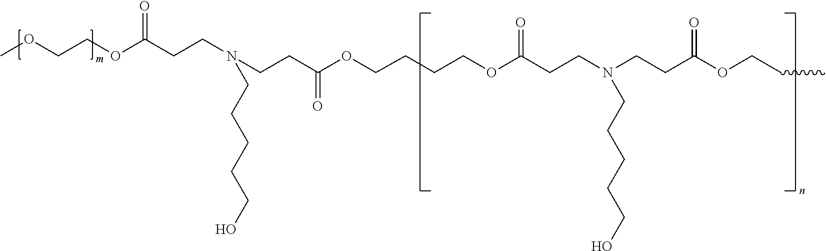 |
| 149 | PEG- 8D | 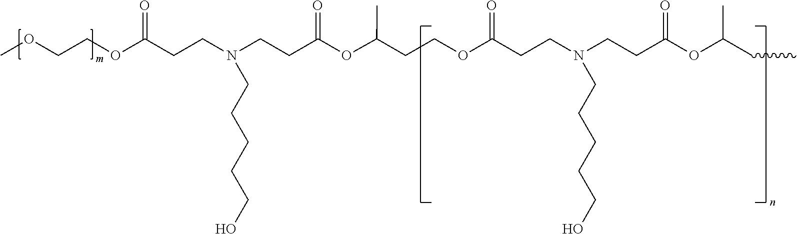 |
| 150 | PEG- 8E | 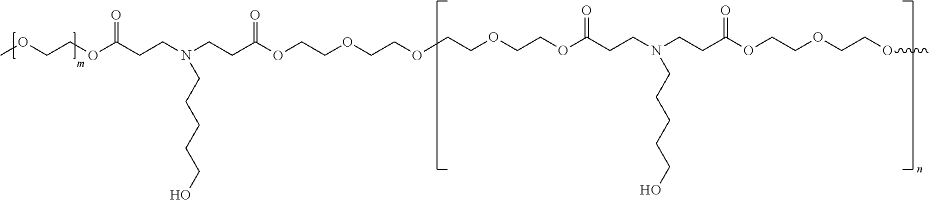 |
| 151 | PEG- 9A | 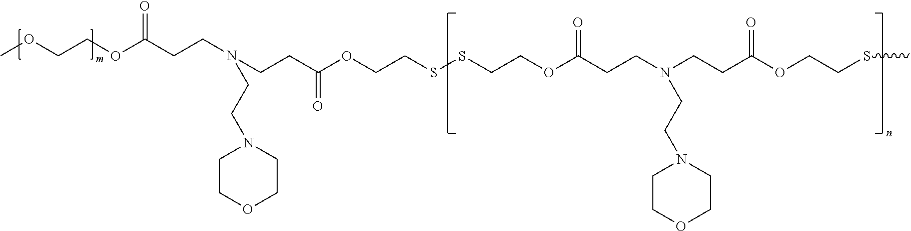 |
| 152 | PEG- 9B | 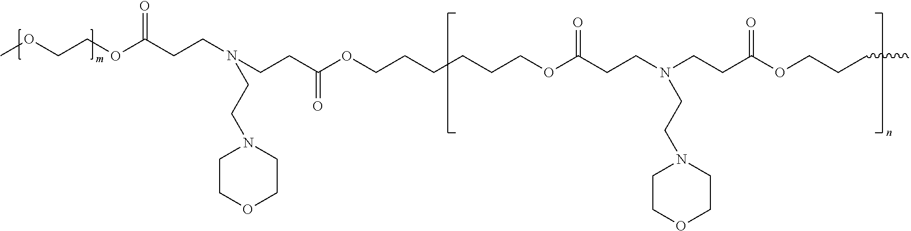 |
| 153 | PEG- 9C | 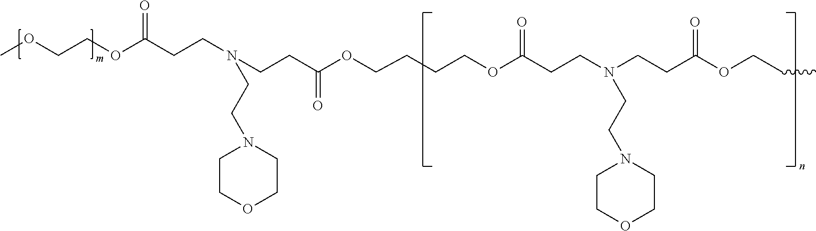 |
| 154 | PEG- 9D | 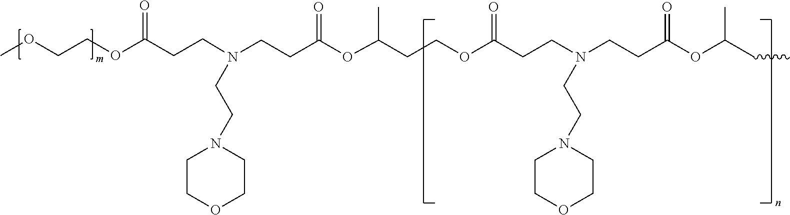 |
| 155 | PEG- 9E | 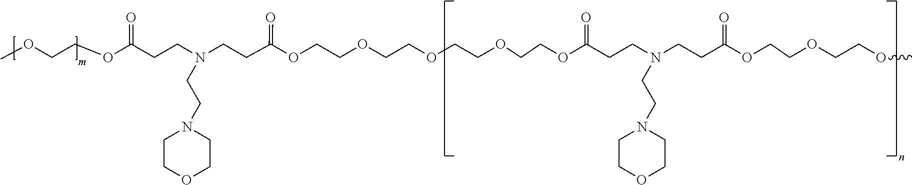 |
| 156 | PEG- 10A | 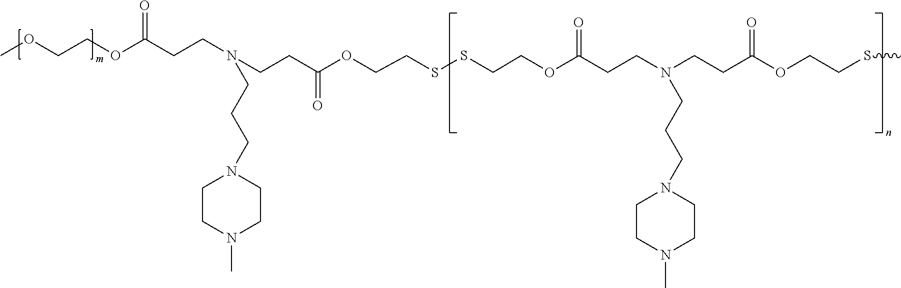 |
| 157 | PEG- 10B | 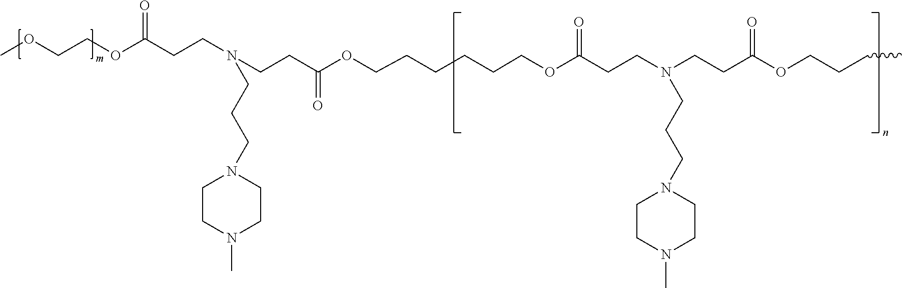 |
| 158 | PEG- 10C | 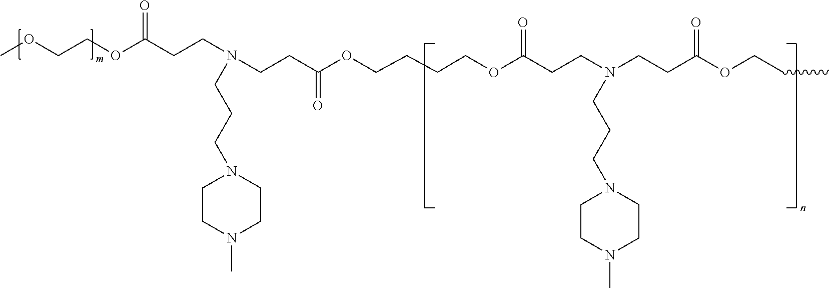 |
| 159 | PEG- 10D | 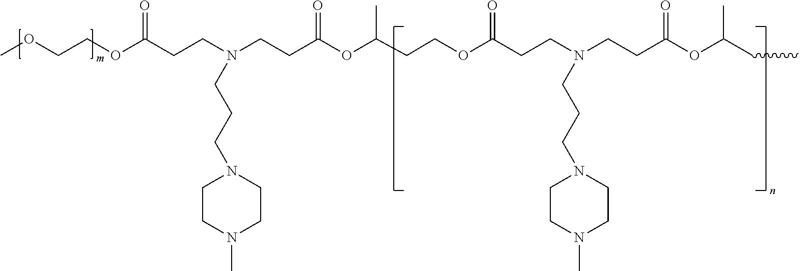 |
| 160 | PEG- 10E | 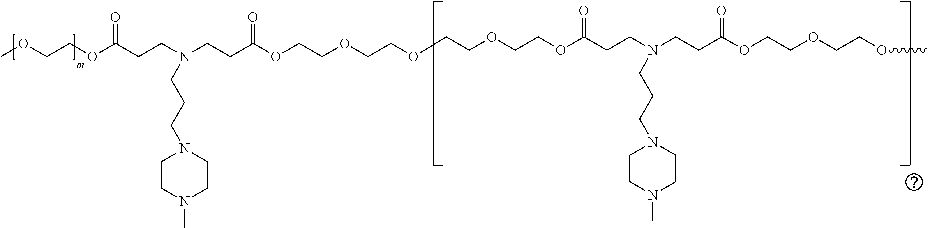 |
| 161 | PEG- 11A | 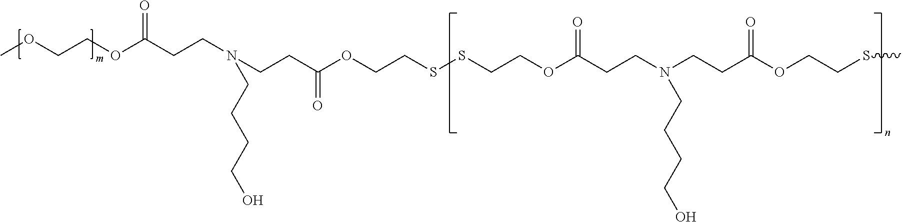 |
| 162 | PEG- 11B | 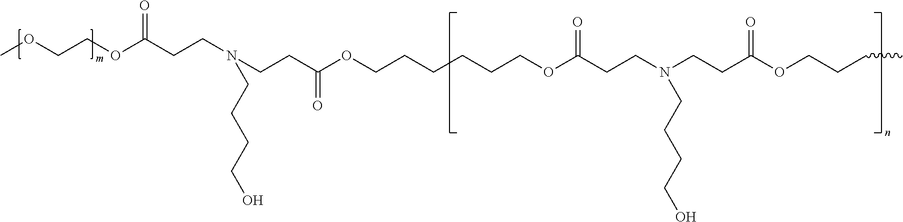 |
| 163 | PEG- 11C | 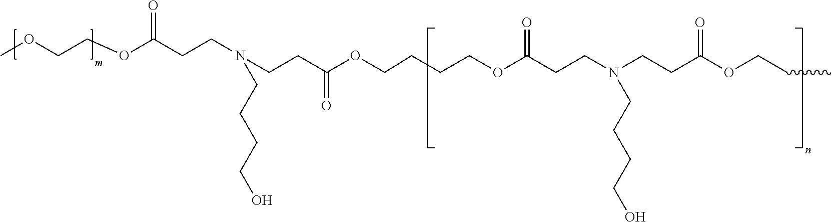 |
| 164 | PEG- 11D | 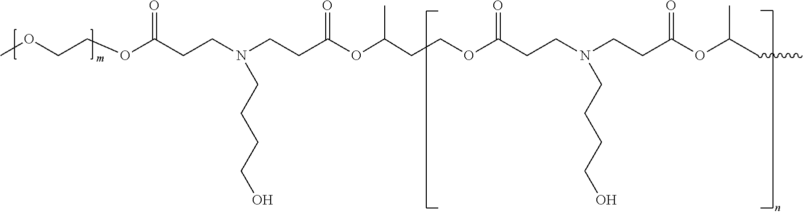 |
| 165 | PEG- 11E | 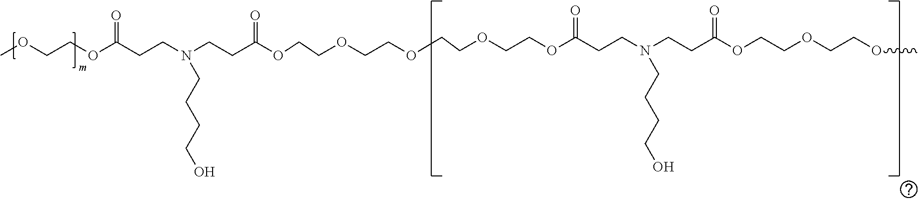 |
| 166 | PEG- 1F | 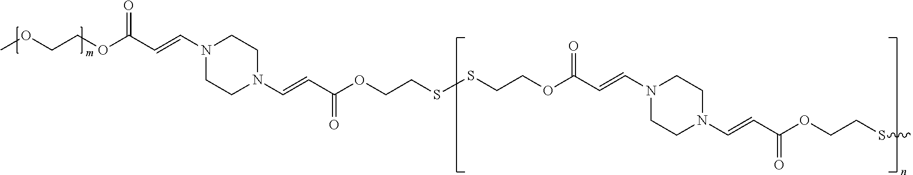 |
| 167 | PEG- 1G | 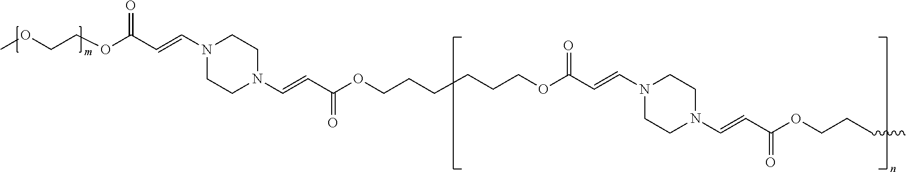 |
| 168 | PEG- 1H | 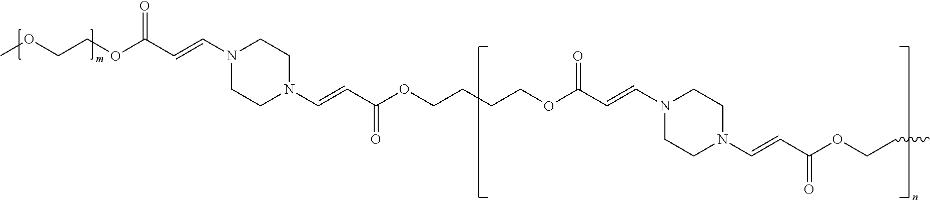 |
| 169 | PEG- 1I | 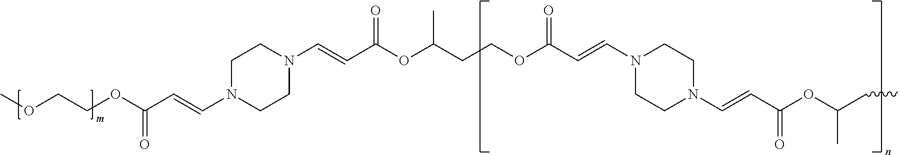 |
| 170 | PEG- 1J | 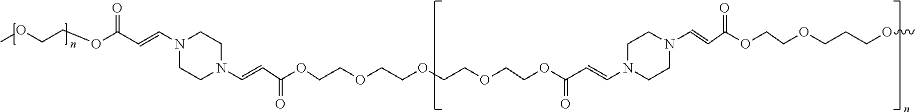 |
| 171 | PEG- 2F | 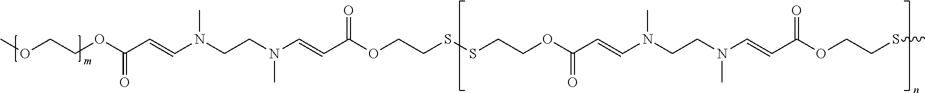 |
| 172 | PEG- 2G | 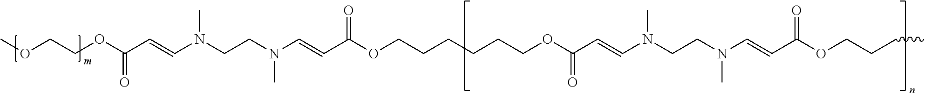 |
| 173 | PEG- 2H | 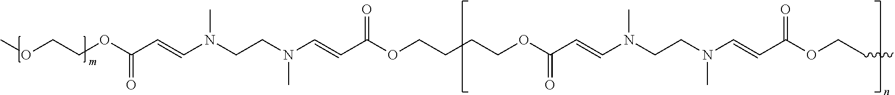 |
| 174 | PEG- 2I | 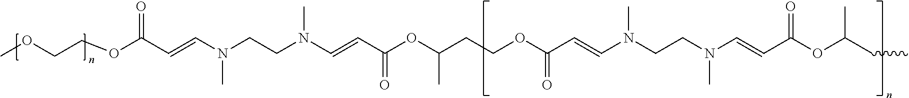 |
| 175 | PEG- 2J | 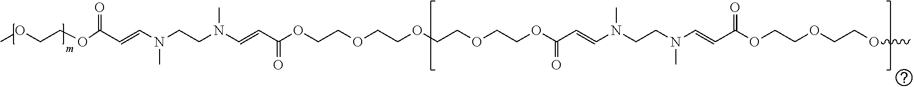 |
| 176 | PEG- 3F | 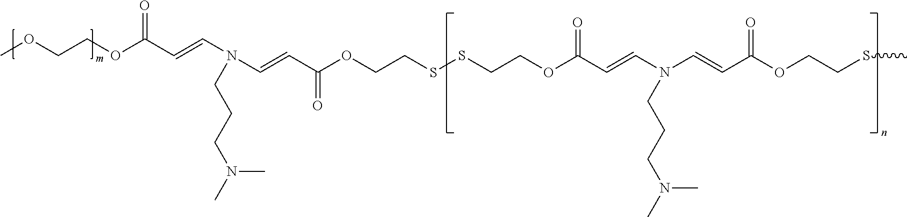 |
| 177 | PEG- 3G | 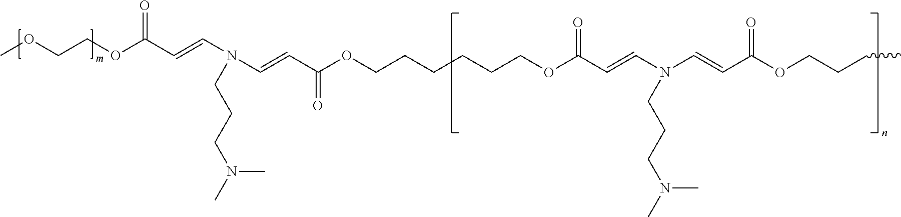 |
| 178 | PEG- 3H | 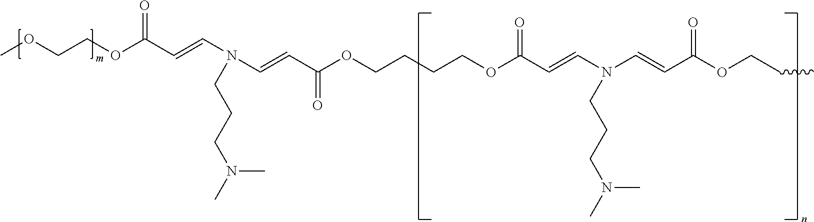 |
| 179 | PEG- 3I | 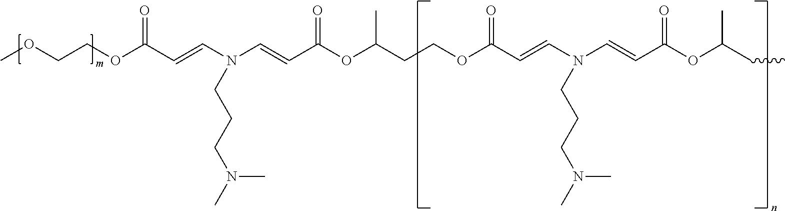 |
| 180 | PEG- 3J | 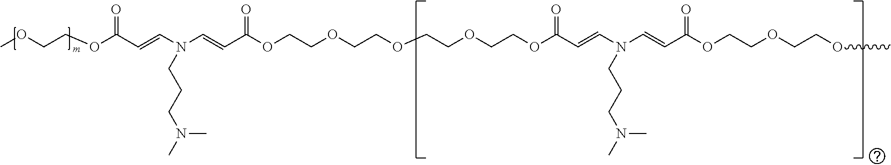 |
| 181 | PEG- 4F | 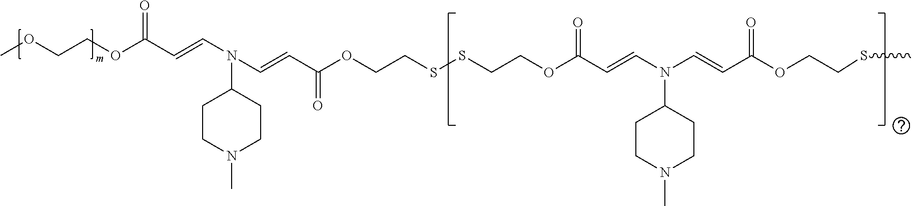 |
| 182 | PEG- 4G | 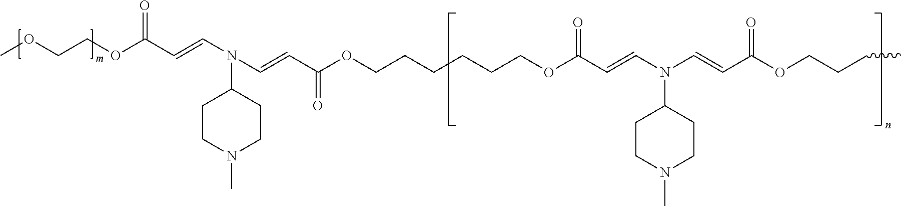 |
| 183 | PEG- 4H | 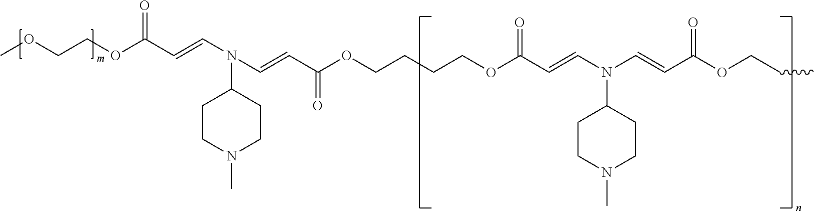 |
| 184 | PEG- 4I | 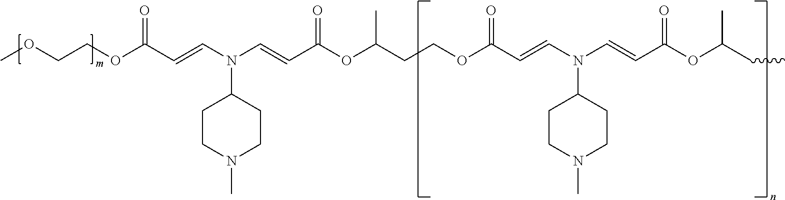 |
| 185 | PEG- 4J | 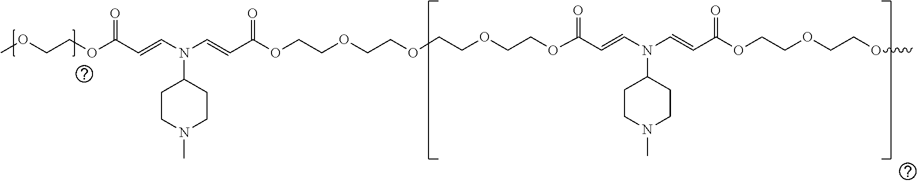 |
| 186 | PEG- 5F | 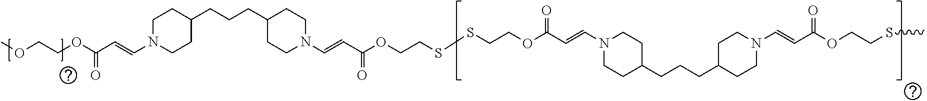 |
| 187 | PEG- 5G | 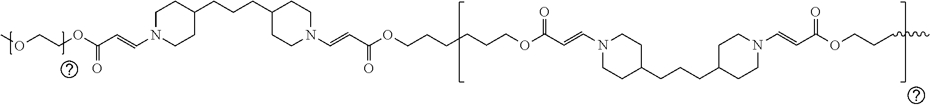 |
| 188 | PEG- 5H | 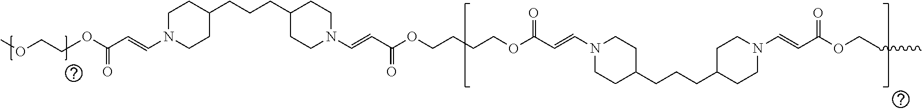 |
| 189 | PEG- 5I | 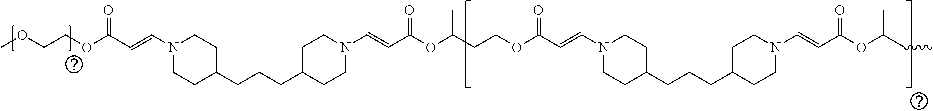 |
| 190 | PEG- 5J | 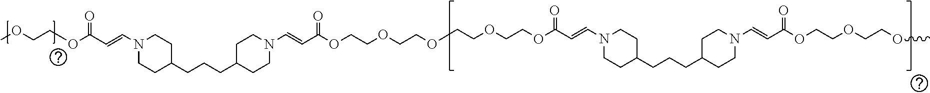 |
| 191 | PEG- 6F | 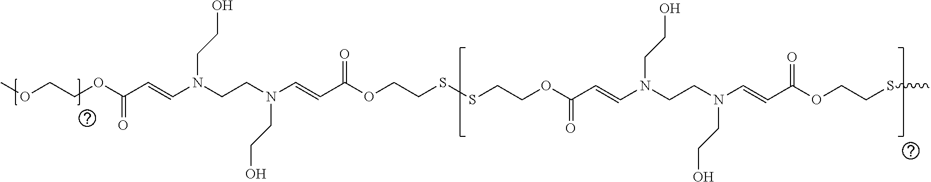 |
| 192 | PEG- 6G | 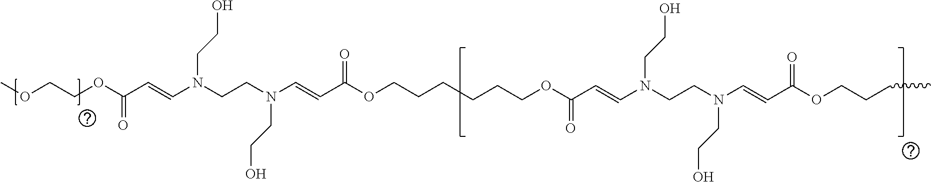 |
| 193 | PEG- 6H | 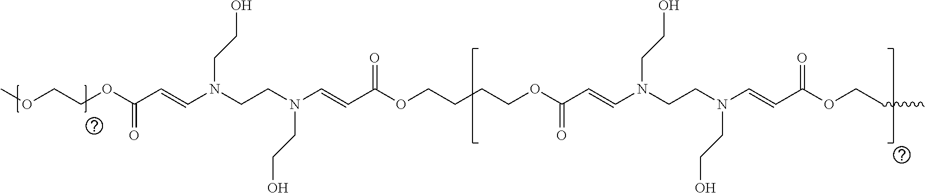 |
| 194 | PEG- 6I | 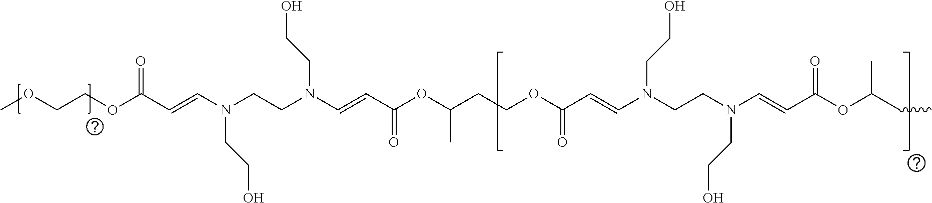 |
| 195 | PEG- 6J | 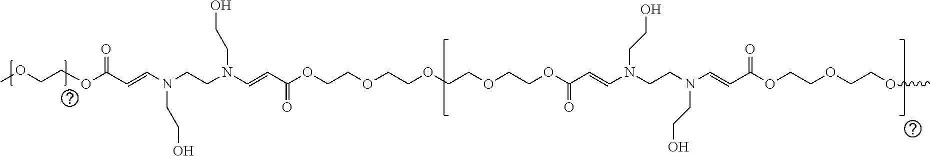 |
| 196 | PEG- 7F | 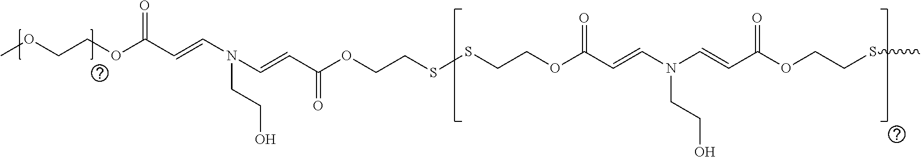 |
| 197 | PEG- 7G | 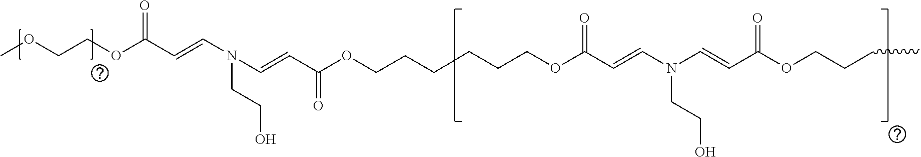 |
| 198 | PEG- 7H | 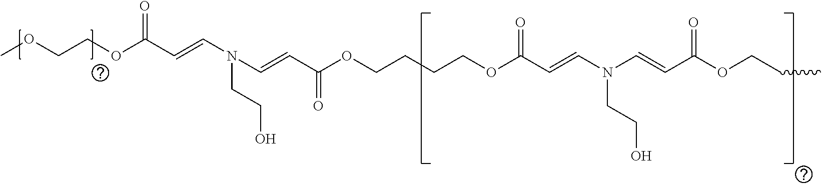 |
| 199 | PEG- 7I | 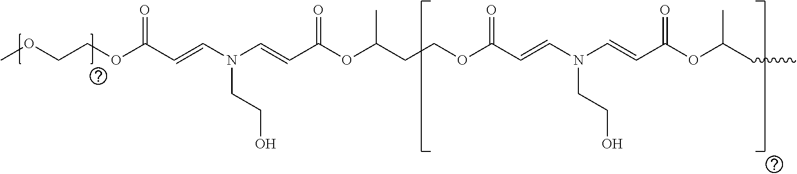 |
| 200 | PEG- 7J | 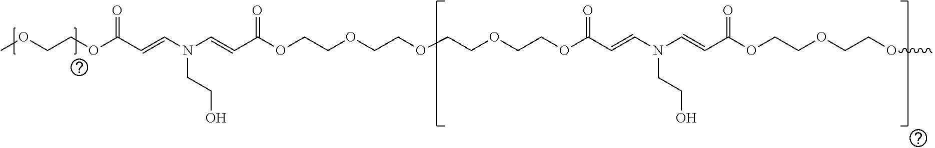 |
| 201 | PEG- 8F | 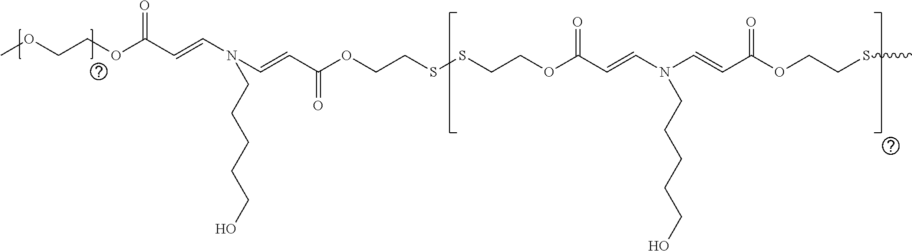 |
| 202 | PEG- 8G | 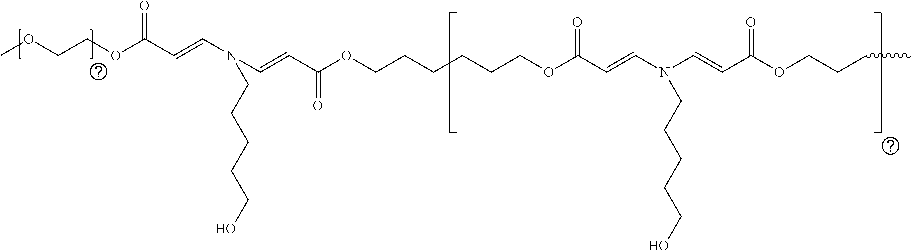 |
| 203 | PEG- 8H | 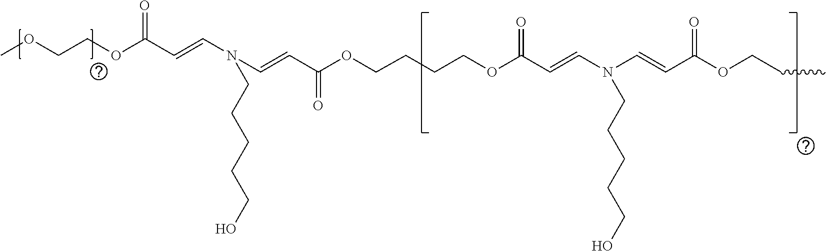 |
| 204 | PEG- 8I | 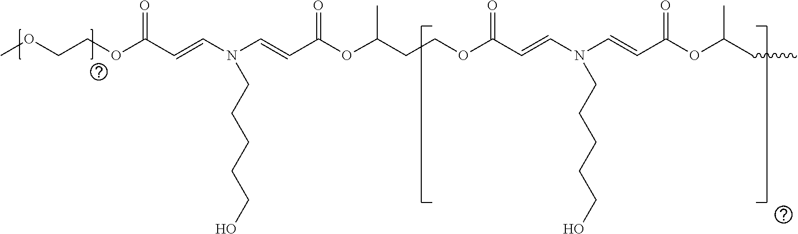 |
| 205 | PEG- 8J | 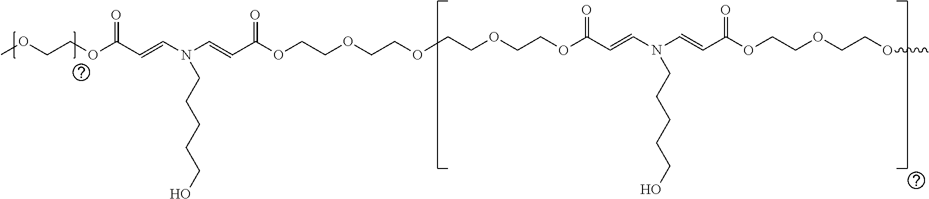 |
| 206 | PEG- 9F | 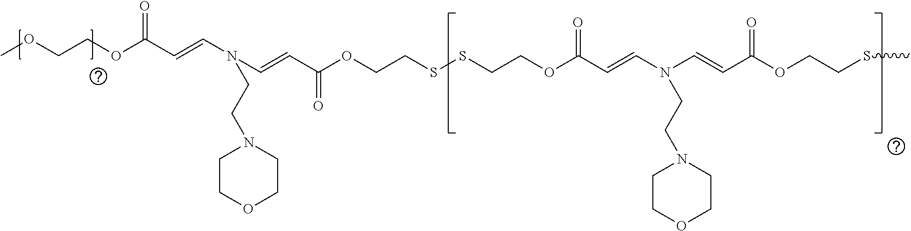 |
| 207 | PEG- 9G | 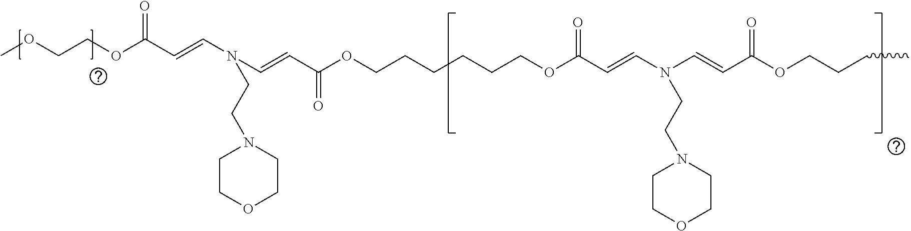 |
| 208 | PEG- 9H | 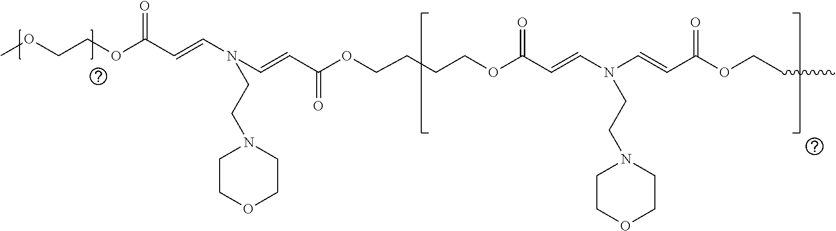 |
| 209 | PEG- 9I | 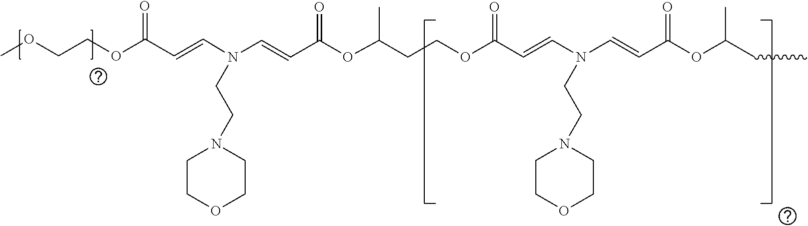 |
| 210 | PEG- 9J | 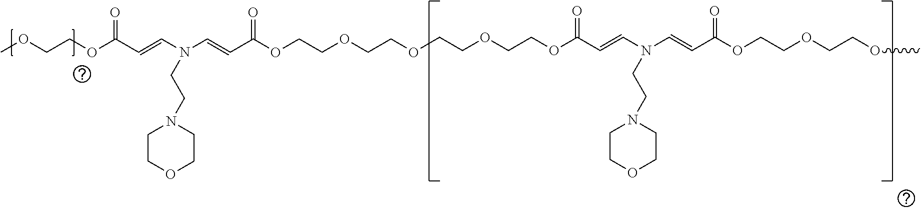 |
| 211 | PEG- 10F | 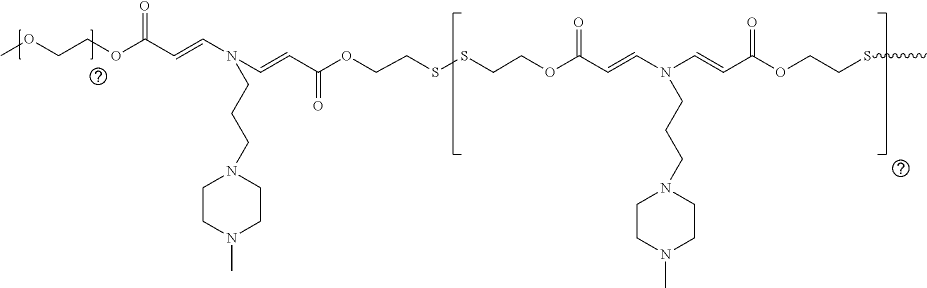 |
| 212 | PEG- 10G | 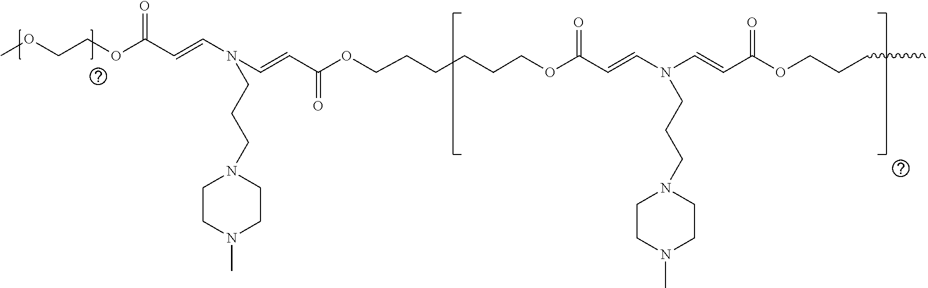 |
| 213 | PEG- 10H | 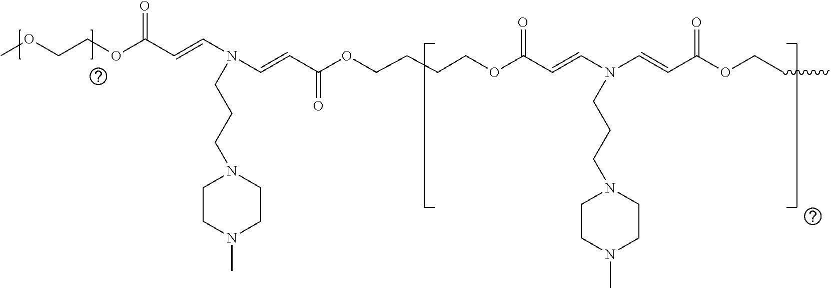 |
| 214 | PEG- 10I | 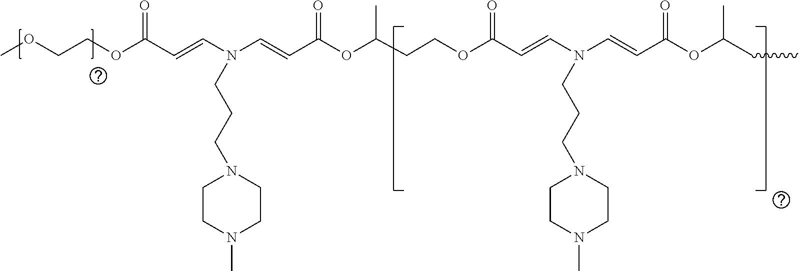 |
| 215 | PEG- 10J | 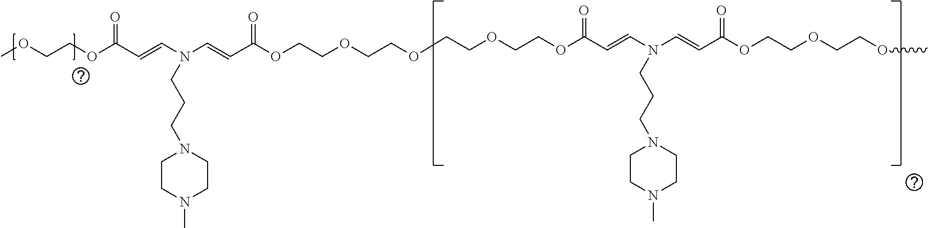 |
| 216 | PEG- 11F | 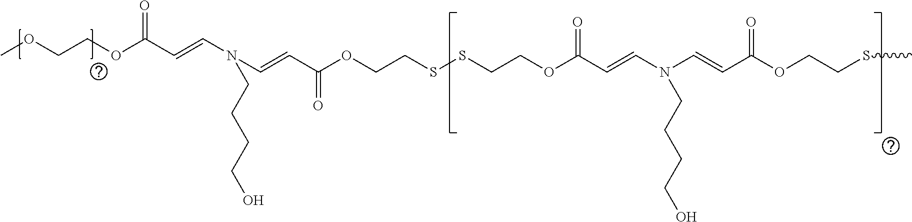 |
| 217 | PEG- 11G | 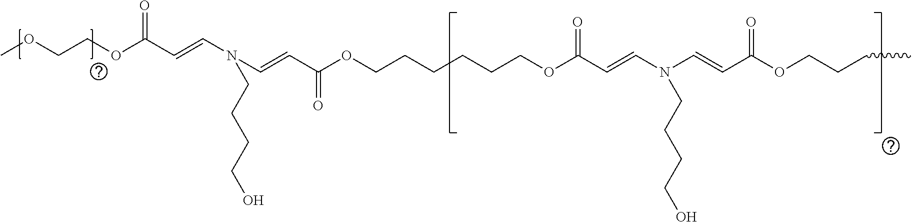 |
| 218 | PEG- 11H | 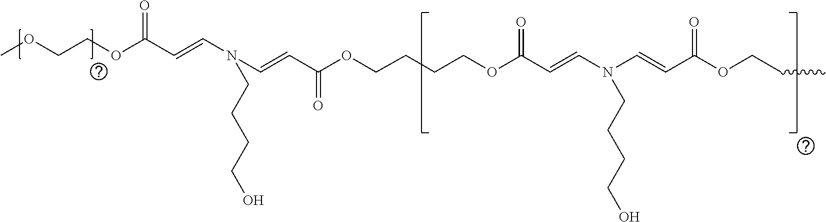 |
| 219 | PEG- 11I | 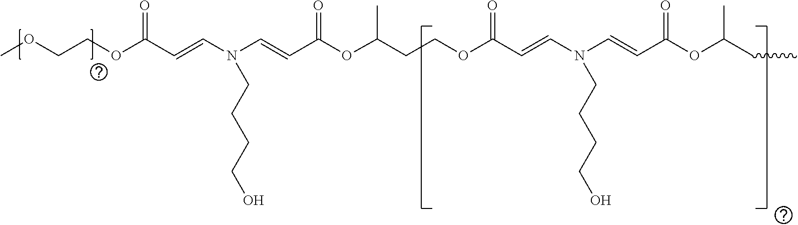 |
| 220 | PEG- 11J | 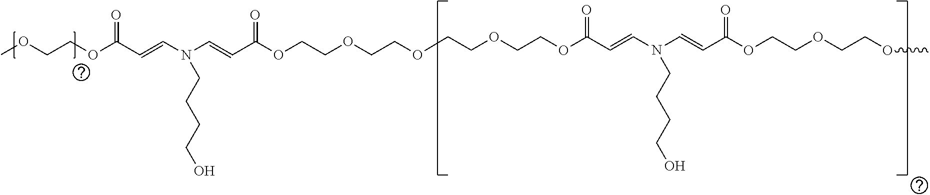 |
| 221 | b3A | 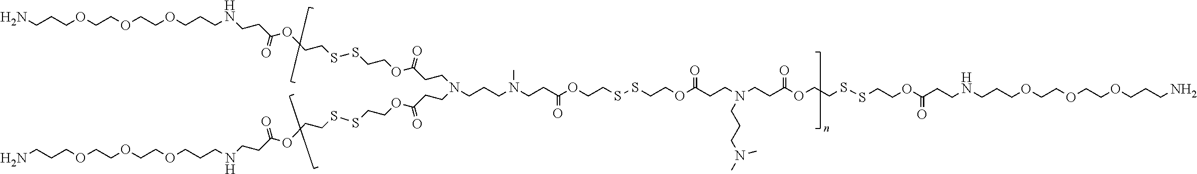 |
| 222 | b3B | 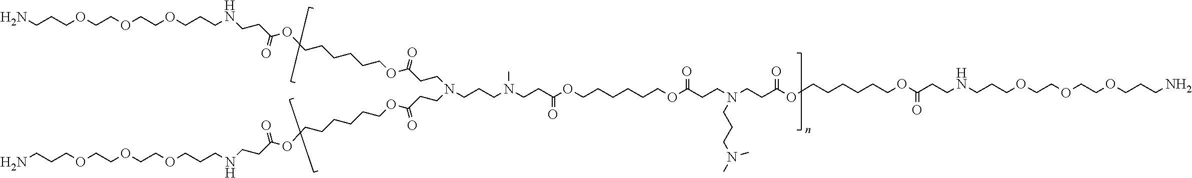 |
| 223 | b3C | 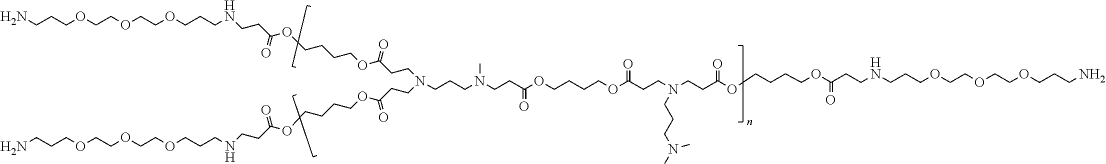 |
| 224 | b3D | 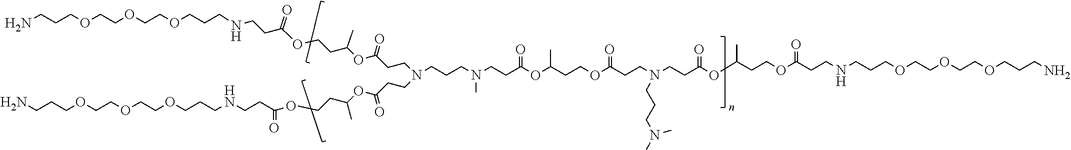 |
| 225 | b3E | 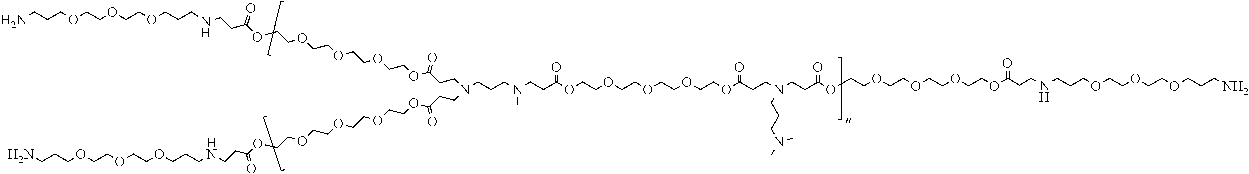 |
| 226 | b4A | 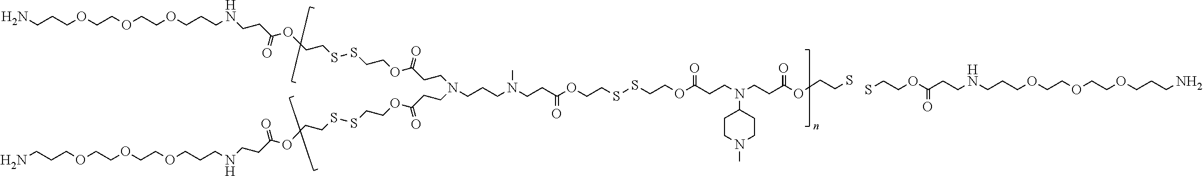 |
| 227 | b4B | 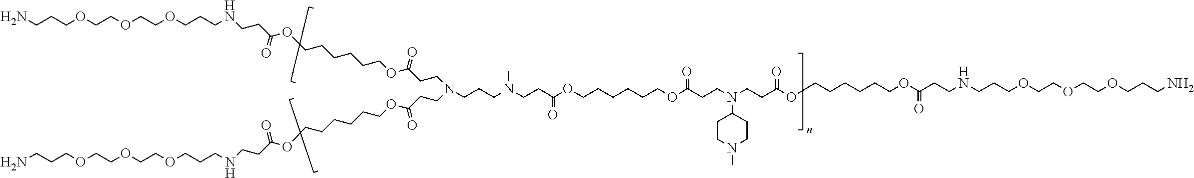 |
| 228 | b4C | 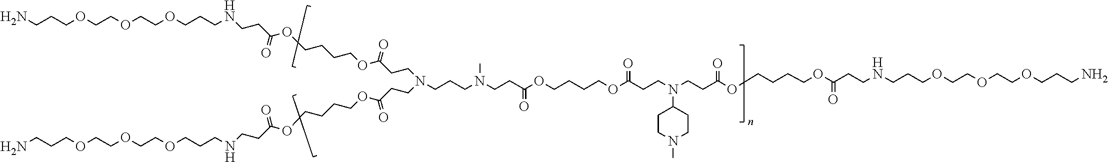 |
| 229 | b4D | 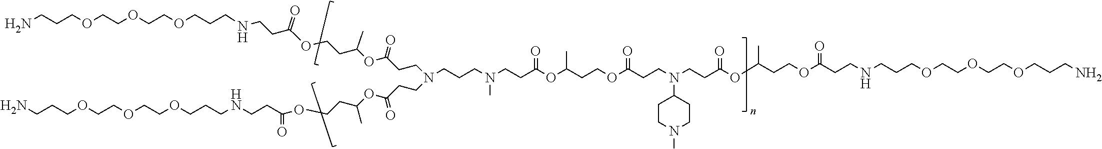 |
| 230 | b4E | 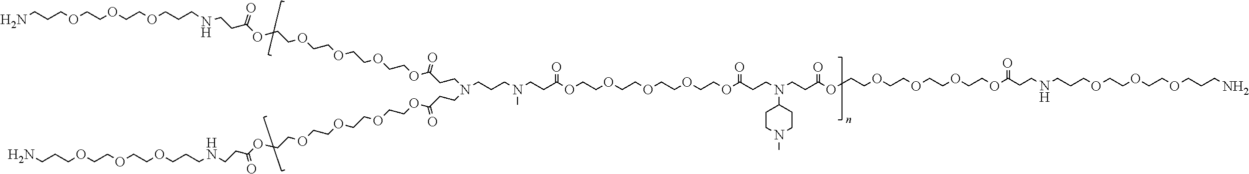 |
| 231 | b7A | 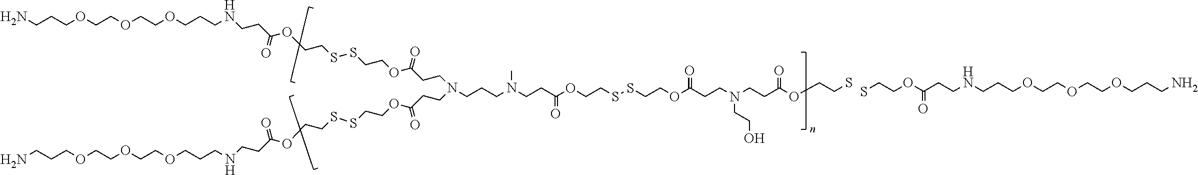 |
| 232 | b7B | 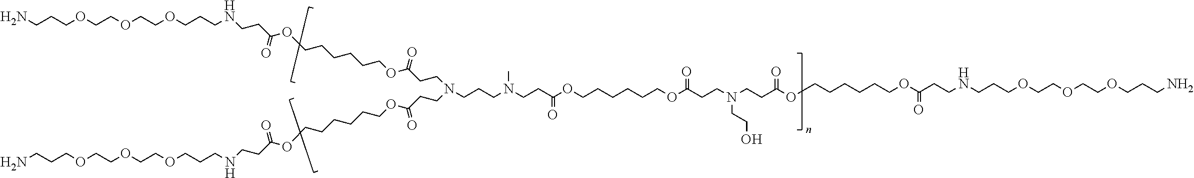 |
| 233 | b7C | 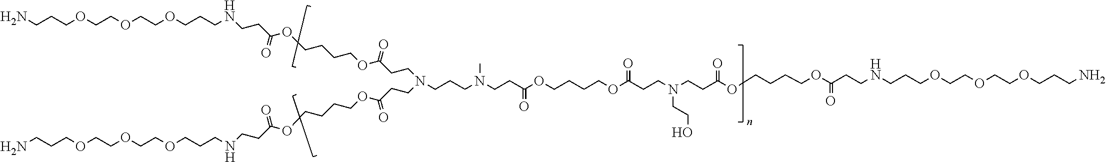 |
| 234 | b7D | 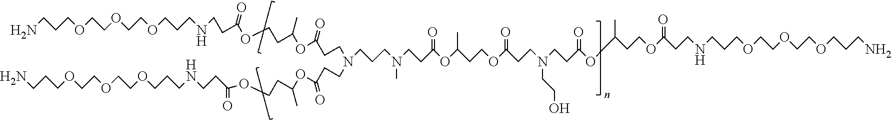 |
| 235 | b7E | 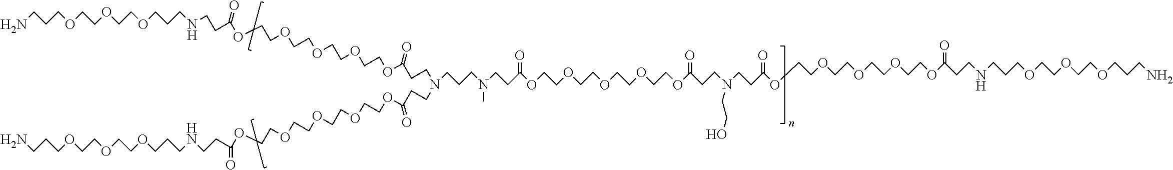 |
| 236 | b8A | 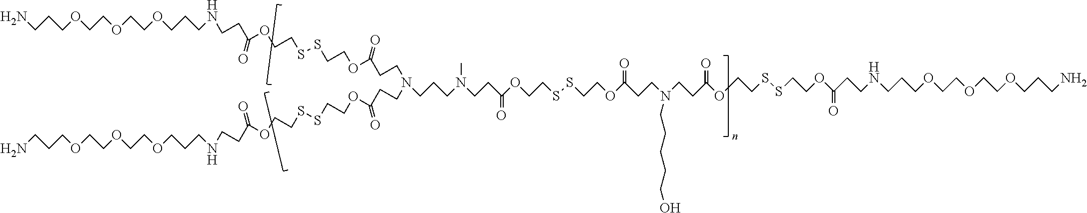 |
| 237 | b8B | 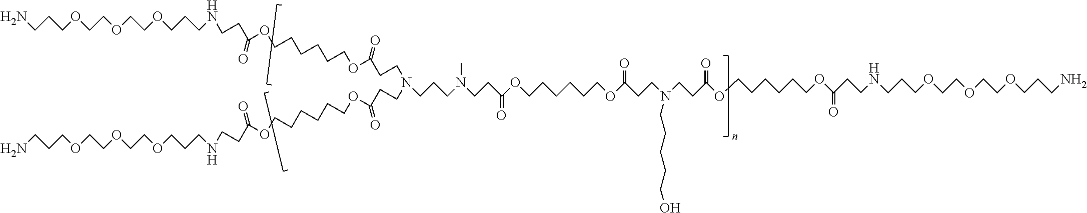 |
| 238 | b8C | 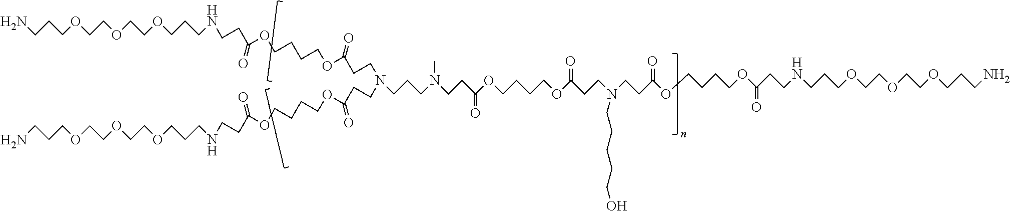 |
| 239 | b8D | 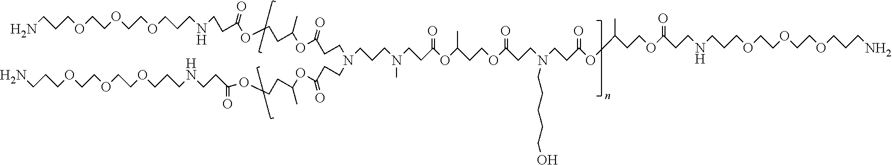 |
| 240 | b8E | 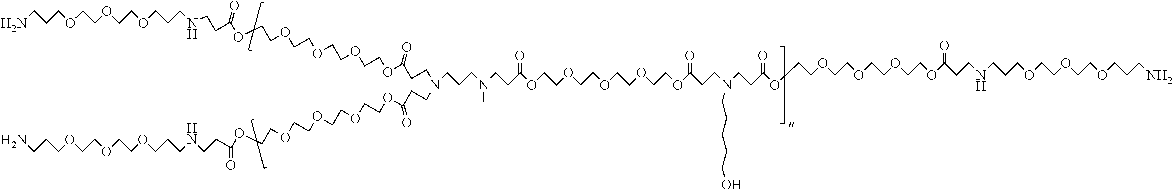 |
| 241 | b9A | 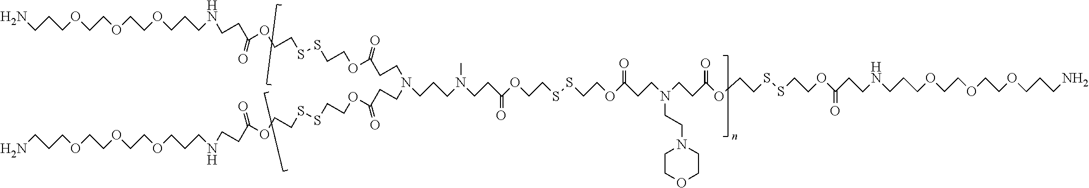 |
| 242 | b9B | 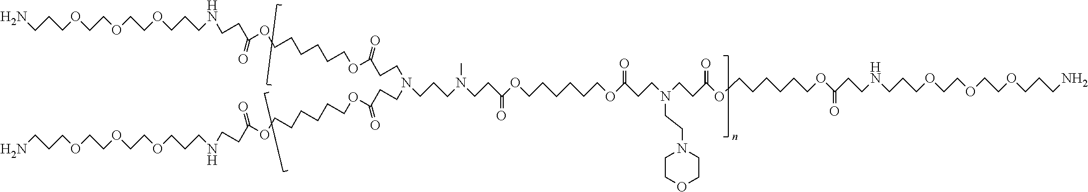 |
| 243 | b9C | 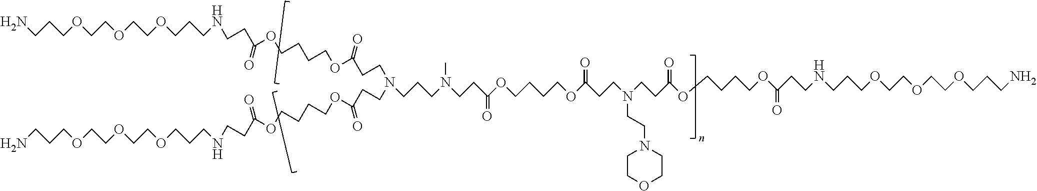 |
| 244 | b9D | 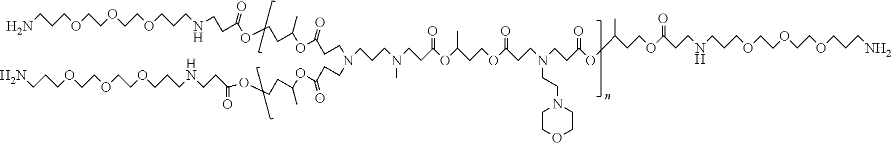 |
| 245 | b9E | 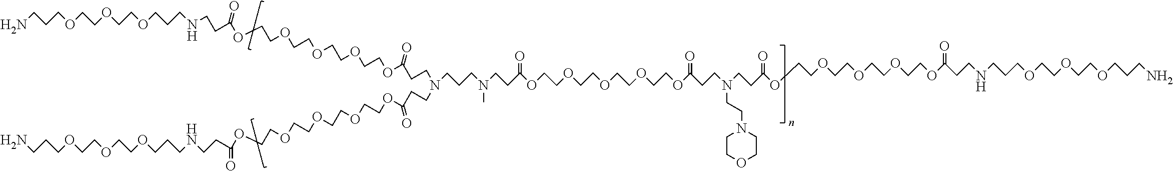 |
| 246 | b10A | 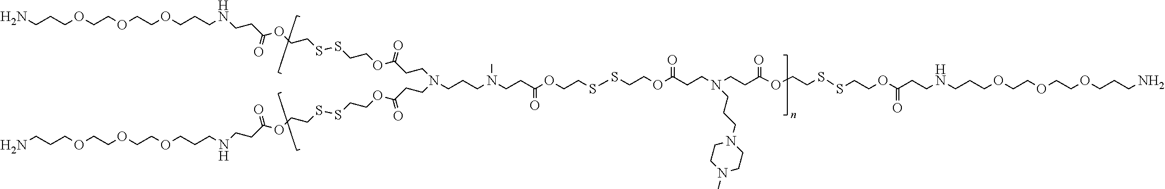 |
| 247 | b10B | 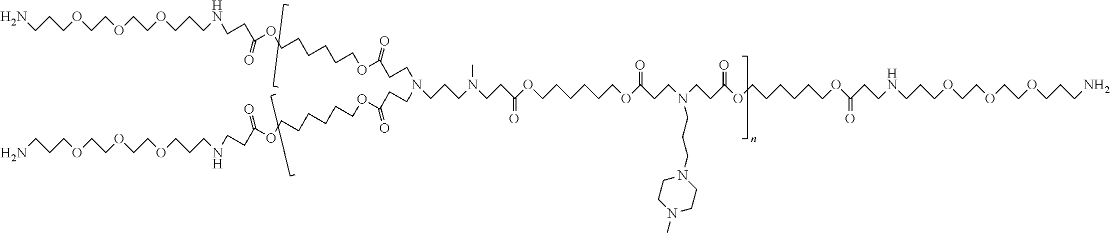 |
| 248 | b10C | 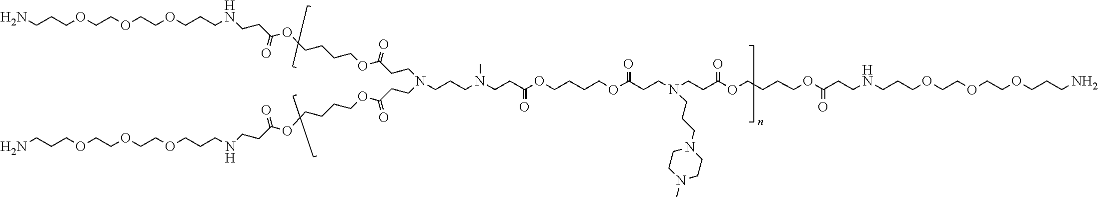 |
| 249 | b10D | 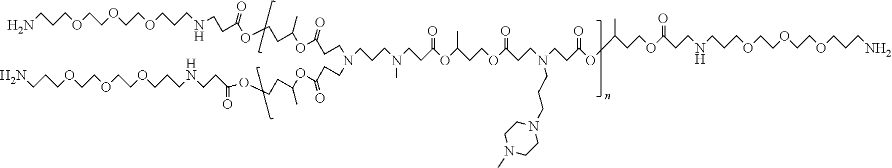 |
| 250 | b10E | 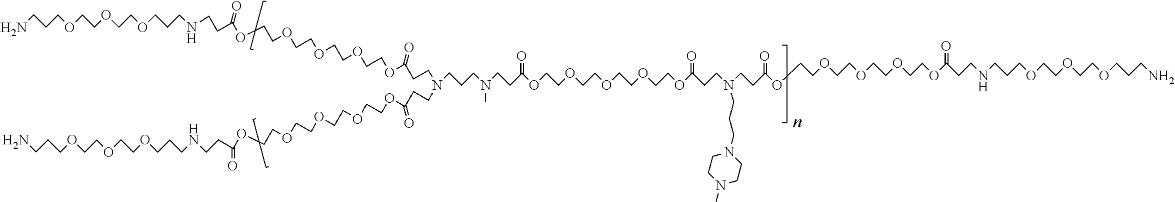 |
| 251 | b11A | 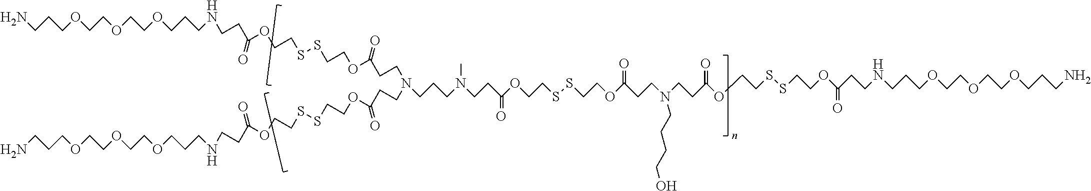 |
| 252 | b11B | 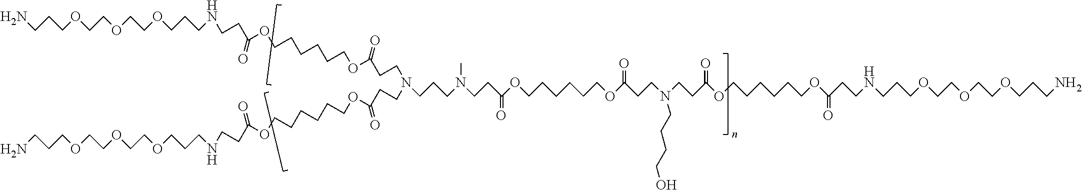 |
| 253 | b11C | 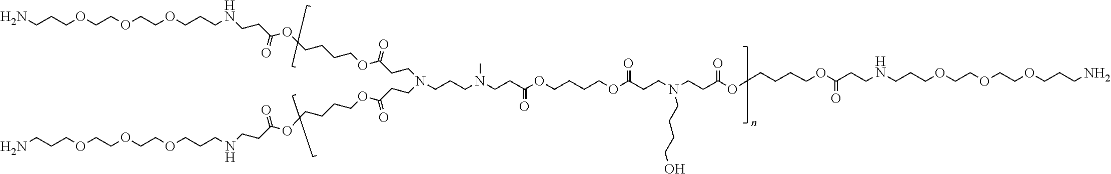 |
| 254 | b11D | 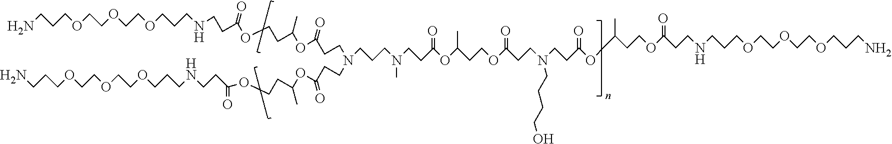 |
| 255 | b11E | 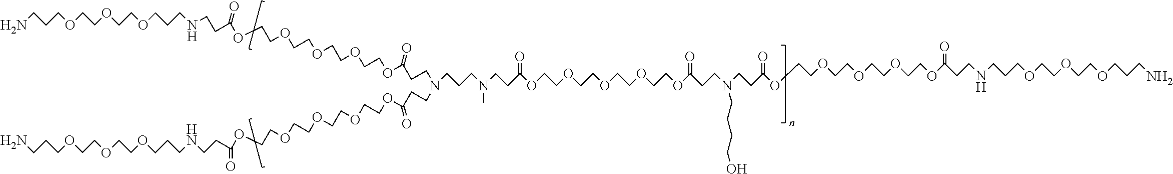 |
| 256 | b12A | 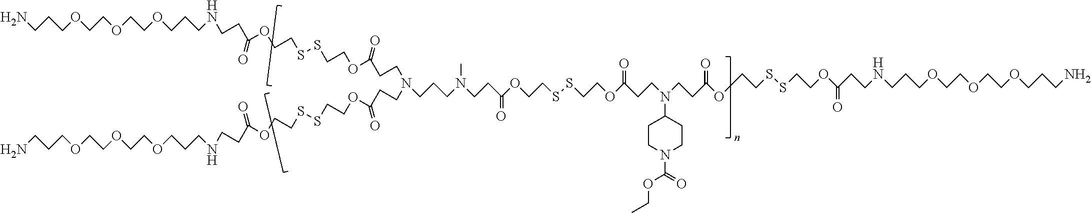 |
| 257 | b12B | 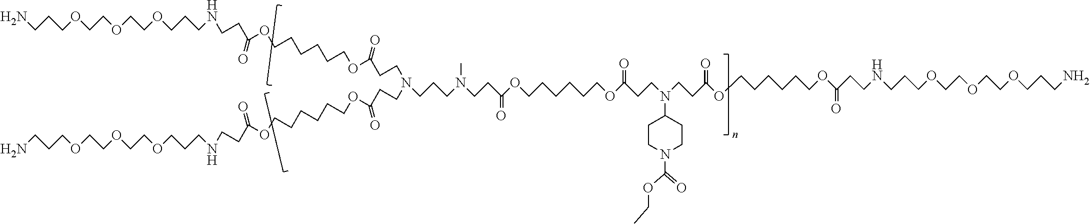 |
| 258 | b12C | 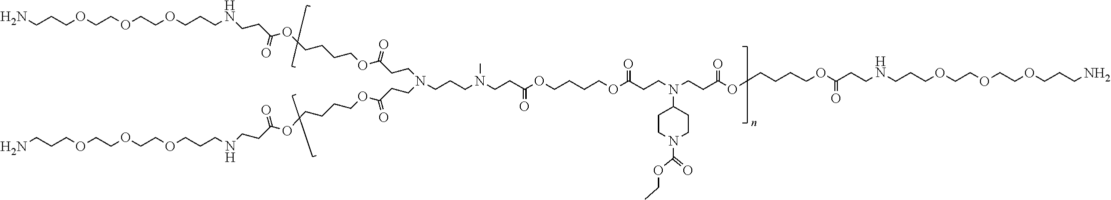 |
| 259 | b12D | 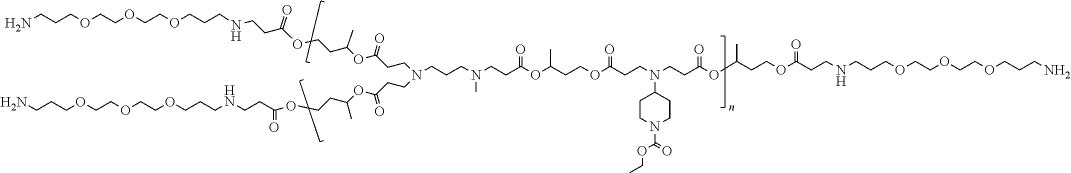 |
| 260 | b12E | 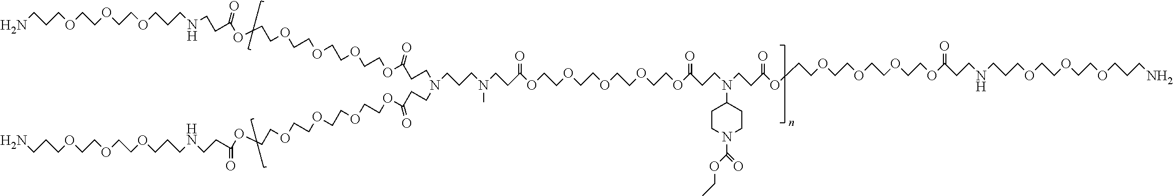 |

(in the above formulas, n is an integer between 1 and 500)

In one embodiment, the polymer may further comprise a branched monomer of Formula 25 below.

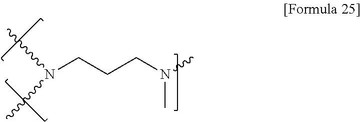

When the polymer according to the present invention comprises a structure of Formula 25 above, the polymer may comprise a structure of Formula 26 below.

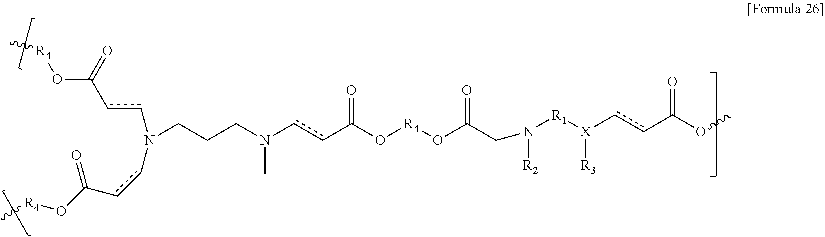

in the formula,
R1 is a bond or a C1-6 alkylene,
R2 and R3 are each independently (i) hydrogen, (ii) C1-6 alkyl which may be substituted with alkylamine or hydroxy, (iii) a 5- or 6-membered carbocycle or heterocycle, or (iv) C1-6 alkyl substituted with a 5- or 6-membered carbocycle or heterocycle,
said R2 and R3 may be linked together to form a C1-6 alkylene,
said 5- or 6-membered carbocycle or heterocycle may be substituted with C1-3 alkyl or C1-3 alkoxyacyl,
X is a nitrogen atom or a bond, and when X is a bond, said R3 is absent,
R4 is a linear or branched C1-15 alkylene, 1 to 5 carbon atoms of said alkylene may be replaced by oxygen or sulfur, and
[symbol] may represent a single bond or a double bond.

The above repeating unit may be n (an integer between 1 and 500), and in this case, Formula 26 may be represented as follows.

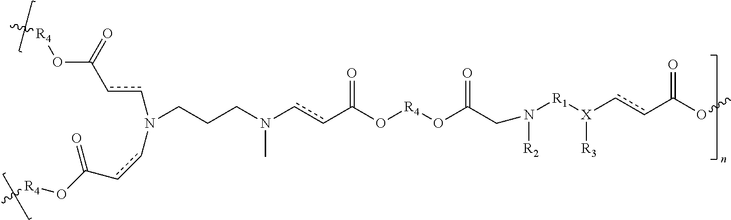

(the definitions of the groups are as defined in relation to Formula 26 above)

When the polymer according to the present invention comprises a structure of Formula 25 above, the polymer may comprise a structure of Formula 27 below.

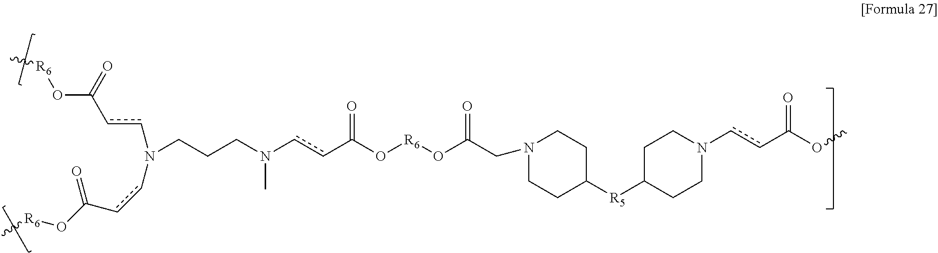

in the formula,
R5 is a C1-6 alkylene,
R6 is a linear or branched C1-15 alkylene, 1 to 5 carbon atoms of said alkylene may be replaced by oxygen or sulfur, and
[symbol] may represent a single bond or a double bond.

The above repeating unit may be n (an integer between 1 and 500), and in this case, Formula 27 may be represented as follows.

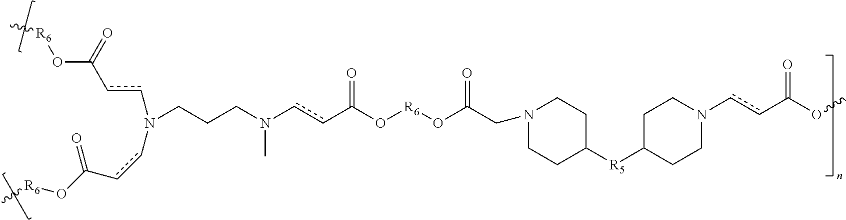

(the definitions of the groups are as defined in relation to Formula 27 above)

In one embodiment, one or both ends of the polymer may be linked to a PEG-containing moiety having a structure of Formula 28 below:

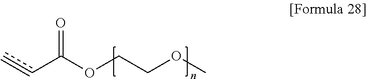

in the formula,
[symbol] may represent a double bond or a triple bond, and
n may be an integer in the range of 1 to 500.

The present invention provides a polymeric nanoparticle in which the polymer and a nucleic acid molecule form a complex. The polymeric nanoparticle according to the present invention preferably does not include a lipid nanoparticle (LNP).

The nucleic acid molecule that can be included in the polymeric nanoparticle according to the present invention may be RNA. The RNA is preferably mRNA.

The nucleic acid molecule that can be included in the polymeric nanoparticle according to the present invention may be DNA.

The polymeric nanoparticle according to the present invention may be one in which the nucleic acid molecule is isolated in a pH-dependent manner and delivered into cells in the body.

The polymeric nanoparticle according to the present invention may be one in which when delivered into the body, the expression of the nucleic acid molecule is maintained for a longer period of time compared to when the same nucleic acid molecule is delivered into the body in the form of a non-polymeric lipid nanoparticle.

The present invention provides a method for delivering a nucleic acid molecule into a cell using the polymeric nanoparticle. The nucleic acid molecule delivery method according to the present invention preferably does not use a lipid nanoparticle (LNP).

The polymeric nanoparticle according to the present invention may be delivered by parenteral, intramuscular, subcutaneous, or intravenous administration.

The present invention provides a pharmaceutical composition comprising the polymeric nanoparticle. The pharmaceutical composition according to the present invention preferably does not include a lipid nanoparticle (LNP).

The pharmaceutical composition comprising the polymeric nanoparticle according to the present invention may be a gene therapeutic agent.

The pharmaceutical composition comprising the polymeric nanoparticle according to the present invention may be a vaccine.

### Effects of Invention

Since the nucleic acid molecule delivery vehicle based on a polymer having pH sensitivity according to the present invention has a high nucleic acid molecule delivery efficiency, it can be used as an excellent delivery vehicle. In particular, the nucleic acid molecule delivery vehicle according to the present invention can be utilized as a therapeutic gene delivery vehicle and an mRNA vaccine.

In addition, depending on the length of the monomer and polymer, the nucleic acid molecule can be delivered to a desired site, and the nucleic acid molecule can be released in response to a specific stimulus through an additional stimulus-sensitive linker.

In addition, the nucleic acid molecule delivery vehicle according to the present invention can be degraded after releasing the nucleic acid molecule within the cell, so it has low side effects.

### BRIEF DESCRIPTION OF DRAWINGS

FIG. 1 is a schematic diagram of the synthesis of the polymer delivery vehicle according to the present invention and a schematic diagram of a polymeric nanoparticle (PNP) comprising the polymer delivery vehicle and mRNA.

FIGS. 2A to 2E are graphs showing the ¹H-NMR results of polymers 1A to 1E.

FIGS. 3A to 3E are graphs showing the ¹H-NMR results of polymers 2A to 2E.

FIGS. 4A to 4E are graphs showing the ¹H-NMR results of polymers 3A to 3E.

FIGS. 5A to 5E are graphs showing the ¹H-NMR results of polymers 4A to 4E.

FIGS. 6A to 6E are graphs showing the ¹H-NMR results of polymers 5A to 5E.

FIGS. 7A to 7E are graphs showing the ¹H-NMR results of polymers 6A to 6E.

FIGS. 8A to 8E are graphs showing the ¹H-NMR results of polymers 7A to 7E.

FIGS. 9A to 9E are graphs showing the ¹H-NMR results of polymers 8A to 8E.

FIGS. 10A to 10E are graphs showing the ¹H-NMR results of polymers 9A to 9E.

FIGS. 11A to 11E are graphs showing the ¹H-NMR results of polymers 10A to 10E.

FIGS. 12A to 12E are graphs showing the ¹H-NMR results of polymers 11A to 11E.

FIGS. 13A and 13B are graphs showing the ¹H-NMR results of polymers PEG-1A and PEG-1B.

FIGS. 14A and 14B are graphs showing the ¹H-NMR results of polymers PEG-2A and PEG-2B.

FIGS. 15A and 15B are graphs showing the ¹H-NMR results of polymers PEG-3A and PEG-3B.

FIGS. 16A and 16B are graphs showing the ¹H-NMR results of polymers PEG-4A and PEG-4B.

FIGS. 17A to 17C are photographs showing the electrophoresis results of the polymeric nanoparticles (PNP) according to the present invention.

FIG. 18 is a graph showing the zeta potential of the polymeric nanoparticles (PNP) according to the present invention.

FIG. 19 is a photograph showing the mRNA delivery of the polymeric nanoparticles (PNP) according to the present invention in vivo.

FIG. 20 is a graph showing the expression duration of mRNA delivered to the polymeric nanoparticles (PNP) according to the present invention.

### BEST MODE FOR CARRYING OUT THE INVENTION

The manufacturing examples and embodiments of the present invention will be described in detail so that those of ordinary skill in the art to which the present invention belongs can easily practice the present invention. However, the present invention may be implemented in various forms and is not limited to the manufacturing examples and embodiments described herein.

Throughout the present specification, when a certain part “includes” a certain component, it means that other components may be further included, rather than excluding other components, unless otherwise specifically stated.

Throughout the present specification, “polymer”, “macromolecule,” and “polymer” are used interchangeably with each other with the same meaning.

The term “repeating unit” used in describing “polymer” in the present specification refers to a structure in which the same structure appears twice or more repeatedly throughout the length of the polymer, and the polymer may not necessarily be composed of only the repeating unit. For example, the polymer may further comprise branched structures and/or end-capping agents in addition to the given repeating units.

As used herein, the term “substituted”, for example “substituted alkyl,” means that one or more hydrogen atoms of the alkyl are each independently replaced by a non-hydrogen substituent. The substituent may include, but is not limited to, any of the substituents described herein, for example, halogen, hydroxyl, alkyl, hydroxyalkyl, haloalkyl, cyanoalkyl, alkoxyalkyl, carbonyl (e.g., carboxyl, alkoxycarbonyl, formyl, or acyl), thiocarbonyl (e.g., thioester, thioacetate, or thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, aryl, or heteroaryl. The substituted moiety on the hydrocarbon chain may itself be substituted, if desired.

In the present invention, an “alkyl group” is a hydrocarbon having substituted or unsubstituted primary, secondary, tertiary and/or quaternary carbon atoms, and includes a saturated aliphatic group which may be straight-chain, branched, cyclic, or a combination thereof. For example, the alkyl group may have 1 to 20 carbon atoms (i.e., a C1-C20 alkyl), 1 to 10 carbon atoms (i.e., a C1-C10 alkyl), or 1 to 6 carbon atoms (i.e., a C1-C6 alkyl). Examples of suitable alkyl groups may include, but are not limited to, methyl (Me, —CH3), ethyl (Et, —CH2CH3), 1-propyl (n-Pr, n-propyl, —CH2CH2CH3), 2-propyl (i-Pr, i-propyl, —CH(CH3)2), 1-butyl (n-Bu, n-butyl, —CH2CH2CH2CH3), 2-methyl-1-propyl (i-Bu, i-butyl, —CH2CH(CH3)2), 2-butyl (s-Bu, s-butyl, —CH(CH3)CH2CH3), 2-methyl-2-propyl (t-Bu, t-butyl, —C(CH3)3), 1-pentyl (n-pentyl, —CH2CH2CH2CH2CH3), 2-pentyl (—CH(CH3)CH2CH2CH3), 3-pentyl (—CH(CH2CH3)2), 2-methyl-2-butyl (—C(CH3)2CH2CH3), 3-methyl-2-butyl (—CH(CH3)CH(CH3)2), 3-methyl-1-butyl (—CH2CH2CH(CH3)2), 2-methyl-1-butyl (—CH2CH(CH3)CH2CH3), 1-hexyl (—CH2CH2CH2CH2CH2CH3), 2-hexyl (—CH(CH3)CH2CH2CH2CH3), 3-hexyl (—CH(CH2CH3)(CH2CH2CH3)), 2-methyl-2-pentyl (—C(CH3)2CH2CH2CH3), 3-methyl-2-pentyl (—CH(CH3)CH(CH3)CH2CH3), 4-methyl-2-pentyl (—CH(CH3)CH2CH(CH3)2), 3-methyl-3-pentyl (—C(CH3)(CH2CH3)2), 2-methyl-3-pentyl (—CH(CH2CH3)CH(CH3)2), 2,3-dimethyl-2-butyl (—C(CH3)2CH(CH3)2), 3,3-dimethyl-2-butyl (—CH(CH3)C(CH3)3), and octyl (—(CH2)7CH3).

Moreover, as used throughout the specification, examples and claims, the term “alkyl” is intended to include both unsubstituted and substituted alkyl groups, the latter of which refers to an alkyl moiety having a substituent that replaces a hydrogen on one or more carbons of the hydrocarbon backbone, which includes a haloalkyl group such as trifluoromethyl.

As used herein, the term “alkylene” refers to a saturated hydrocarbon group having two valences, which may be branched, straight-chain, cyclic, or a combination thereof, and which is derived by removing two hydrogen atoms from the same carbon atom or two different carbon atoms of a parent alkane. For example, an alkylene group may have 1 to 20 carbon atoms, 1 to 10 carbon atoms, or 1 to 6 carbon atoms. Examples of suitable alkylene groups may include, but are not limited to, methylene (—CH2-) and 1,2-ethylene (—CH2-CH2-).

As used herein, the term “heterocycle” refers to a substituted or unsubstituted, monovalent or divalent, saturated or partially saturated non-aromatic ring structure, preferably a 3- to 10-membered ring, more preferably a 3- to 7-membered ring, wherein the ring structure comprises one or more heteroatoms, preferably 1 to 4 heteroatoms, and more preferably 1 to 2 heteroatoms. The terms “heterocyclyl,” “heterocycle,” “heterocyclic,” and “heterocycloalkyl” also include polycyclic ring systems having two or more cyclic rings in which two or more carbons are common to two adjacent rings, wherein at least one of the rings is heterocyclic, for example, the other cyclic rings may be cycloalkyl, cycloalkenyl, cycloalkynyl, aryl, heteroaryl, and/or heterocyclyl. The bicyclic and polycyclic heterocyclic ring systems may be fused, bridged, or spiro ring systems. The substituted heterocycles include heterocyclic rings substituted with any of the substituents disclosed herein, including, for example, a carbonyl group. The heterocyclyl groups include, for example, piperidine, piperazine, pyrrolidine, morpholine, lactones, lactams, and the like. Additionally exemplary heterocycles may include, but are not limited to, dihydropyridyl, dihydroindolyl, tetrahydropyridyl (piperidyl), tetrahydrothiophenyl, sulfur-oxidized tetrahydrothiophenyl, indolenyl, piperidinyl, 4-piperidinyl, pyrrolidinyl, 2-pyrrolidonyl, pyrrolinyl, tetrahydrofuranyl, tetrahydroquinolinyl, tetrahydroisoquinolinyl, decahydroquinolinyl, octahydroisoquinolinyl, 6H-1,2,5-thiadiazinyl, 2H,6H-1,5,2-dithiazinyl, pyranyl, chromenyl, xanthenyl, phenoxathinyl, 2H-pyrrolyl, 3H-indolyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, quinuclidinyl, morpholinyl, and oxazolidinyl (each of which may be substituted or unsubstituted).

As used herein, the term “carbocycle” refers to a non-aromatic saturated or unsaturated, monovalent or divalent ring which may be monocyclic, bicyclic, or polycyclic and in which each of the ring atoms is carbon. Cycloalkyl groups may have from 3 to 7 carbon atoms as a monocycle, from 7 to 12 carbon atoms as a bicycle, and up to about 20 carbon atoms as a polycycle. Monocyclic cycloalkyls have from 3 to 7 ring atoms, more typically 5 or 6 ring atoms. Bicyclic cycloalkyls may have from 7 to 12 ring atoms and may be fused ring systems, spirocyclic ring systems, or bridged ring systems. In exemplary cycloalkyl groups, the atoms may be arranged in a bicyclo[4,5], [5,5], [5,6], or [6,6] system. In certain embodiments, the cycloalkyl contains from 3 to 20 atoms, or from 3 to 10 atoms, or more preferably from 3 to 7 atoms. Examples of cycloalkyls may include cyclopropyl, cyclobutyl, cyclopentyl, cyclohexyl, cycloheptyl, cyclooctyl, and the like. Unless otherwise specified, cycloalkyl may be substituted by one or more substituents described herein.

As used herein, the term “alkoxyacyl” refers to a group of —C(═O)OR. R is alkyl as defined herein, which may be substituted or unsubstituted.

[symbol] as used in the chemical structural formulas herein represents a single bond or a double bond, according to the convention used in the art.

[symbol] as used in the chemical structural formulas herein represents a bond connected to another unit, according to the convention used in the art.

As used herein, the term “amine” refers to —NH2, in which the hydrogen atom is unsubstituted or substituted with a substituent such as alkyl or aryl, wherein the substituent such as alkyl or aryl that substitutes the hydrogen atom is as defined herein, and may be substituted or unsubstituted.

As used herein, the term “amine monomer” is a monomer containing an amine functional group, and preferably, may be selected from the group consisting of amine monomers described in Table 1 below.

TABLE 1

| No. | Structural formula |
|---|---|
| 1 | 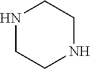 |
| 2 | 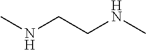 |
| 3 | 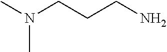 |
| 4 | 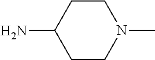 |
| 5 | 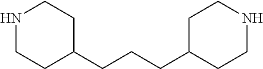 [SMILES: C(CC1CCNCC1)CC1CCNCC1] |
| 6 | 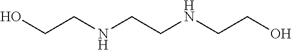 |
| 7 | 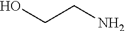 |
| 8 | 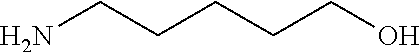 |
| 9 | 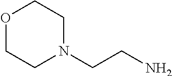 |
| 10 | 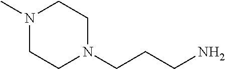 |
| 11 | 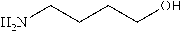 |
| 12 | 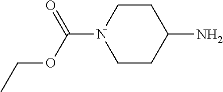 |

As used herein, the term “acrylate monomer” is a monomer in the form of an ester derivative of acrylic acid, and may be selected from the group consisting of acrylate monomers described in Table 2 below.

TABLE 2

| No. | Stuctural formula |
|---|---|
| A | 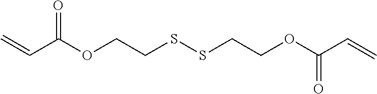 [SMILES: C=CC(=O)OCCSSCCOC(=O)C=C] |
| B | 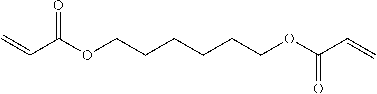 [SMILES: C=CC(=O)OCCCCCCOC(=O)C=C] |
| C | 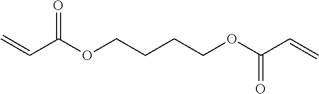 |
| D | 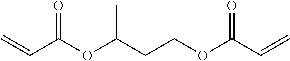 |
| E | 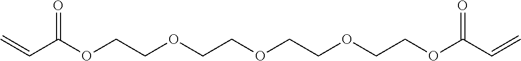 [SMILES: C=CC(=O)OCCOCCOCCOCCOC(=O)C=C] |

As used herein, the term “alkynoate monomer” is a monomer in the form of an ester derivative of alkynoic acid, and may be selected from the group consisting of alkynoate monomers described in Table 3 below.

TABLE 3

| No. | Structural formula |
|---|---|
| F | 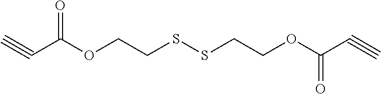 [SMILES: C#CC(=O)OCCSSCCOC(=O)C#C] |
| G | 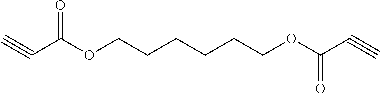 [SMILES: C#CC(=O)OCCCCCCOC(=O)C#C] |
| H | 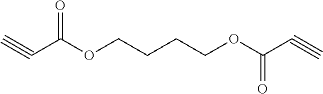 |
| I | 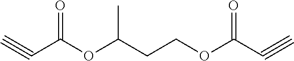 |
| J | 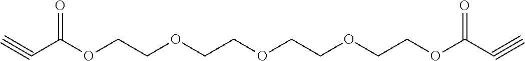 [SMILES: C#CC(=O)OCCOCCOCCOCCOC(=O)C#C] |

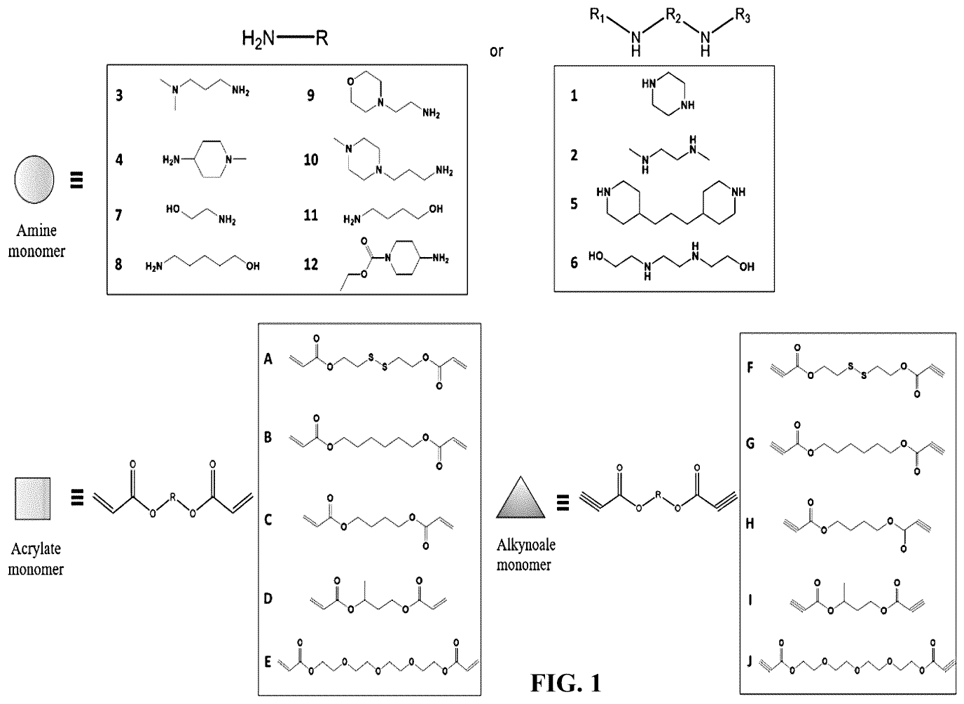

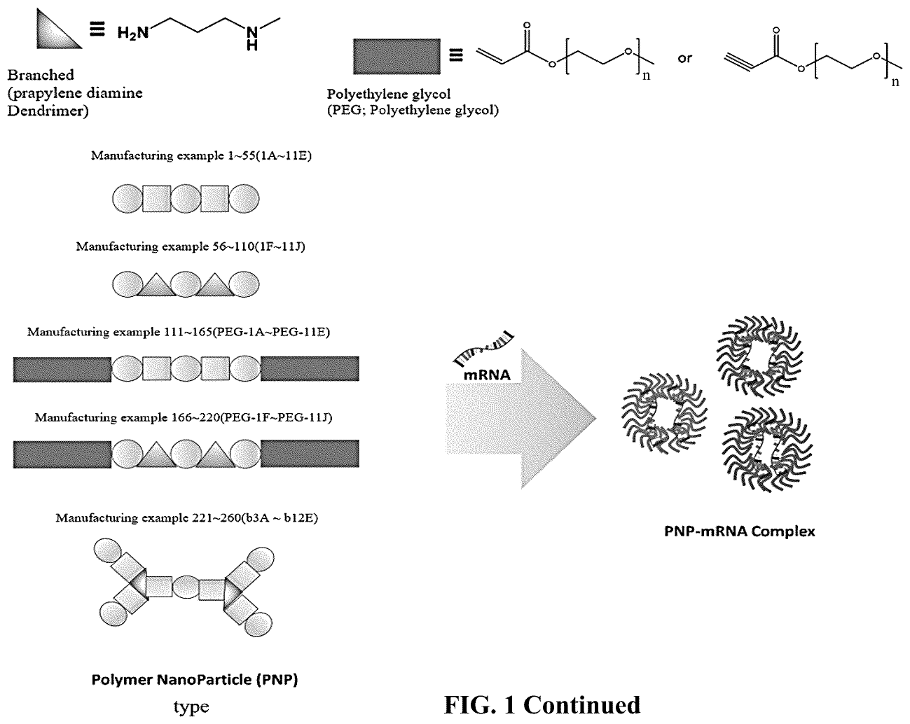

The polymer according to the present invention is characterized by including an amine monomer and an acrylate or alkynoate monomer together, and the two monomers are alternately connected (see schematic diagram of FIG. 1).

The polymer according to the present invention is characterized by having an ionizable moiety, where the “ionizable moiety” means a part of the polymer that is ionized depending on the pH. For example, it may be a tertiary amine present in the polymer chemical structure, and those of ordinary skill in the art are well aware of which moiety can be ionized depending on the pH.

Hereinafter, the present invention will be described in more detail through the examples, but the following examples are for illustrative purposes only and are not intended to limit the scope of the present invention.

### Manufacturing Example 1

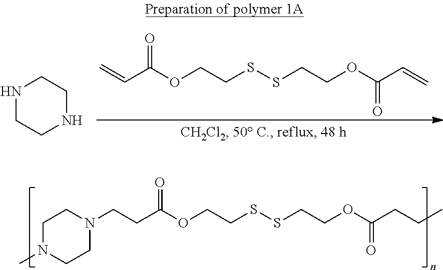

Acrylate monomer A (1000 mg) was placed in a 2-neck round bottom flask, and then amine monomer 1 was added to the flask so that the molar ratio with the acrylate monomer was 1.04. Dichloromethane (CH₂Cl₂, DCM) was added to the flask so that the final concentration was 1000 mg/ml. Thereafter, the reaction was carried out at 50° C. for 48 hours with reflux, and then polymer 1A having the ionizable moiety of the present invention was obtained.

### Manufacturing Example 2

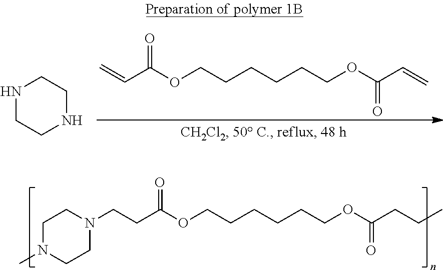

Acrylate monomer B (1000 mg) was placed in a 2-neck round bottom flask, and then amine monomer 1 was added to the flask so that the molar ratio with the acrylate monomer was 1.04. DCM was added to the flask so that the final concentration was 1000 mg/ml. Thereafter, the reaction was carried out at 50° C. for 48 hours with reflux, and then polymer 1B having the ionizable moiety of the present invention was obtained.

### Manufacturing Example 3

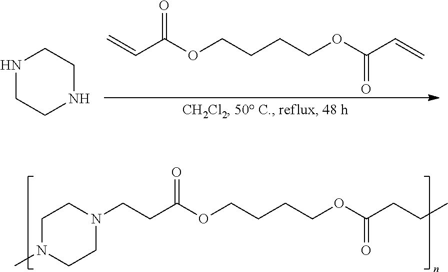

Acrylate monomer C (1000 mg) was placed in a 2-neck round bottom flask, and then amine monomer 1 was added to the flask so that the molar ratio with the acrylate monomer was 1.04. DCM was added to the flask so that the final concentration was 1000 mg/ml. Thereafter, the reaction was carried out at 50° C. for 48 hours with reflux, and then polymer 1C having the ionizable moiety of the present invention was obtained.

### Manufacturing Example 4

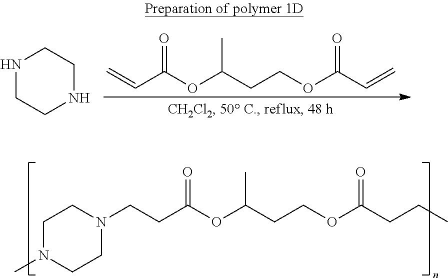

Acrylate monomer D (1000 mg) was placed in a 2-neck round bottom flask, and then amine monomer 1 was added to the flask so that the molar ratio with the acrylate monomer was 1.04. DCM was added to the flask so that the final concentration was 1000 mg/ml. Thereafter, the reaction was carried out at 50° C. for 48 hours with reflux, and then polymer 1D having the ionizable moiety of the present invention was obtained.

### Manufacturing Example 5

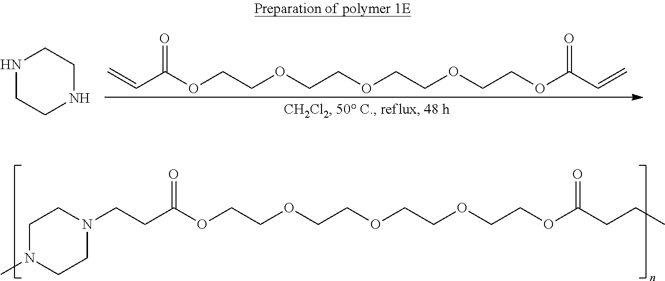

Acrylate monomer E (1000 mg) was placed in a 2-neck round bottom flask, and then amine monomer 1 was added to the flask so that the molar ratio with the acrylate monomer was 1.04. DCM was added to the flask so that the final concentration was 2500 mg/ml. Thereafter, the reaction was carried out at 50° C. for 48 hours with reflux, and then polymer 1E having the ionizable moiety of the present invention was obtained.

### Manufacturing Example 6

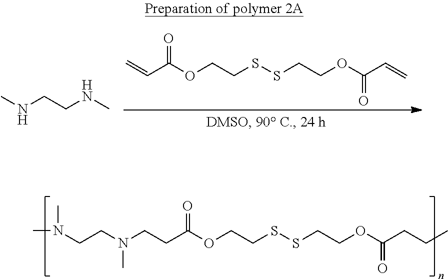

Acrylate monomer A (1000 mg) was placed in a 20 ml vial, and then amine monomer 2 was added to the vial so that the molar ratio with the acrylate monomer was 1.04. Dimethylsulfoxide (DMSO) was added to the vial so that the final concentration was 1000 mg/ml. Thereafter, the reaction was carried out at 90° C. for 24 hours, and then polymer 2A having the ionizable moiety of the present invention was obtained.

### Manufacturing Example 7

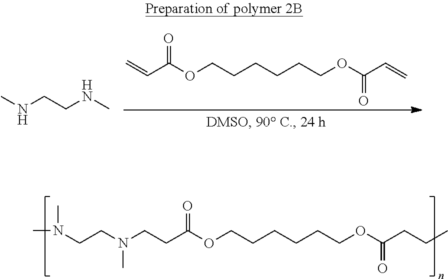

Acrylate monomer B (1000 mg) was placed in a 20 ml vial, and then amine monomer 2 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 2B having the ionizable moiety of the present invention was obtained.

### Manufacturing Example 8

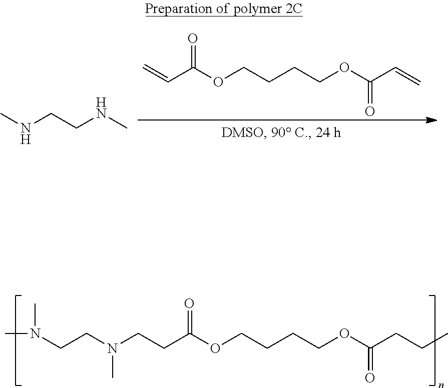

Acrylate monomer C (1000 mg) was placed in a 20 ml vial, and then amine monomer 2 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 2C having the ionizable moiety of the present invention was obtained.

### Manufacturing Example 9

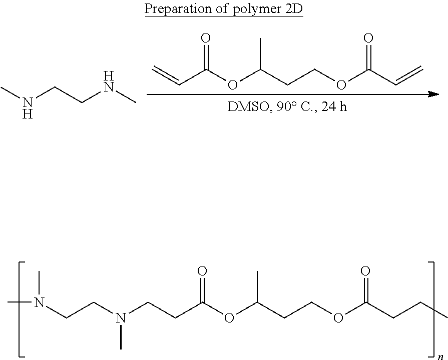

Acrylate monomer D (1000 mg) was placed in a 20 ml vial, and then amine monomer 2 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 2D having the ionizable moiety of the present invention was obtained.

### Manufacturing Example 10

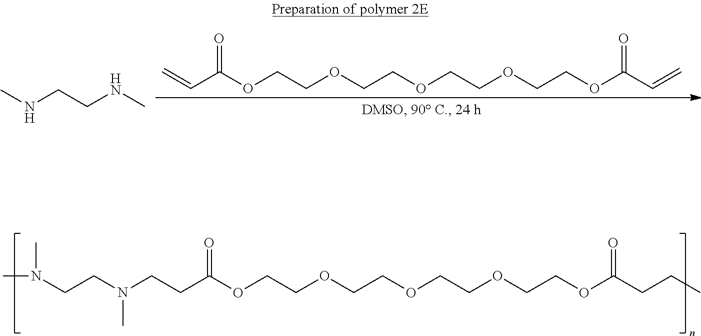

Acrylate monomer E (1000 mg) was placed in a 20 ml vial, and then amine monomer 2 was added to the vial so that the molar ratio with the acrylate monomer was 1.04. DMSO was added to the vial so that the final concentration was 2500 mg/ml. Thereafter, the reaction was carried out at 90° C. for 24 hours, and then polymer 2E having the ionizable moiety of the present invention was obtained.

### Manufacturing Example 11

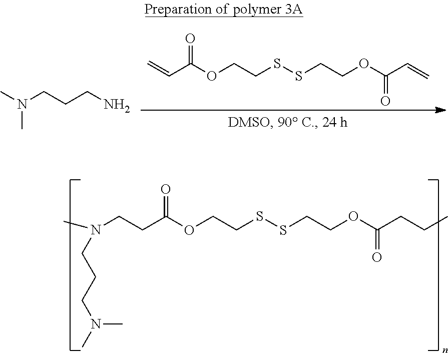

Acrylate monomer A (1000 mg) was placed in a 20 ml vial, and then amine monomer 3 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 3A having the ionizable moiety of the present invention was obtained.

### Manufacturing Example 12

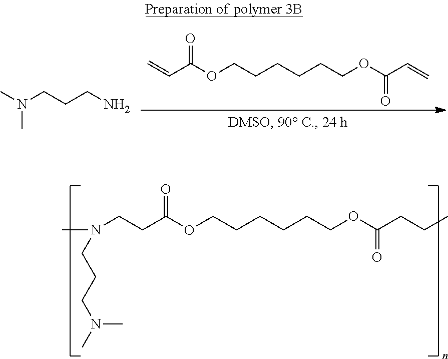

Acrylate monomer B (1000 mg) was placed in a 20 ml vial, and then amine monomer 3 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 3B having the ionizable moiety of the present invention was obtained.

### Manufacturing Example 13

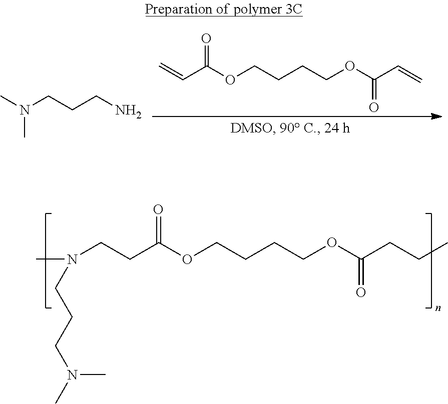

Acrylate monomer C (1000 mg) was placed in a 20 ml vial, and then amine monomer 3 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 3C having the ionizable moiety of the present invention was obtained.

### Manufacturing Example 14

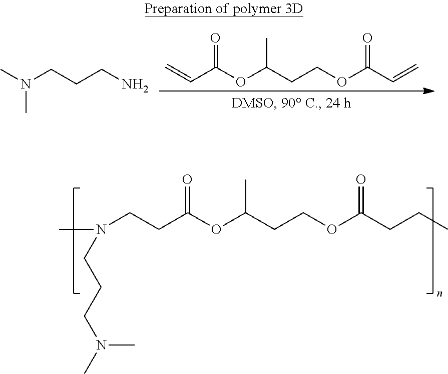

Acrylate monomer D (1000 mg) was placed in a 20 ml vial, and then amine monomer 3 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 3D having the ionizable moiety of the present invention was obtained.

### Manufacturing Example 15

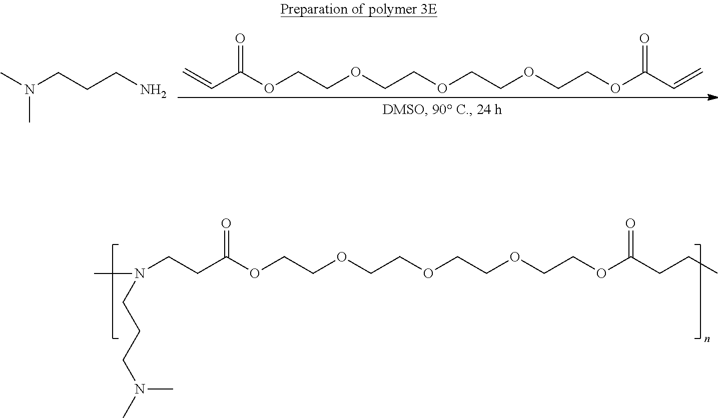

Acrylate monomer E (1000 mg) was placed in a 20 ml vial, and then amine monomer 3 was added to the vial so that the molar ratio with the acrylate monomer was 1.04. DMSO was added to the vial so that the final concentration was 2500 mg/ml. Thereafter, the reaction was carried out at 90° C. for 24 hours, and then polymer 3E having the ionizable moiety of the present invention was obtained.

### Manufacturing Example 16

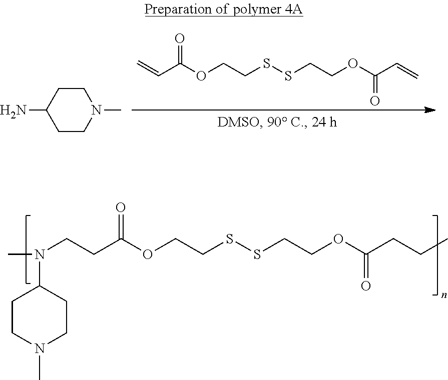

Acrylate monomer A (1000 mg) was placed in a 20 ml vial, and then amine monomer 4 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 4A having the ionizable moiety of the present invention was obtained.

### Manufacturing Example 17

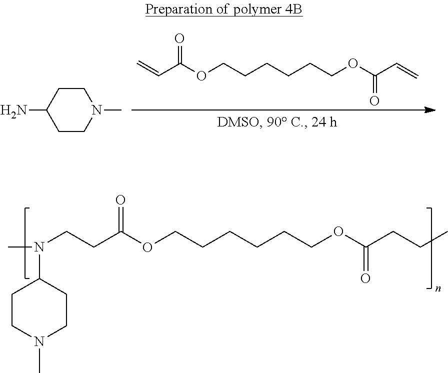

Acrylate monomer B (1000 mg) was placed in a 20 ml vial, and then amine monomer 4 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 4B having the ionizable moiety of the present invention was obtained.

### Manufacturing Example 18

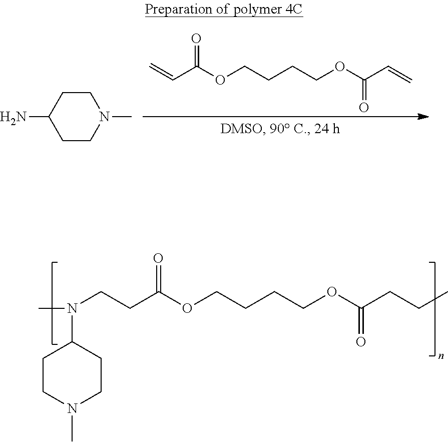

Acrylate monomer C (1000 mg) was placed in a 20 ml vial, and then amine monomer 4 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 4C having the ionizable moiety of the present invention was obtained.

### Manufacturing Example 19

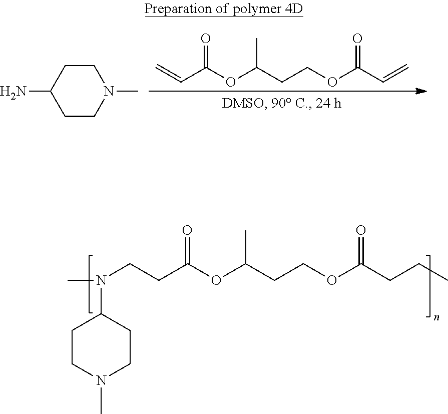

Acrylate monomer D (1000 mg) was placed in a 20 ml vial, and then amine monomer 4 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 4D having the ionizable moiety of the present invention was obtained.

### Manufacturing Example 20

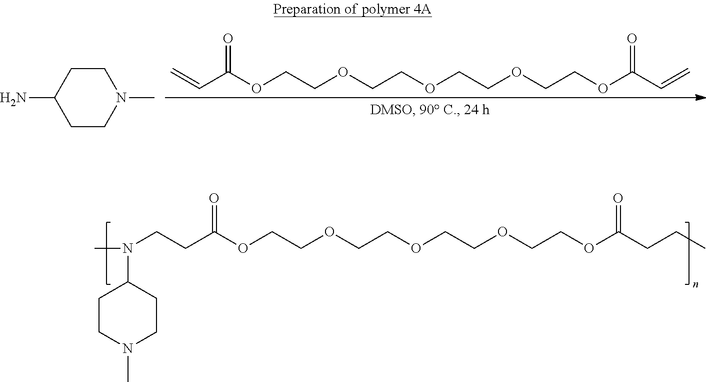

Acrylate monomer E (1000 mg) was placed in a 20 ml vial, and then amine monomer 4 was added to the vial so that the molar ratio with the acrylate monomer was 1.04. DMSO was added to the vial so that the final concentration was 2500 mg/ml. Thereafter, the reaction was carried out at 90° C. for 24 hours, and then polymer 4E having the ionizable moiety of the present invention was obtained.

### Manufacturing Example 21

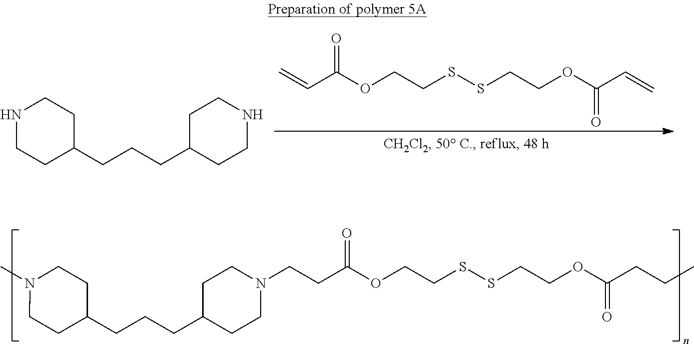

Acrylate monomer A (1000 mg) was placed in a 2-neck round bottom flask, and then amine monomer 5 was added to the flask so that the molar ratio with the acrylate monomer was 1.04. DCM was added to the flask so that the final concentration was 1000 mg/ml. Thereafter, the reaction was carried out at 50° C. for 48 hours with reflux, and then polymer 5A having the ionizable moiety of the present invention was obtained.

### Manufacturing Example 22

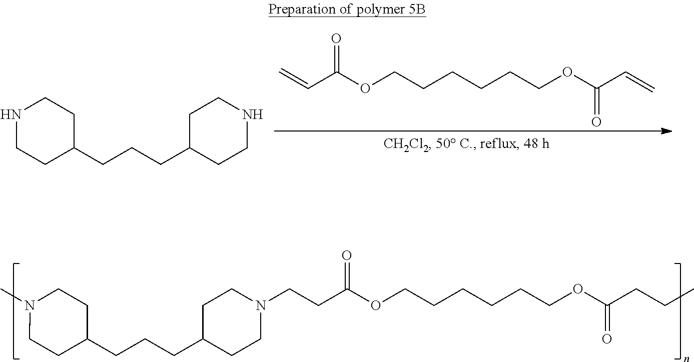

Acrylate monomer B (1000 mg) was placed in a 2-neck round bottom flask, and then amine monomer 5 was added to the flask so that the molar ratio with the acrylate monomer was 1.04. DCM was added to the flask so that the final concentration was 1000 mg/ml. Thereafter, the reaction was carried out at 50° C. for 48 hours with reflux, and then polymer 5B having the ionizable moiety of the present invention was obtained.

### Manufacturing Example 23

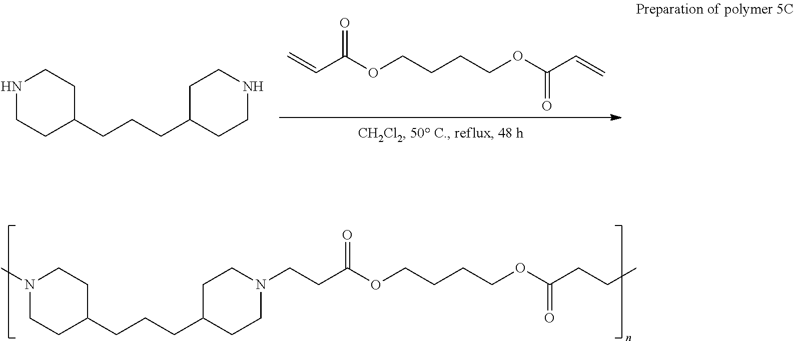

Acrylate monomer C (1000 mg) was placed in a 2-neck round bottom flask, and then amine monomer 5 was added to the flask so that the molar ratio with the acrylate monomer was 1.04. DCM was added to the flask so that the final concentration was 1000 mg/ml. Thereafter, the reaction was carried out at 50° C. for 48 hours with reflux, and then polymer 5C having the ionizable moiety of the present invention was obtained.

### Manufacturing Example 24

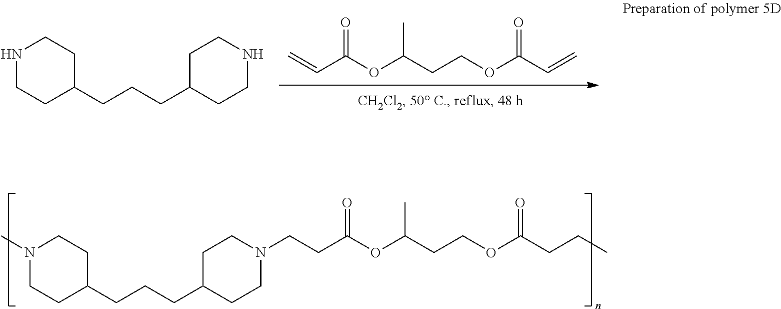

Acrylate monomer D (1000 mg) was placed in a 2-neck round bottom flask, and then amine monomer 5 was added to the flask so that the molar ratio with the acrylate monomer was 1.04. DCM was added to the flask so that the final concentration was 1000 mg/ml. Thereafter, the reaction was carried out at 50° C. for 48 hours with reflux, and then polymer 5D having the ionizable moiety of the present invention was obtained.

### Manufacturing Example 25

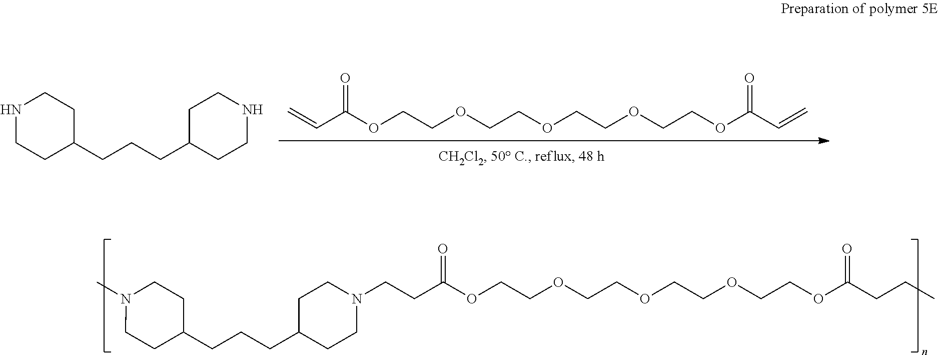

Acrylate monomer E (1000 mg) was placed in a 2-neck round bottom flask, and then amine monomer 5 was added to the flask so that the molar ratio with the acrylate monomer was 1.04. DCM was added to the flask so that the final concentration was 1000 mg/ml. Thereafter, the reaction was carried out at 50° C. for 48 hours with reflux, and then polymer 5E having the ionizable moiety of the present invention was obtained.

### Manufacturing Example 26

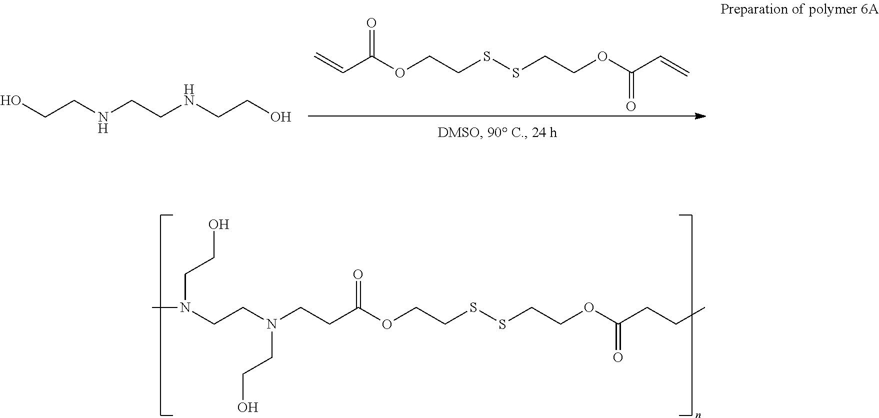

Acrylate monomer A (1000 mg) was placed in a 20 ml vial, and then amine monomer 6 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 6A having the ionizable moiety of the present invention was obtained.

### Manufacturing Example 27

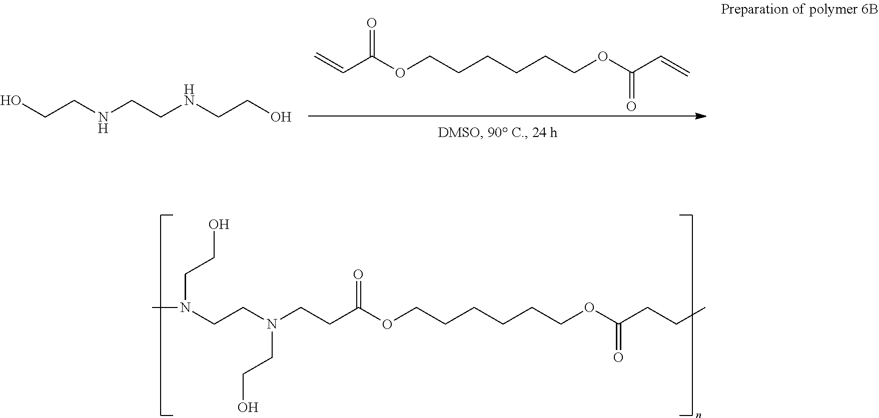

Acrylate monomer B (1000 mg) was placed in a 20 ml vial, and then amine monomer 6 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 6B having the ionizable moiety of the present invention was obtained.

### Manufacturing Example 28

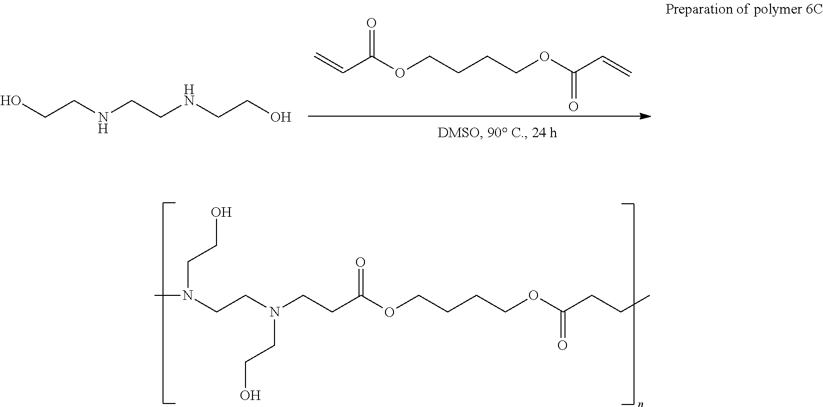

Acrylate monomer C (1000 mg) was placed in a 20 ml vial, and then amine monomer 6 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 6C having the ionizable moiety of the present invention was obtained.

### Manufacturing Example 29

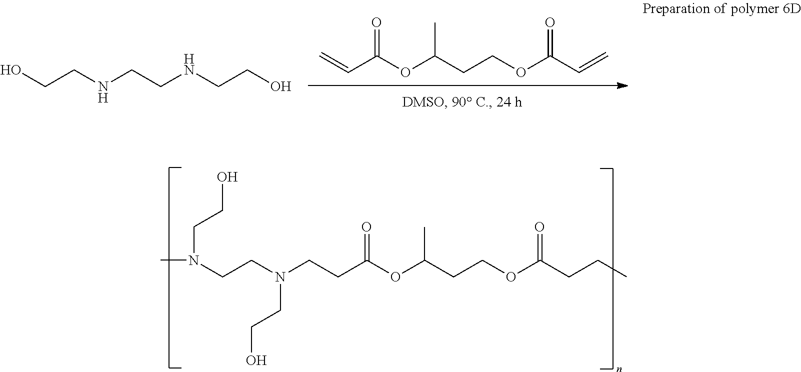

Acrylate monomer D (1000 mg) was placed in a 20 ml vial, and then amine monomer 6 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 6D having the ionizable moiety of the present invention was obtained.

### Manufacturing Example 30

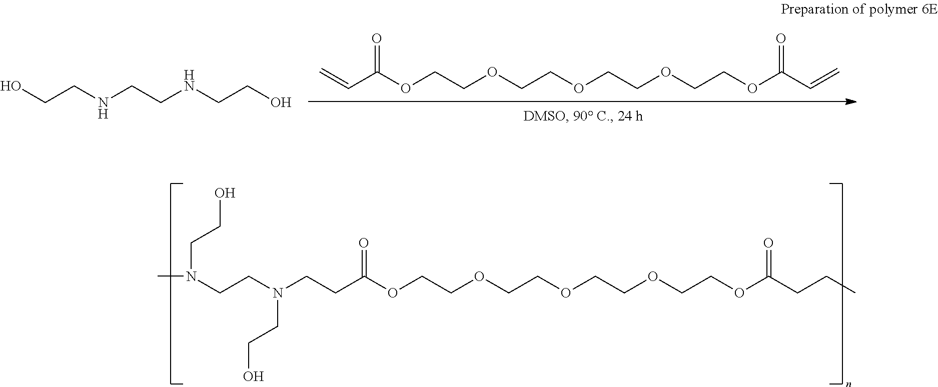

Acrylate monomer E (1000 mg) was placed in a 20 ml vial, and then amine monomer 6 was added to the vial so that the molar ratio with the acrylate monomer was 1.04. DMSO was added to the vial so that the final concentration was 2500 mg/ml. Thereafter, the reaction was carried out at 90° C. for 24 hours, and then polymer 6E having the ionizable moiety of the present invention was obtained.

### Manufacturing Example 31

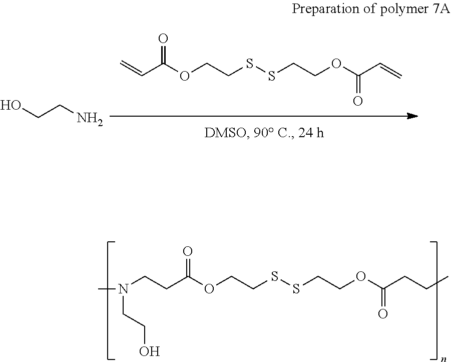

Acrylate monomer A (1000 mg) was placed in a 20 ml vial, and then amine monomer 7 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 7A having the ionizable moiety of the present invention was obtained.

### Manufacturing Example 32

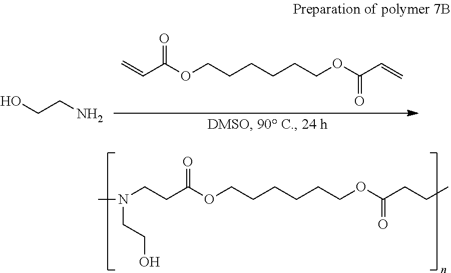

Acrylate monomer B (1000 mg) was placed in a 20 ml vial, and then amine monomer 7 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 7B having the ionizable moiety of the present invention was obtained.

### Manufacturing Example 33

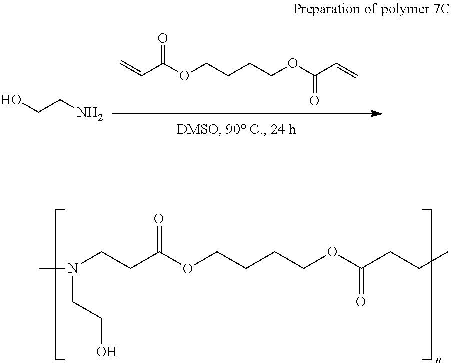

Acrylate monomer C (1000 mg) was placed in a 20 ml vial, and then amine monomer 7 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 7C having the ionizable moiety of the present invention was obtained.

### Manufacturing Example 34

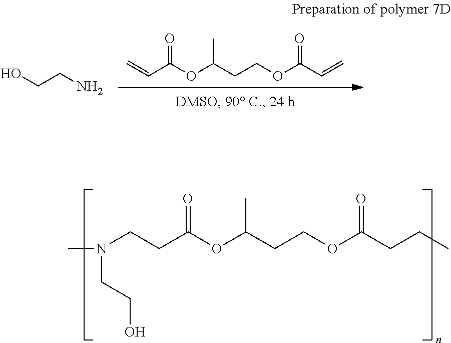

Acrylate monomer D (1000 mg) was placed in a 20 ml vial, and then amine monomer 7 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 7D having the ionizable moiety of the present invention was obtained.

### Manufacturing Example 35

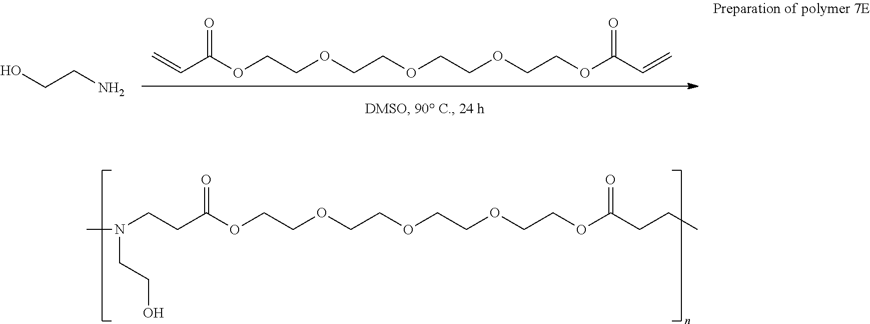

Acrylate monomer E (1000 mg) was placed in a 20 ml vial, and then amine monomer 7 was added to the vial so that the molar ratio with the acrylate monomer was 1.04. DMSO was added to the vial so that the final concentration was 2500 mg/ml. Thereafter, the reaction was carried out at 90° C. for 24 hours, and then polymer 7E having the ionizable moiety of the present invention was obtained.

### Manufacturing Example 36

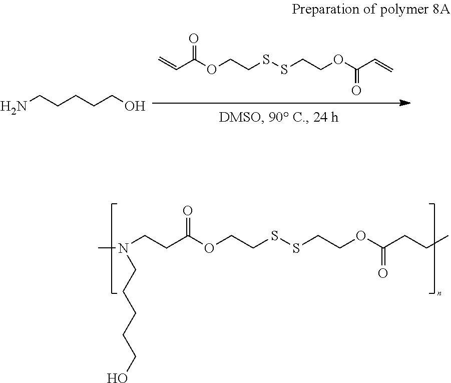

Acrylate monomer A (1000 mg) was placed in a 20 ml vial, and then amine monomer 8 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 8A having the ionizable moiety of the present invention was obtained.

### Manufacturing Example 37

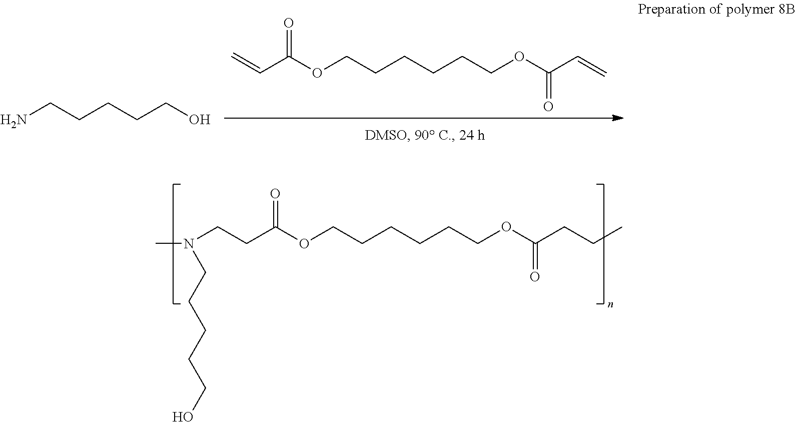

Acrylate monomer B (1000 mg) was placed in a 20 ml vial, and then amine monomer 8 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 8B having the ionizable moiety of the present invention was obtained.

### Manufacturing Example 38

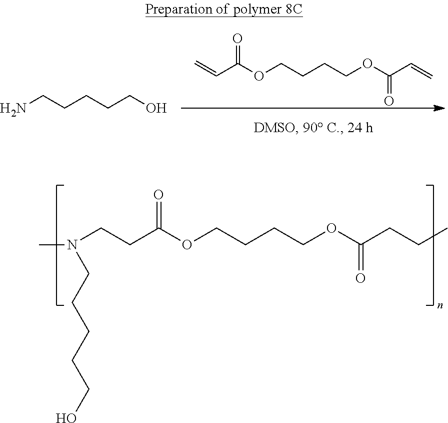

Acrylate monomer C (1000 mg) was placed in a 20 ml vial, and then amine monomer 8 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 8C having the ionizable moiety of the present invention was obtained.

### Manufacturing Example 39

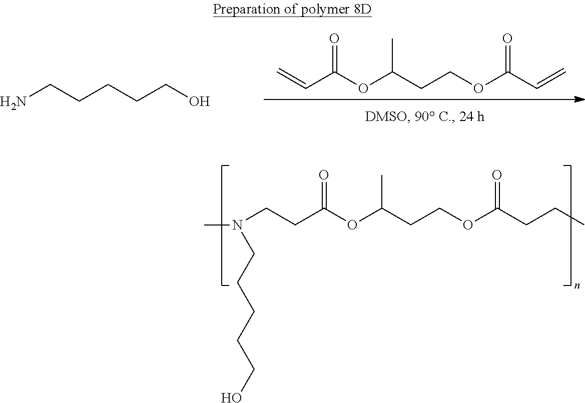

Acrylate monomer D (1000 mg) was placed in a 20 ml vial, and then amine monomer 8 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 8D having the ionizable moiety of the present invention was obtained.

### Manufacturing Example 40

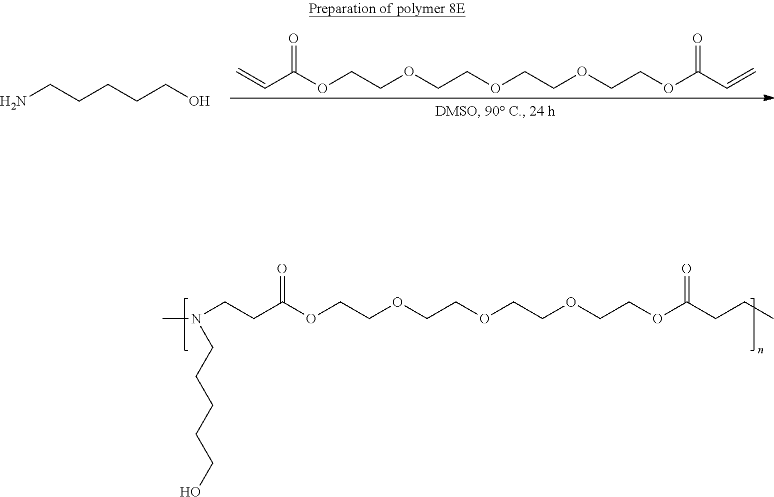

Acrylate monomer E (1000 mg) was placed in a 20 ml vial, and then amine monomer 8 was added to the vial so that the molar ratio with the acrylate monomer was 1.04. DMSO was added to the vial so that the final concentration was 2500 mg/ml. Thereafter, the reaction was carried out at 90° C. for 24 hours, and then polymer 8E having the ionizable moiety of the present invention was obtained.

### Manufacturing Example 41

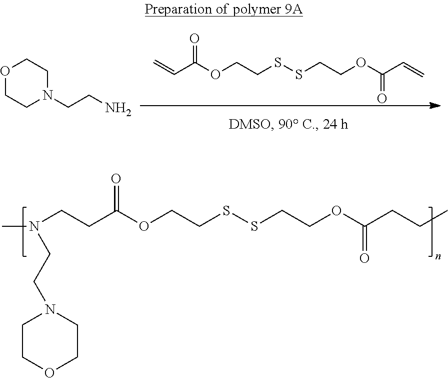

Acrylate monomer A (1000 mg) was placed in a 20 ml vial, and then amine monomer 9 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 9A having the ionizable moiety of the present invention was obtained.

### Manufacturing Example 42

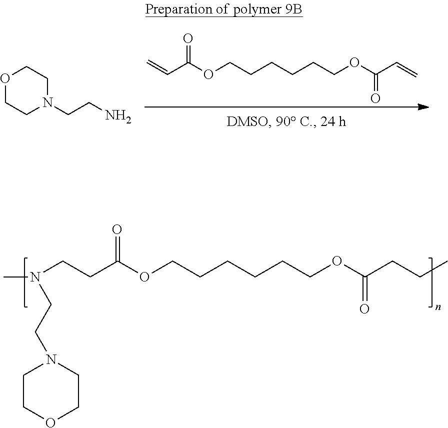

Acrylate monomer B (1000 mg) was placed in a 20 ml vial, and then amine monomer 9 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 9B having the ionizable moiety of the present invention was obtained.

### Manufacturing Example 43

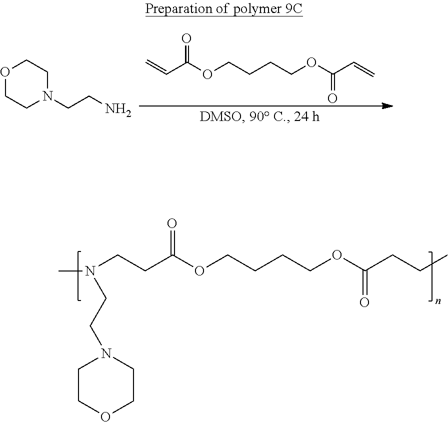

Acrylate monomer C (1000 mg) was placed in a 20 ml vial, and then amine monomer 9 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 9C having the ionizable moiety of the present invention was obtained.

### Manufacturing Example 44

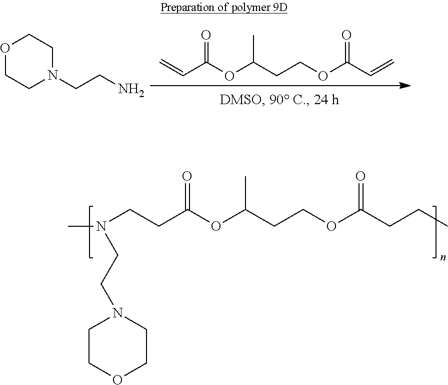

Acrylate monomer D (1000 mg) was placed in a 20 ml vial, and then amine monomer 9 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 9D having the ionizable moiety of the present invention was obtained.

### Manufacturing Example 45

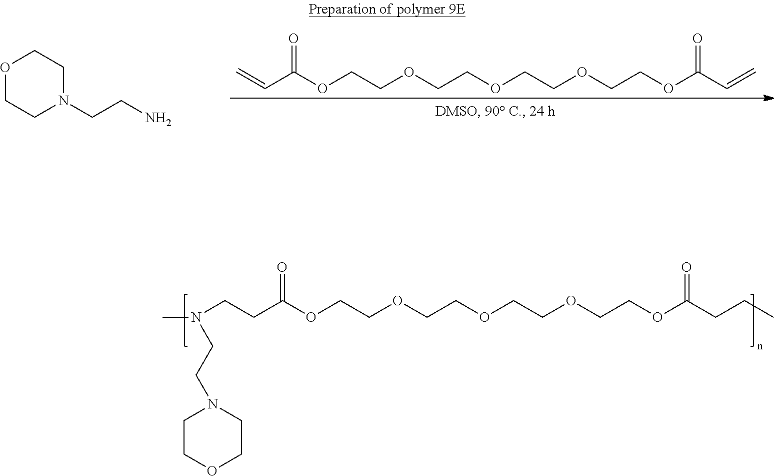

Acrylate monomer E (1000 mg) was placed in a 20 ml vial, and then amine monomer 9 was added to the vial so that the molar ratio with the acrylate monomer was 1.04. DMSO was added to the vial so that the final concentration was 2500 mg/ml. Thereafter, the reaction was carried out at 90° C. for 24 hours, and then polymer 9E having the ionizable moiety of the present invention was obtained.

### Manufacturing Example 46

### Preparation of Polymer 10A

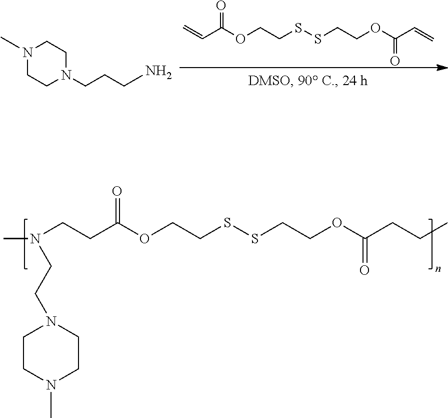

Acrylate monomer A (1000 mg) was placed in a 20 ml vial, and then amine monomer 10 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 10A having the ionizable moiety of the present invention was obtained.

### Manufacturing Example 47

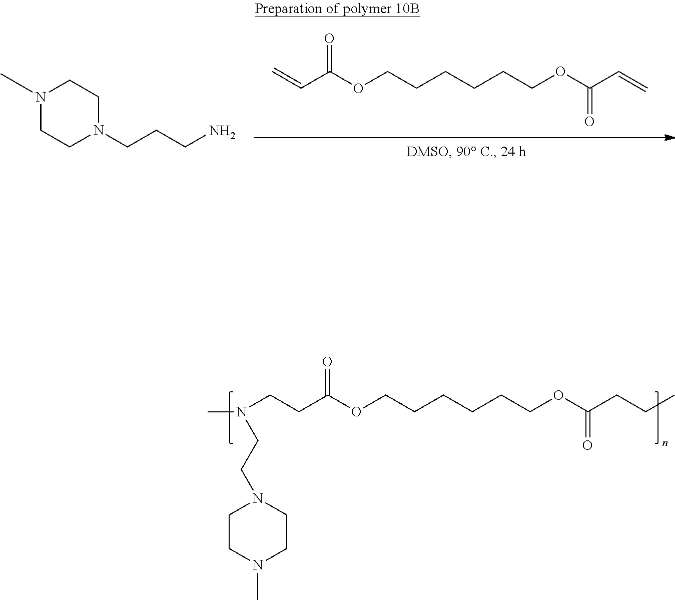

Acrylate monomer B (1000 mg) was placed in a 20 ml vial, and then amine monomer 10 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 10B having the ionizable moiety of the present invention was obtained.

### Manufacturing Example 48

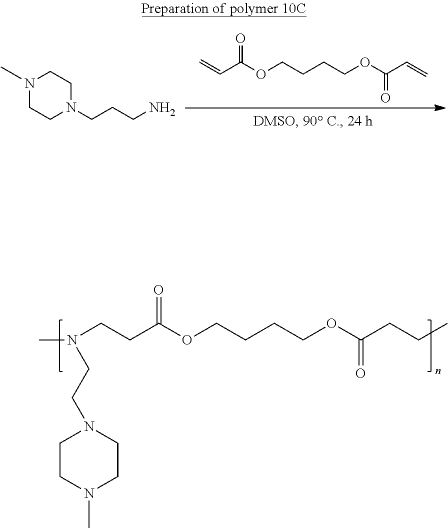

Acrylate monomer C (1000 mg) was placed in a 20 ml vial, and then amine monomer 10 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 10C having the ionizable moiety of the present invention was obtained.

### Manufacturing Example 49

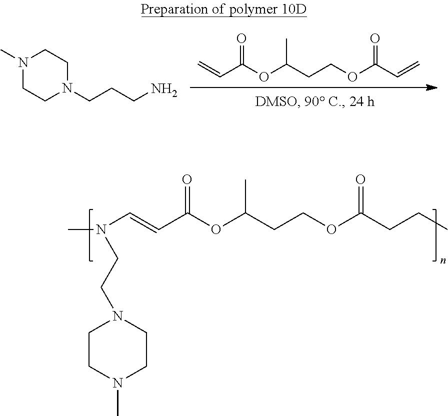

Acrylate monomer D (1000 mg) was placed in a 20 ml vial, and then amine monomer 10 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 10D having the ionizable moiety of the present invention was obtained.

### Manufacturing Example 50

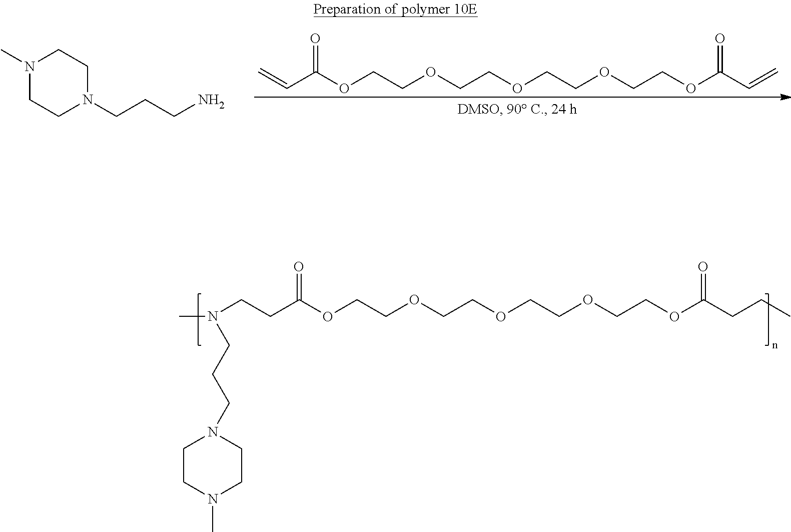

Acrylate monomer E (1000 mg) was placed in a 20 ml vial, and then amine monomer 10 was added to the vial so that the molar ratio with the acrylate monomer was 1.04. DMSO was added to the vial so that the final concentration was 2500 mg/ml. Thereafter, the reaction was carried out at 90° C. for 24 hours, and then polymer 10E having the ionizable moiety of the present invention was obtained.

### Manufacturing Example 51

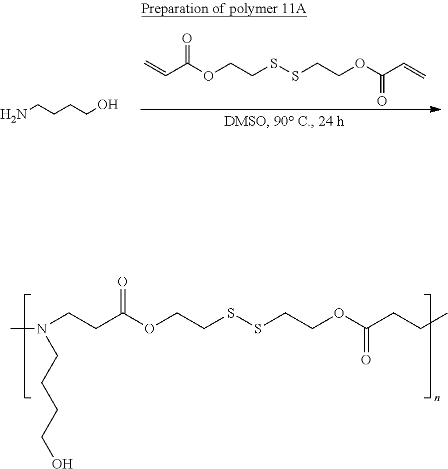

Acrylate monomer A (1000 mg) was placed in a 20 ml vial, and then amine monomer 11 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 11A having the ionizable moiety of the present invention was obtained.

### Manufacturing Example 52

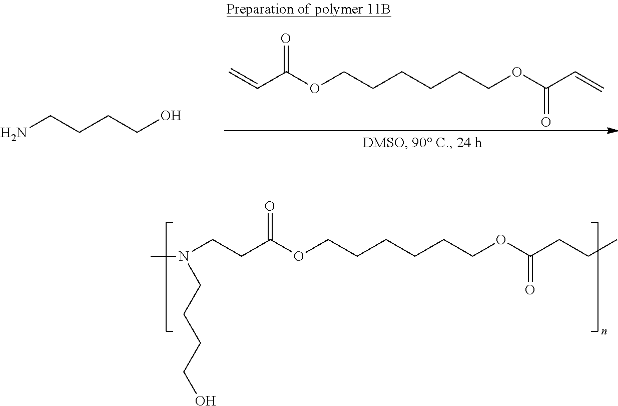

Acrylate monomer B (1000 mg) was placed in a 20 ml vial, and then amine monomer 11 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 11B having the ionizable moiety of the present invention was obtained.

### Manufacturing Example 53

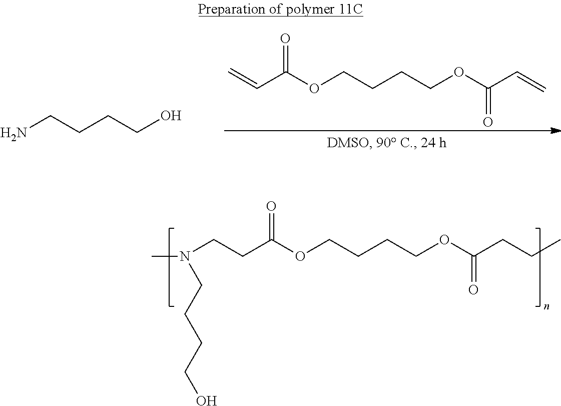

Acrylate monomer C (1000 mg) was placed in a 20 ml vial, and then amine monomer 11 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 11C having the ionizable moiety of the present invention was obtained.

### Manufacturing Example 54

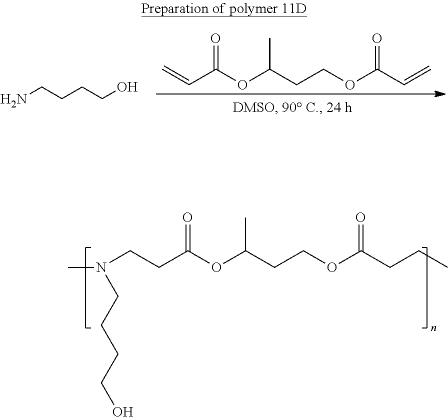

Acrylate monomer D (1000 mg) was placed in a 20 ml vial, and then amine monomer 11 was added to the vial so that the molar ratio with the acrylate monomer was 1.04. DMSO was added to the vial so that the final concentration was 1000 mg/ml. Thereafter, the reaction was carried out at 90° C. for 24 hours, and then polymer 11D having the ionizable moiety of the present invention was obtained.

### Manufacturing Example 55

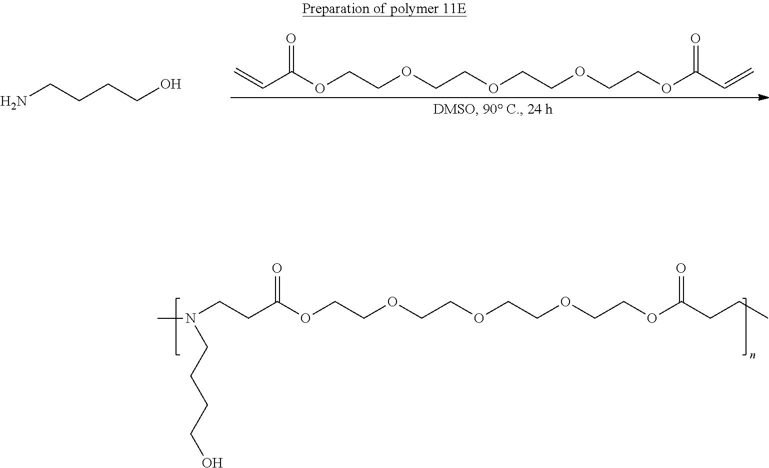

Acrylate monomer E (1000 mg) was placed in a 20 ml vial, and then amine monomer 11 was added to the vial so that the molar ratio with the acrylate monomer was 1.04. DMSO was added to the vial so that the final concentration was 2500 mg/ml. Thereafter, the reaction was carried out at 90° C. for 24 hours, and then polymer 11E having the ionizable moiety of the present invention was obtained.

### Manufacturing Example 56

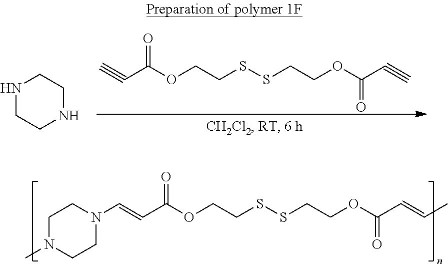

Alkynoate monomer F (500 mg) was placed in a round bottom flask, and then amine monomer 1 was added to the flask so that the molar ratio with the acrylate monomer was 1.04, and 4 mL of dichloromethane was added to the flask. Thereafter, the reaction was carried out at room temperature for 6 hours, and then polymer 1F having the ionizable moiety of the present invention was obtained.

### Manufacturing Example 57

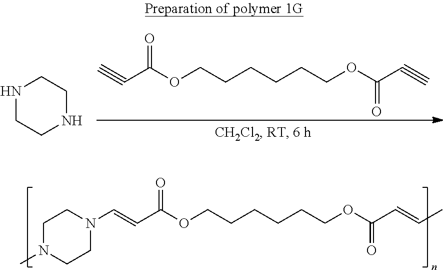

Alkynoate monomer G (500 mg) was placed in a round bottom flask, and then amine monomer 1 was added to the flask so that the molar ratio with the alkynoate monomer was 1.04, and 4 mL of dichloromethane was added to the flask. Thereafter, the reaction was carried out at room temperature for 6 hours, and then polymer 1G having the ionizable moiety of the present invention was obtained.

### Manufacturing Example 58

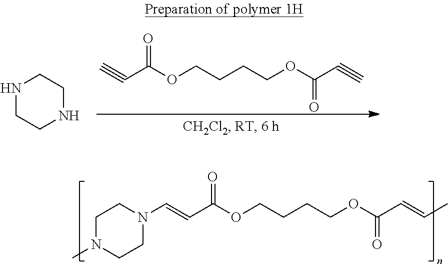

Alkynoate monomer H (500 mg) was placed in a round bottom flask, and then amine monomer 1 was added to the flask so that the molar ratio with the alkynoate monomer was 1.04, and 4 mL of dichloromethane was added to the flask. Thereafter, the reaction was carried out at room temperature for 6 hours, and then polymer 1H having the ionizable moiety of the present invention was obtained.

### Manufacturing Example 59

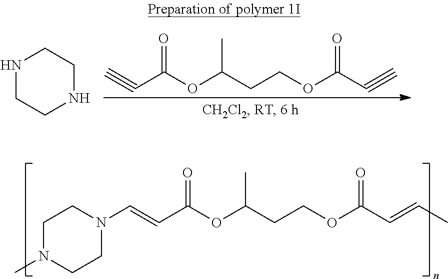

Alkynoate monomer I (500 mg) was placed in a round bottom flask, and then amine monomer 1 was added to the flask so that the molar ratio with the alkynoate monomer was 1.04, and 4 mL of dichloromethane was added to the flask. Thereafter, the reaction was carried out at room temperature for 6 hours, and then polymer 1I having the ionizable moiety of the present invention was obtained.

### Manufacturing Example 60

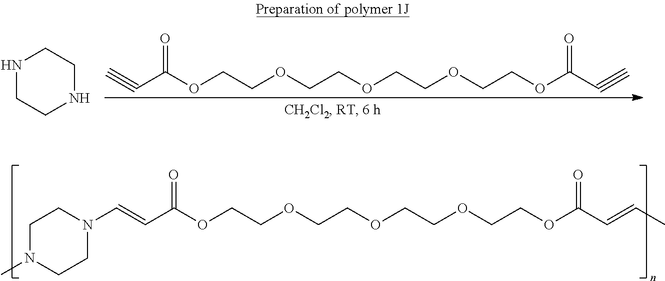

monomer 1 was added to the flask so that the molar ratio with the alkynoate monomer was 1.04, and 4 mL of dichloromethane was added to the flask. Thereafter, the reaction was carried out at room temperature for 6 hours, and then polymer 1J having the ionizable moiety of the present invention was obtained.

### Manufacturing Example 61

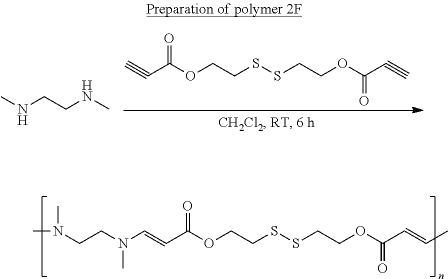

Alkynoate monomer F (500 mg) was placed in a round bottom flask, and then amine monomer 2 was added to the flask so that the molar ratio with the alkynoate monomer was 1.04, and 4 mL of dichloromethane was added to the flask. Thereafter, the reaction was carried out at room temperature for 6 hours, and then polymer 2F having the ionizable moiety of the present invention was obtained.

### Manufacturing Example 62

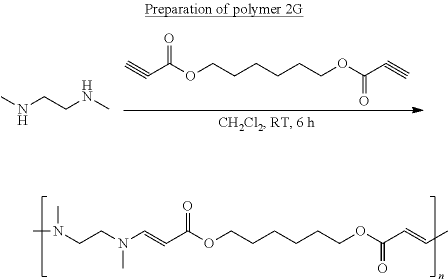

Alkynoate monomer G (500 mg) was placed in a round bottom flask, and then amine monomer 2 was added to the flask so that the molar ratio with the alkynoate monomer was 1.04, and 4 mL of dichloromethane was added to the flask. Thereafter, the reaction was carried out at room temperature for 6 hours, and then polymer 2G having the ionizable moiety of the present invention was obtained.

### Manufacturing Example 63

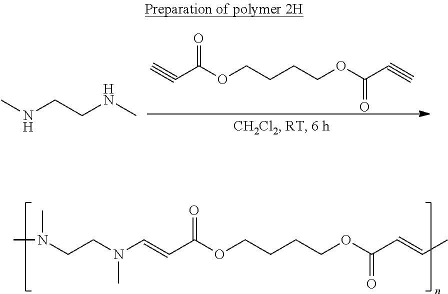

Alkynoate monomer H (500 mg) was placed in a round bottom flask, and then amine monomer 2 was added to the flask so that the molar ratio with the alkynoate monomer was 1.04, and 4 mL of dichloromethane was added to the flask. Thereafter, the reaction was carried out at room temperature for 6 hours, and then polymer 2H having the ionizable moiety of the present invention was obtained.

### Manufacturing Example 64

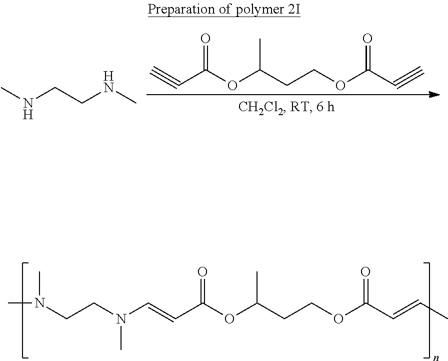

Alkynoate monomer I (500 mg) was placed in a round bottom flask, and then amine monomer 2 was added to the flask so that the molar ratio with the alkynoate monomer was 1.04, and 4 mL of dichloromethane was added to the flask. Thereafter, the reaction was carried out at room temperature for 6 hours, and then polymer 2I having the ionizable moiety of the present invention was obtained.

### Manufacturing Example 65

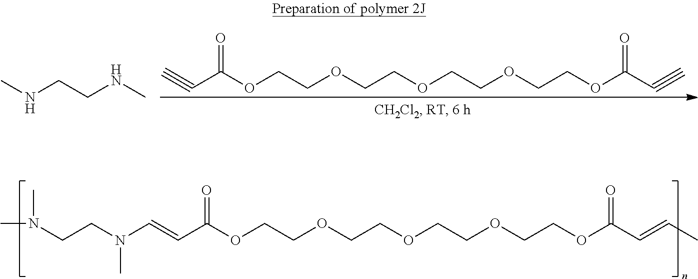

Alkynoate monomer J (500 mg) was placed in a round bottom flask, and then amine monomer 2 was added to the flask so that the molar ratio with the alkynoate monomer was 1.04, and 4 mL of dichloromethane was added to the flask. Thereafter, the reaction was carried out at room temperature for 6 hours, and then polymer 2J having the ionizable moiety of the present invention was obtained.

### Manufacturing Example 66

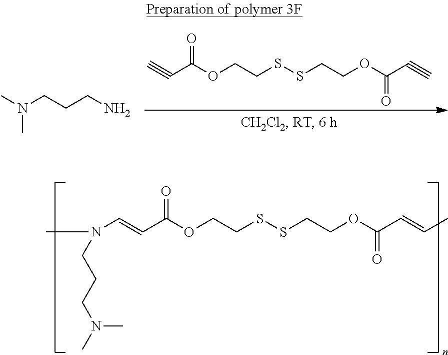

Alkynoate monomer F (500 mg) was placed in a round bottom flask, and then amine monomer 3 was added to the flask so that the molar ratio with the alkynoate monomer was 1.04, and 4 mL of dichloromethane was added to the flask. Thereafter, the reaction was carried out at room temperature for 6 hours, and then polymer 3F having the ionizable moiety of the present invention was obtained.

### Manufacturing Example 67

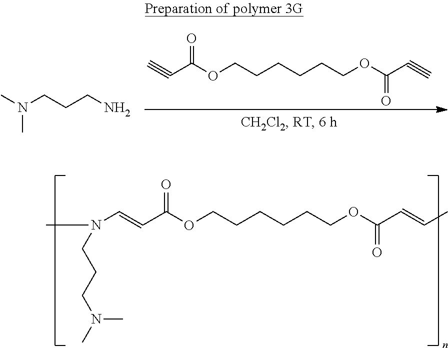

Alkynoate monomer G (500 mg) was placed in a round bottom flask, and then amine monomer 3 was added to the flask so that the molar ratio with the alkynoate monomer was 1.04, and 4 mL of dichloromethane was added to the flask. Thereafter, the reaction was carried out at room temperature for 6 hours, and then polymer 3G having the ionizable moiety of the present invention was obtained.

### Manufacturing Example 68

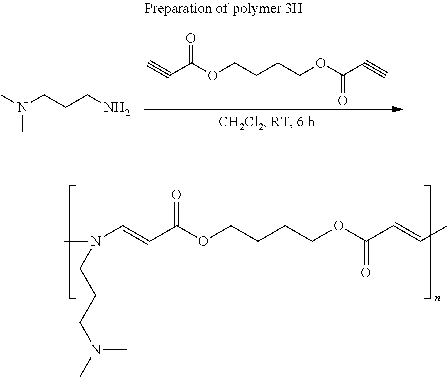

Alkynoate monomer H (500 mg) was placed in a round bottom flask, and then amine monomer 3 was added to the flask so that the molar ratio with the alkynoate monomer was 1.04, and 4 mL of dichloromethane was added to the flask. Thereafter, the reaction was carried out at room temperature for 6 hours, and then polymer 3H having the ionizable moiety of the present invention was obtained.

### Manufacturing Example 69

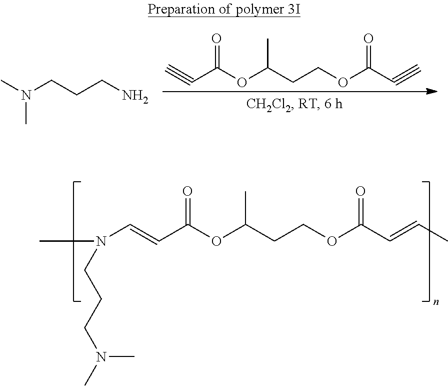

Alkynoate monomer I (500 mg) was placed in a round bottom flask, and then amine monomer 3 was added to the flask so that the molar ratio with the alkynoate monomer was 1.04, and 4 mL of dichloromethane was added to the flask. Thereafter, the reaction was carried out at room temperature for 6 hours, and then polymer 3I having the ionizable moiety of the present invention was obtained.

### Manufacturing Example 70

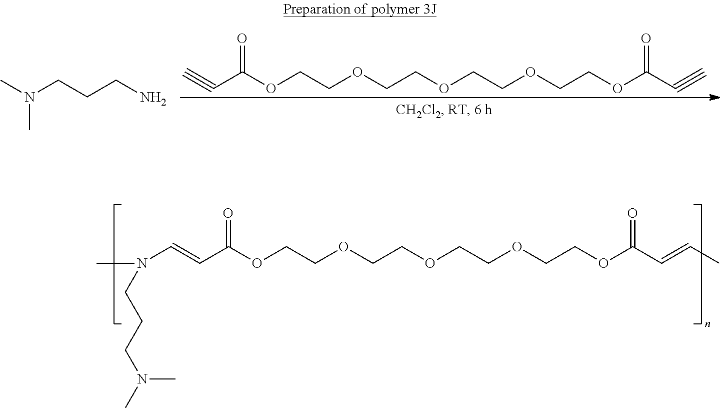

Alkynoate monomer J (500 mg) was placed in a round bottom flask, and then amine monomer 3 was added to the flask so that the molar ratio with the alkynoate monomer was 1.04, and 4 mL of dichloromethane was added to the flask. Thereafter, the reaction was carried out at room temperature for 6 hours, and then polymer 3J having the ionizable moiety of the present invention was obtained.

### Manufacturing Example 71

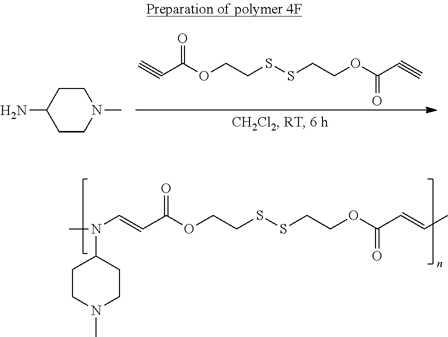

Alkynoate monomer F (500 mg) was placed in a round bottom flask, and then amine monomer 4 was added to the flask so that the molar ratio with the alkynoate monomer was 1.04, and 4 mL of dichloromethane was added to the flask. Thereafter, the reaction was carried out at room temperature for 6 hours, and then polymer 4F having the ionizable moiety of the present invention was obtained.

### Manufacturing Example 72

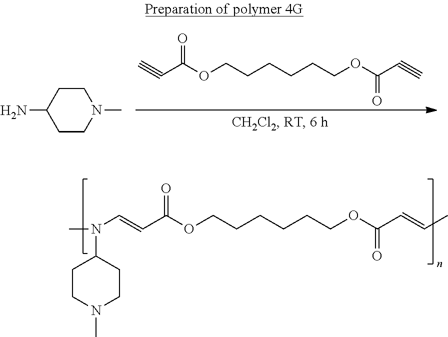

Alkynoate monomer G (500 mg) was placed in a round bottom flask, and then amine monomer 4 was added to the flask so that the molar ratio with the alkynoate monomer was 1.04, and 4 mL of dichloromethane was added to the flask. Thereafter, the reaction was carried out at room temperature for 6 hours, and then polymer 4G having the ionizable moiety of the present invention was obtained.

### Manufacturing Example 73

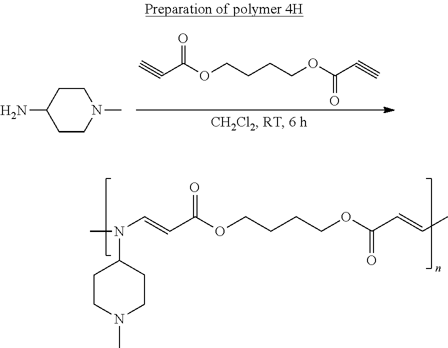

Alkynoate monomer H (500 mg) was placed in a round bottom flask, and then amine monomer 4 was added to the flask so that the molar ratio with the alkynoate monomer was 1.04, and 4 mL of dichloromethane was added to the flask. Thereafter, the reaction was carried out at room temperature for 6 hours, and then polymer 4H having the ionizable moiety of the present invention was obtained.

### Manufacturing Example 74

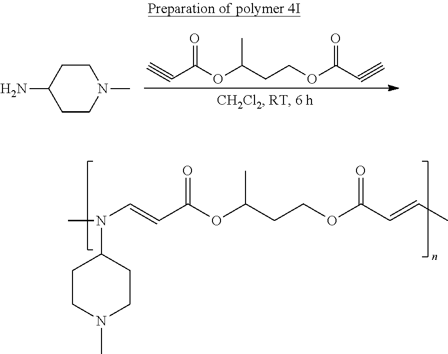

Alkynoate monomer I (500 mg) was placed in a round bottom flask, and then amine monomer 4 was added to the flask so that the molar ratio with the alkynoate monomer was 1.04, and 4 mL of dichloromethane was added to the flask. Thereafter, the reaction was carried out at room temperature for 6 hours, and then polymer 4I having the ionizable moiety of the present invention was obtained.

### Manufacturing Example 75

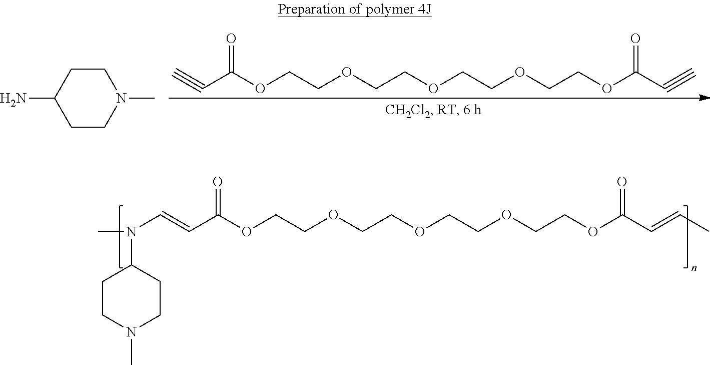

Alkynoate monomer J (500 mg) was placed in a round bottom flask, and then amine monomer 4 was added to the flask so that the molar ratio with the alkynoate monomer was 1.04, and 4 mL of dichloromethane was added to the flask. Thereafter, the reaction was carried out at room temperature for 6 hours, and then polymer 4J having the ionizable moiety of the present invention was obtained.

### Manufacturing Example 76

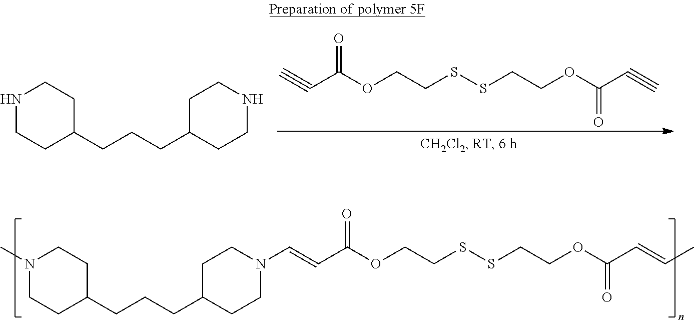

Alkynoate monomer F (500 mg) was placed in a round bottom flask, and then amine monomer 5 was added to the flask so that the molar ratio with the alkynoate monomer was 1.04, and 4 mL of dichloromethane was added to the flask. Thereafter, the reaction was carried out at room temperature for 6 hours, and then polymer 5F having the ionizable moiety of the present invention was obtained.

### Manufacturing Example 77

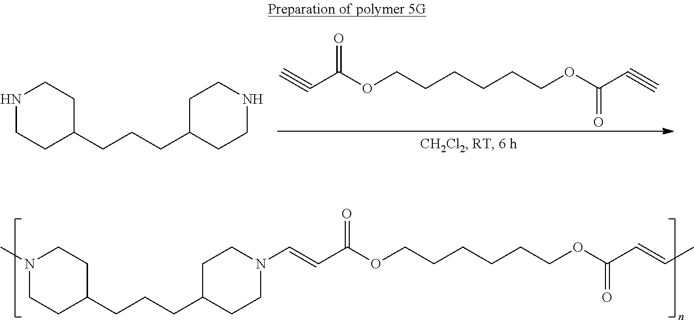

Alkynoate monomer G (500 mg) was placed in a round bottom flask, and then amine monomer 5 was added to the flask so that the molar ratio with the alkynoate monomer was 1.04, and 4 mL of dichloromethane was added to the flask. Thereafter, the reaction was carried out at room temperature for 6 hours, and then polymer 5G having the ionizable moiety of the present invention was obtained.

### Manufacturing Example 78

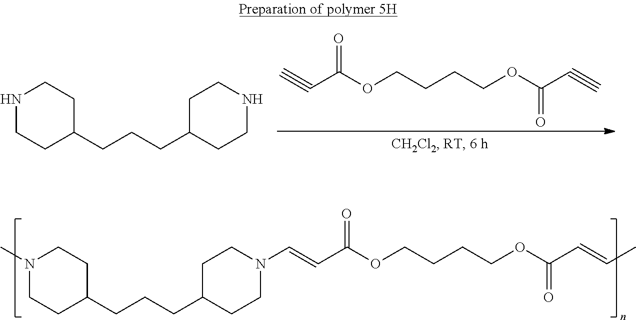

Alkynoate monomer H (500 mg) was placed in a round bottom flask, and then amine monomer 5 was added to the flask so that the molar ratio with the alkynoate monomer was 1.04, and 4 mL of dichloromethane was added to the flask. Thereafter, the reaction was carried out at room temperature for 6 hours, and then polymer 5H having the ionizable moiety of the present invention was obtained.

### Manufacturing Example 79

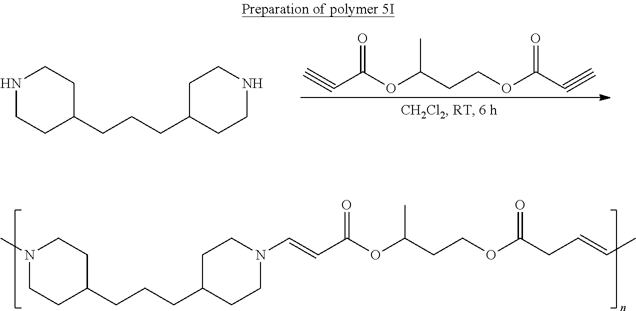

Alkynoate monomer I (500 mg) was placed in a round bottom flask, and then amine monomer 5 was added to the flask so that the molar ratio with the alkynoate monomer was 1.04, and 4 mL of dichloromethane was added to the flask. Thereafter, the reaction was carried out at room temperature for 6 hours, and then polymer 5I having the ionizable moiety of the present invention was obtained.

### Manufacturing Example 80

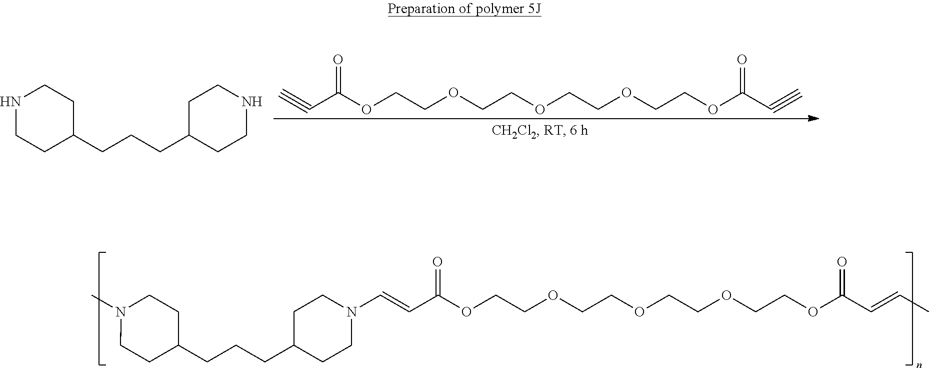

Alkynoate monomer J (500 mg) was placed in a round bottom flask, and then amine monomer 5 was added to the flask so that the molar ratio with the alkynoate monomer was 1.04, and 4 mL of dichloromethane was added to the flask. Thereafter, the reaction was carried out at room temperature for 6 hours, and then polymer 5J having the ionizable moiety of the present invention was obtained.

### Manufacturing Example 81

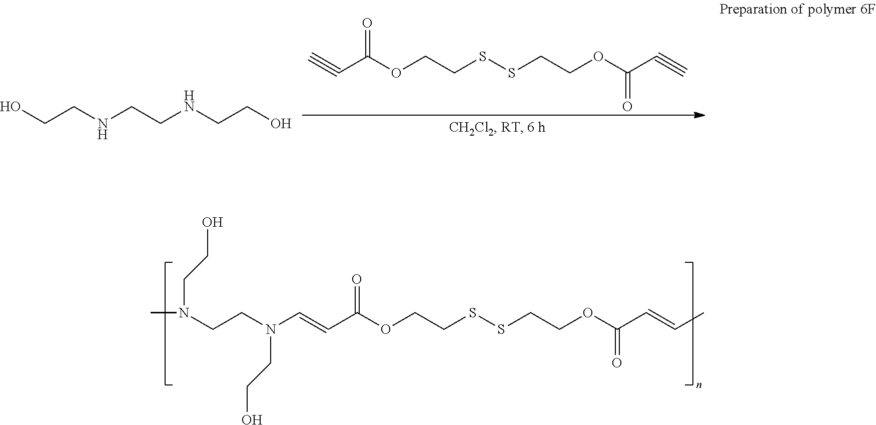

Alkynoate monomer F (500 mg) was placed in a round bottom flask, and then amine monomer 6 was added to the flask so that the molar ratio with the alkynoate monomer was 1.04, and 4 mL of dichloromethane was added to the flask. Thereafter, the reaction was carried out at room temperature for 6 hours, and then polymer 6F having the ionizable moiety of the present invention was obtained.

### Manufacturing Example 82

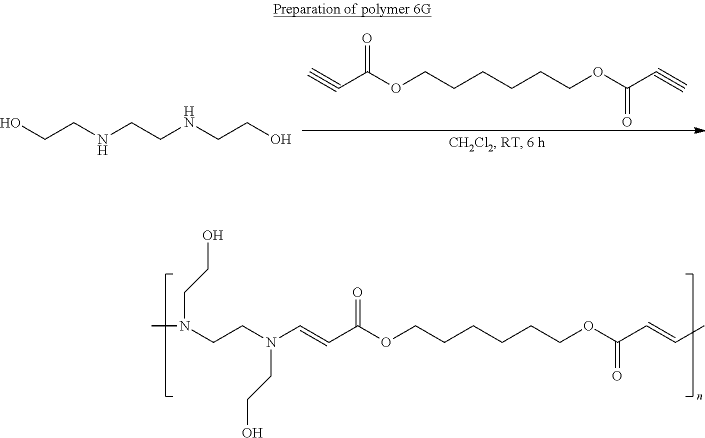

Alkynoate monomer G (500 mg) was placed in a round bottom flask, and then amine monomer 6 was added to the flask so that the molar ratio with the alkynoate monomer was 1.04, and 4 mL of dichloromethane was added to the flask. Thereafter, the reaction was carried out at room temperature for 6 hours, and then polymer 6G having the ionizable moiety of the present invention was obtained.

### Manufacturing Example 83

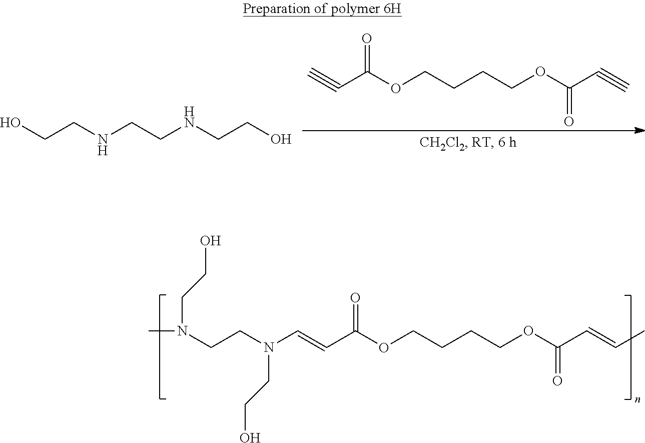

Alkynoate monomer H (500 mg) was placed in a round bottom flask, and then amine monomer 6 was added to the flask so that the molar ratio with the alkynoate monomer was 1.04, and 4 mL of dichloromethane was added to the flask. Thereafter, the reaction was carried out at room temperature for 6 hours, and then polymer 6H having the ionizable moiety of the present invention was obtained.

### Manufacturing Example 84

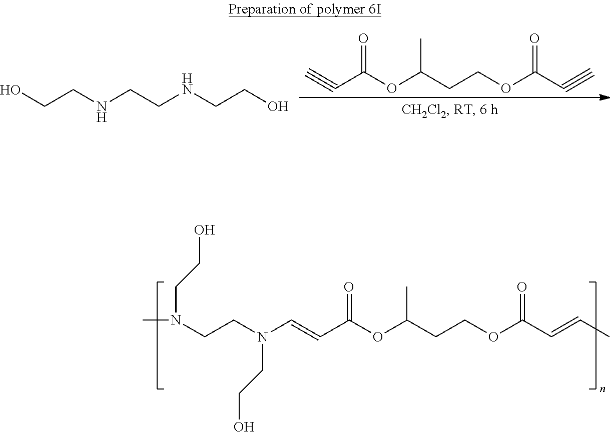

Alkynoate monomer I (500 mg) was placed in a round bottom flask, and then amine monomer 6 was added to the flask so that the molar ratio with the alkynoate monomer was 1.04, and 4 mL of dichloromethane was added to the flask. Thereafter, the reaction was carried out at room temperature for 6 hours, and then polymer 6I having the ionizable moiety of the present invention was obtained.

### Manufacturing Example 85

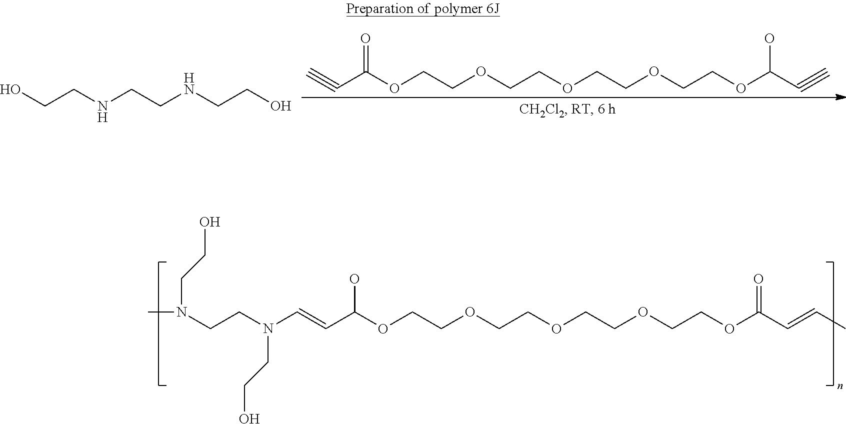

Alkynoate monomer J (500 mg) was placed in a round bottom flask, and then amine monomer 6 was added to the flask so that the molar ratio with the alkynoate monomer was 1.04, and 4 mL of dichloromethane was added to the flask. Thereafter, the reaction was carried out at room temperature for 6 hours, and then polymer 6J having the ionizable moiety of the present invention was obtained.

### Manufacturing Example 86

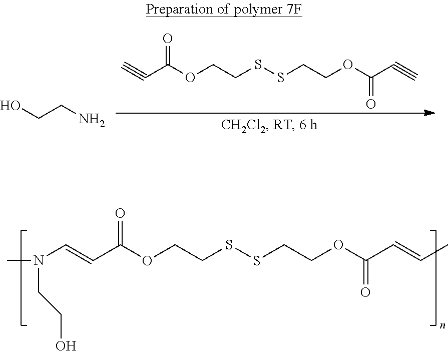

Alkynoate monomer F (500 mg) was placed in a round bottom flask, and then amine monomer 7 was added to the flask so that the molar ratio with the alkynoate monomer was 1.04, and 4 mL of dichloromethane was added to the flask. Thereafter, the reaction was carried out at room temperature for 6 hours, and then polymer 7F having the ionizable moiety of the present invention was obtained.

### Manufacturing Example 87

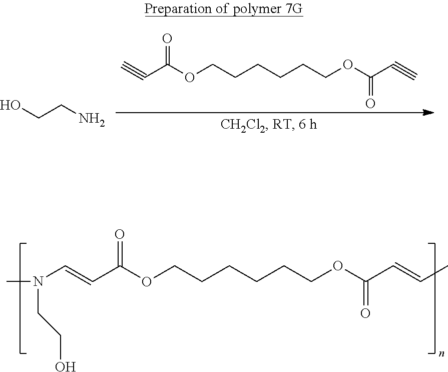

Alkynoate monomer G (500 mg) was placed in a round bottom flask, and then amine monomer 7 was added to the flask so that the molar ratio with the alkynoate monomer was 1.04, and 4 mL of dichloromethane was added to the flask. Thereafter, the reaction was carried out at room temperature for 6 hours, and then polymer 7G having the ionizable moiety of the present invention was obtained.

### Manufacturing Example 88

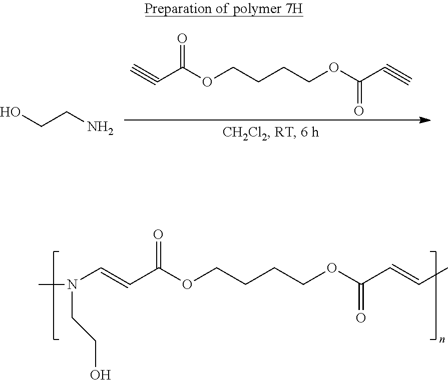

Alkynoate monomer H (500 mg) was placed in a round bottom flask, and then amine monomer 7 was added to the flask so that the molar ratio with the alkynoate monomer was 1.04, and 4 mL of dichloromethane was added to the flask. Thereafter, the reaction was carried out at room temperature for 6 hours, and then polymer 7H having the ionizable moiety of the present invention was obtained.

### Manufacturing Example 89

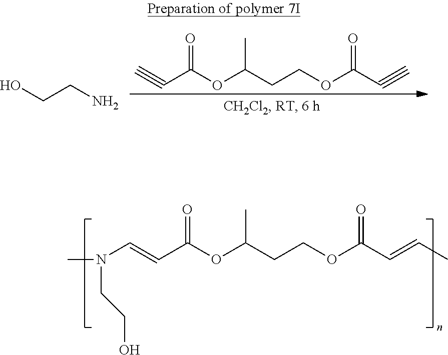

Alkynoate monomer I (500 mg) was placed in a round bottom flask, and then amine monomer 7 was added to the flask so that the molar ratio with the alkynoate monomer was 1.04, and 4 mL of dichloromethane was added to the flask. Thereafter, the reaction was carried out at room temperature for 6 hours, and then polymer 7I having the ionizable moiety of the present invention was obtained.

### Manufacturing Example 90

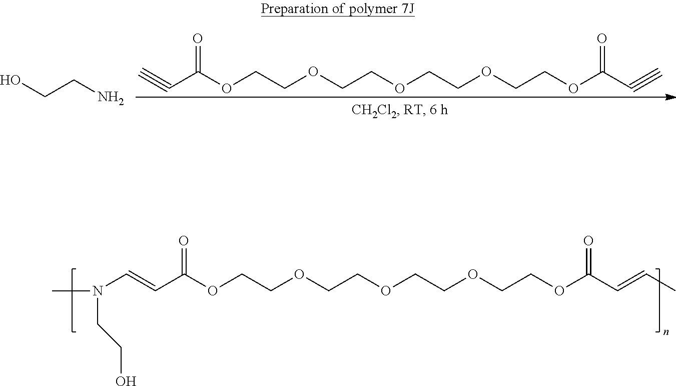

Alkynoate monomer J (500 mg) was placed in a round bottom flask, and then amine monomer 7 was added to the flask so that the molar ratio with the alkynoate monomer was 1.04, and 4 mL of dichloromethane was added to the flask. Thereafter, the reaction was carried out at room temperature for 6 hours, and then polymer 7J having the ionizable moiety of the present invention was obtained.

### Manufacturing Example 91

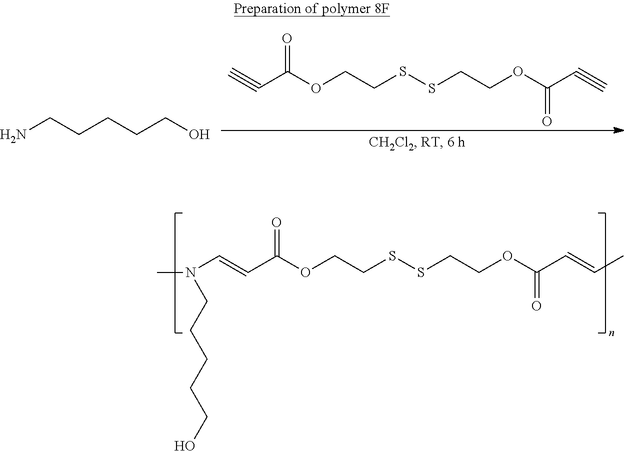

Alkynoate monomer F (500 mg) was placed in a round bottom flask, and then amine monomer 8 was added to the flask so that the molar ratio with the alkynoate monomer was 1.04, and 4 mL of dichloromethane was added to the flask. Thereafter, the reaction was carried out at room temperature for 6 hours, and then polymer 8F having the ionizable moiety of the present invention was obtained.

### Manufacturing Example 92

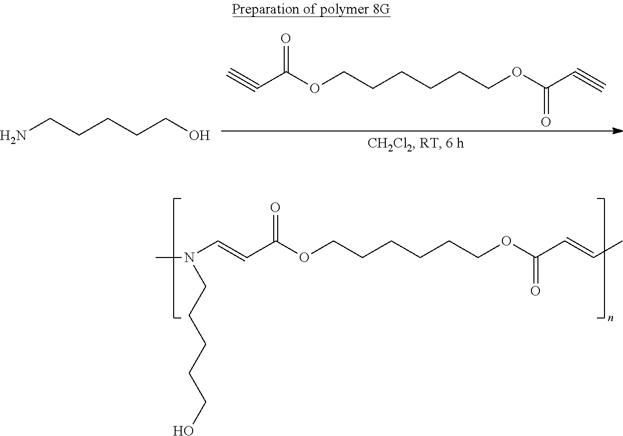

Alkynoate monomer G (500 mg) was placed in a round bottom flask, and then amine monomer 8 was added to the flask so that the molar ratio with the alkynoate monomer was 1.04, and 4 mL of dichloromethane was added to the flask. Thereafter, the reaction was carried out at room temperature for 6 hours, and then polymer 8G having the ionizable moiety of the present invention was obtained.

### Manufacturing Example 93

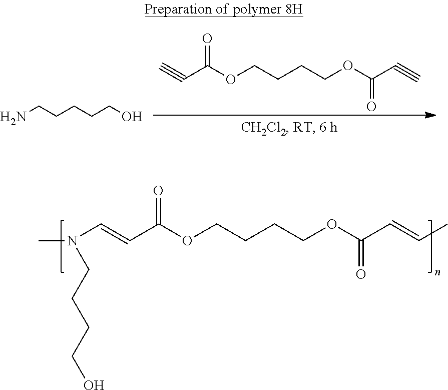

Alkynoate monomer H (500 mg) was placed in a round bottom flask, and then amine monomer 8 was added to the flask so that the molar ratio with the alkynoate monomer was 1.04, and 4 mL of dichloromethane was added to the flask. Thereafter, the reaction was carried out at room temperature for 6 hours, and then polymer 8H having the ionizable moiety of the present invention was obtained.

### Manufacturing Example 94

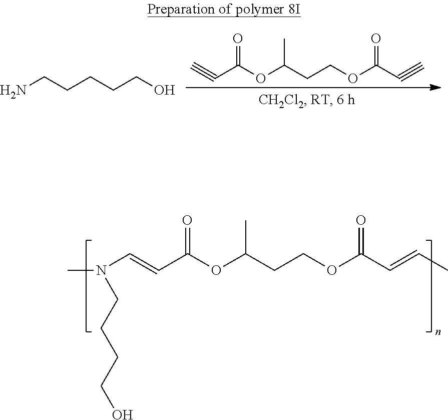

Alkynoate monomer I (500 mg) was placed in a round bottom flask, and then amine monomer 8 was added to the flask so that the molar ratio with the alkynoate monomer was 1.04, and 4 mL of dichloromethane was added to the flask. Thereafter, the reaction was carried out at room temperature for 6 hours, and then polymer 8I having the ionizable moiety of the present invention was obtained.

### Manufacturing Example 95

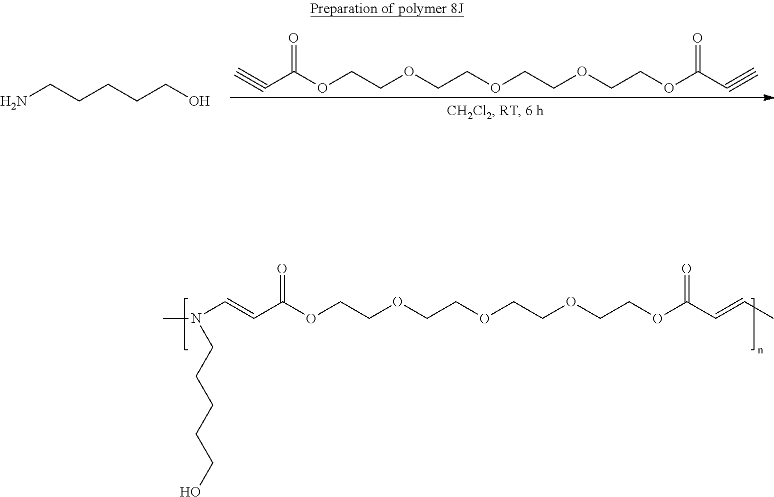

Alkynoate monomer J (500 mg) was placed in a round bottom flask, and then amine monomer 8 was added to the flask so that the molar ratio with the alkynoate monomer was 1.04, and 4 mL of dichloromethane was added to the flask. Thereafter, the reaction was carried out at room temperature for 6 hours, and then polymer 8J having the ionizable moiety of the present invention was obtained.

### Manufacturing Example 96

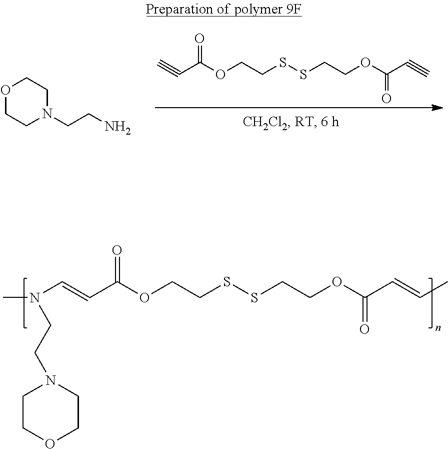

Alkynoate monomer F (500 mg) was placed in a round bottom flask, and then amine monomer 9 was added to the flask so that the molar ratio with the alkynoate monomer was 1.04, and 4 mL of dichloromethane was added to the flask. Thereafter, the reaction was carried out at room temperature for 6 hours, and then polymer 9F having the ionizable moiety of the present invention was obtained.

### Manufacturing Example 97

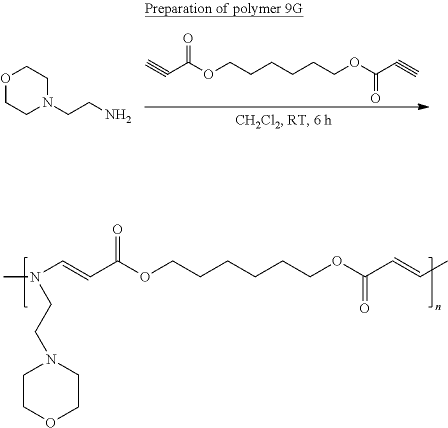

Alkynoate monomer G (500 mg) was placed in a round bottom flask, and then amine monomer 9 was added to the flask so that the molar ratio with the alkynoate monomer was 1.04, and 4 mL of dichloromethane was added to the flask. Thereafter, the reaction was carried out at room temperature for 6 hours, and then polymer 9G having the ionizable moiety of the present invention was obtained.

### Manufacturing Example 98

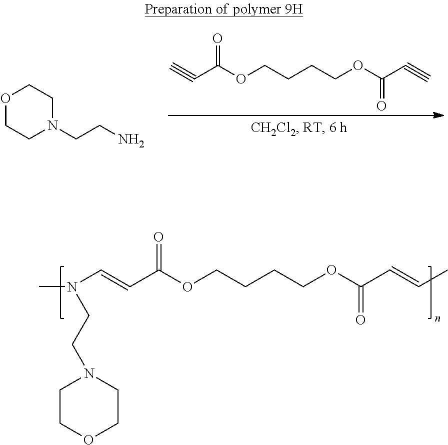

Alkynoate monomer H (500 mg) was placed in a round bottom flask, and then amine monomer 9 was added to the flask so that the molar ratio with the alkynoate monomer was 1.04, and 4 mL of dichloromethane was added to the flask. Thereafter, the reaction was carried out at room temperature for 6 hours, and then polymer 9H having the ionizable moiety of the present invention was obtained.

### Manufacturing Example 99

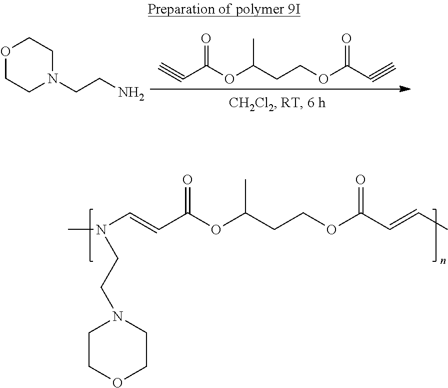

Alkynoate monomer I (500 mg) was placed in a round bottom flask, and then amine monomer 9 was added to the flask so that the molar ratio with the alkynoate monomer was 1.04, and 4 mL of dichloromethane was added to the flask. Thereafter, the reaction was carried out at room temperature for 6 hours, and then polymer 9I having the ionizable moiety of the present invention was obtained.

### Manufacturing Example 100

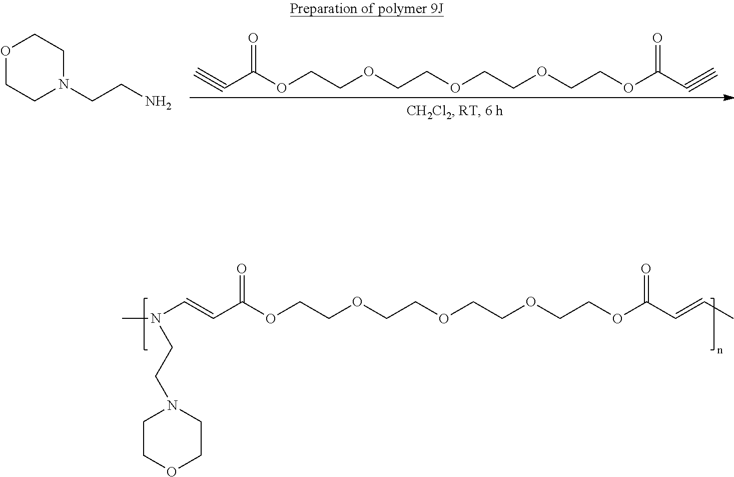

Alkynoate monomer J (500 mg) was placed in a round bottom flask, and then amine monomer 9 was added to the flask so that the molar ratio with the alkynoate monomer was 1.04, and 4 mL of dichloromethane was added to the flask. Thereafter, the reaction was carried out at room temperature for 6 hours, and then polymer 9J having the ionizable moiety of the present invention was obtained.

### Manufacturing Example 101

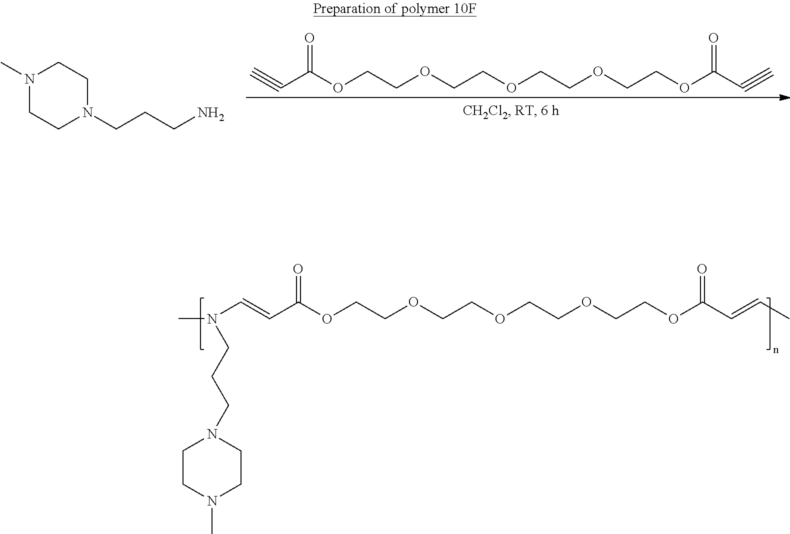

Alkynoate monomer F (500 mg) was placed in a round bottom flask, and then amine monomer 10 was added to the flask so that the molar ratio with the alkynoate monomer was 1.04, and 4 mL of dichloromethane was added to the flask. Thereafter, the reaction was carried out at room temperature for 6 hours, and then polymer 10F having the ionizable moiety of the present invention was obtained.

### Manufacturing Example 102

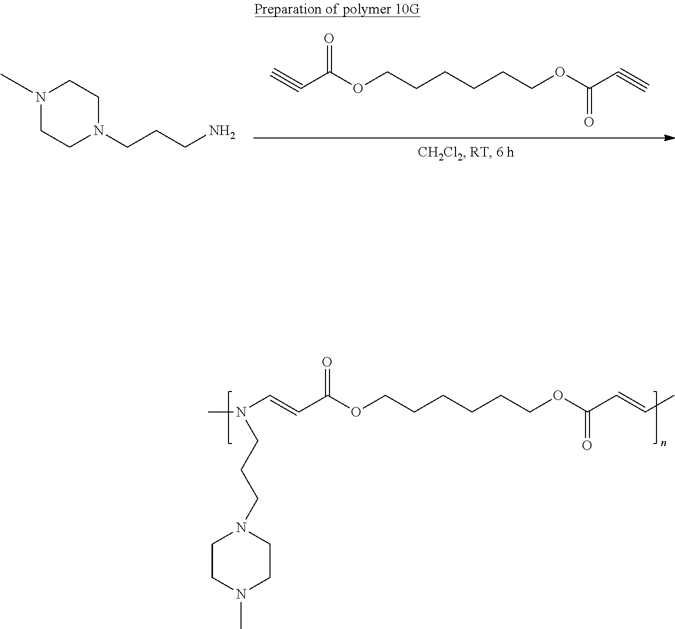

Alkynoate monomer G (500 mg) was placed in a round bottom flask, and then amine monomer 10 was added to the flask so that the molar ratio with the alkynoate monomer was 1.04, and 4 mL of dichloromethane was added to the flask. Thereafter, the reaction was carried out at room temperature for 6 hours, and then polymer 10G having the ionizable moiety of the present invention was obtained.

### Manufacturing Example 103

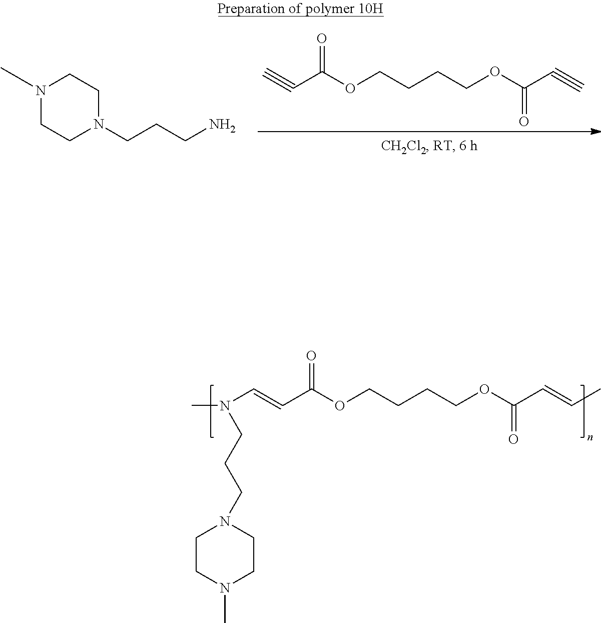

Alkynoate monomer H (500 mg) was placed in a round bottom flask, and then amine monomer 10 was added to the flask so that the molar ratio with the alkynoate monomer was 1.04, and 4 mL of dichloromethane was added to the flask. Thereafter, the reaction was carried out at room temperature for 6 hours, and then polymer 10H having the ionizable moiety of the present invention was obtained.

### Manufacturing Example 104

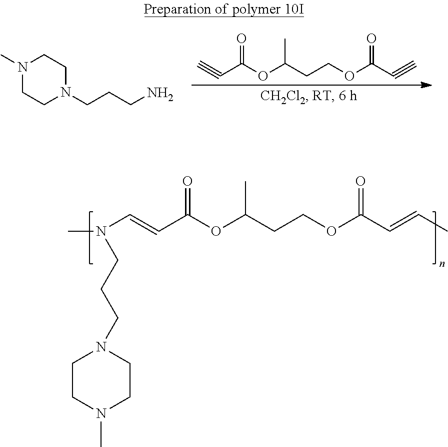

Alkynoate monomer I (500 mg) was placed in a round bottom flask, and then amine monomer 10 was added to the flask so that the molar ratio with the alkynoate monomer was 1.04, and 4 mL of dichloromethane was added to the flask. Thereafter, the reaction was carried out at room temperature for 6 hours, and then polymer 10I having the ionizable moiety of the present invention was obtained.

### Manufacturing Example 105

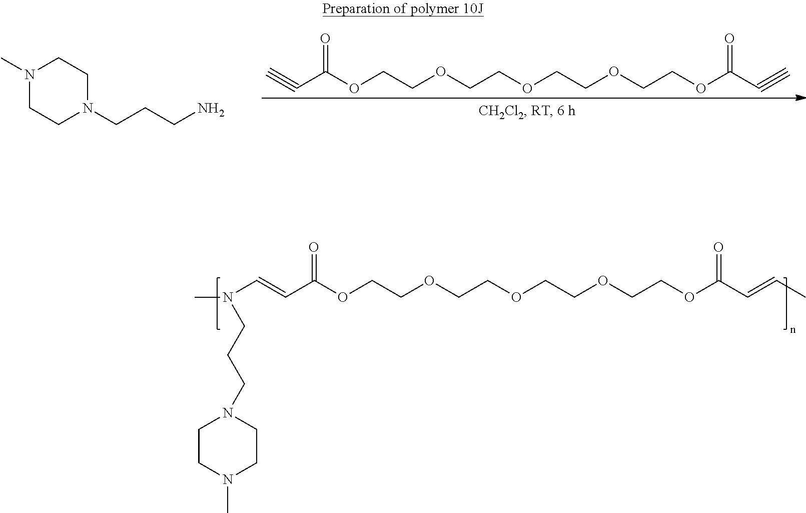

Alkynoate monomer J (500 mg) was placed in a round bottom flask, and then amine monomer 10 was added to the flask so that the molar ratio with the alkynoate monomer was 1.04, and 4 mL of dichloromethane was added to the flask. Thereafter, the reaction was carried out at room temperature for 6 hours, and then polymer 10J having the ionizable moiety of the present invention was obtained.

### Manufacturing Example 106

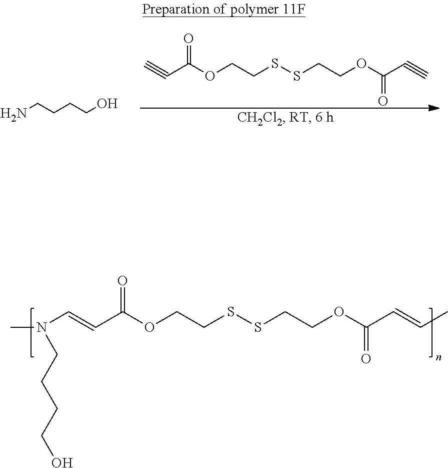

Alkynoate monomer F (500 mg) was placed in a round bottom flask, and then amine monomer 11 was added to the flask so that the molar ratio with the alkynoate monomer was 1.04, and 4 mL of dichloromethane was added to the flask. Thereafter, the reaction was carried out at room temperature for 6 hours, and then polymer 11F having the ionizable moiety of the present invention was obtained.

### Manufacturing Example 107

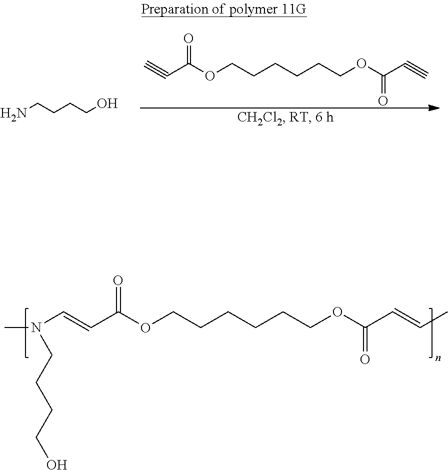

Alkynoate monomer G (500 mg) was placed in a round bottom flask, and then amine monomer 11 was added to the flask so that the molar ratio with the alkynoate monomer was 1.04, and 4 mL of dichloromethane was added to the flask. Thereafter, the reaction was carried out at room temperature for 6 hours, and then polymer 11G having the ionizable moiety of the present invention was obtained.

### Manufacturing Example 108

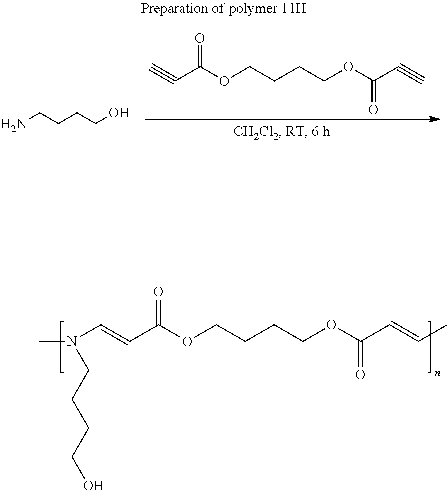

Alkynoate monomer H (500 mg) was placed in a round bottom flask, and then amine monomer 11 was added to the flask so that the molar ratio with the alkynoate monomer was 1.04, and 4 mL of dichloromethane was added to the flask. Thereafter, the reaction was carried out at room temperature for 6 hours, and then polymer 11H having the ionizable moiety of the present invention was obtained.

### Manufacturing Example 109

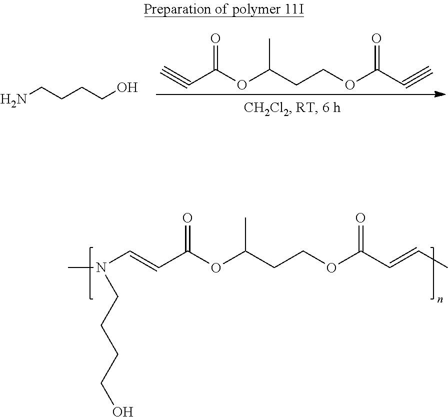

Alkynoate monomer I (500 mg) was placed in a round bottom flask, and then amine monomer 11 was added to the flask so that the molar ratio with the alkynoate monomer was 1.04, and 4 mL of dichloromethane was added to the flask. Thereafter, the reaction was carried out at room temperature for 6 hours, and then polymer 11I having the ionizable moiety of the present invention was obtained.

### Manufacturing Example 110

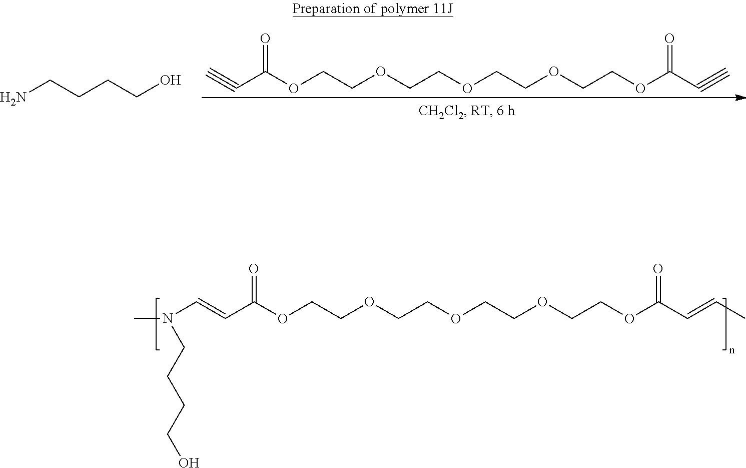

Alkynoate monomer J (500 mg) was placed in a round bottom flask, and then amine monomer 11 was added to the flask so that the molar ratio with the alkynoate monomer was 1.04, and 4 mL of dichloromethane was added to the flask. Thereafter, the reaction was carried out at room temperature for 6 hours, and then polymer 11J having the ionizable moiety of the present invention was obtained.

### Manufacturing Example 111

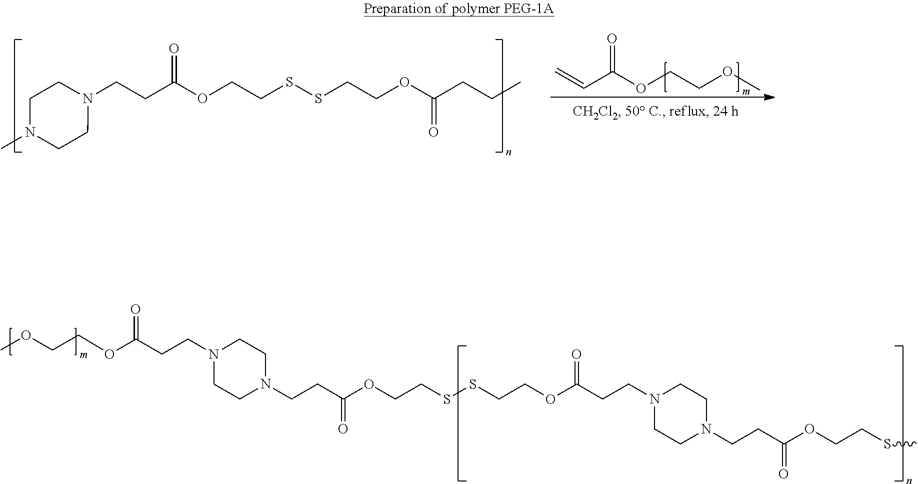

Polymer 1A prepared in the same manner as in Manufacturing Example 1 above and 648.8 mg of PEG-acrylate were dissolved in CH₂Cl₂ to a concentration of 1000 mg/ml and added to a flask. The reaction was carried out at 50° C. for 24 hours with reflux, and then PEG-1A was obtained.

### Manufacturing Example 112

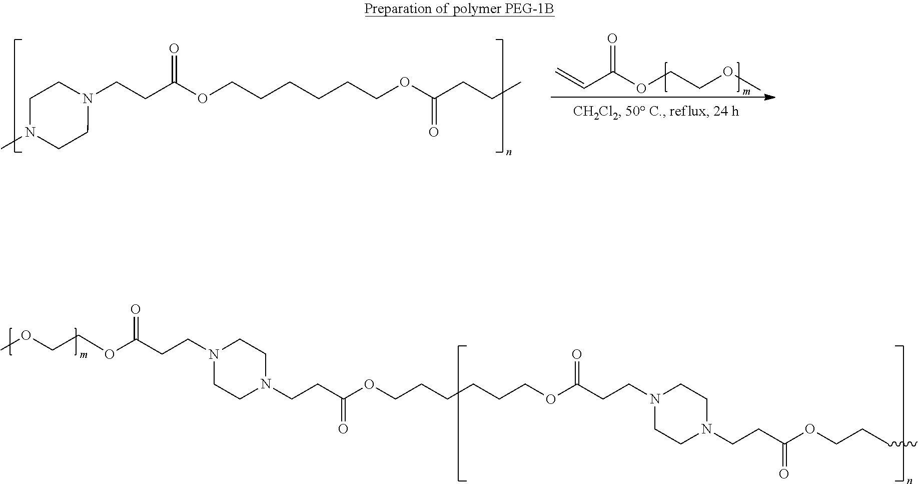

Polymer 1B prepared in the same manner as in Manufacturing Example 2 above and 752.3 mg of PEG-acrylate were dissolved in CH₂Cl₂ to a concentration of 1000 mg/ml and added to a flask. The reaction was carried out at 50° C. for 24 hours with reflux, and then PEG-1B was obtained.

### Manufacturing Example 113

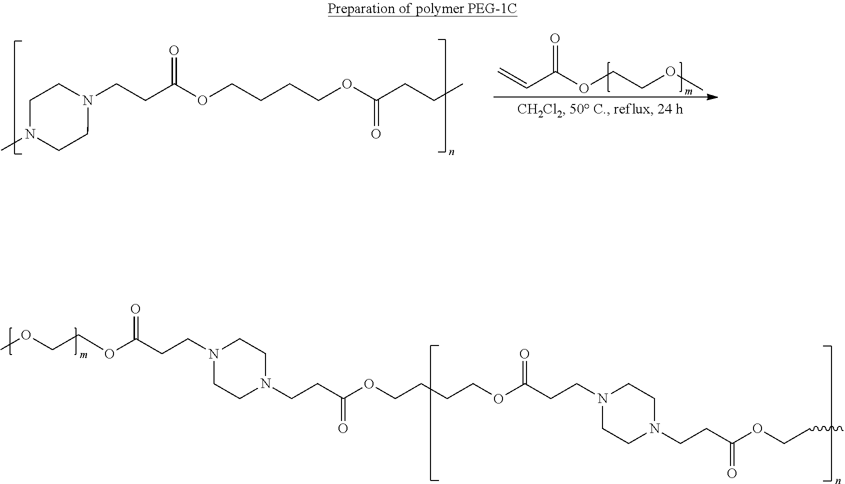

Polymer 1C prepared in the same manner as in Manufacturing Example 3 above and 858.7 mg of PEG-acrylate were dissolved in CH₂Cl₂ to a concentration of 1000 mg/ml and added to a flask. The reaction was carried out at 50° C. for 24 hours with reflux, and then PEG-1C was obtained.

### Manufacturing Example 114

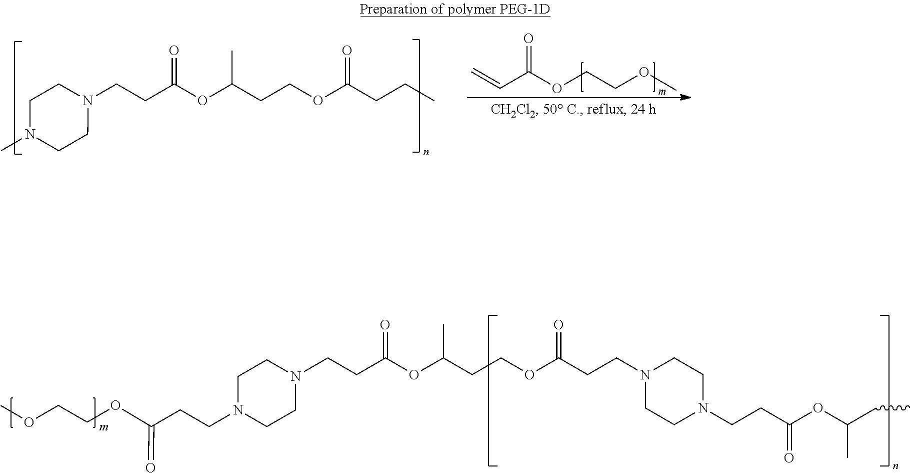

Polymer 1D prepared in the same manner as in Manufacturing Example 4 above and 858.7 mg of PEG-acrylate were dissolved in CH₂Cl₂ to a concentration of 1000 mg/ml and added to a flask. The reaction was carried out at 50° C. for 24 hours with reflux, and then PEG-1D was obtained.

### Manufacturing Example 115

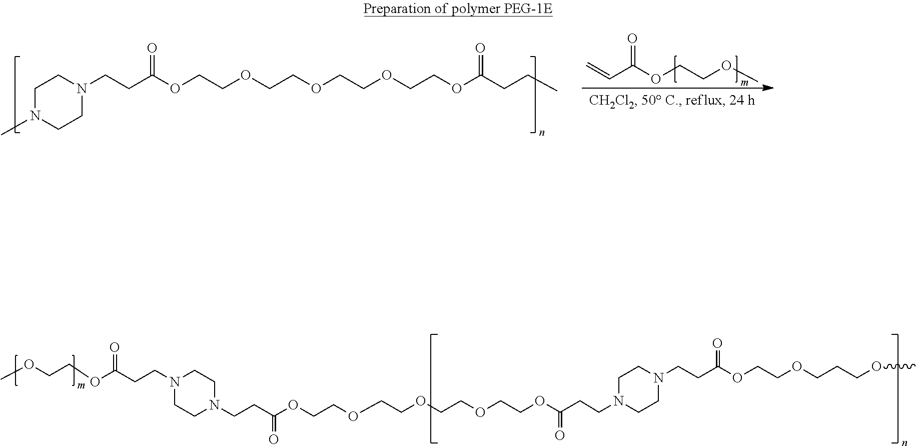

Polymer 1E prepared in the same manner as in Manufacturing Example 5 above and 563.0 mg of PEG-acrylate were dissolved in CH₂Cl₂ to a concentration of 2500 mg/ml and added to a flask. The reaction was carried out at 50° C. for 24 hours with reflux, and then PEG-1E was obtained.

### Manufacturing Example 116

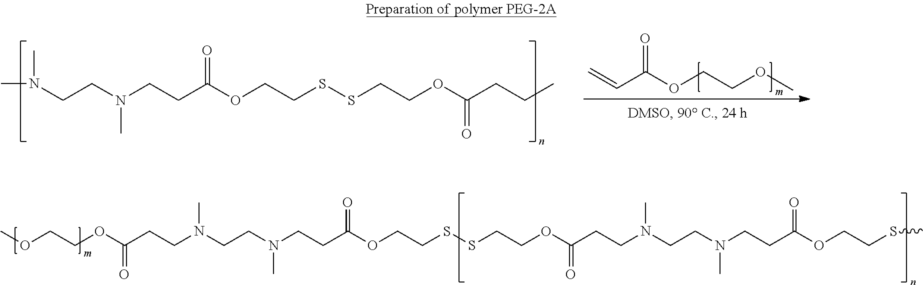

Polymer 2A prepared in the same manner as in Manufacturing Example 6 above and 648.8 mg of PEG-acrylate were dissolved in DMSO to a concentration of 1000 mg/ml and added to a flask. The reaction was carried out at 90° C. for 24 hours, and then PEG-2A was obtained.

### Manufacturing Example 117

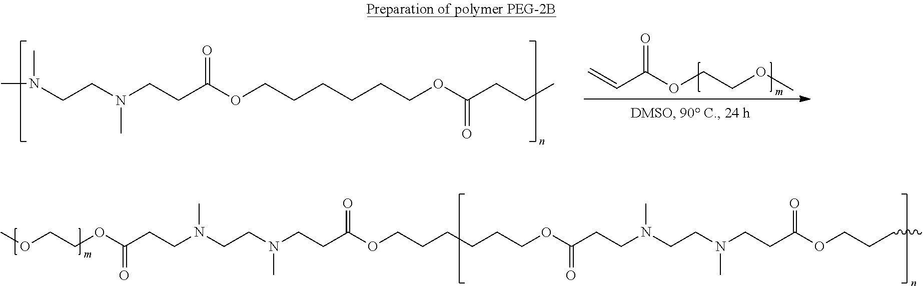

Polymer 2B prepared in the same manner as in Manufacturing Example 7 above and 752.3 mg of PEG-acrylate were dissolved in DMSO to a concentration of 1000 mg/ml and added to a flask. The reaction was carried out at 90° C. for 24 hours, and then PEG-2B was obtained.

### Manufacturing Example 118

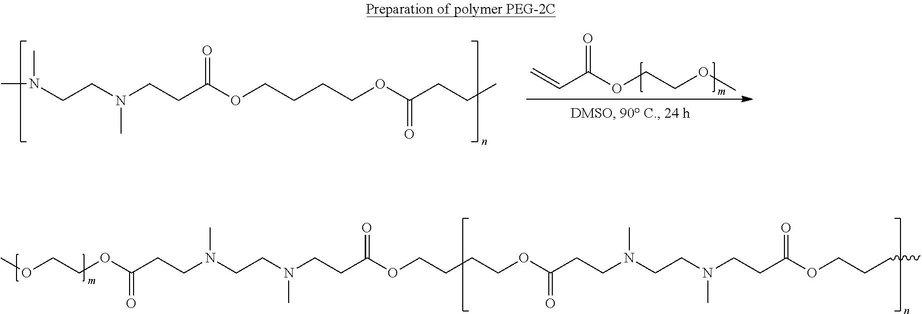

Polymer 2C prepared in the same manner as in Manufacturing Example 8 above and 858.7 mg of PEG-acrylate were dissolved in DMSO to a concentration of 1000 mg/ml and added to a flask. The reaction was carried out at 90° C. for 24 hours, and then PEG-2C was obtained.

### Manufacturing Example 119

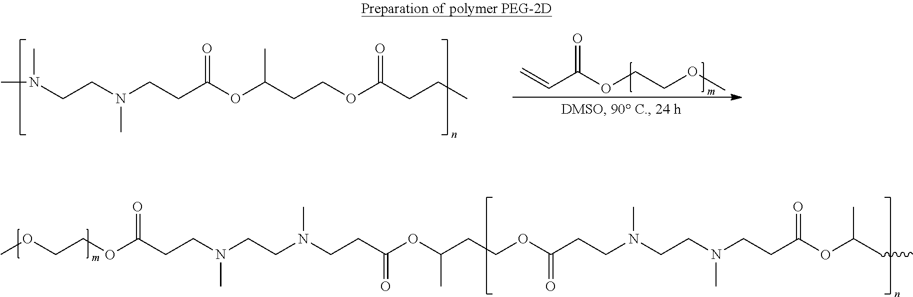

Polymer 2D prepared in the same manner as in Manufacturing Example 9 above and 858.7 mg of PEG-acrylate were dissolved in DMSO to a concentration of 1000 mg/ml and added to a flask. The reaction was carried out at 90° C. for 24 hours, and then PEG-2D was obtained.

### Manufacturing Example 120

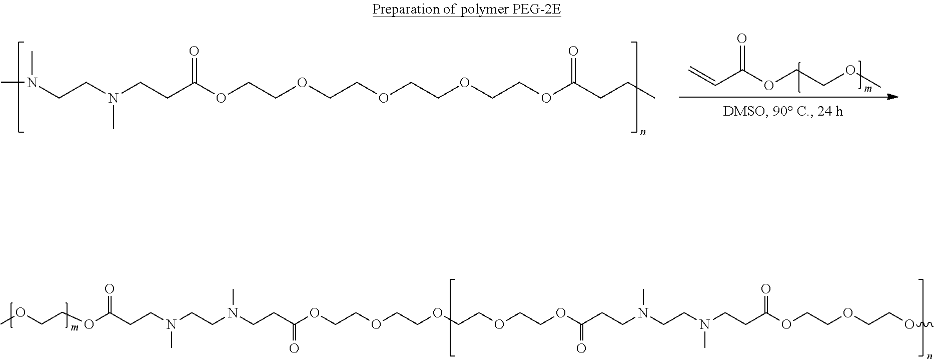

Polymer 2E prepared in the same manner as in Manufacturing Example 10 above and 563.0 mg of PEG-acrylate were dissolved in DMSO to a concentration of 2500 mg/ml and added to a flask. The reaction was carried out at 90° C. for 24 hours, and then PEG-2E was obtained.

### Manufacturing Example 121

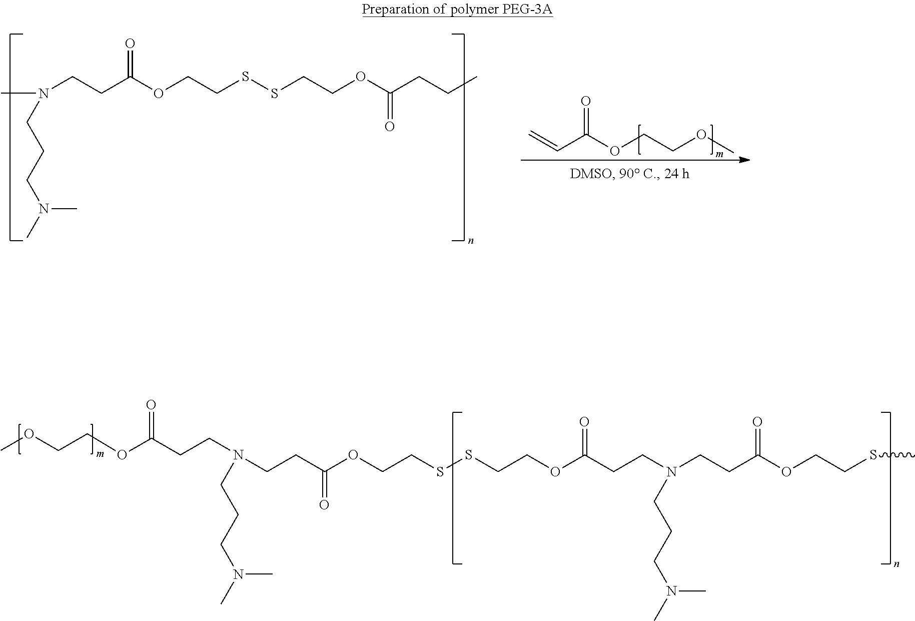

Polymer 3A prepared in the same manner as in Manufacturing Example 11 above and 648.8 mg of PEG-acrylate were dissolved in DMSO to a concentration of 1000 mg/ml and added to a flask. The reaction was carried out at 90° C. for 24 hours, and then PEG-3A was obtained.

### Manufacturing Example 122

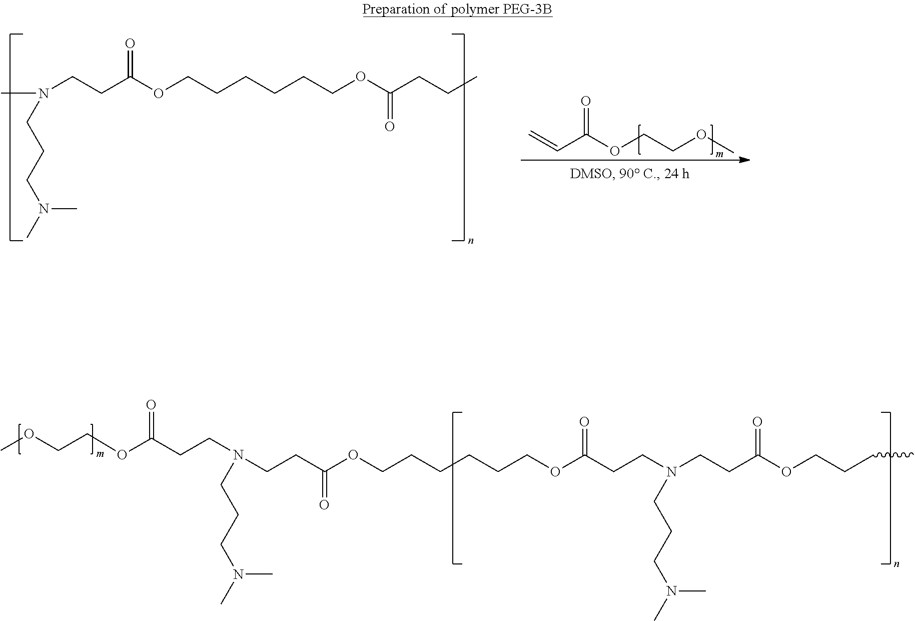

Polymer 3B prepared in the same manner as in Manufacturing Example 12 above and 752.3 mg of PEG-acrylate were dissolved in DMSO to a concentration of 1000 mg/ml and added to a flask. The reaction was carried out at 90° C. for 24 hours, and then PEG-3B was obtained.

### Manufacturing Example 123

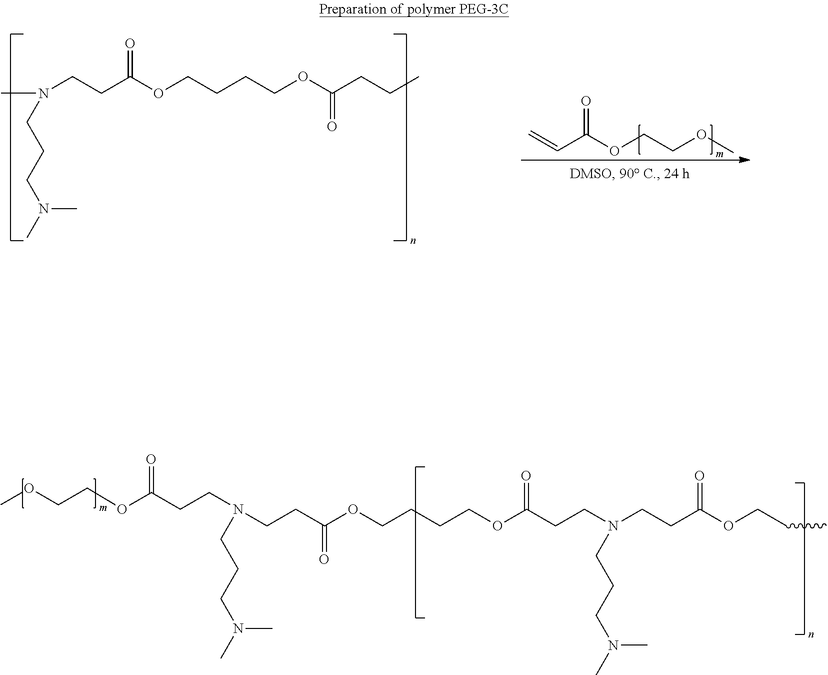

Polymer 3C prepared in the same manner as in Manufacturing Example 13 above and 858.7 mg of PEG-acrylate were dissolved in DMSO to a concentration of 1000 mg/ml and added to a flask. The reaction was carried out at 90° C. for 24 hours, and then PEG-3C was obtained.

### Manufacturing Example 124

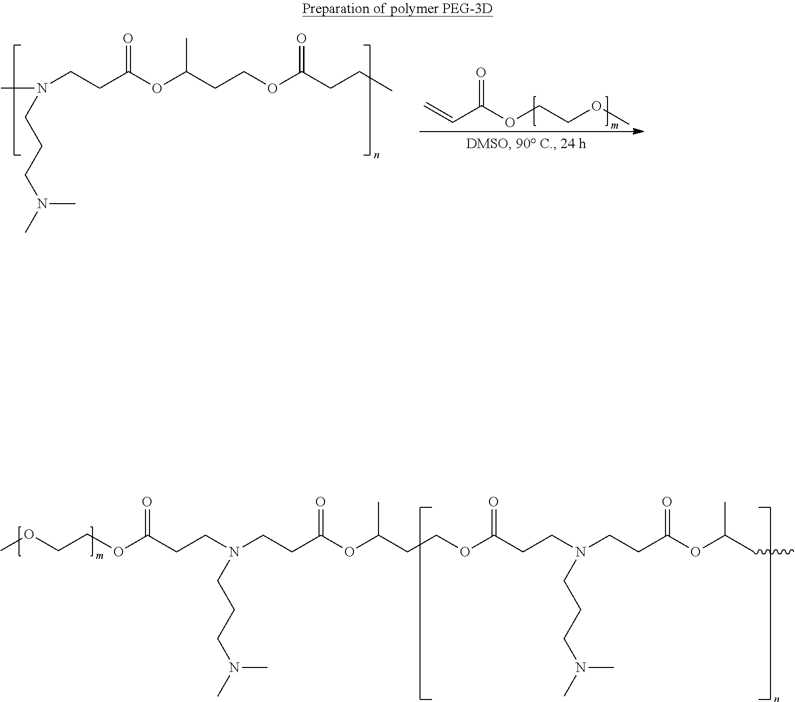

Polymer 3D prepared in the same manner as in Manufacturing Example 14 above and 858.7 mg of PEG-acrylate were dissolved in DMSO to a concentration of 1000 mg/ml and added to a flask. The reaction was carried out at 90° C. for 24 hours, and then PEG-3D was obtained.

### Manufacturing Example 125

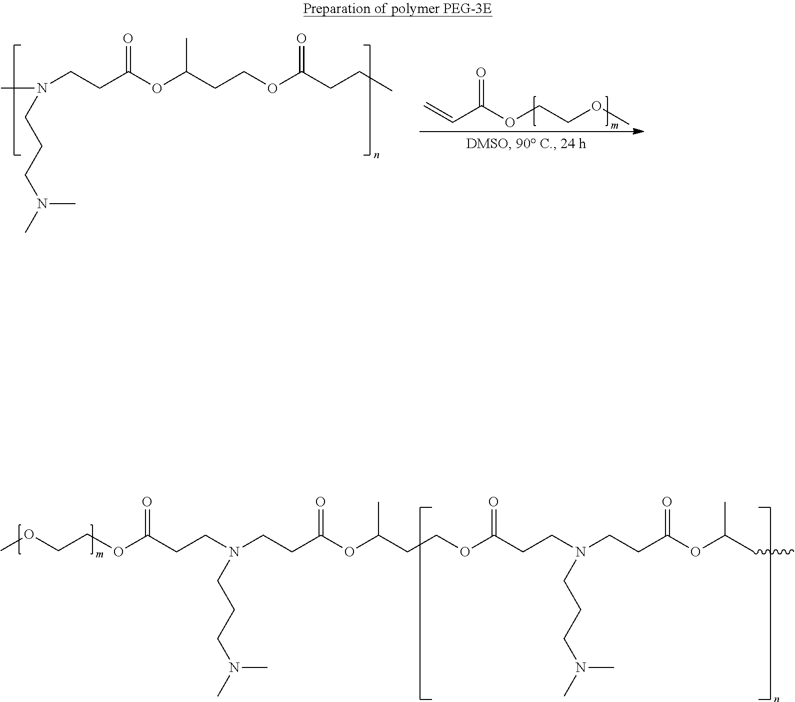

Polymer 3E prepared in the same manner as in Manufacturing Example 15 above and 563.0 mg of PEG-acrylate were dissolved in DMSO to a concentration of 2500 mg/ml and added to a flask. The reaction was carried out at 90° C. for 24 hours, and then PEG-3E was obtained.

### Manufacturing Example 126

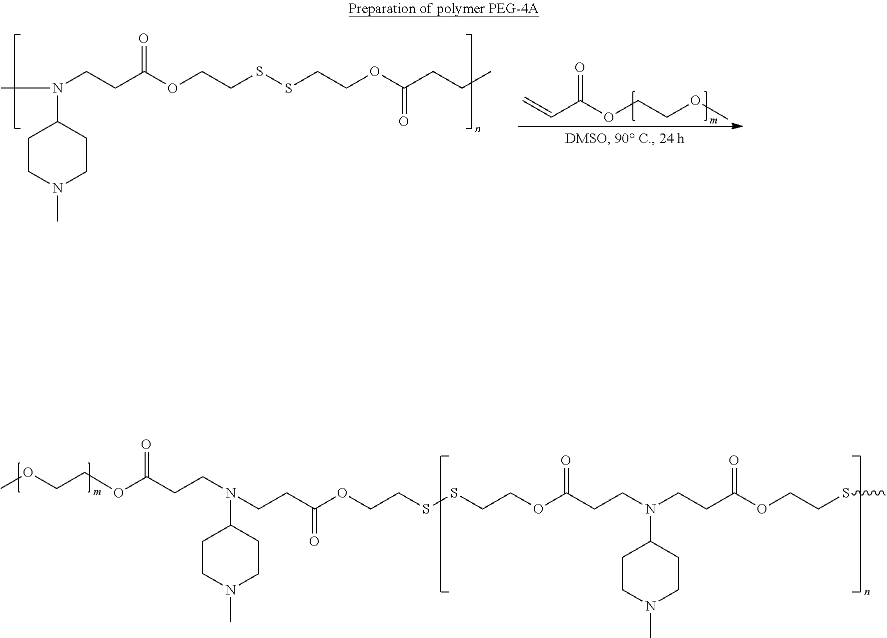

Polymer 4A prepared in the same manner as in Manufacturing Example 16 above and 648.8 mg of PEG-acrylate were dissolved in DMSO to a concentration of 1000 mg/ml and added to a flask. The reaction was carried out at 90° C. for 24 hours, and then PEG-4A was obtained.

### Manufacturing Example 127

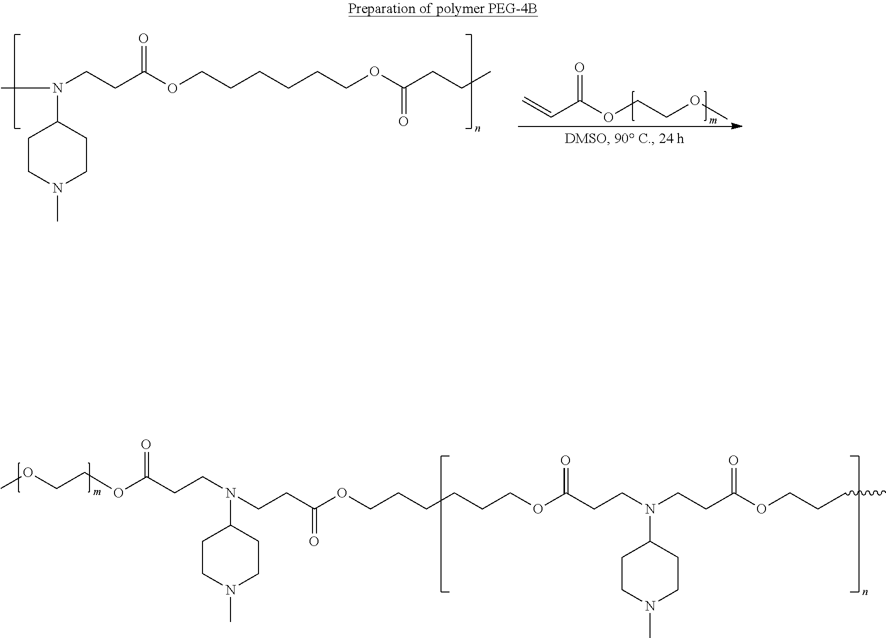

Polymer 4B prepared in the same manner as in Manufacturing Example 17 above and 752.3 mg of PEG-acrylate were dissolved in DMSO to a concentration of 1000 mg/ml and added to a flask. The reaction was carried out at 90° C. for 24 hours, and then PEG-4B was obtained.

### Manufacturing Example 128

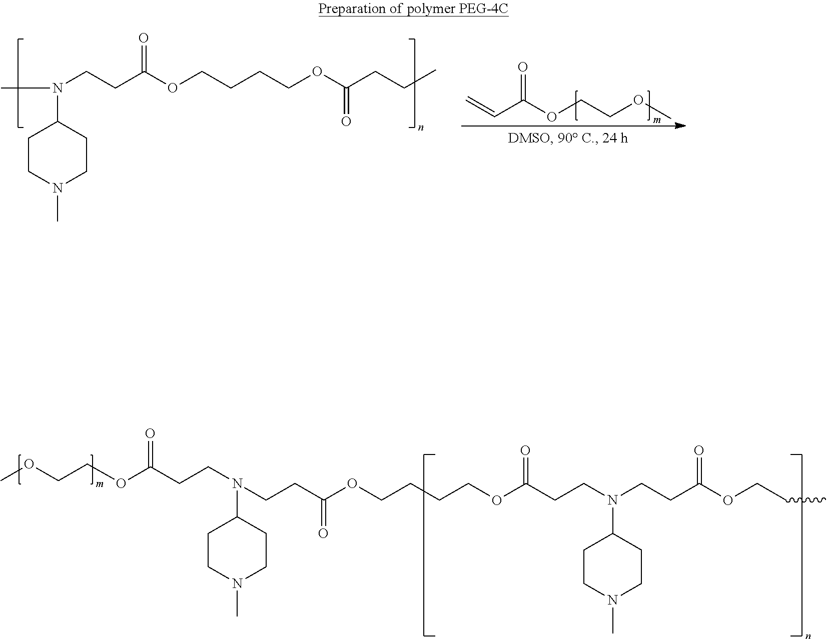

Polymer 4C prepared in the same manner as in Manufacturing Example 18 above and 858.7 mg of PEG-acrylate were dissolved in DMSO to a concentration of 1000 mg/ml and added to a flask. The reaction was carried out at 90° C. for 24 hours, and then PEG-4C was obtained.

### Manufacturing Example 129

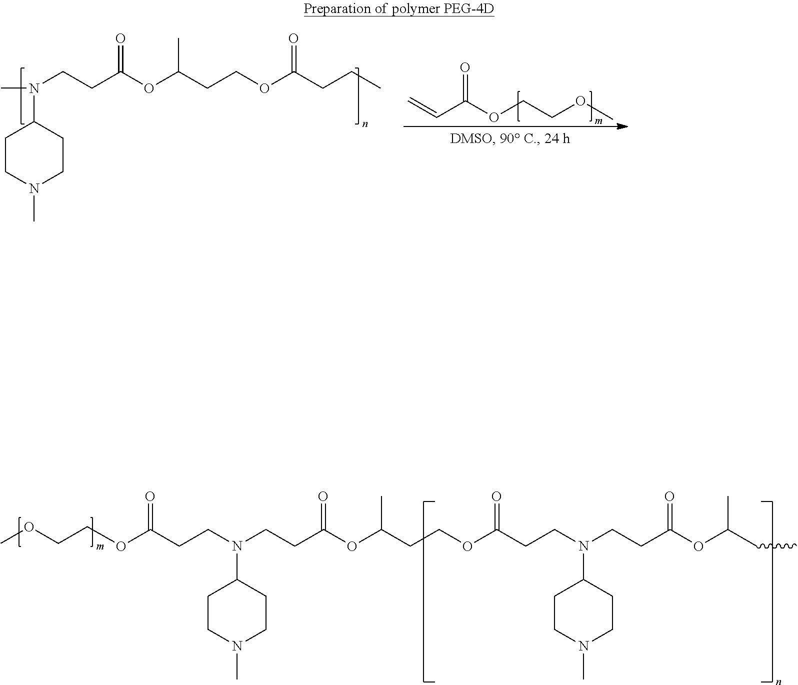

Polymer 4D prepared in the same manner as in Manufacturing Example 19 above and 858.7 mg of PEG-acrylate were dissolved in DMSO to a concentration of 1000 mg/ml and added to a flask. The reaction was carried out at 90° C. for 24 hours, and then PEG-4D was obtained.

### Manufacturing Example 130

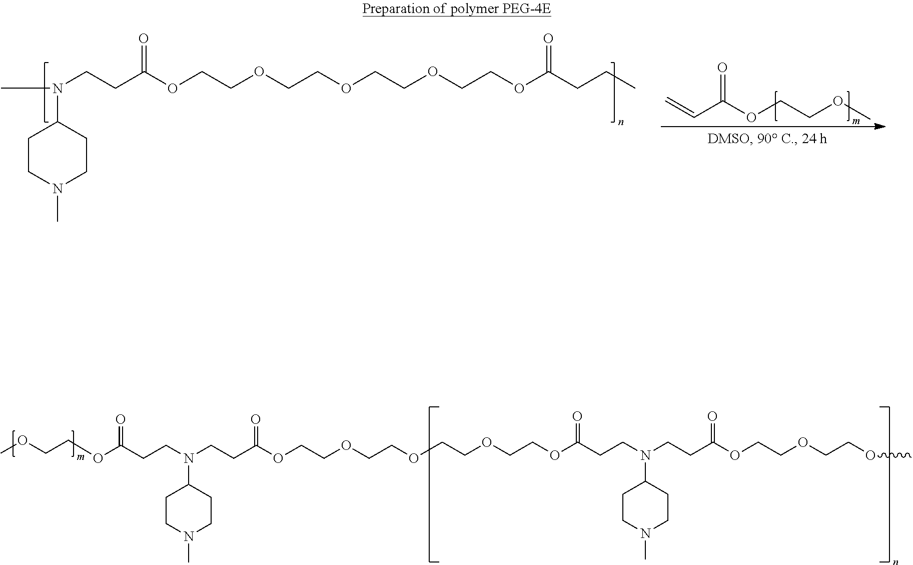

Polymer 4E prepared in the same manner as in Manufacturing Example 20 above and 563.0 mg of PEG-acrylate were dissolved in DMSO to a concentration of 2500 mg/ml and added to a flask. The reaction was carried out at 90° C. for 24 hours, and then PEG-4E was obtained.

### Manufacturing Example 131

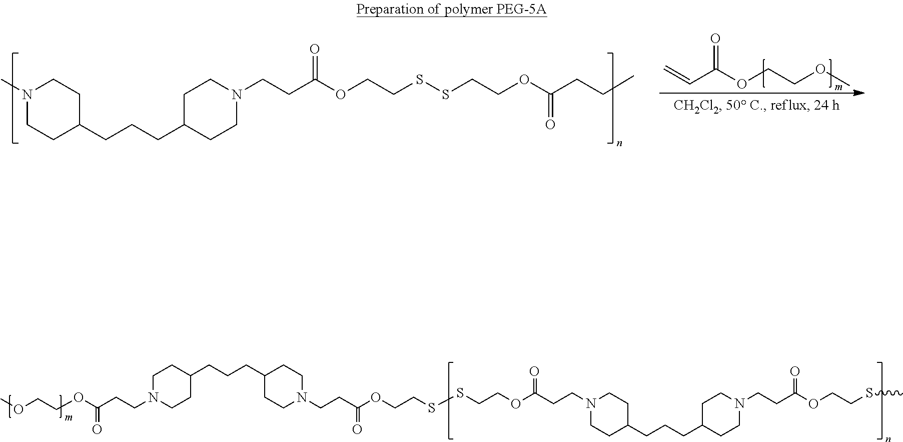

Polymer 5A prepared in the same manner as in Manufacturing Example 21 above and 648.8 mg of PEG-acrylate were dissolved in CH₂Cl₂ to a concentration of 1000 mg/ml and added to a flask. The reaction was carried out at 50° C. for 24 hours with reflux, and then PEG-5A was obtained.

### Manufacturing Example 132

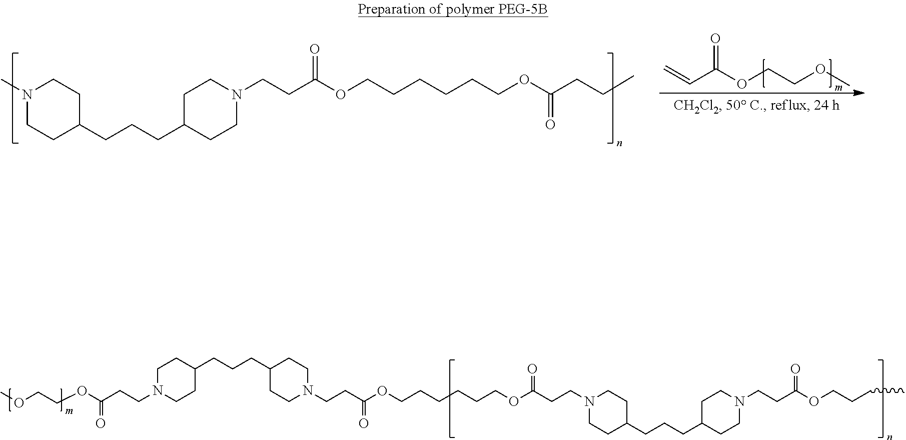

Polymer 5B prepared in the same manner as in Manufacturing Example 22 above and 752.3 mg of PEG-acrylate were dissolved in CH₂Cl₂ to a concentration of 1000 mg/ml and added to a flask. The reaction was carried out at 50° C. for 24 hours with reflux, and then PEG-5B was obtained.

### Manufacturing Example 133

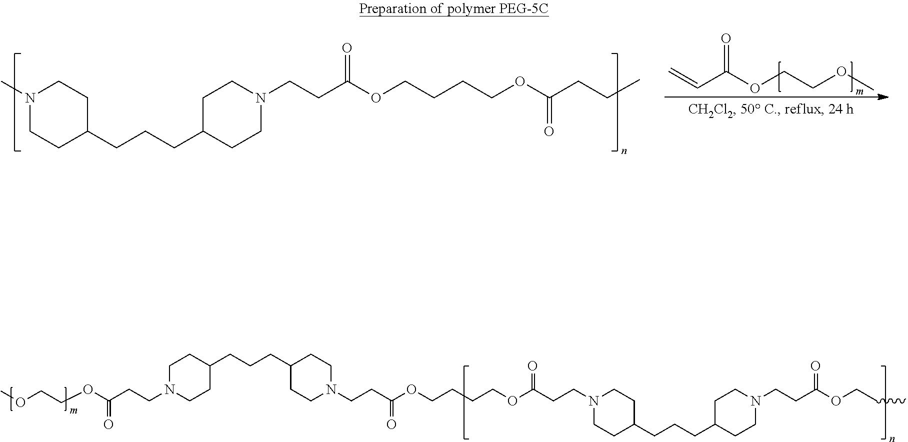

Polymer 5C prepared in the same manner as in Manufacturing Example 23 above and 858.7 mg of PEG-acrylate were dissolved in CH₂Cl₂ to a concentration of 1000 mg/ml and added to a flask. The reaction was carried out at 50° C. for 24 hours with reflux, and then PEG-5C was obtained.

### Manufacturing Example 134

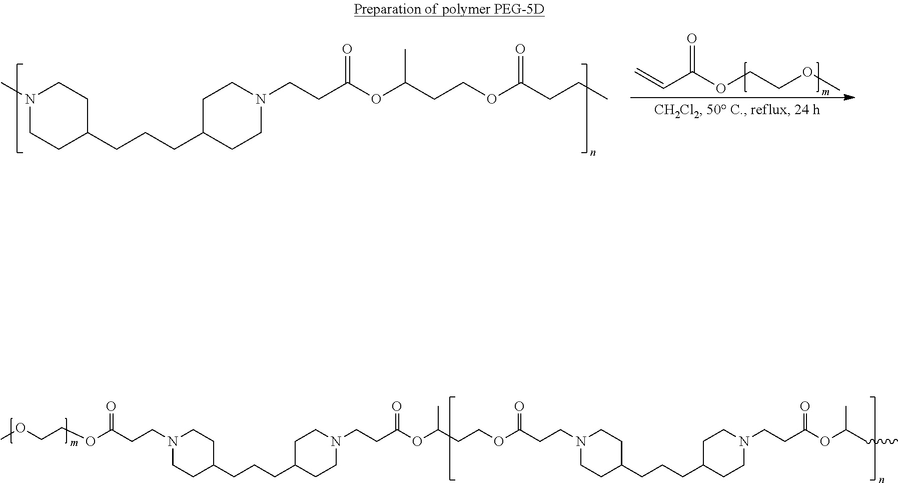

Polymer 5D prepared in the same manner as in Manufacturing Example 24 above and 858.7 mg of PEG-acrylate were dissolved in CH₂Cl₂ to a concentration of 1000 mg/ml and added to a flask. The reaction was carried out at 50° C. for 24 hours with reflux, and then PEG-5D was obtained.

### Manufacturing Example 135

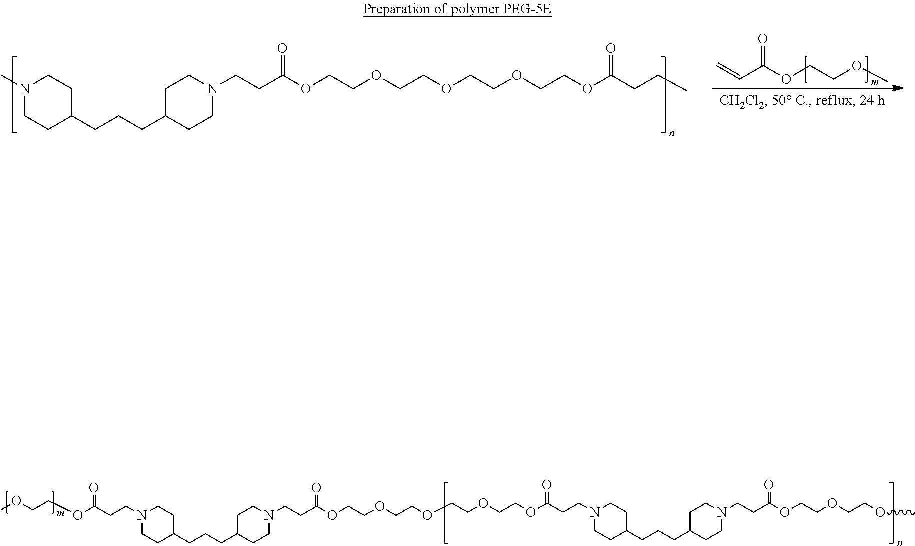

Polymer 5E prepared in the same manner as in Manufacturing Example 25 above and 563.0 mg of PEG-acrylate were dissolved in CH₂Cl₂ to a concentration of 2500 mg/ml and added to a flask. The reaction was carried out at 50° C. for 24 hours with reflux, and then PEG-5E was obtained.

### Manufacturing Example 136

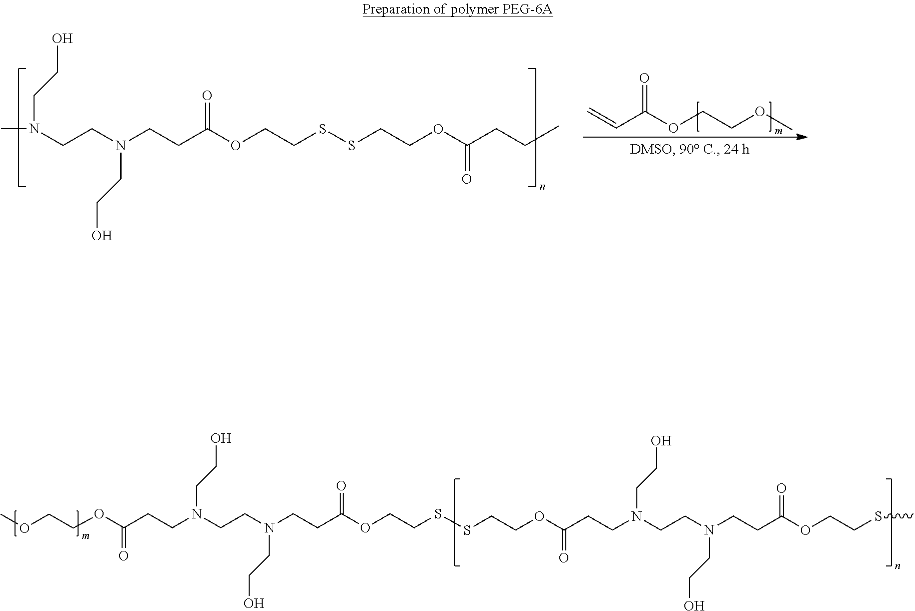

Polymer 6A prepared in the same manner as in Manufacturing Example 26 above and 648.8 mg of PEG-acrylate were dissolved in DMSO to a concentration of 1000 mg/ml and added to a flask. The reaction was carried out at 90° C. for 24 hours, and then PEG-6A was obtained.

### Manufacturing Example 137

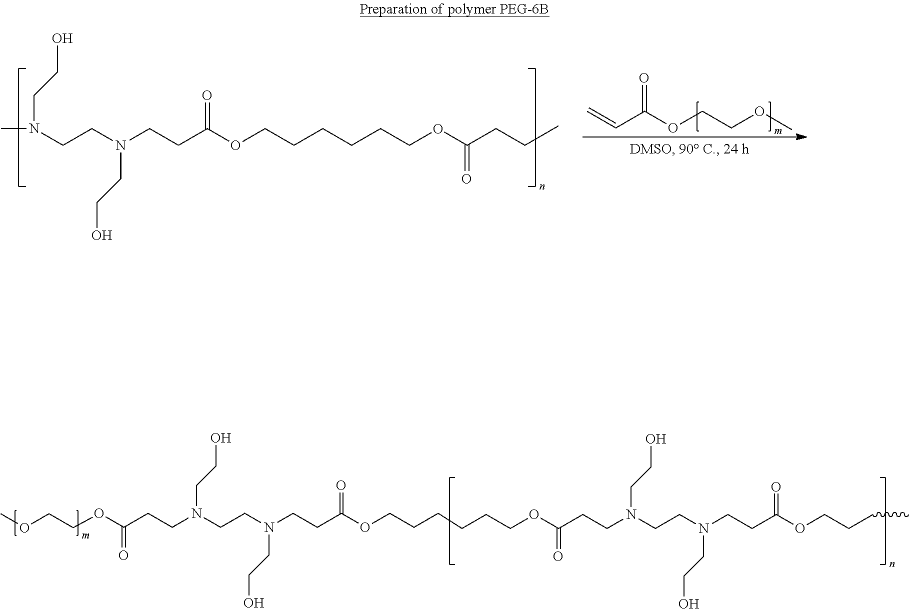

Polymer 6B prepared in the same manner as in Manufacturing Example 27 above and 752.3 mg of PEG-acrylate were dissolved in DMSO to a concentration of 1000 mg/ml and added to a flask. The reaction was carried out at 90° C. for 24 hours, and then PEG-6B was obtained.

### Manufacturing Example 138

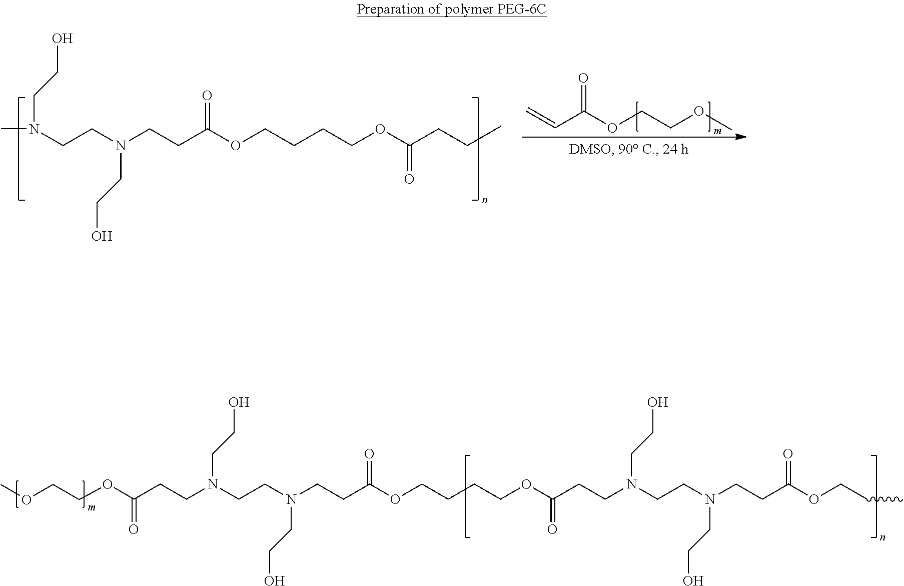

Polymer 6C prepared in the same manner as in Manufacturing Example 28 above and 858.7 mg of PEG-acrylate were dissolved in DMSO to a concentration of 1000 mg/ml and added to a flask. The reaction was carried out at 90° C. for 24 hours, and then PEG-6C was obtained.

### Manufacturing Example 139

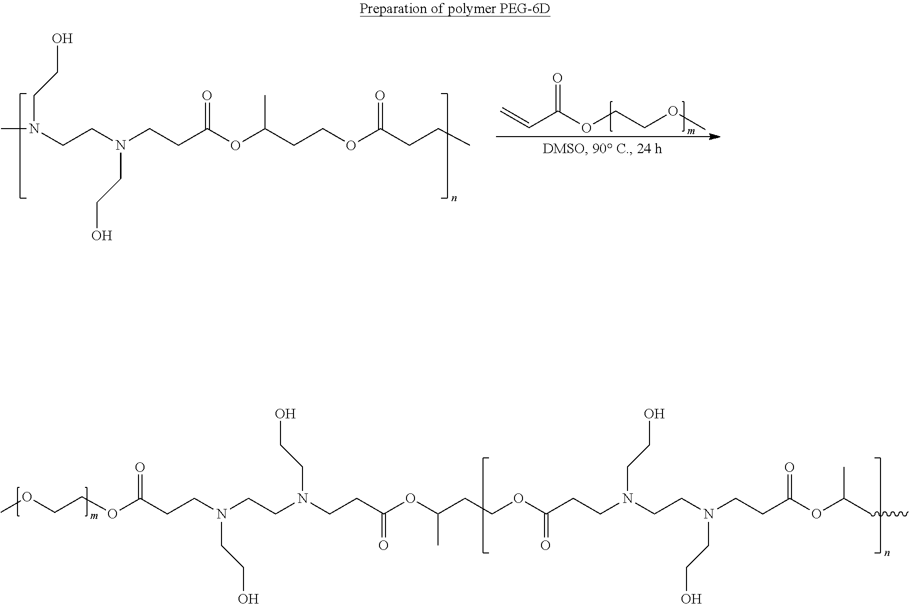

Polymer 6D prepared in the same manner as in Manufacturing Example 29 above and 858.7 mg of PEG-acrylate were dissolved in DMSO to a concentration of 1000 mg/ml and added to a flask. The reaction was carried out at 90° C. for 24 hours, and then PEG-6D was obtained.

### Manufacturing Example 140

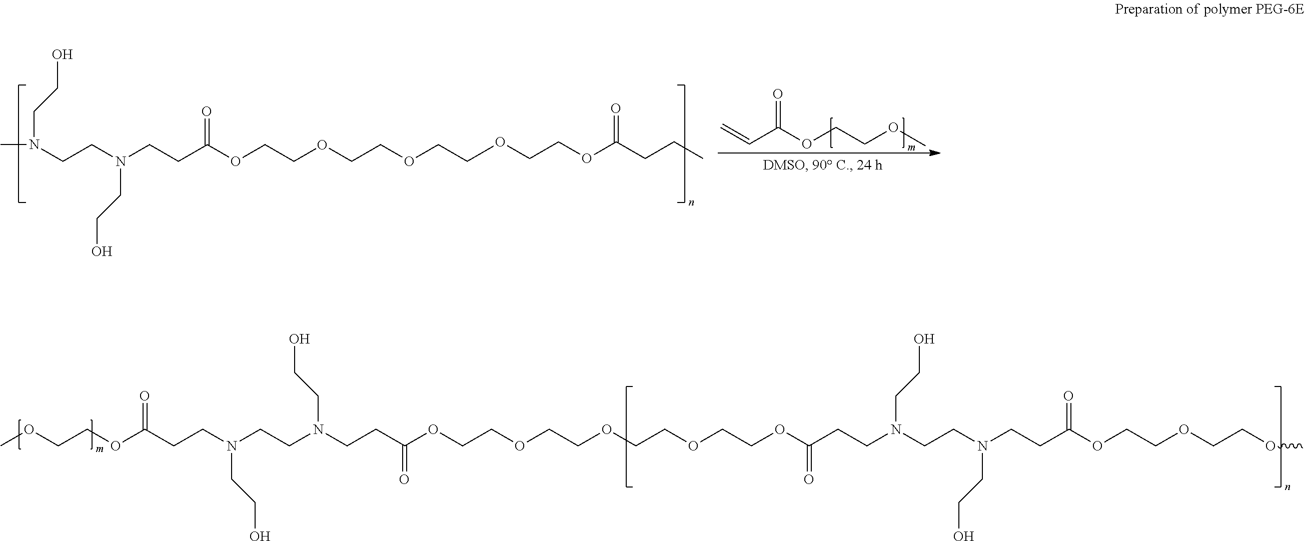

Polymer 6E prepared in the same manner as in Manufacturing Example 30 above and 563.0 mg of PEG-acrylate were dissolved in DMSO to a concentration of 2500 mg/ml and added to a flask. The reaction was carried out at 90° C. for 24 hours, and then PEG-6E was obtained.

### Manufacturing Example 141

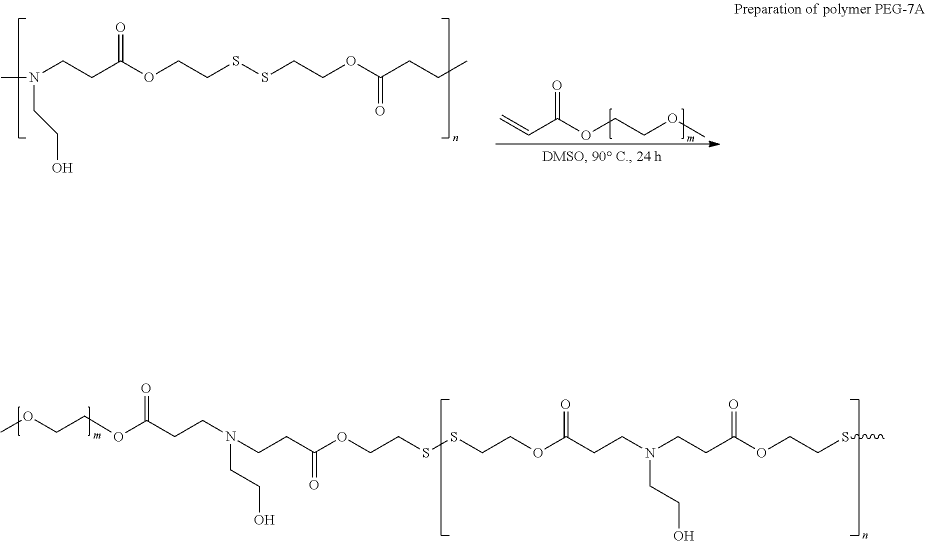

Polymer 7A prepared in the same manner as in Manufacturing Example 31 above and 648.8 mg of PEG-acrylate were dissolved in DMSO to a concentration of 1000 mg/ml and added to a flask. The reaction was carried out at 90° C. for 24 hours, and then PEG-7A was obtained.

### Manufacturing Example 142

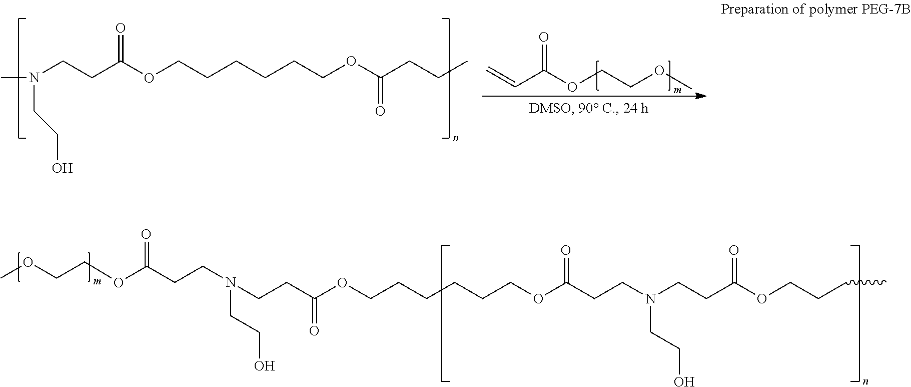

Polymer 7B prepared in the same manner as in Manufacturing Example 32 above and 752.3 mg of PEG-acrylate were dissolved in DMSO to a concentration of 1000 mg/ml and added to a flask. The reaction was carried out at 90° C. for 24 hours, and then PEG-7B was obtained.

### Manufacturing Example 143

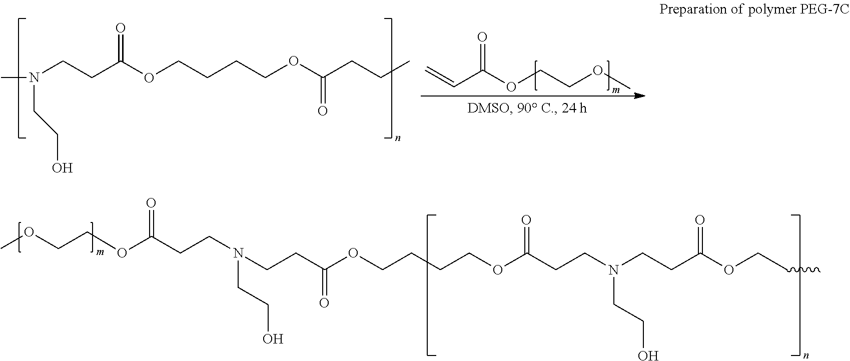

Polymer 7C prepared in the same manner as in Manufacturing Example 33 above and 858.7 mg of PEG-acrylate were dissolved in DMSO to a concentration of 1000 mg/ml and added to a flask. The reaction was carried out at 90° C. for 24 hours, and then PEG-7C was obtained.

### Manufacturing Example 144

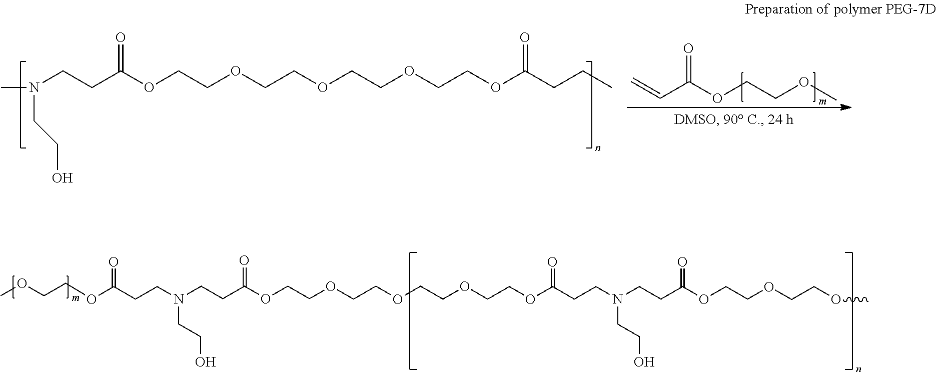

Polymer 7D prepared in the same manner as in Manufacturing Example 34 above and 858.7 mg of PEG-acrylate were dissolved in DMSO to a concentration of 1000 mg/ml and added to a flask. The reaction was carried out at 90° C. for 24 hours, and then PEG-7D was obtained.

### Manufacturing Example 145

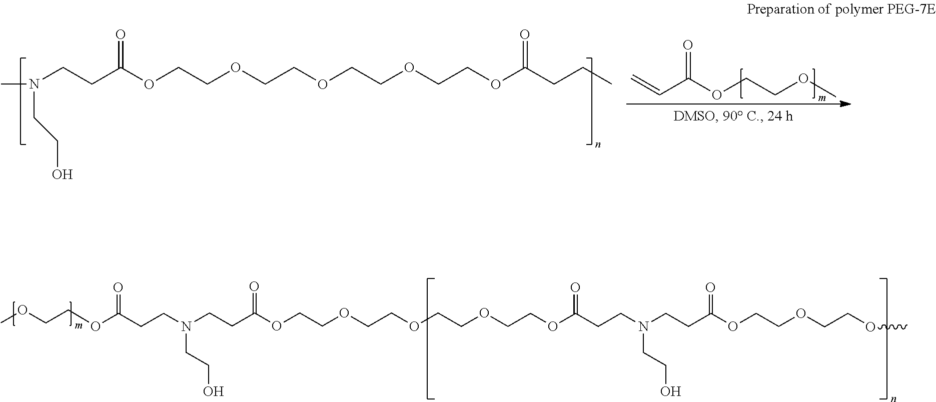

Polymer 7E prepared in the same manner as in Manufacturing Example 35 above and 563.0 mg of PEG-acrylate were dissolved in DMSO to a concentration of 2500 mg/ml and added to a flask. The reaction was carried out at 90° C. for 24 hours, and then PEG-7E was obtained.

### Manufacturing Example 146

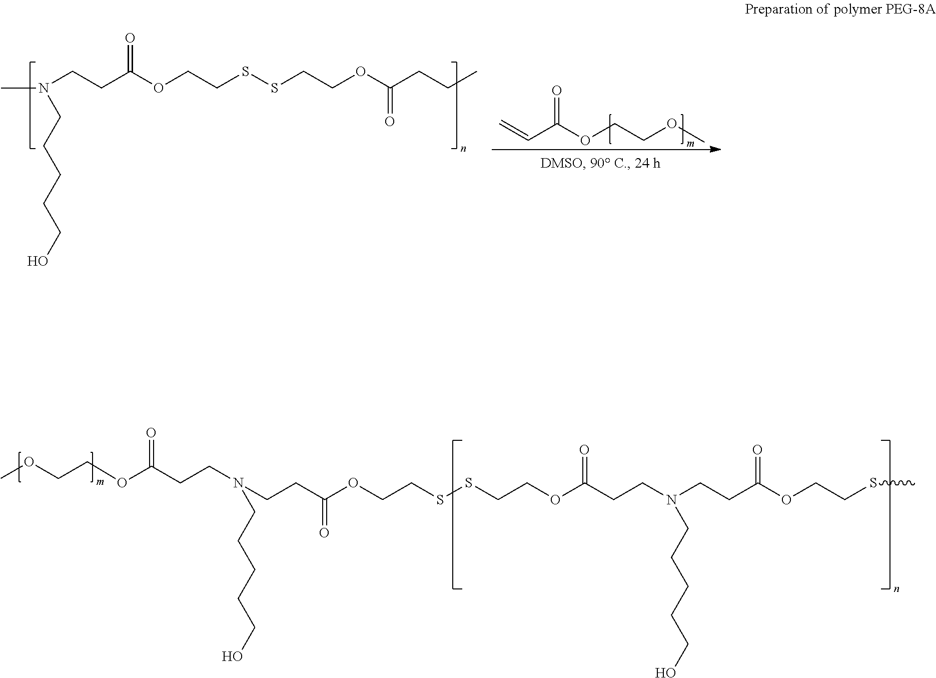

Polymer 8A prepared in the same manner as in Manufacturing Example 36 above and 648.8 mg of PEG-acrylate were dissolved in DMSO to a concentration of 1000 mg/ml and added to a flask. The reaction was carried out at 90° C. for 24 hours, and then PEG-8A was obtained.

### Manufacturing Example 147

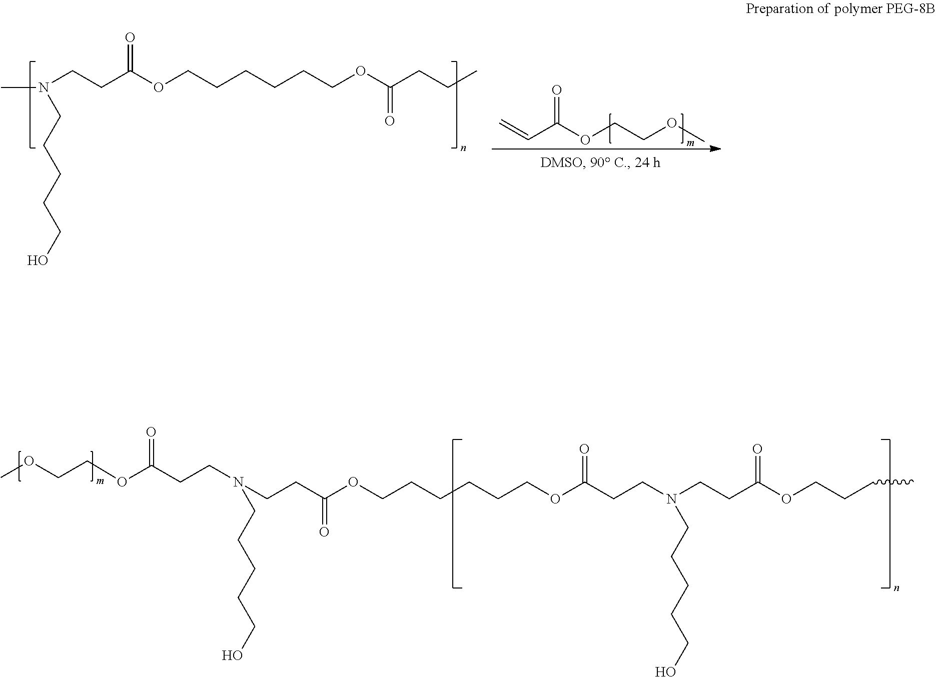

Polymer 8B prepared in the same manner as in Manufacturing Example 37 above and 752.3 mg of PEG-acrylate were dissolved in DMSO to a concentration of 1000 mg/ml and added to a flask. The reaction was carried out at 90° C. for 24 hours, and then PEG-8B was obtained.

### Manufacturing Example 148

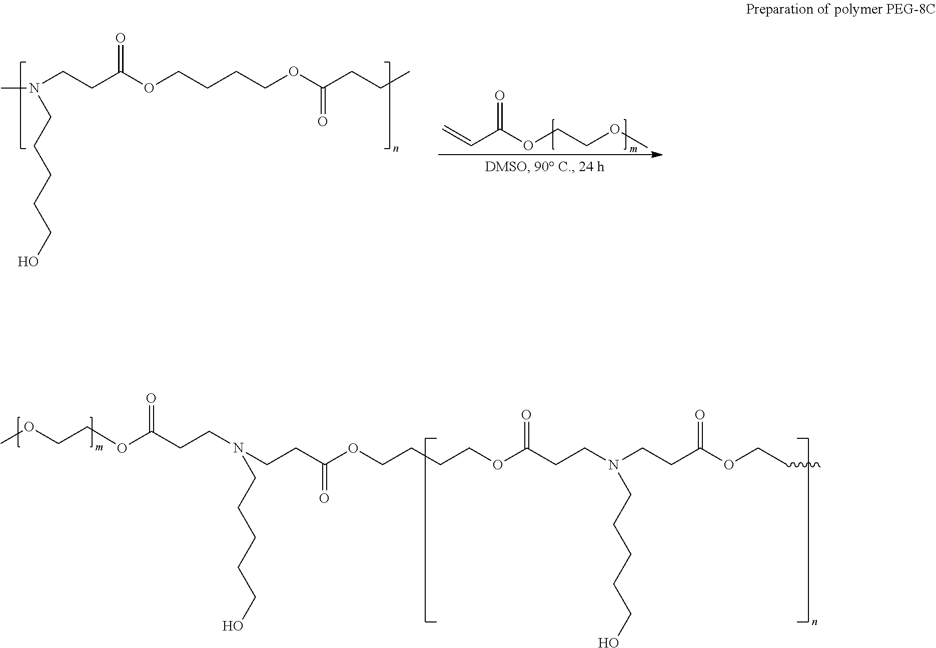

Polymer 8C prepared in the same manner as in Manufacturing Example 38 above and 858.7 mg of PEG-acrylate were dissolved in DMSO to a concentration of 1000 mg/ml and added to a flask. The reaction was carried out at 90° C. for 24 hours, and then PEG-8C was obtained.

### Manufacturing Example 149

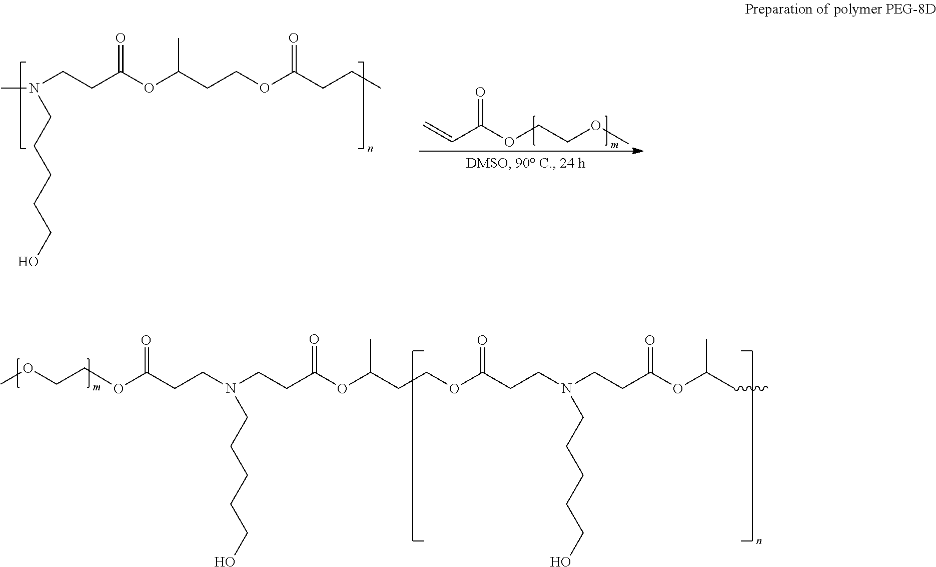

Polymer 8D prepared in the same manner as in Manufacturing Example 39 above and 858.7 mg of PEG-acrylate were dissolved in DMSO to a concentration of 1000 mg/ml and added to a flask. The reaction was carried out at 90° C. for 24 hours, and then PEG-8D was obtained.

### Manufacturing Example 150

### Preparation of Polymer PEG-8E

Polymer 8E prepared in the same manner as in Manufacturing Example 40 above and 563.0 mg of PEG-acrylate were dissolved in DMSO to a concentration of 2500 mg/ml and added to a flask. The reaction was carried out at 90° C. for 24 hours, and then PEG-8E was obtained.

### Manufacturing Example 151

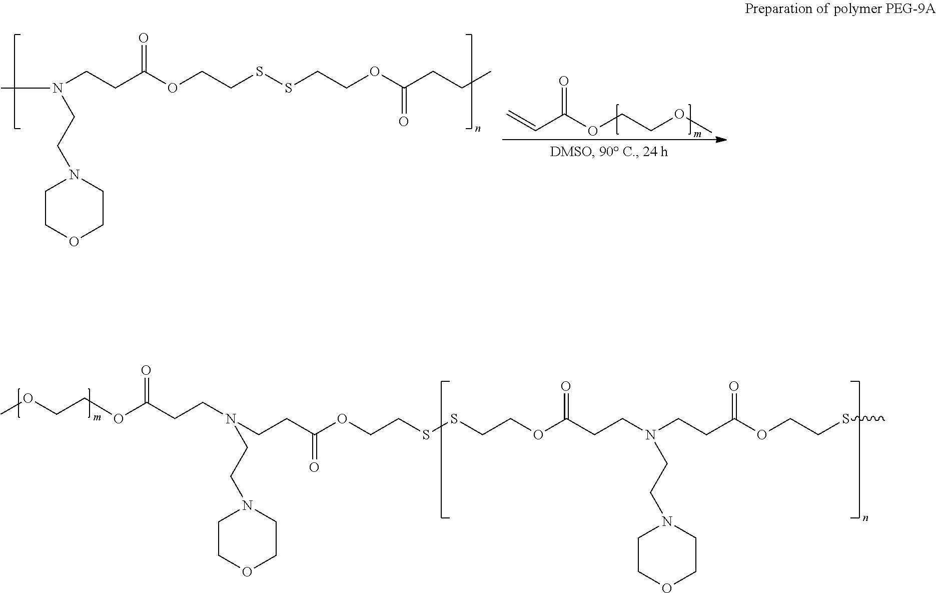

Polymer 9A prepared in the same manner as in Manufacturing Example 41 above and 648.8 mg of PEG-acrylate were dissolved in DMSO to a concentration of 1000 mg/ml and added to a flask. The reaction was carried out at 90° C. for 24 hours, and then PEG-9A was obtained.

### Manufacturing Example 152

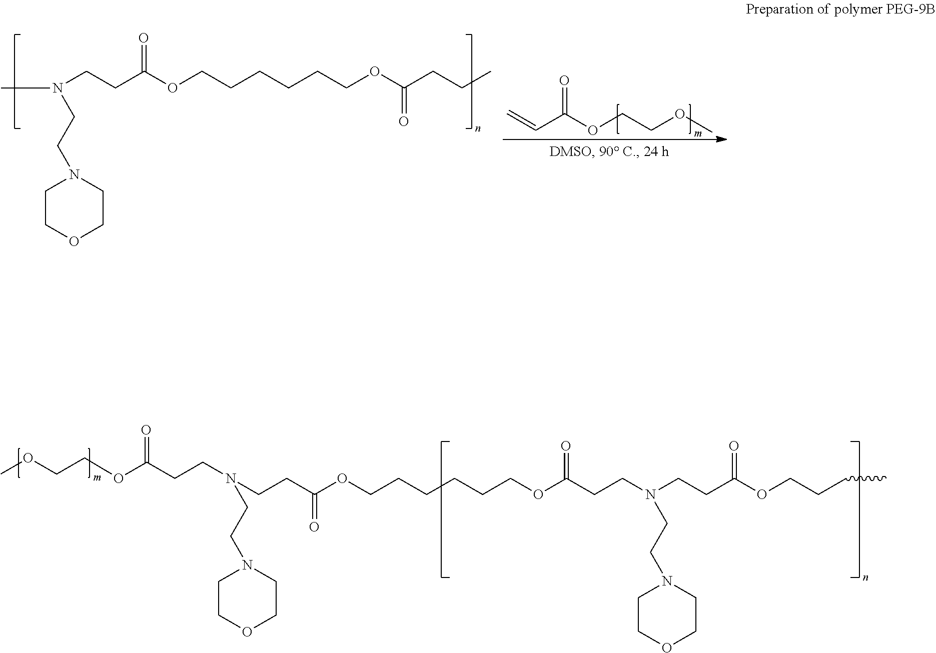

Polymer 9B prepared in the same manner as in Manufacturing Example 42 above and 752.3 mg of PEG-acrylate were dissolved in DMSO to a concentration of 1000 mg/ml and added to a flask. The reaction was carried out at 90° C. for 24 hours, and then PEG-9B was obtained.

### Manufacturing Example 153

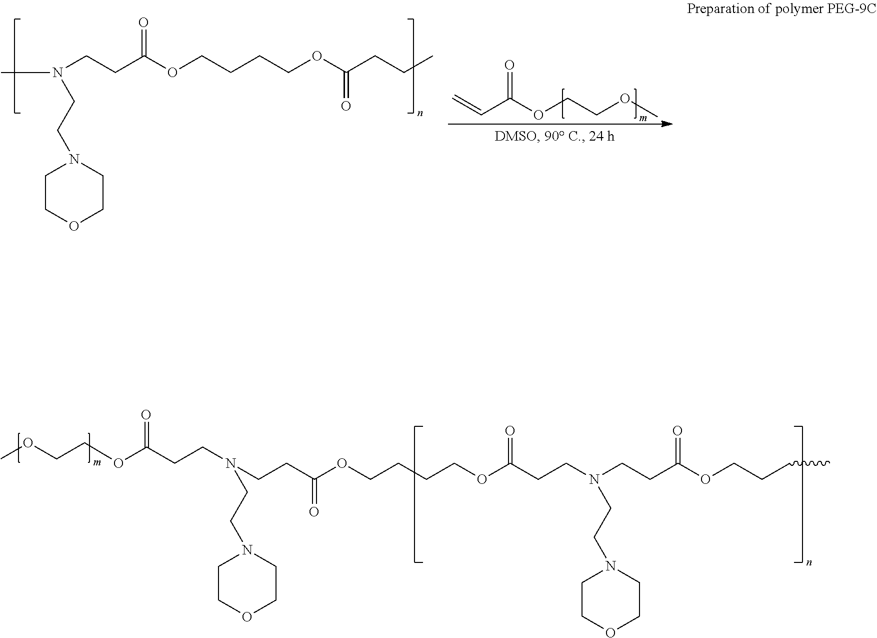

Polymer 9C prepared in the same manner as in Manufacturing Example 43 above and 858.7 mg of PEG-acrylate were dissolved in DMSO to a concentration of 1000 mg/ml and added to a flask. The reaction was carried out at 90° C. for 24 hours, and then PEG-9C was obtained.

### Manufacturing Example 154

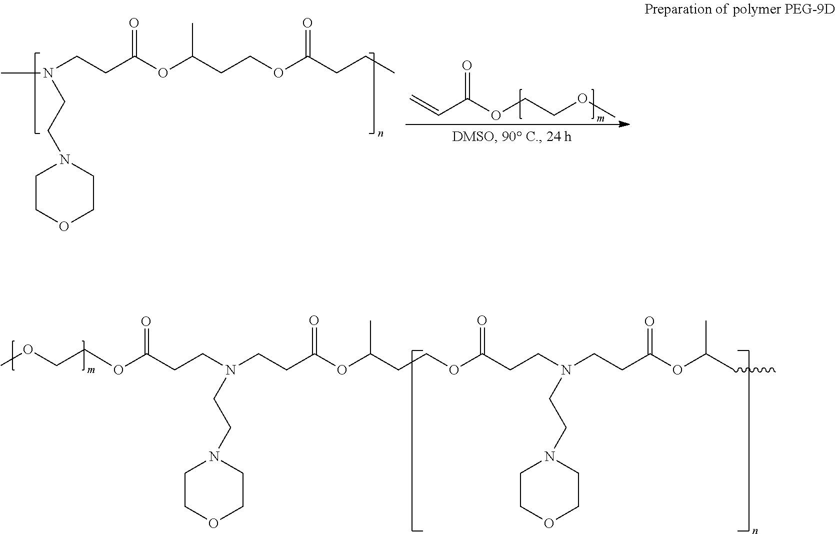

Polymer 9D prepared in the same manner as in Manufacturing Example 44 above and 858.7 mg of PEG-acrylate were dissolved in DMSO to a concentration of 1000 mg/ml and added to a flask. The reaction was carried out at 90° C. for 24 hours, and then PEG-9D was obtained.

### Manufacturing Example 155

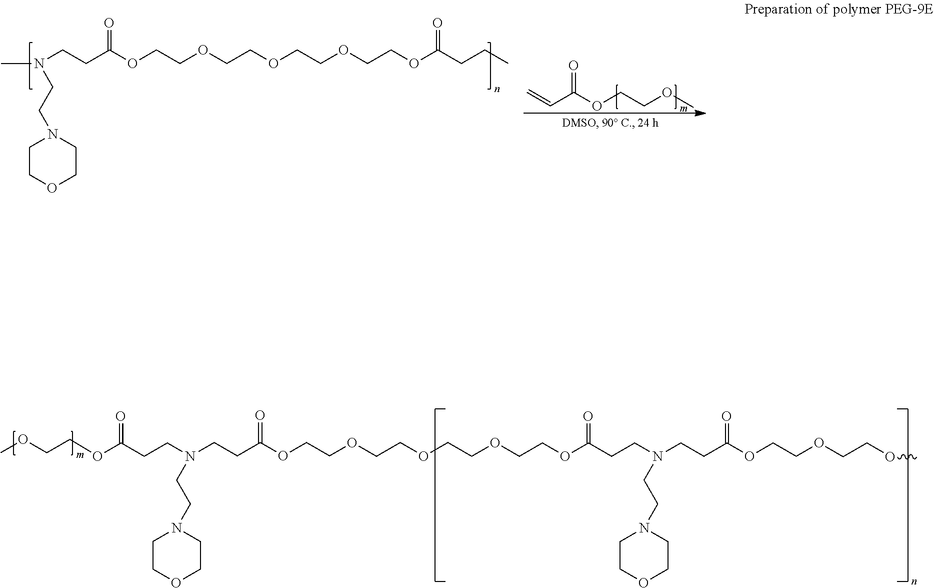

Polymer 9E prepared in the same manner as in Manufacturing Example 45 above and 563.0 mg of PEG-acrylate were dissolved in DMSO to a concentration of 2500 mg/ml and added to a flask. The reaction was carried out at 90° C. for 24 hours, and then PEG-9E was obtained.

### Manufacturing Example 156

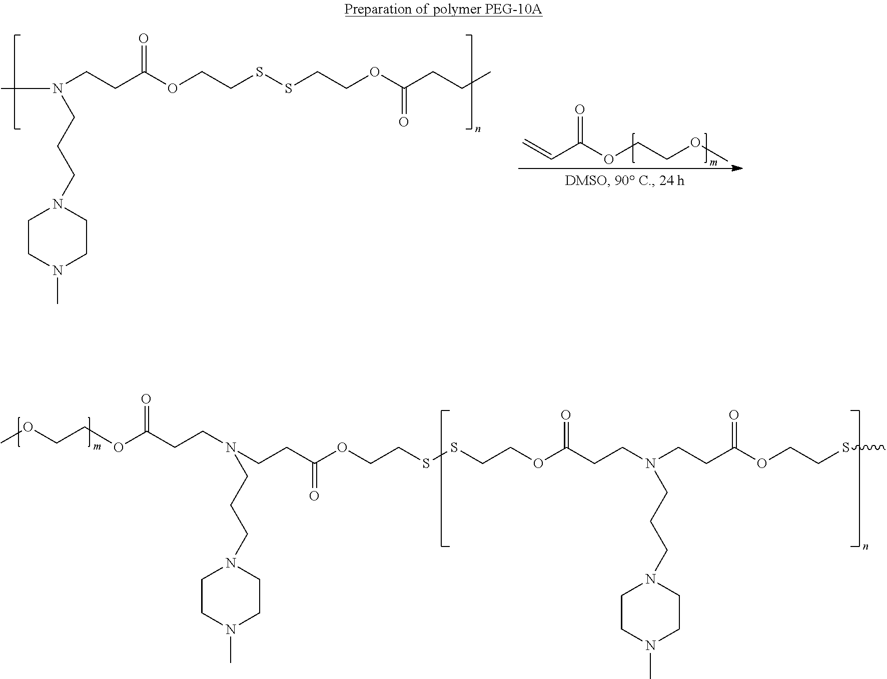

Polymer 10A prepared in the same manner as in Manufacturing Example 46 above and 648.8 mg of PEG-acrylate were dissolved in DMSO to a concentration of 1000 mg/ml and added to a flask. The reaction was carried out at 90° C. for 24 hours, and then PEG-10A was obtained.

### Manufacturing Example 157

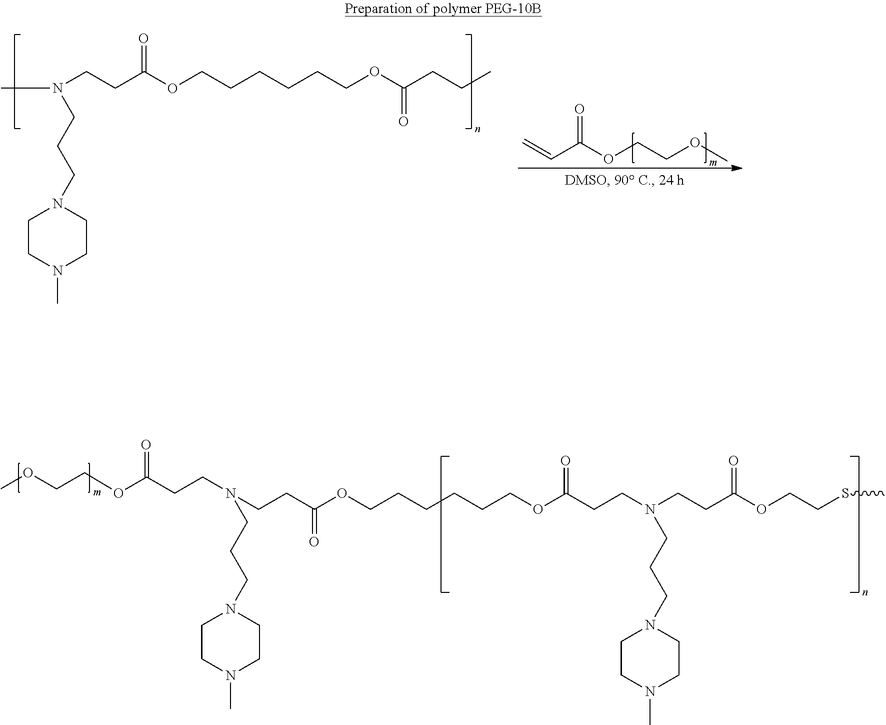

Polymer 10B prepared in the same manner as in Manufacturing Example 47 above and 752.3 mg of PEG-acrylate were dissolved in DMSO to a concentration of 1000 mg/ml and added to a flask. The reaction was carried out at 90° C. for 24 hours, and then PEG-10B was obtained.

### Manufacturing Example 158

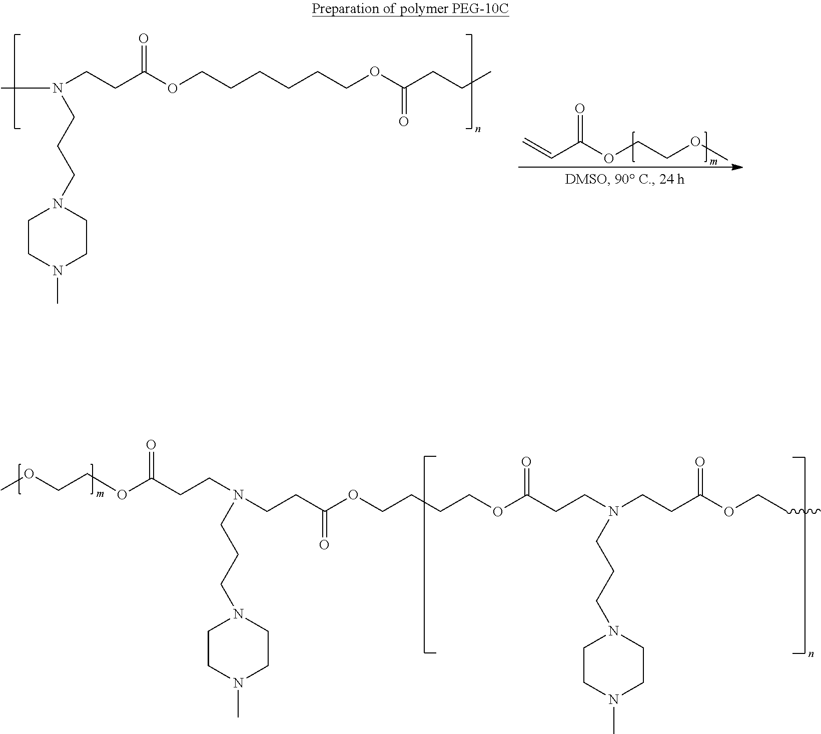

Polymer 10C prepared in the same manner as in Manufacturing Example 48 above and 858.7 mg of PEG-acrylate were dissolved in DMSO to a concentration of 1000 mg/ml and added to a flask. The reaction was carried out at 90° C. for 24 hours, and then PEG-10C was obtained.

### Manufacturing Example 159

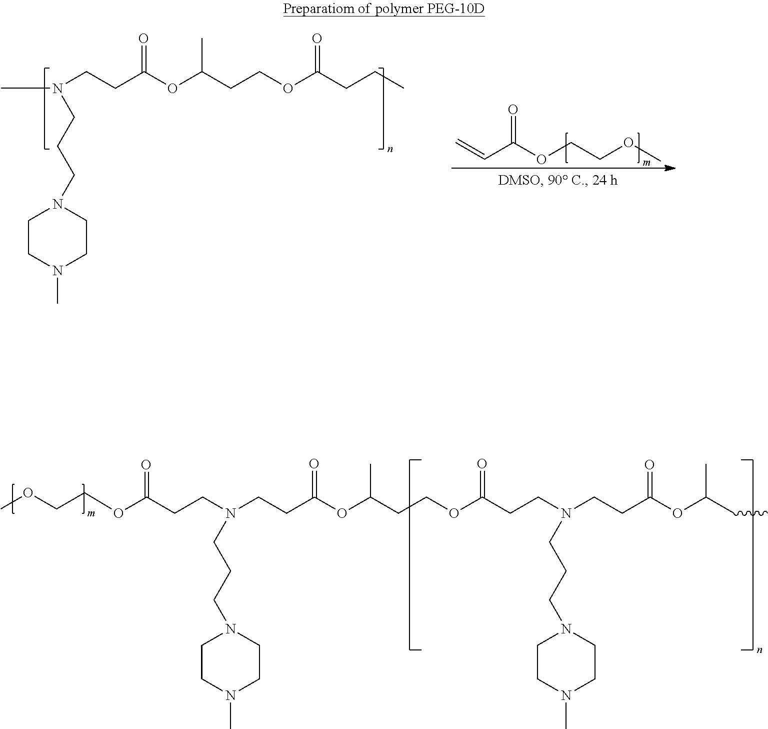

Polymer 10D prepared in the same manner as in Manufacturing Example 49 above and 858.7 mg of PEG-acrylate were dissolved in DMSO to a concentration of 1000 mg/ml and added to a flask. The reaction was carried out at 90° C. for 24 hours, and then PEG-10D was obtained.

### Manufacturing Example 160

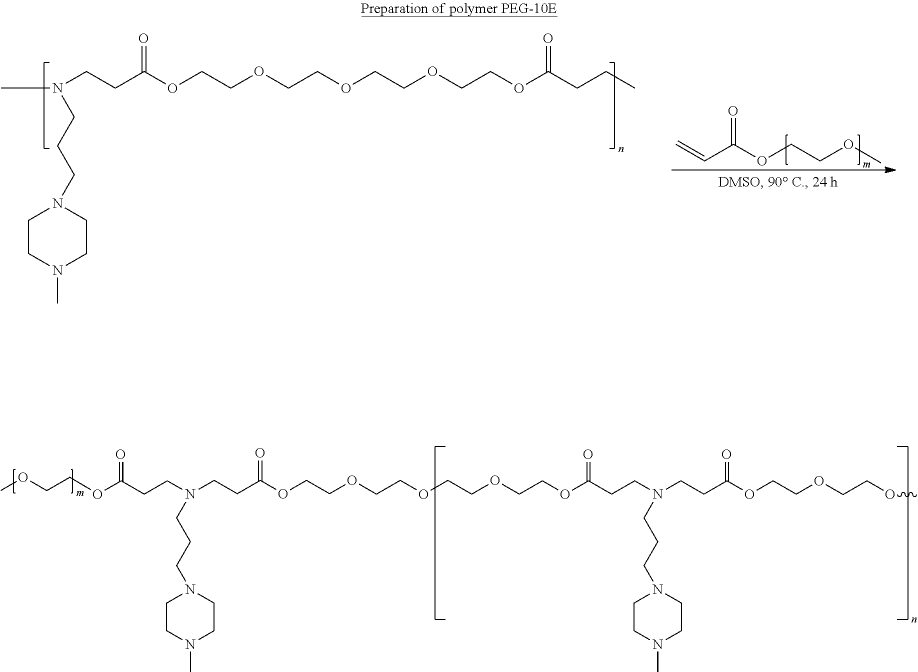

Polymer 10E prepared in the same manner as in Manufacturing Example 50 above and 563.0 mg of PEG-acrylate were dissolved in DMSO to a concentration of 2500 mg/ml and added to a flask. The reaction was carried out at 90° C. for 24 hours, and then PEG-10E was obtained.

### Manufacturing Example 161

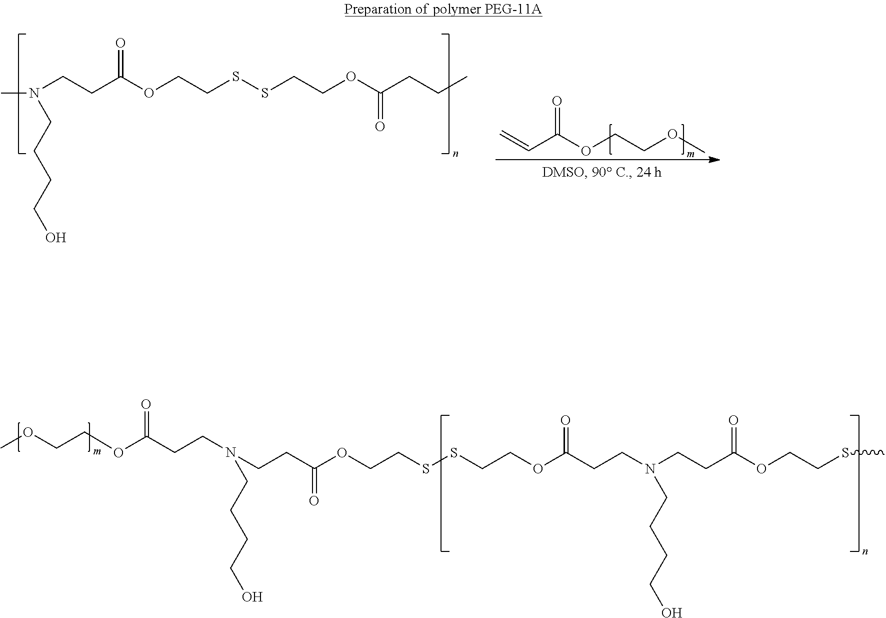

Polymer 11A prepared in the same manner as in Manufacturing Example 51 above and 648.8 mg of PEG-acrylate were dissolved in DMSO to a concentration of 1000 mg/ml and added to a flask. The reaction was carried out at 90° C. for 24 hours, and then PEG-11A was obtained.

### Manufacturing Example 162

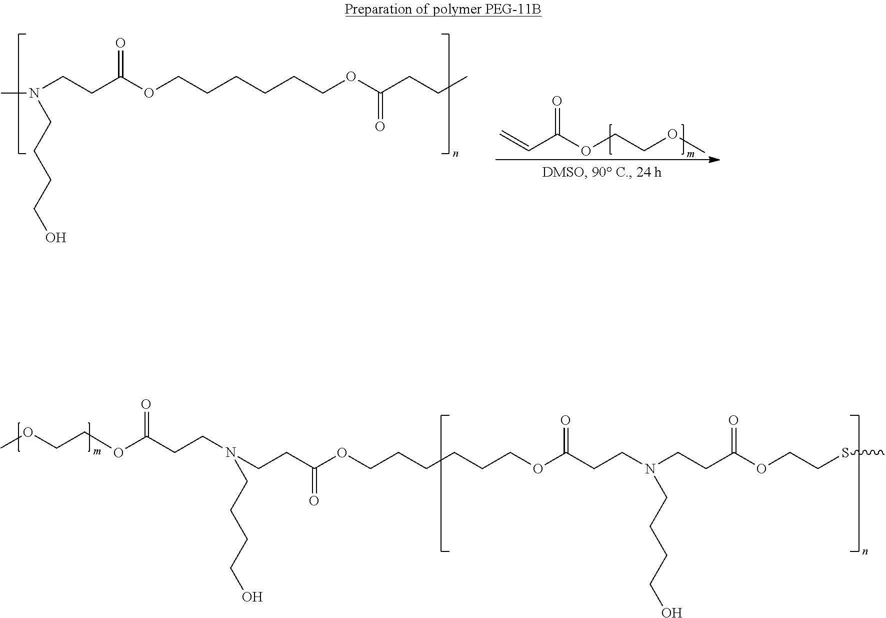

Polymer 11B prepared in the same manner as in Manufacturing Example 52 above and 752.3 mg of PEG-acrylate were dissolved in DMSO to a concentration of 1000 mg/ml and added to a flask. The reaction was carried out at 90° C. for 24 hours, and then PEG-11B was obtained.

### Manufacturing Example 163

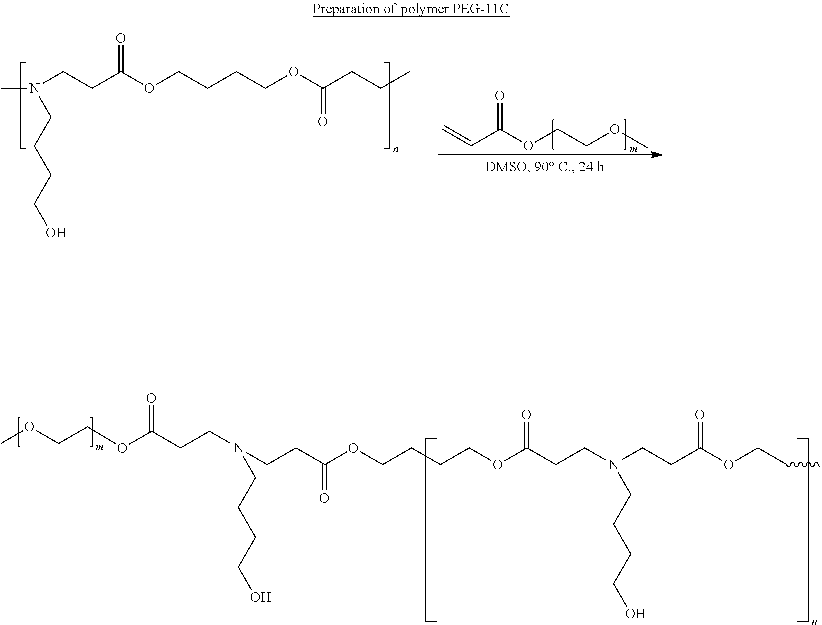

Polymer 11C prepared in the same manner as in Manufacturing Example 53 above and 858.7 mg of PEG-acrylate were dissolved in DMSO to a concentration of 1000 mg/ml and added to a flask. The reaction was carried out at 90° C. for 24 hours, and then PEG-11C was obtained.

### Manufacturing Example 164

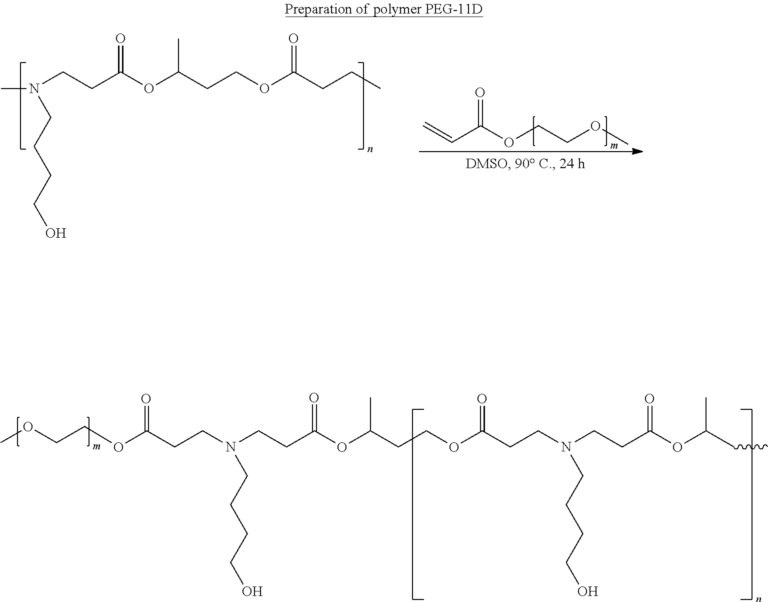

Polymer 11D prepared in the same manner as in Manufacturing Example 54 above and 858.7 mg of PEG-acrylate were dissolved in DMSO to a concentration of 1000 mg/ml and added to a flask. The reaction was carried out at 90° C. for 24 hours, and then PEG-11D was obtained.

### Manufacturing Example 165

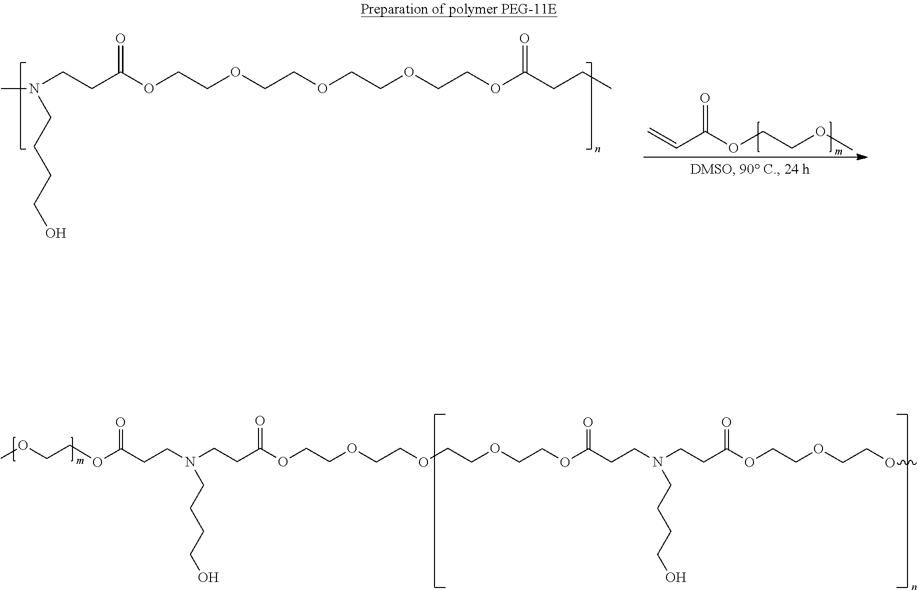

Polymer 11E prepared in the same manner as in Manufacturing Example 55 above and 563.0 mg of PEG-acrylate were dissolved in DMSO to a concentration of 2500 mg/ml and added to a flask. The reaction was carried out at 90° C. for 24 hours, and then PEG-11E was obtained.

### Manufacturing Example 166

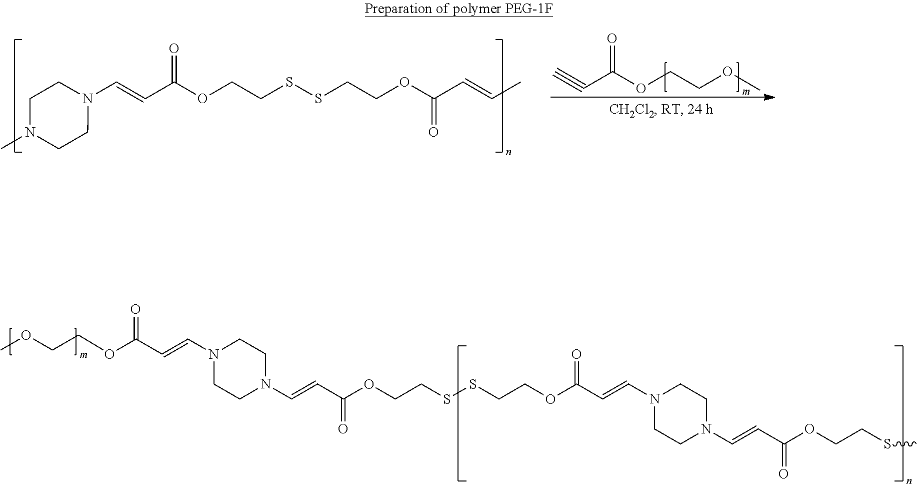

Polymer 1F prepared in the same manner as in Manufacturing Example 56 above and 329.5 mg of PEG-alkynoate were dissolved in 4 mL of CH2C12 and added to a flask. The reaction was carried out at room temperature for 24 hours, and then PEG-1F was obtained.

### Manufacturing Example 167

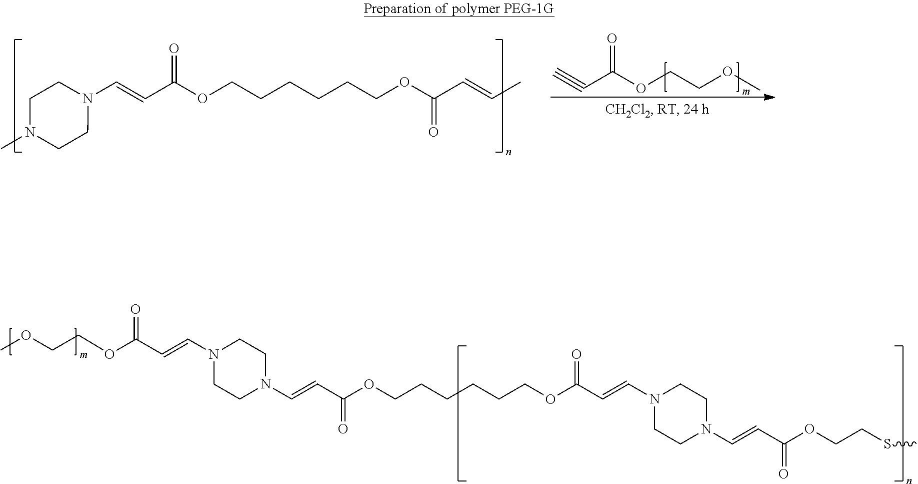

Polymer 1G prepared in the same manner as in Manufacturing Example 57 above and 383.0 mg of PEG-alkynoate were dissolved in 4 mL of CH₂Cl₂ and added to a flask. The reaction was carried out at room temperature for 24 hours, and then PEG-1G was obtained.

### Manufacturing Example 168

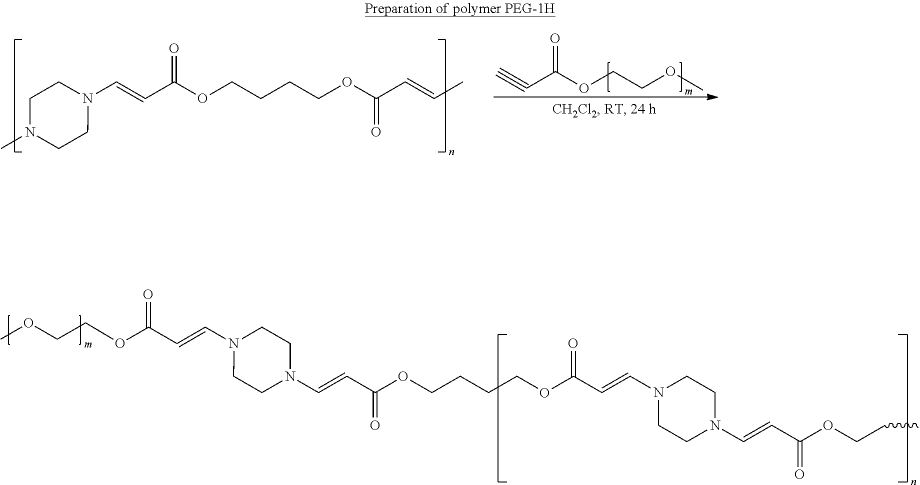

Polymer 1H prepared in the same manner as in Manufacturing Example 58 above and 438.3 mg of PEG-alkynoate were dissolved in 4 mL of CH₂Cl₂ and added to a flask. The reaction was carried out at room temperature for 24 hours, and then PEG-1H was obtained.

### Manufacturing Example 169

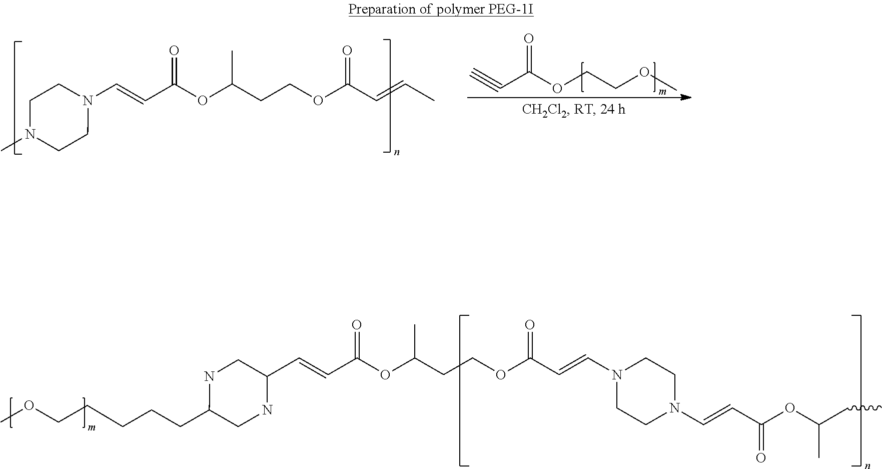

Polymer 1I prepared in the same manner as in Manufacturing Example 59 above and 438.3 mg of PEG-alkynoate were dissolved in 4 mL of CH₂Cl₂ and added to a flask. The reaction was carried out at room temperature for 24 hours, and then PEG-1I was obtained.

### Manufacturing Example 170

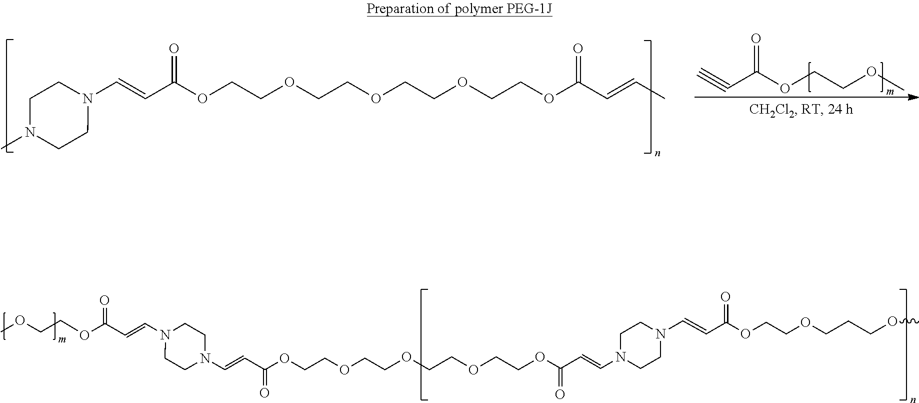

Polymer 1J prepared in the same manner as in Manufacturing Example 60 above and 285.3 mg of PEG-alkynoate were dissolved in 4 mL of CH₂Cl₂ and added to a flask. The reaction was carried out at room temperature for 24 hours, and then PEG-1J was obtained.

### Manufacturing Example 171

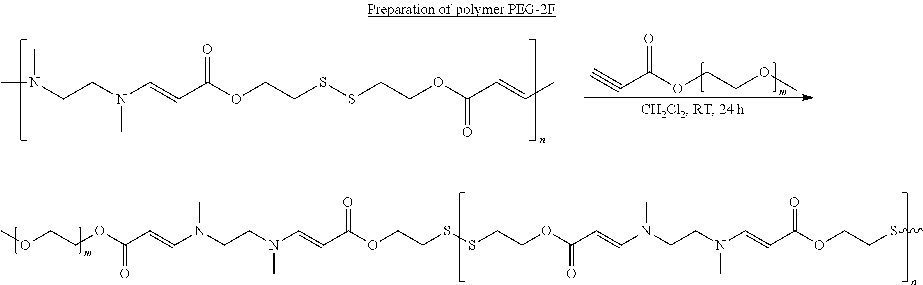

Polymer 2F prepared in the same manner as in Manufacturing Example 61 above and 329.5 mg of PEG-alkynoate were dissolved in 4 mL of CH₂Cl₂ and added to a flask. The reaction was carried out at room temperature for 24 hours, and then PEG-2F was obtained.

### Manufacturing Example 172

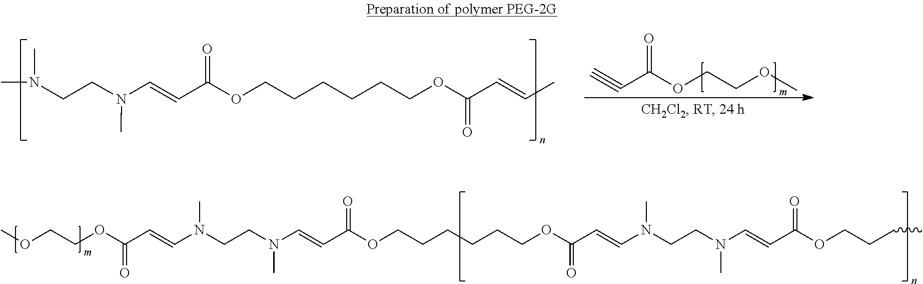

Polymer 2G prepared in the same manner as in Manufacturing Example 62 above and 383.0 mg of PEG-alkynoate were dissolved in 4 mL of CH₂Cl₂ and added to a flask. The reaction was carried out at room temperature for 24 hours, and then PEG-2G was obtained.

### Manufacturing Example 173

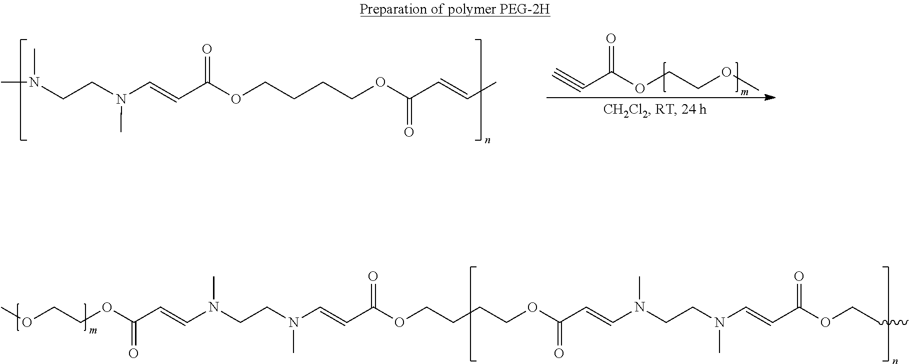

Polymer 2H prepared in the same manner as in Manufacturing Example 63 above and 438.3 mg of PEG-alkynoate were dissolved in 4 mL of CH₂Cl₂ and added to a flask. The reaction was carried out at room temperature for 24 hours, and then PEG-2H was obtained.

### Manufacturing Example 174

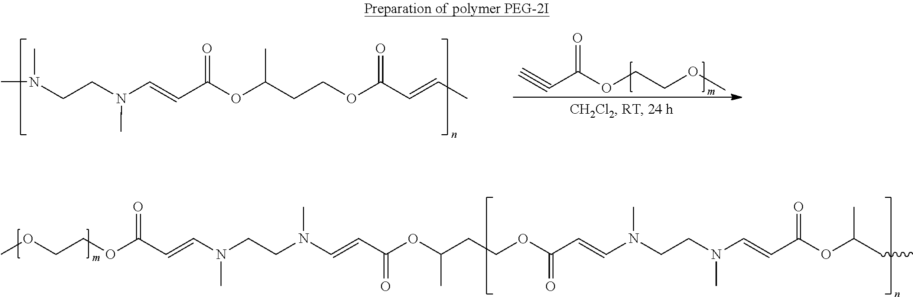

Polymer 2I prepared in the same manner as in Manufacturing Example 64 above and 438.3 mg of PEG-alkynoate were dissolved in 4 mL of CH₂Cl₂ and added to a flask. The reaction was carried out at room temperature for 24 hours, and then PEG-2I was obtained.

### Manufacturing Example 175

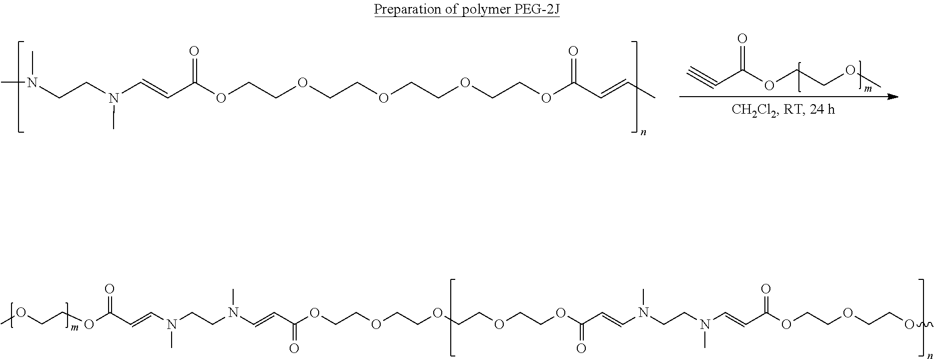

Polymer 2J prepared in the same manner as in Manufacturing Example 65 above and 285.3 mg of PEG-alkynoate were dissolved in 4 mL of CH₂Cl₂ and added to a flask. The reaction was carried out at room temperature for 24 hours, and then PEG-2J was obtained.

### Manufacturing Example 176

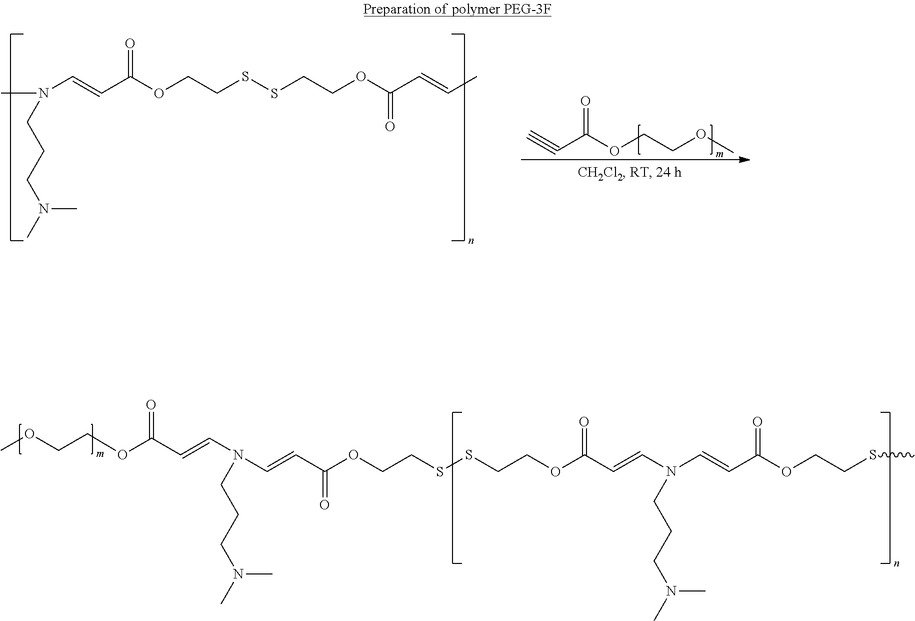

Polymer 3F prepared in the same manner as in Manufacturing Example 66 above and 329.5 mg of PEG-alkynoate were dissolved in 4 mL of CH₂Cl₂ and added to a flask. The reaction was carried out at room temperature for 24 hours, and then PEG-3F was obtained.

### Manufacturing Example 177

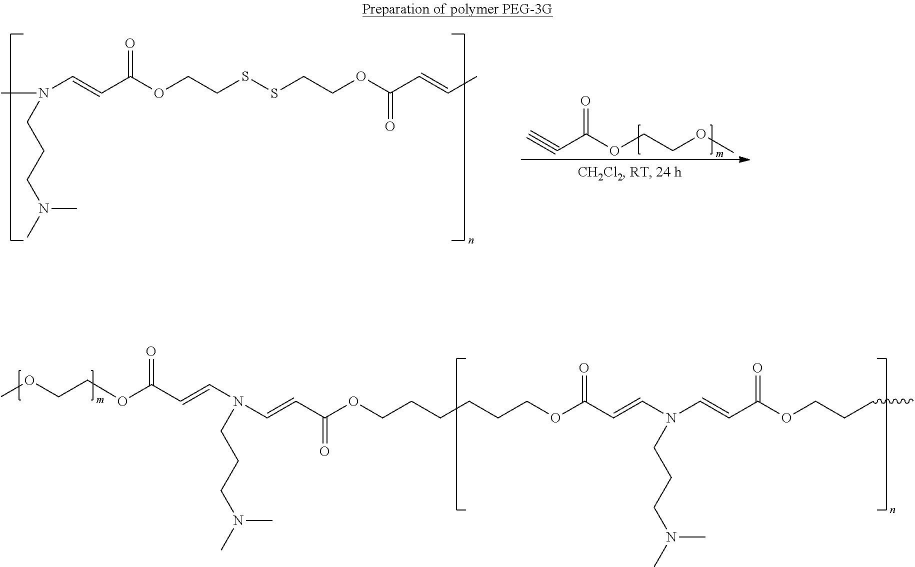

Polymer 3G prepared in the same manner as in Manufacturing Example 67 above and 383.0 mg of PEG-alkynoate were dissolved in 4 mL of CH₂Cl₂ and added to a flask. The reaction was carried out at room temperature for 24 hours, and then PEG-3G was obtained.

### Manufacturing Example 178

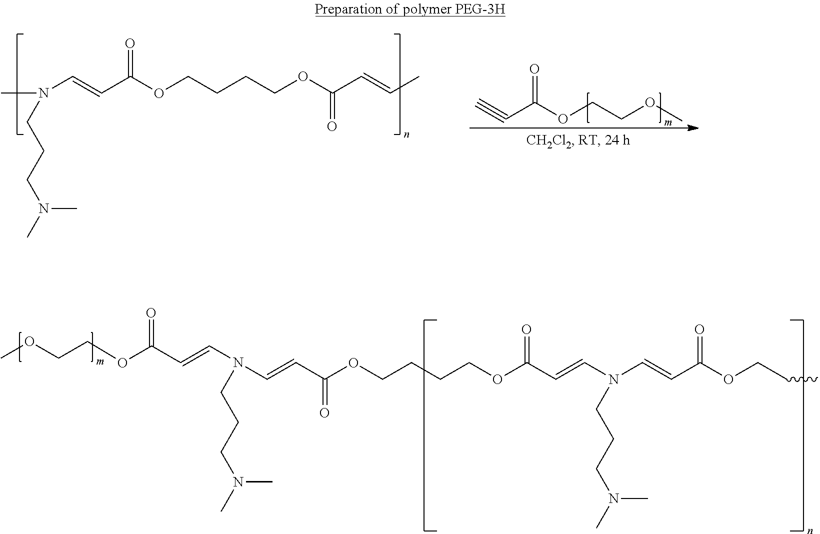

Polymer 3H prepared in the same manner as in Manufacturing Example 68 above and 438.3 mg of PEG-alkynoate were dissolved in 4 mL of CH₂Cl₂ and added to a flask. The reaction was carried out at room temperature for 24 hours, and then PEG-3H was obtained.

### Manufacturing Example 179

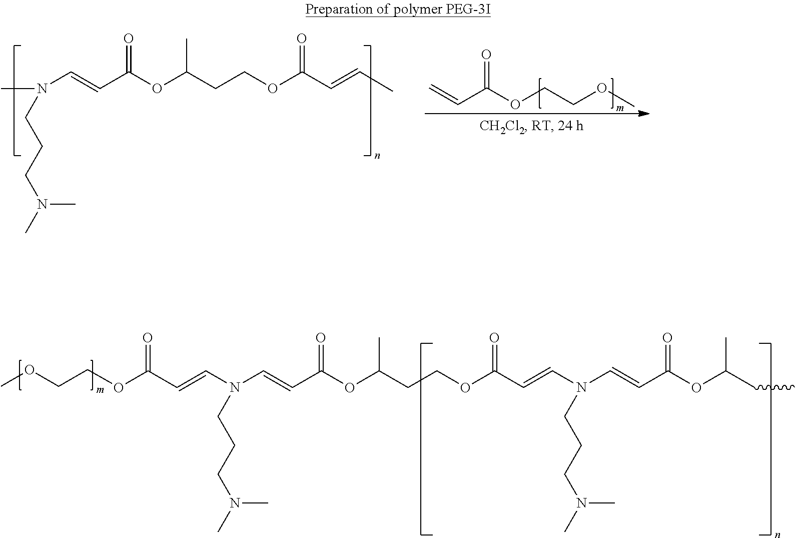

Polymer 3I prepared in the same manner as in Manufacturing Example 69 above and 438.3 mg of PEG-alkynoate were dissolved in 4 mL of CH₂Cl₂ and added to a flask. The reaction was carried out at room temperature for 24 hours, and then PEG-3I was obtained.

### Manufacturing Example 180

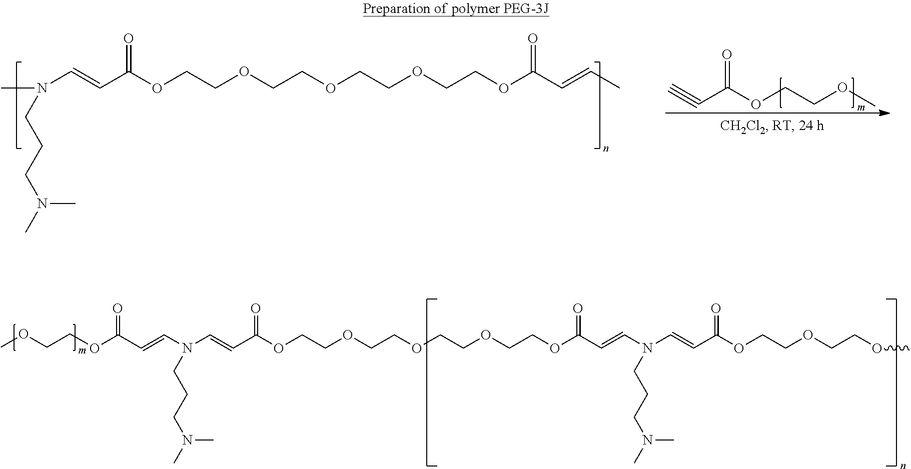

Polymer 3J prepared in the same manner as in Manufacturing Example 70 above and 285.3 mg of PEG-alkynoate were dissolved in 4 mL of CH₂Cl₂ and added to a flask. The reaction was carried out at room temperature for 24 hours, and then PEG-3J was obtained.

### Manufacturing Example 181

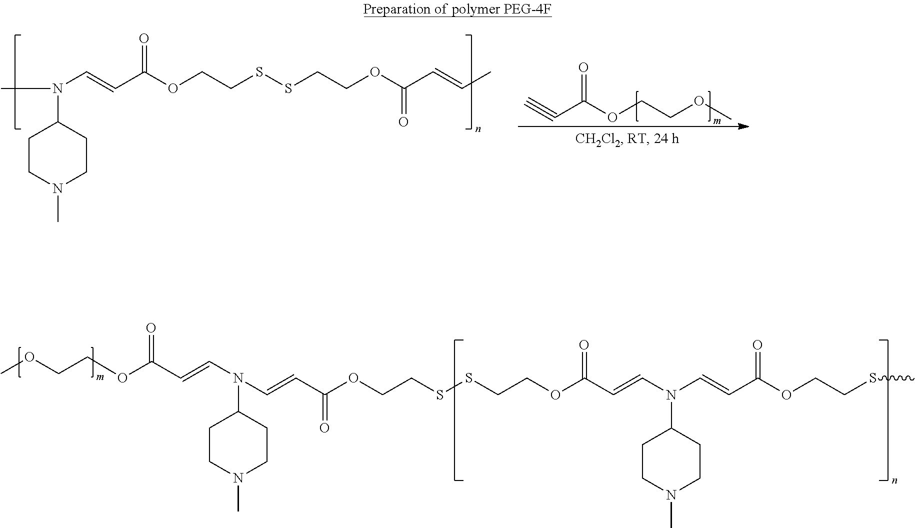

Polymer 4F prepared in the same manner as in Manufacturing Example 71 above and 329.5 mg of PEG-alkynoate were dissolved in 4 mL of CH₂Cl₂ and added to a flask. The reaction was carried out at room temperature for 24 hours, and then PEG-4F was obtained.

### Manufacturing Example 182

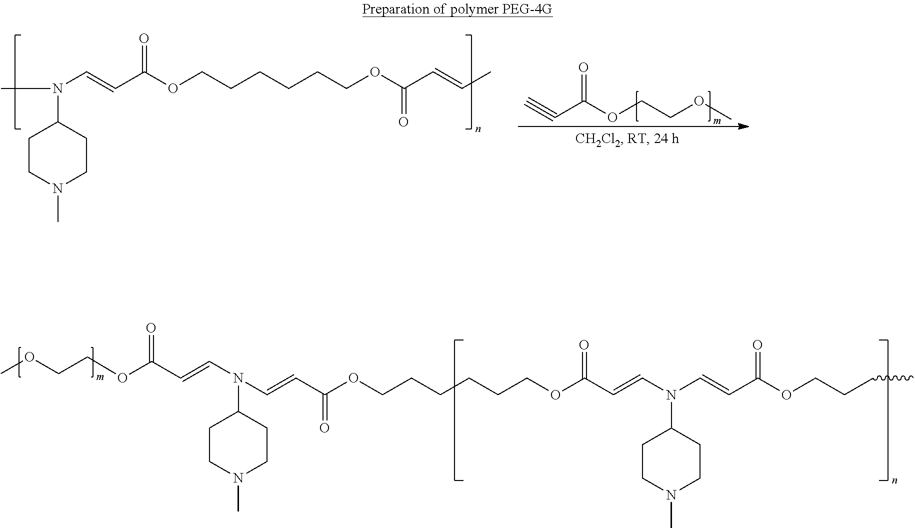

Polymer 4G prepared in the same manner as in Manufacturing Example 72 above and 383.0 mg of PEG-alkynoate were dissolved in 4 mL of CH₂Cl₂ and added to a flask. The reaction was carried out at room temperature for 24 hours, and then PEG-4G was obtained.

### Manufacturing Example 183

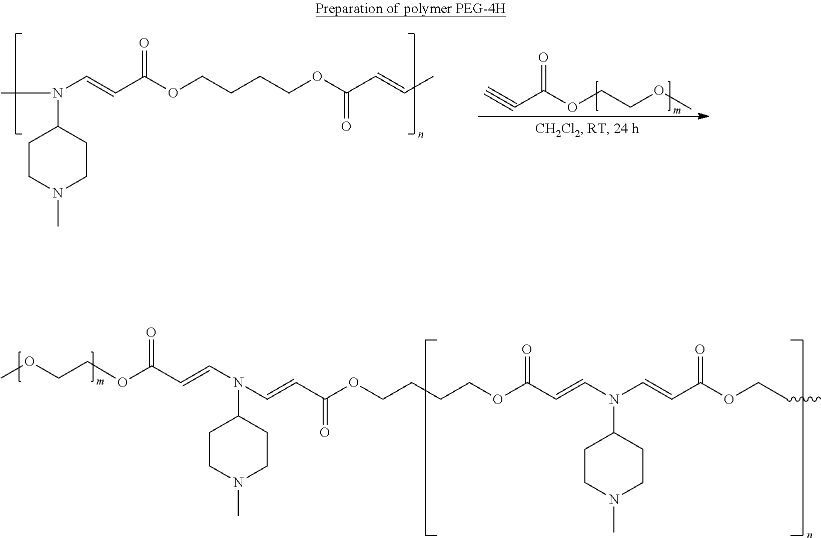

Polymer 4H prepared in the same manner as in Manufacturing Example 73 above and 438.3 mg of PEG-alkynoate were dissolved in 4 mL of CH₂Cl₂ and added to a flask. The reaction was carried out at room temperature for 24 hours, and then PEG-4H was obtained.

### Manufacturing Example 184

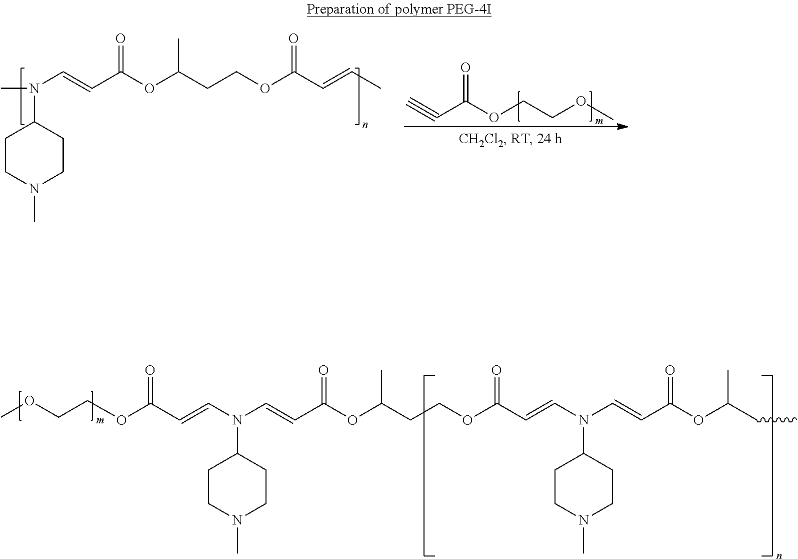

Polymer 4I prepared in the same manner as in Manufacturing Example 74 above and 438.3 mg of PEG-alkynoate were dissolved in 4 mL of CH₂Cl₂ and added to a flask. The reaction was carried out at room temperature for 24 hours, and then PEG-4I was obtained.

### Manufacturing Example 185

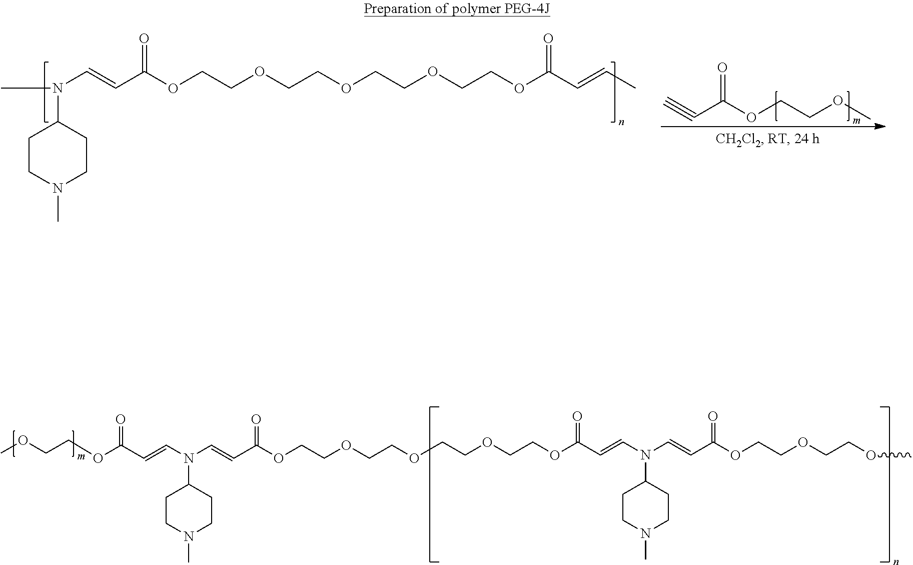

Polymer 4J prepared in the same manner as in Manufacturing Example 75 above and 285.3 mg of PEG-alkynoate were dissolved in 4 mL of CH₂Cl₂ and added to a flask. The reaction was carried out at room temperature for 24 hours, and then PEG-4J was obtained.

### Manufacturing Example 186

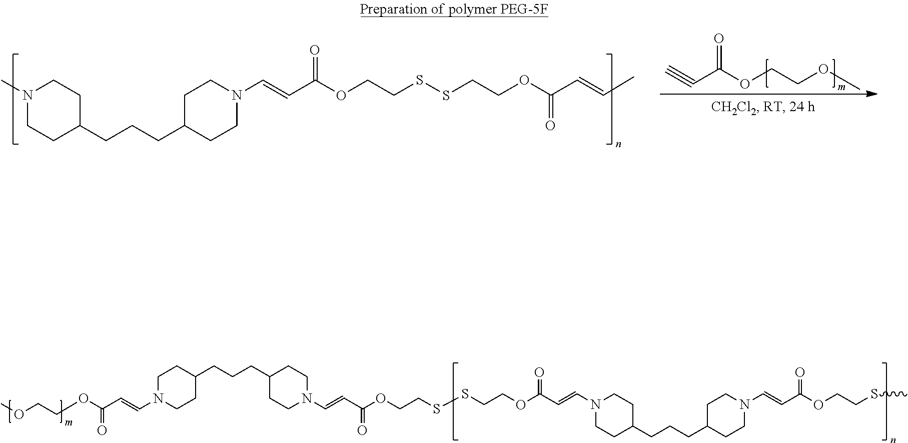

Polymer 5F prepared in the same manner as in Manufacturing Example 76 above and 329.5 mg of PEG-alkynoate were dissolved in 4 mL of CH₂Cl₂ and added to a flask. The reaction was carried out at room temperature for 24 hours, and then PEG-5F was obtained.

### Manufacturing Example 187

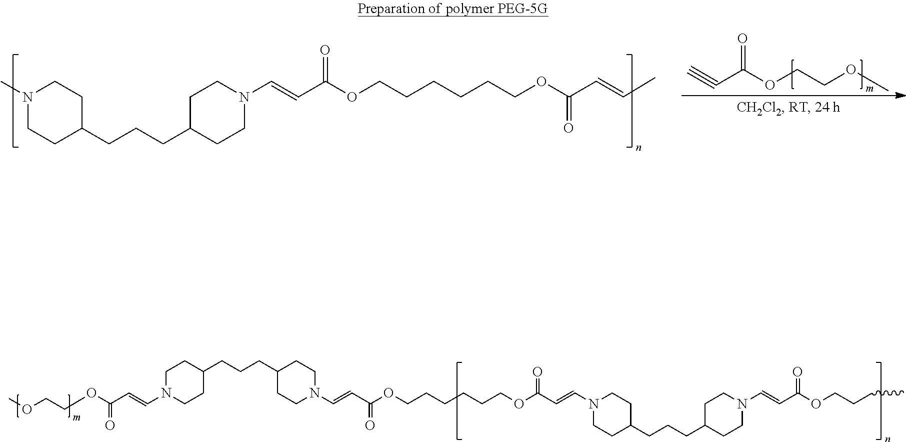

Polymer 5G prepared in the same manner as in Manufacturing Example 77 above and 383.0 mg of PEG-alkynoate were dissolved in 4 mL of CH2C12 and added to a flask. The reaction was carried out at room temperature for 24 hours, and then PEG-5G was obtained.

### Manufacturing Example 188

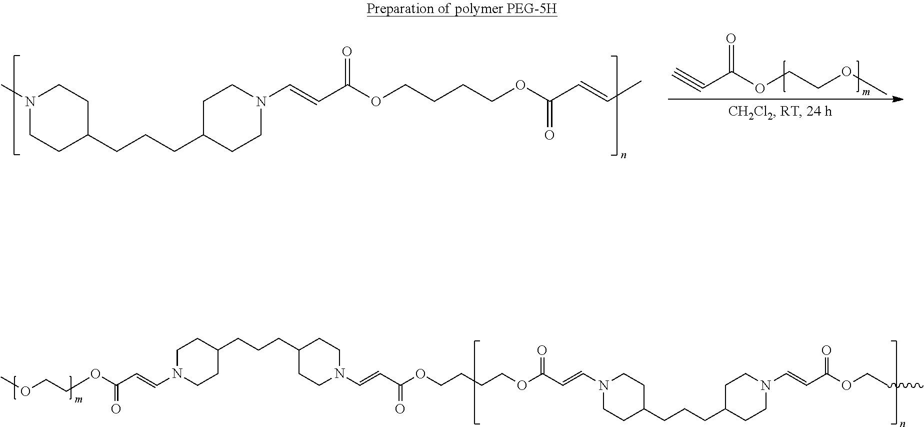

Polymer 5H prepared in the same manner as in Manufacturing Example 78 above and 438.3 mg of PEG-alkynoate were dissolved in 4 mL of CH₂Cl₂ and added to a flask. The reaction was carried out at room temperature for 24 hours, and then PEG-5H was obtained.

### Manufacturing Example 189

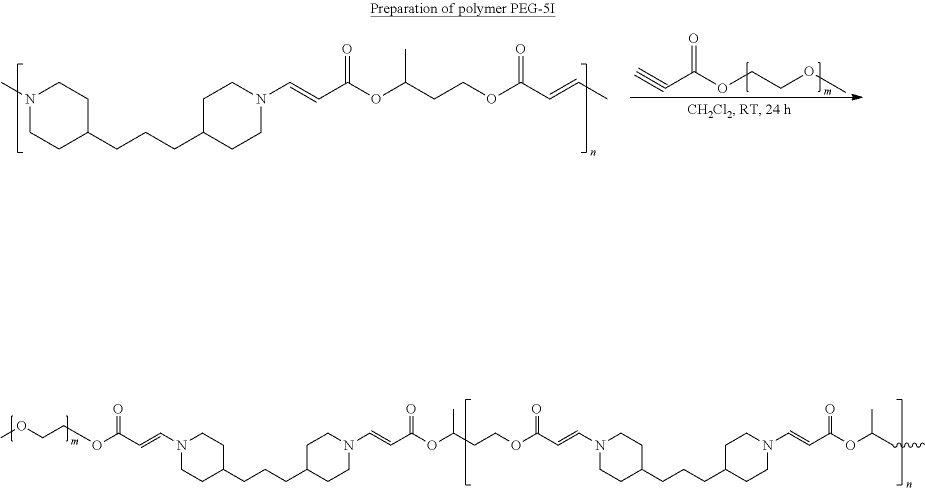

Polymer 5I prepared in the same manner as in Manufacturing Example 79 above and 438.3 mg of PEG-alkynoate were dissolved in 4 mL of CH₂Cl₂ and added to a flask. The reaction was carried out at room temperature for 24 hours, and then PEG-5I was obtained.

### Manufacturing Example 190

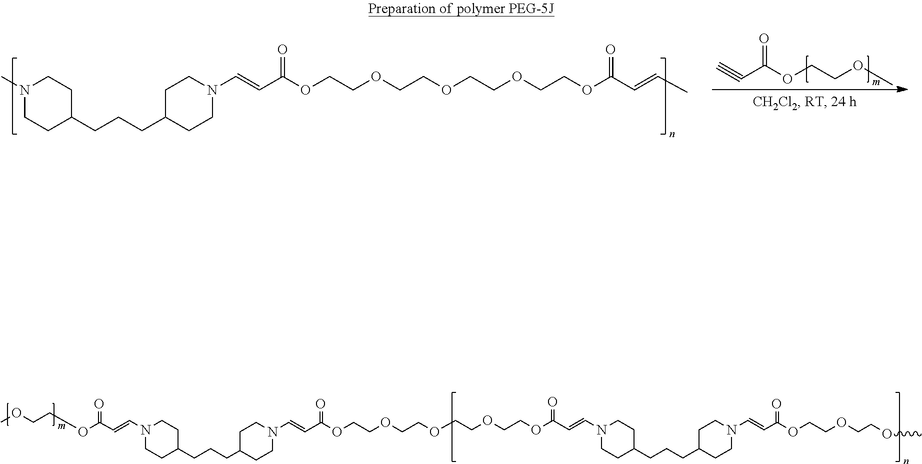

Polymer 5J prepared in the same manner as in Manufacturing Example 80 above and 285.3 mg of PEG-alkynoate were dissolved in 4 mL of CH₂Cl₂ and added to a flask. The reaction was carried out at room temperature for 24 hours, and then PEG-5J was obtained.

### Manufacturing Example 191

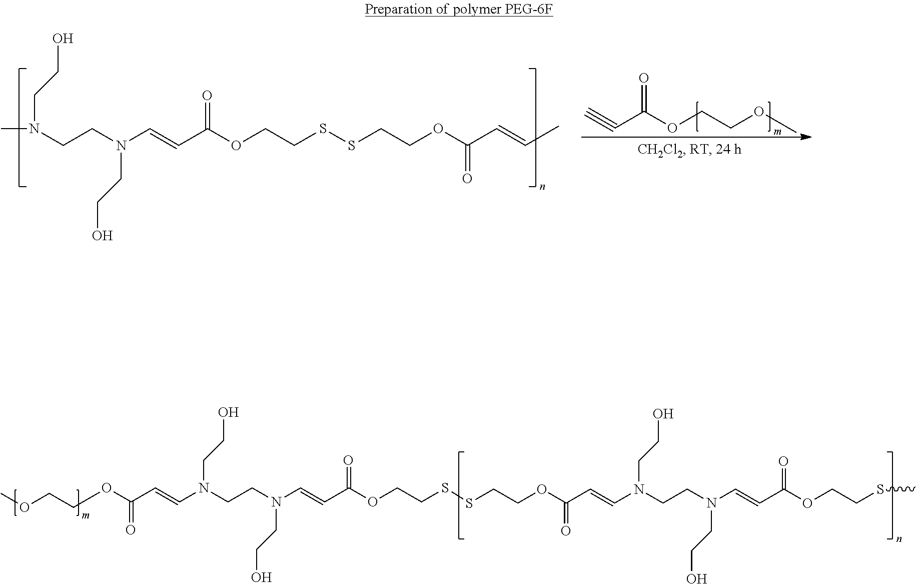

Polymer 6F prepared in the same manner as in Manufacturing Example 81 above and 329.5 mg of PEG-alkynoate were dissolved in 4 mL of CH₂Cl₂ and added to a flask. The reaction was carried out at room temperature for 24 hours, and then PEG-6F was obtained.

### Manufacturing Example 192

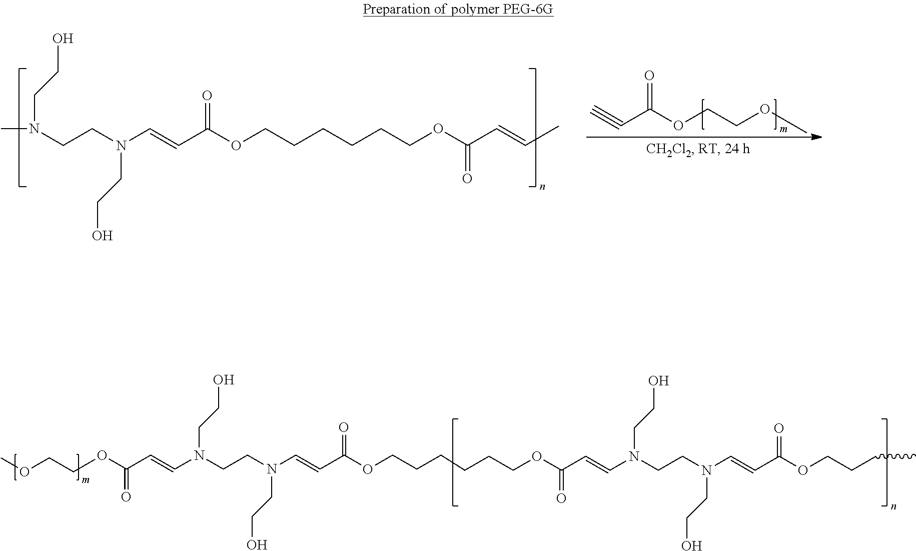

Polymer 6G prepared in the same manner as in Manufacturing Example 82 above and 383.0 mg of PEG-alkynoate were dissolved in 4 mL of CH₂Cl₂ and added to a flask. The reaction was carried out at room temperature for 24 hours, and then PEG-6G was obtained.

### Manufacturing Example 193

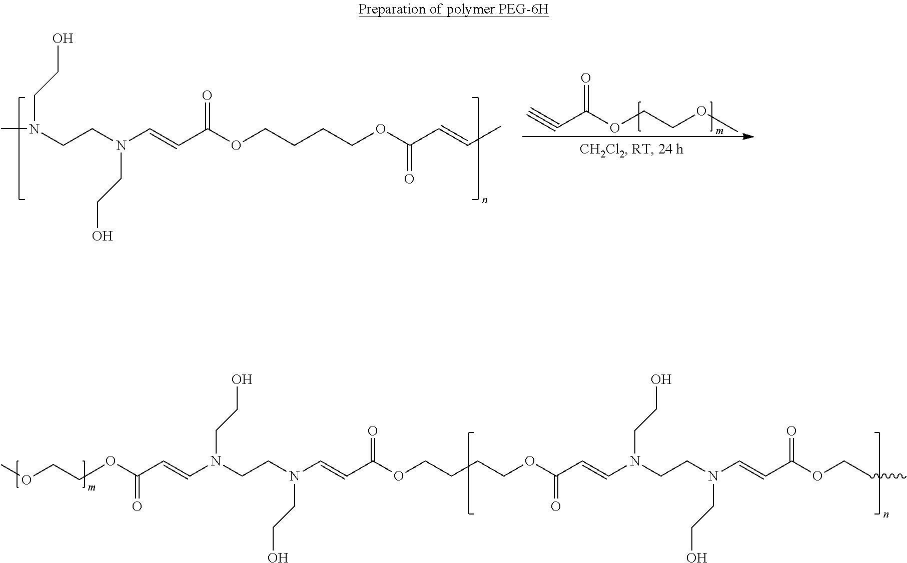

Polymer 6H prepared in the same manner as in Manufacturing Example 83 above and 438.3 mg of PEG-alkynoate were dissolved in 4 mL of CH₂Cl₂ and added to a flask. The reaction was carried out at room temperature for 24 hours, and then PEG-6H was obtained.

### Manufacturing Example 194

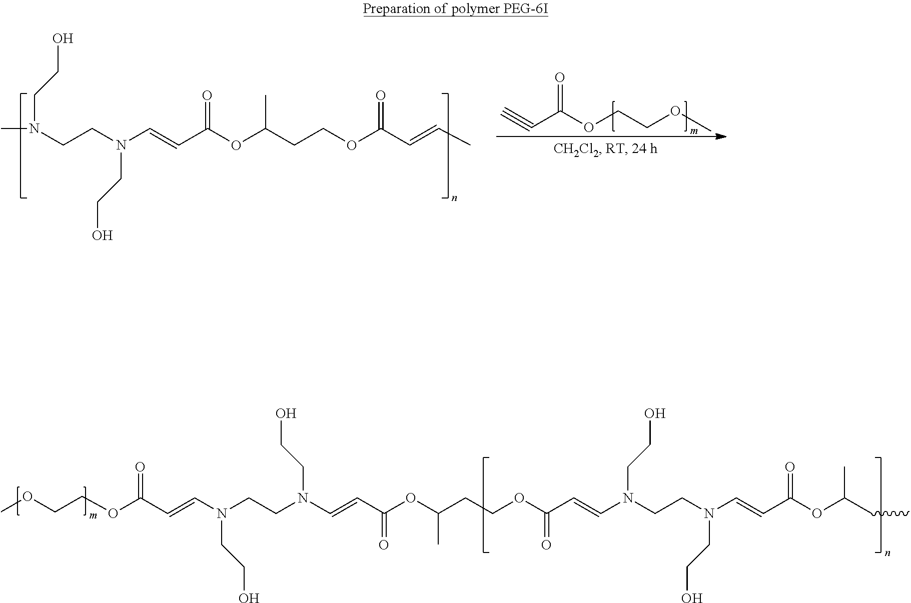

Polymer 6I prepared in the same manner as in Manufacturing Example 84 above and 438.3 mg of PEG-alkynoate were dissolved in 4 mL of CH₂Cl₂ and added to a flask. The reaction was carried out at room temperature for 24 hours, and then PEG-6I was obtained.

Manufacturing Example 195

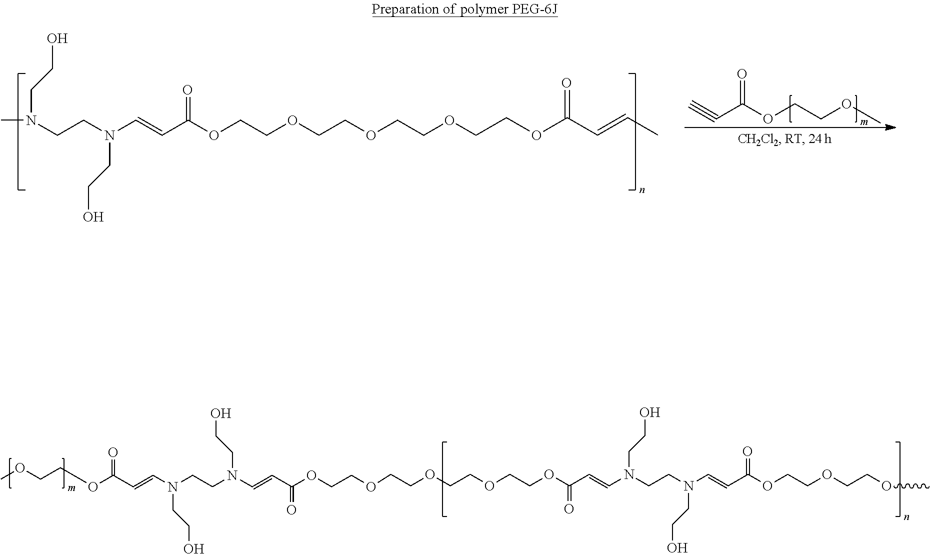

Polymer 6J prepared in the same manner as in Manufacturing Example 85 above and 285.3 mg of PEG-alkynoate were dissolved in 4 mL of CH₂Cl₂ and added to a flask. The reaction was carried out at room temperature for 24 hours, and then PEG-6J was obtained.

### Manufacturing Example 196

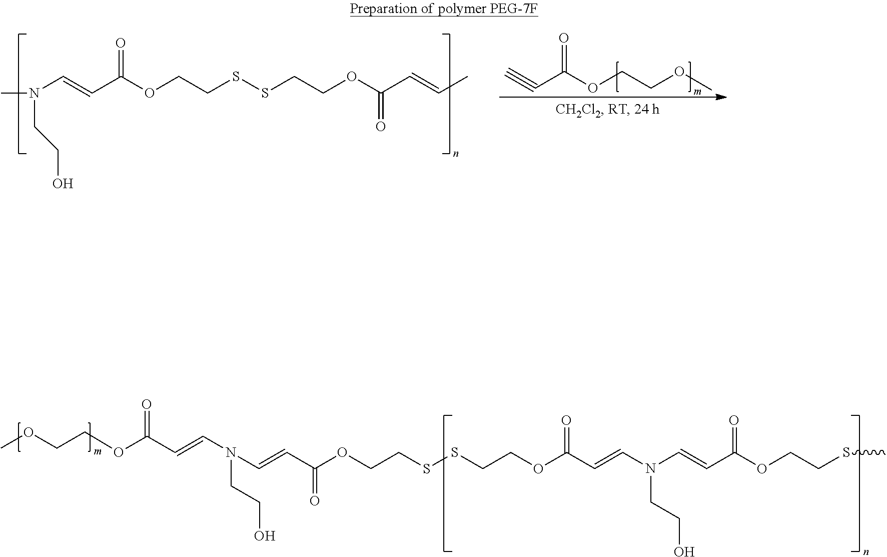

Polymer 7F prepared in the same manner as in Manufacturing Example 86 above and 329.5 mg of PEG-alkynoate were dissolved in 4 mL of CH₂Cl₂ and added to a flask. The reaction was carried out at room temperature for 24 hours, and then PEG-7F was obtained.

### Manufacturing Example 197

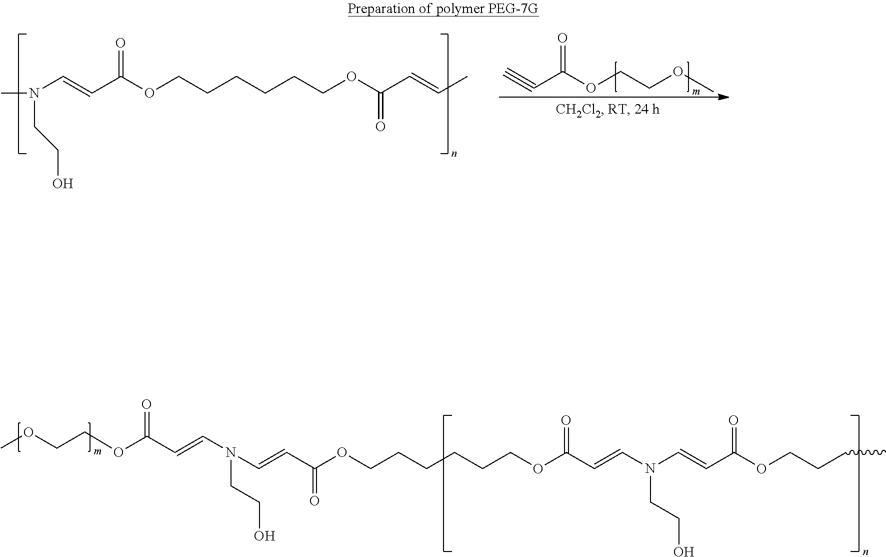

Polymer 7G prepared in the same manner as in Manufacturing Example 87 above and 383.0 mg of PEG-alkynoate were dissolved in 4 mL of CH₂Cl₂ and added to a flask. The reaction was carried out at room temperature for 24 hours, and then PEG-7G was obtained.

### Manufacturing Example 198

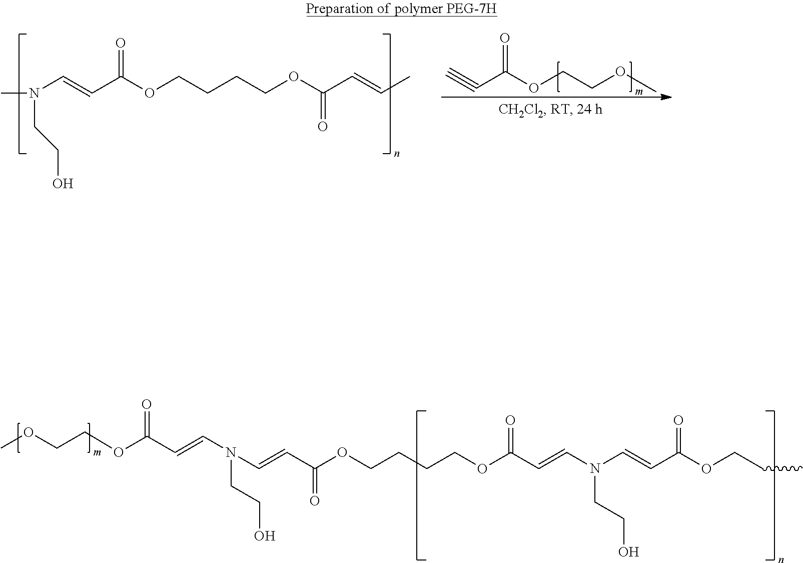

Polymer 7H prepared in the same manner as in Manufacturing Example 88 above and 438.3 mg of PEG-alkynoate were dissolved in 4 mL of CH₂Cl₂ and added to a flask. The reaction was carried out at room temperature for 24 hours, and then PEG-7H was obtained.

### Manufacturing Example 199

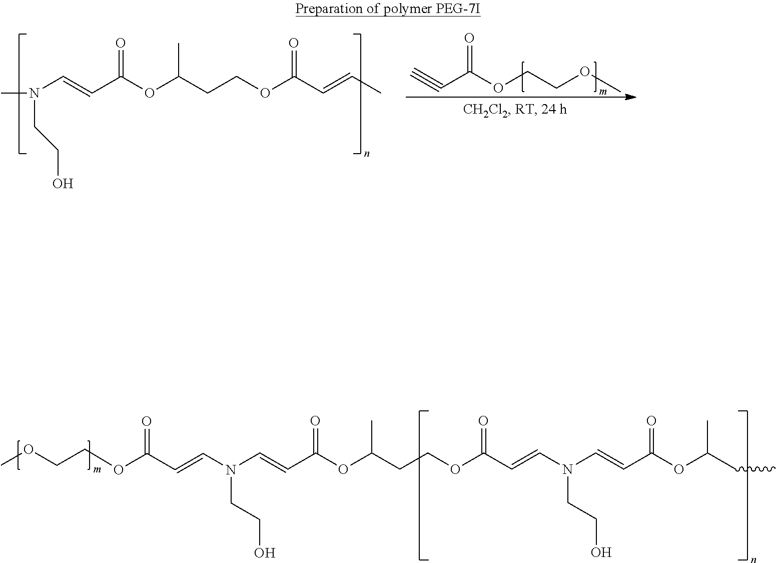

Polymer 7I prepared in the same manner as in Manufacturing Example 89 above and 438.3 mg of PEG-alkynoate were dissolved in 4 mL of CH₂Cl₂ and added to a flask. The reaction was carried out at room temperature for 24 hours, and then PEG-7I was obtained.

### Manufacturing Example 200

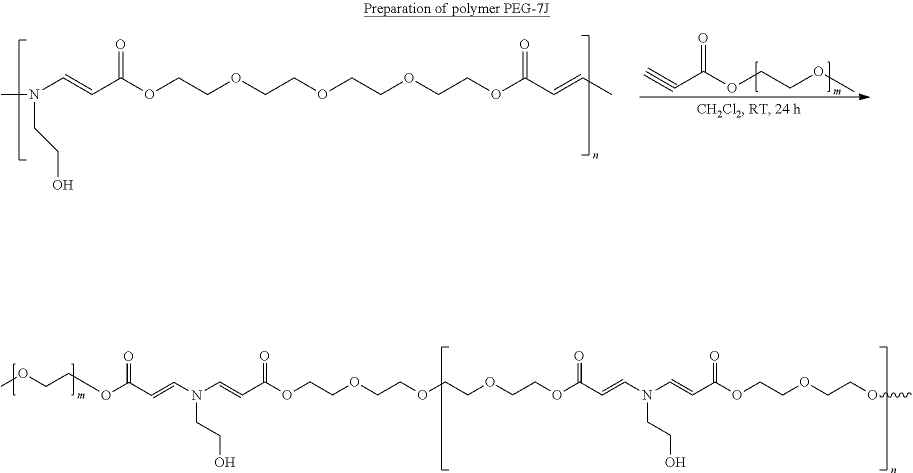

Polymer 7J prepared in the same manner as in Manufacturing Example 90 above and 285.3 mg of PEG-alkynoate were dissolved in 4 mL of CH₂Cl₂ and added to a flask. The reaction was carried out at room temperature for 24 hours, and then PEG-7J was obtained.

### Manufacturing Example 201

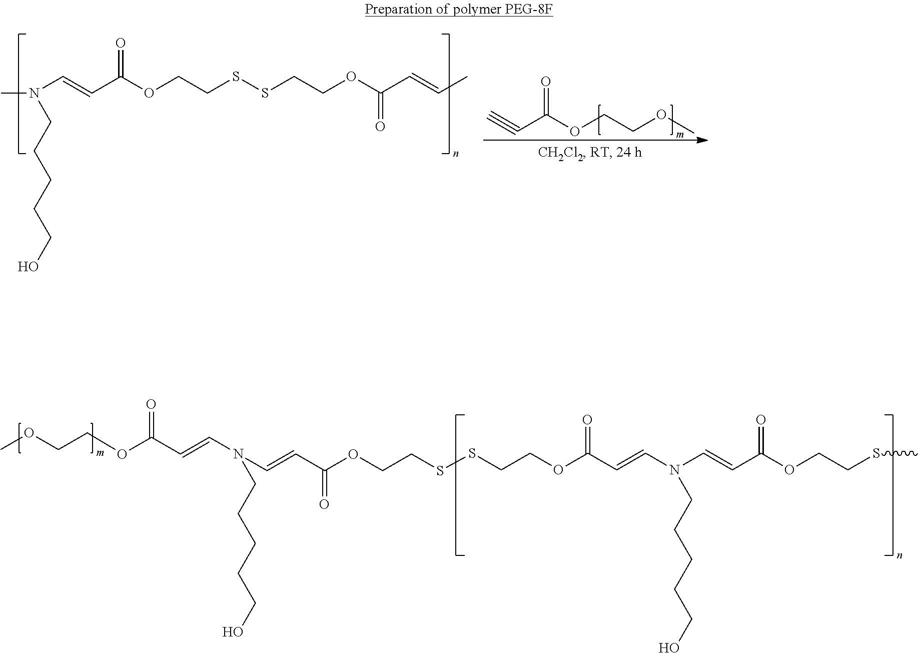

Polymer 8F prepared in the same manner as in Manufacturing Example 91 above and 329.5 mg of PEG-alkynoate were dissolved in 4 mL of CH₂Cl₂ and added to a flask. The reaction was carried out at room temperature for 24 hours, and then PEG-8F was obtained.

### Manufacturing Example 202

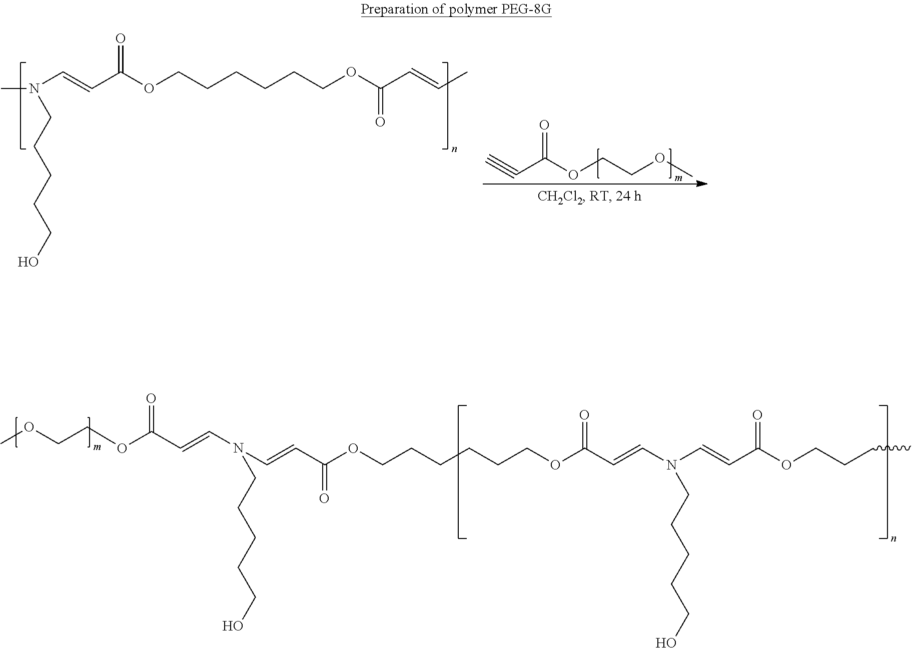

Polymer 8G prepared in the same manner as in Manufacturing Example 92 above and 383.0 mg of PEG-alkynoate were dissolved in 4 mL of CH₂Cl₂ and added to a flask. The reaction was carried out at room temperature for 24 hours, and then PEG-8G was obtained.

### Manufacturing Example 203

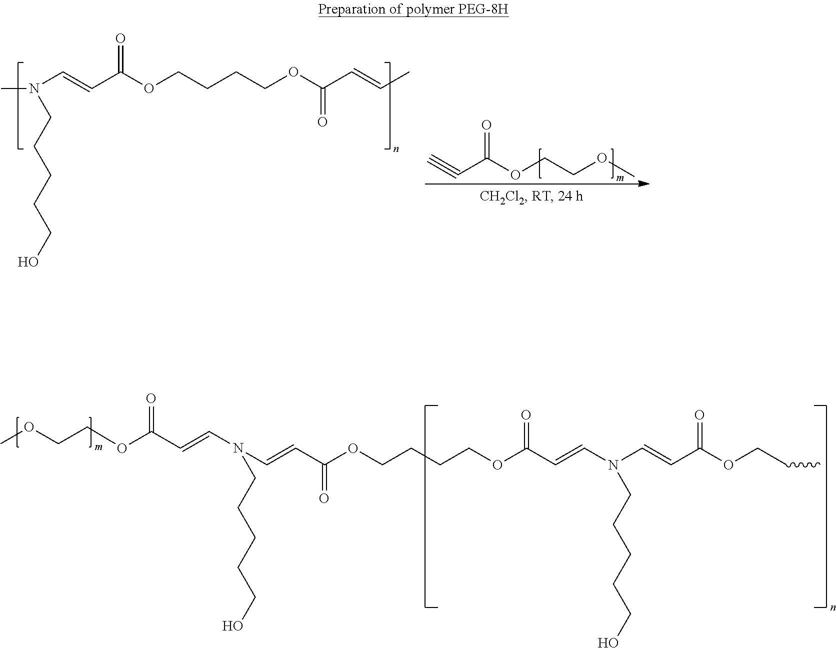

Polymer 8H prepared in the same manner as in Manufacturing Example 93 above and 438.3 mg of PEG-alkynoate were dissolved in 4 mL of CH₂Cl₂ and added to a flask. The reaction was carried out at room temperature for 24 hours, and then PEG-8H was obtained.

### Manufacturing Example 204

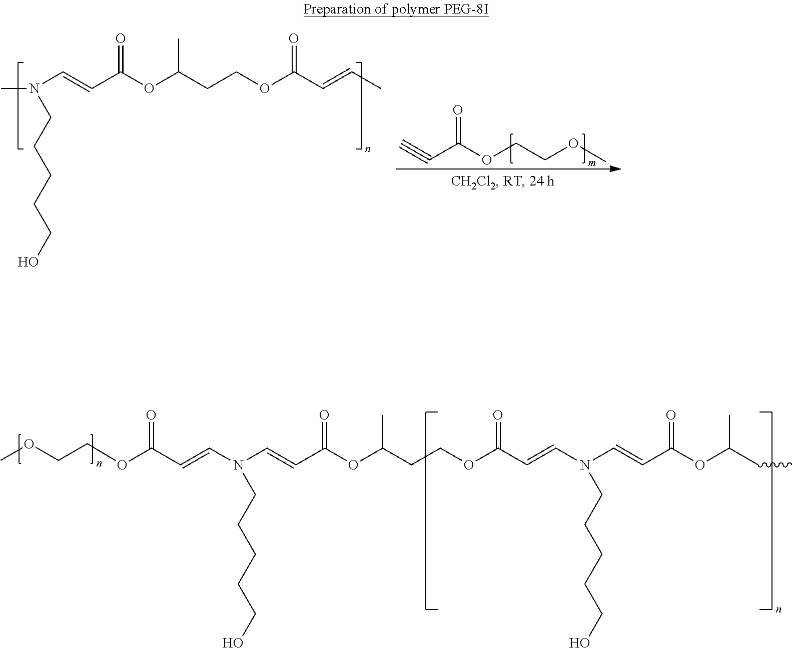

Polymer 8I prepared in the same manner as in Manufacturing Example 94 above and 438.3 mg of PEG-alkynoate were dissolved in 4 mL of CH₂Cl₂ and added to a flask. The reaction was carried out at room temperature for 24 hours, and then PEG-8I was obtained.

### Manufacturing Example 205

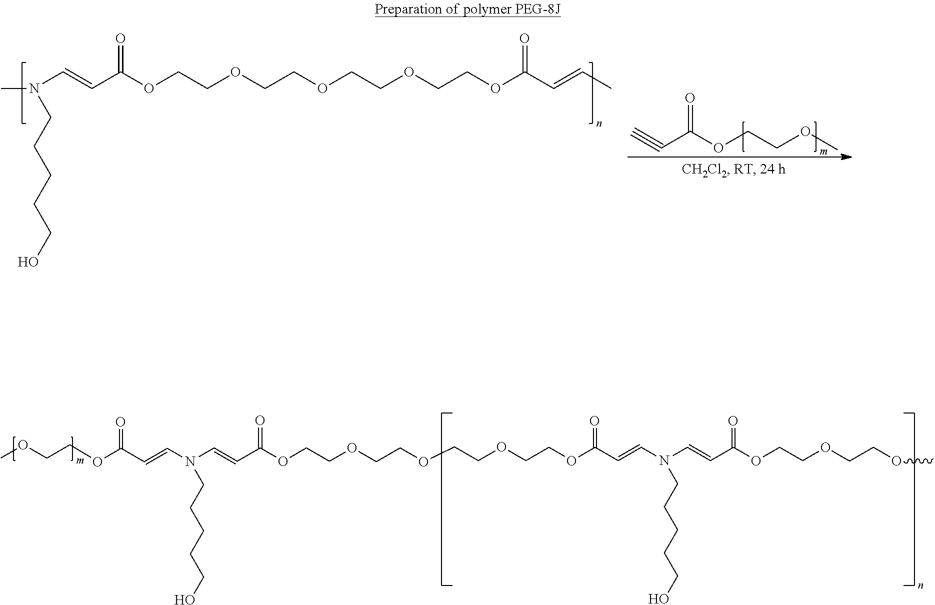

Polymer 8J prepared in the same manner as in Manufacturing Example 95 above and 285.3 mg of PEG-alkynoate were dissolved in 4 mL of CH₂Cl₂ and added to a flask. The reaction was carried out at room temperature for 24 hours, and then PEG-8J was obtained.

### Manufacturing Example 206

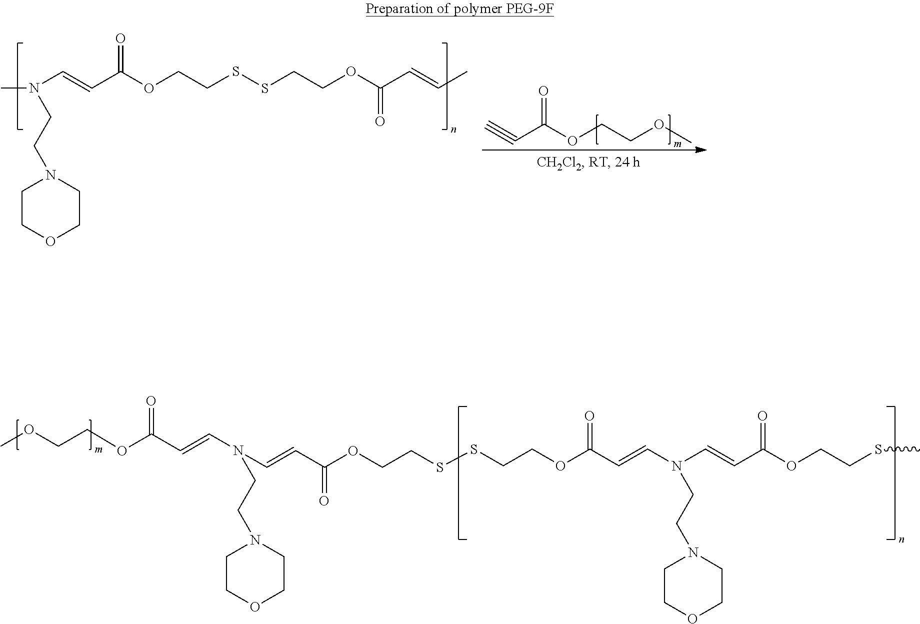

Polymer 9F prepared in the same manner as in Manufacturing Example 96 above and 329.5 mg of PEG-alkynoate were dissolved in 4 mL of CH₂Cl₂ and added to a flask. The reaction was carried out at room temperature for 24 hours, and then PEG-9F was obtained.

### Manufacturing Example 207

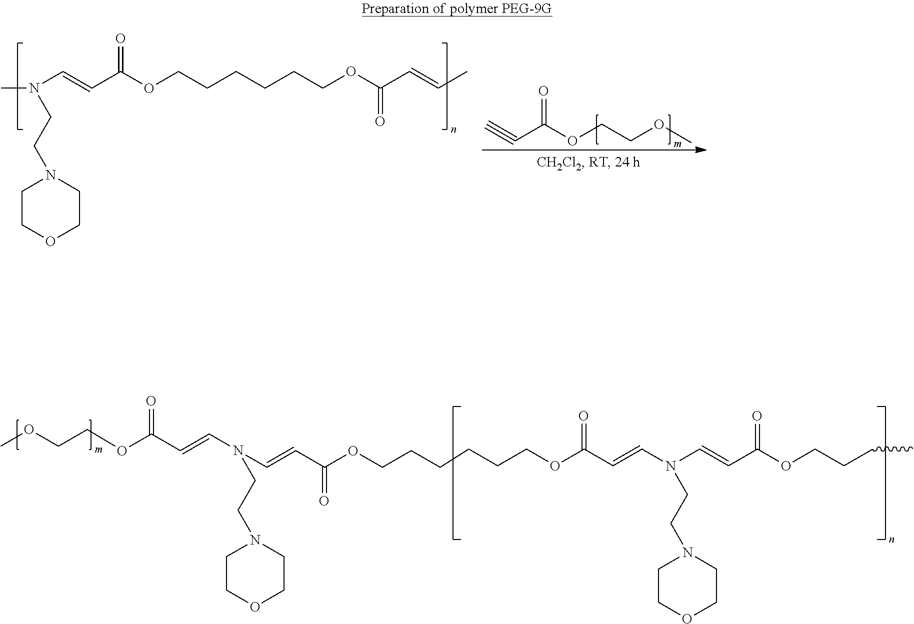

Polymer 9G prepared in the same manner as in Manufacturing Example 97 above and 383.0 mg of PEG-alkynoate were dissolved in 4 mL of CH₂Cl₂ and added to a flask. The reaction was carried out at room temperature for 24 hours, and then PEG-9G was obtained.

### Manufacturing Example 208

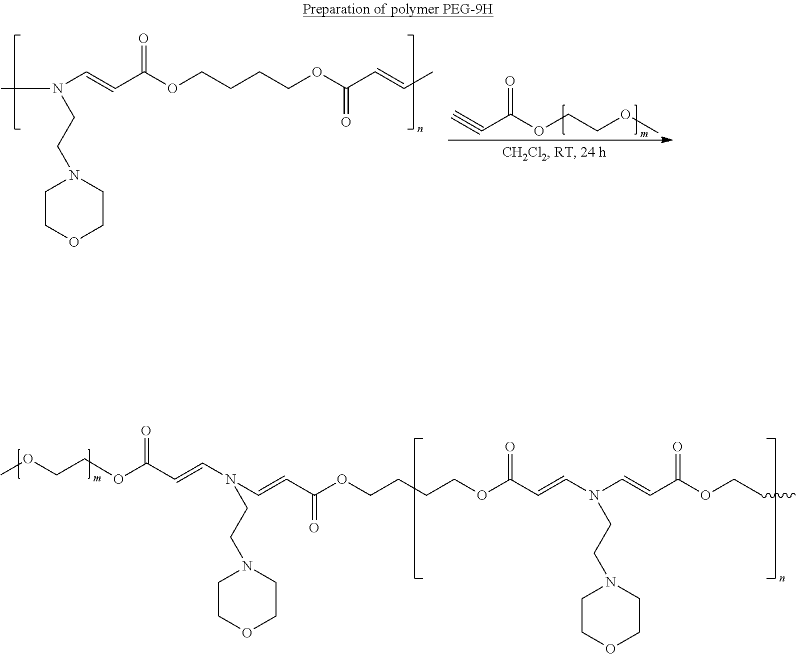

Polymer 9H prepared in the same manner as in Manufacturing Example 98 above and 438.3 mg of PEG-alkynoate were dissolved in 4 mL of CH₂Cl₂ and added to a flask. The reaction was carried out at room temperature for 24 hours, and then PEG-9H was obtained.

### Manufacturing Example 209

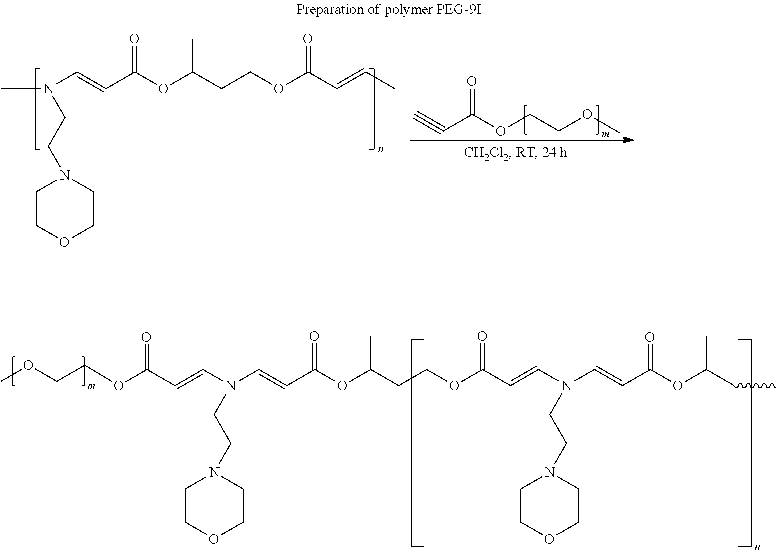

Polymer 9I prepared in the same manner as in Manufacturing Example 99 above and 438.3 mg of PEG-alkynoate were dissolved in 4 mL of CH₂Cl₂ and added to a flask. The reaction was carried out at room temperature for 24 hours, and then PEG-9I was obtained.

### Manufacturing Example 210

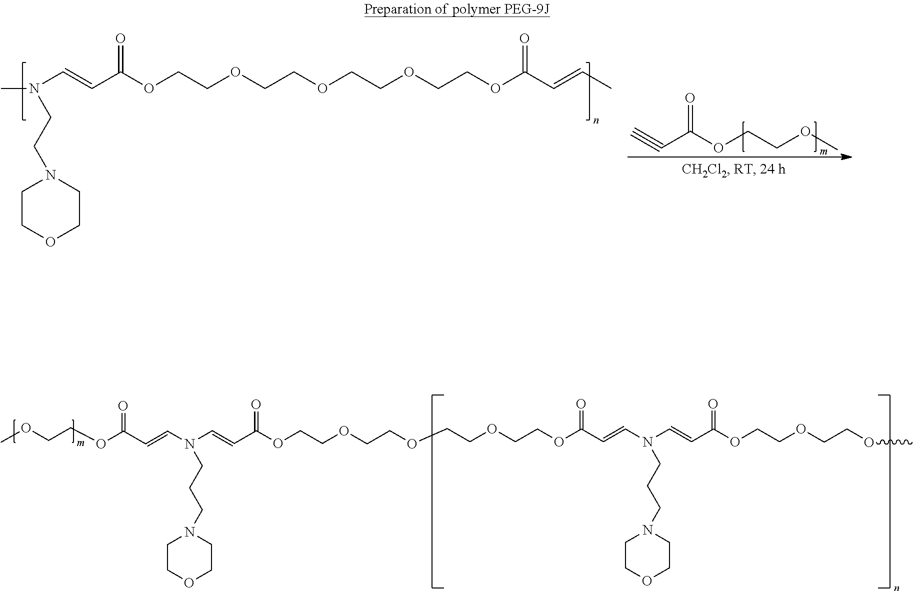

Polymer 9J prepared in the same manner as in Manufacturing Example 100 above and 285.3 mg of PEG-alkynoate were dissolved in 4 mL of CH₂Cl₂ and added to a flask. The reaction was carried out at room temperature for 24 hours, and then PEG-9J was obtained.

### Manufacturing Example 211

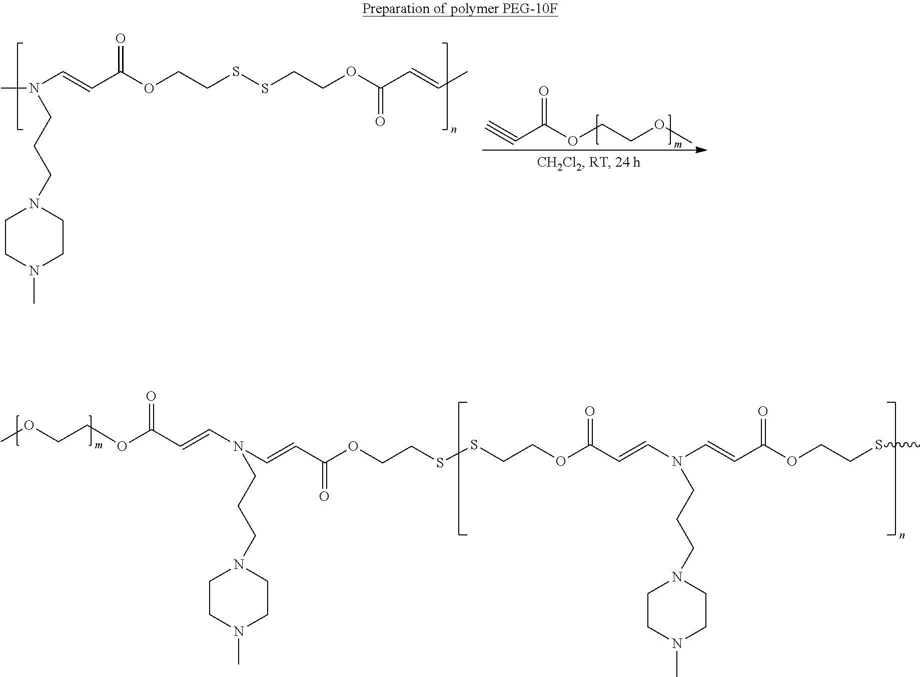

Polymer 10F prepared in the same manner as in Manufacturing Example 101 above and 329.5 mg of PEG-alkynoate were dissolved in 4 mL of CH₂Cl₂ and added to a flask. The reaction was carried out at room temperature for 24 hours, and then PEG-10F was obtained.

### Manufacturing Example 212

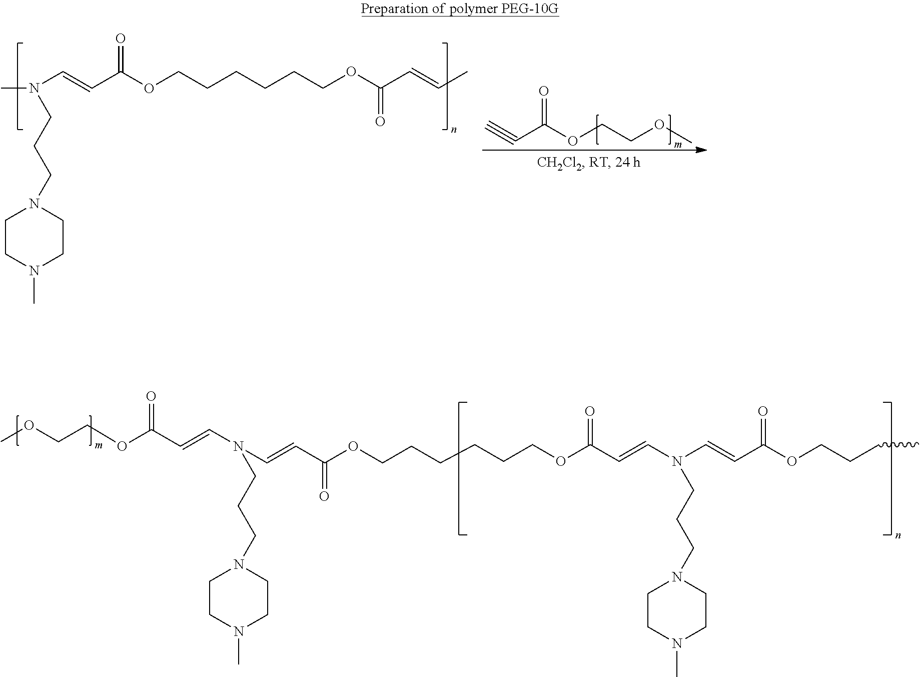

Polymer 10G prepared in the same manner as in Manufacturing Example 102 above and 383.0 mg of PEG-alkynoate were dissolved in 4 mL of CH₂Cl₂ and added to a flask. The reaction was carried out at room temperature for 24 hours, and then PEG-10G was obtained.

### Manufacturing Example 213

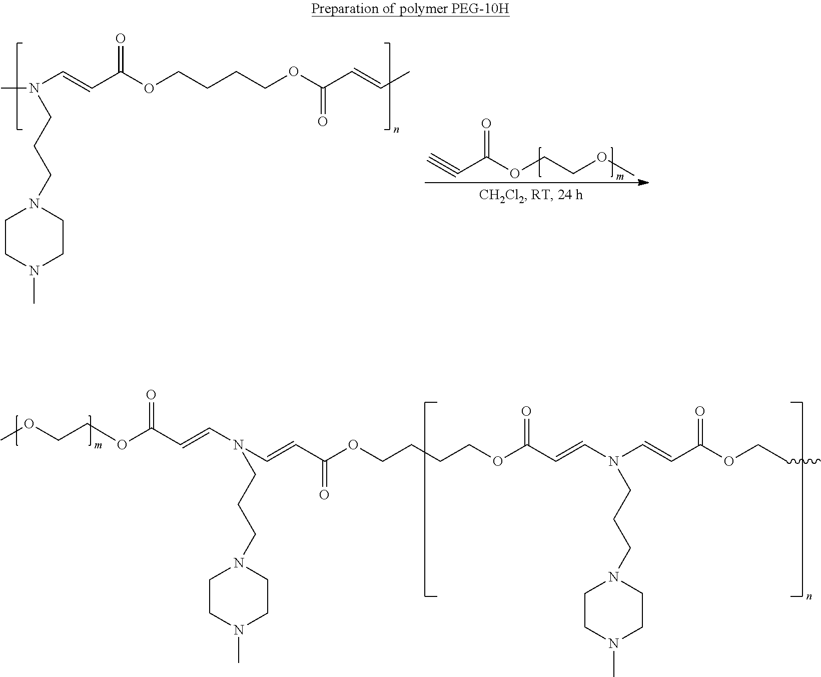

Polymer 10H prepared in the same manner as in Manufacturing Example 103 above and 438.3 mg of PEG-alkynoate were dissolved in 4 mL of CH₂Cl₂ and added to a flask. The reaction was carried out at room temperature for 24 hours, and then PEG-10H was obtained.

### Manufacturing Example 214

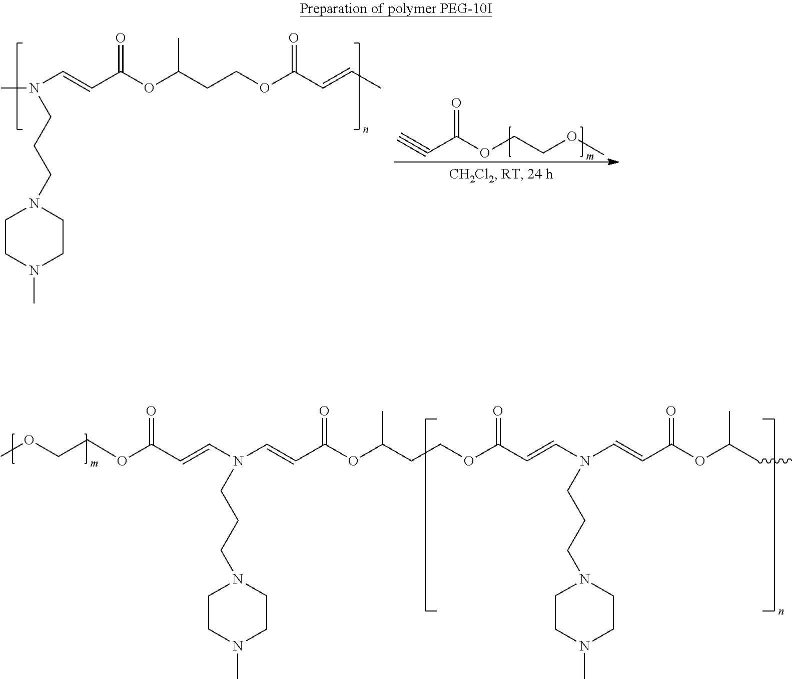

Polymer 10I prepared in the same manner as in Manufacturing Example 104 above and 438.3 mg of PEG-alkynoate were dissolved in 4 mL of CH₂Cl₂ and added to a flask. The reaction was carried out at room temperature for 24 hours, and then PEG-10I was obtained.

### Manufacturing Example 215

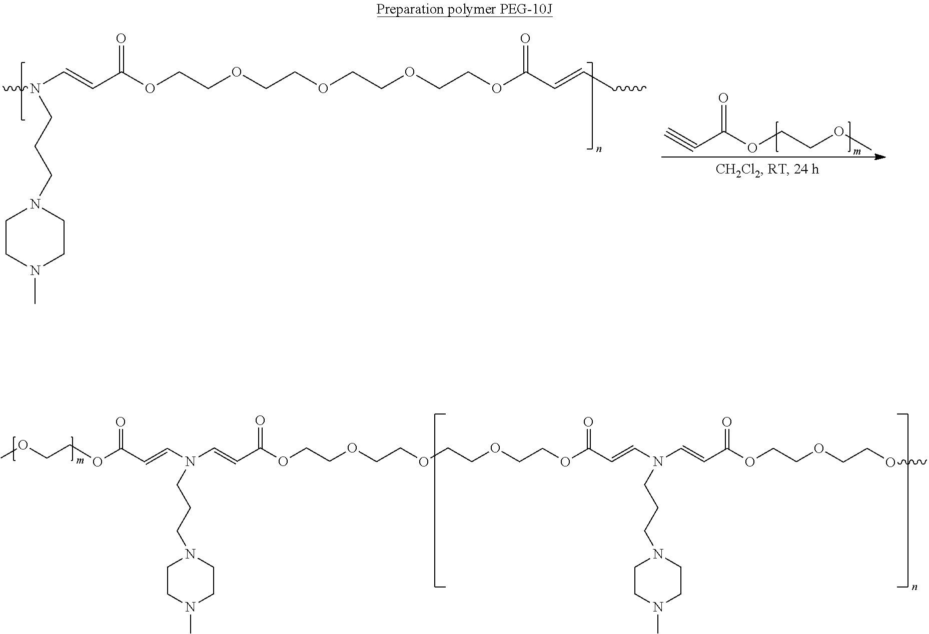

Polymer 10J prepared in the same manner as in Manufacturing Example 105 above and 285.3 mg of PEG-alkynoate were dissolved in 4 mL of CH₂Cl₂ and added to a flask. The reaction was carried out at room temperature for 24 hours, and then PEG-10J was obtained.

### Manufacturing Example 216

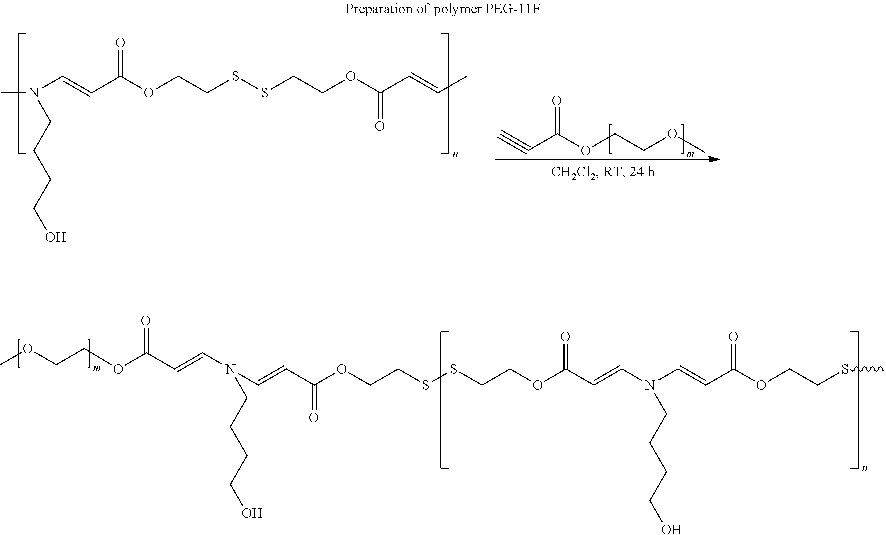

Polymer 11F prepared in the same manner as in Manufacturing Example 106 above and 329.5 mg of PEG-alkynoate were dissolved in 4 mL of CH₂Cl₂ and added to a flask. The reaction was carried out at room temperature for 24 hours, and then PEG-11F was obtained.

### Manufacturing Example 217

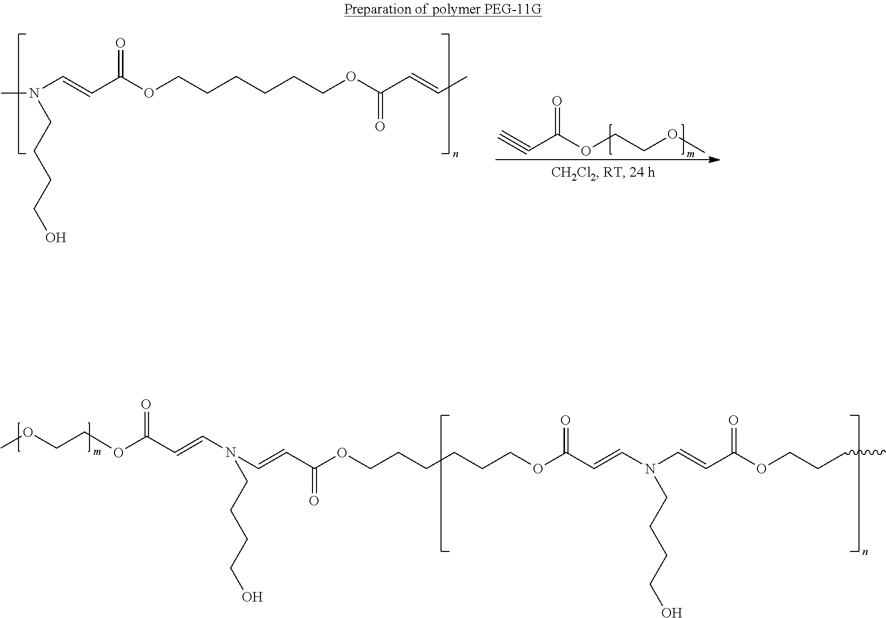

Polymer 11G prepared in the same manner as in Manufacturing Example 107 above and 383.0 mg of PEG-alkynoate were dissolved in 4 mL of CH₂Cl₂ and added to a flask. The reaction was carried out at room temperature for 24 hours, and then PEG-11G was obtained.

### Manufacturing Example 218

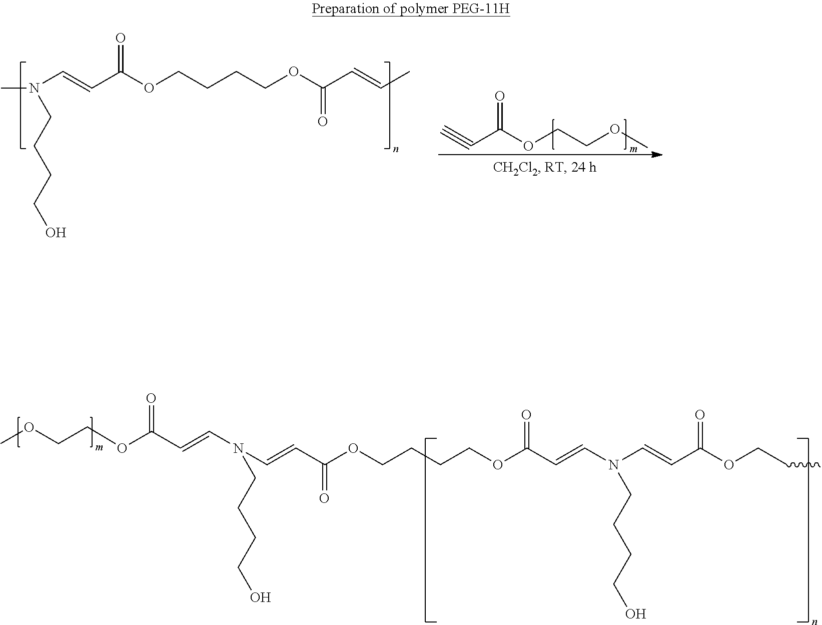

Polymer 11H prepared in the same manner as in Manufacturing Example 108 above and 438.3 mg of PEG-alkynoate were dissolved in 4 mL of CH₂Cl₂ and added to a flask. The reaction was carried out at room temperature for 24 hours, and then PEG-11H was obtained.

### Manufacturing Example 219

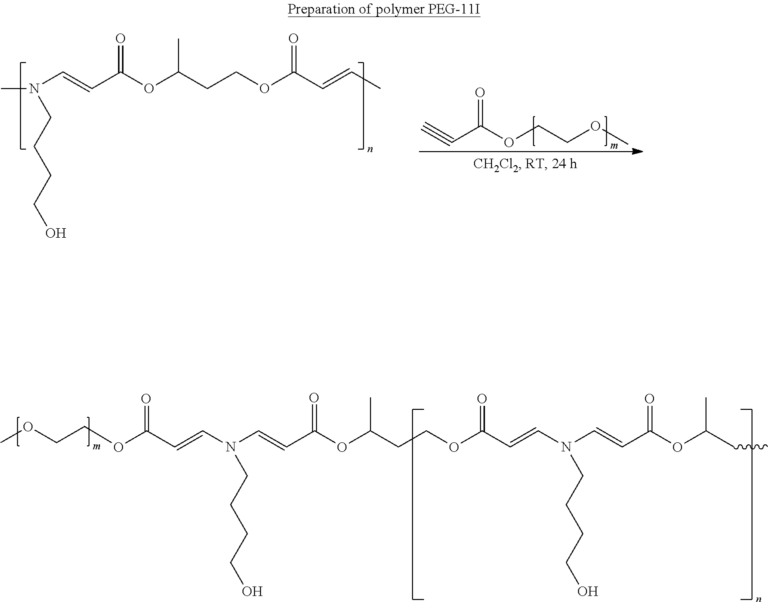

Polymer 11I prepared in the same manner as in Manufacturing Example 109 above and 438.3 mg of PEG-alkynoate were dissolved in 4 mL of CH₂Cl₂ and added to a flask. The reaction was carried out at room temperature for 24 hours, and then PEG-11I was obtained.

### Manufacturing Example 220

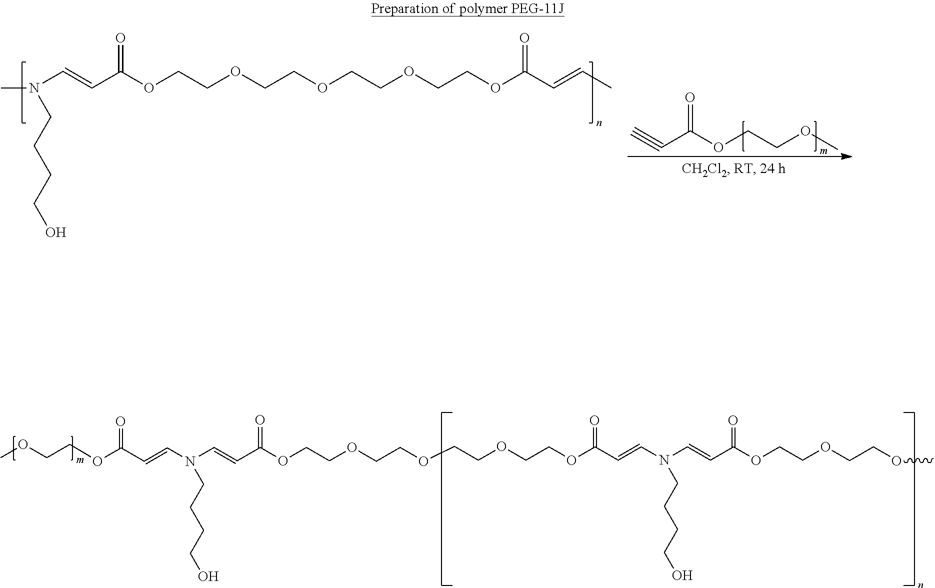

Polymer 11J prepared in the same manner as in Manufacturing Example 110 above and 285.3 mg of PEG-alkynoate were dissolved in 4 mL of CH₂Cl₂ and added to a flask. The reaction was carried out at room temperature for 24 hours, and then PEG-11J was obtained.

### Manufacturing Example 221

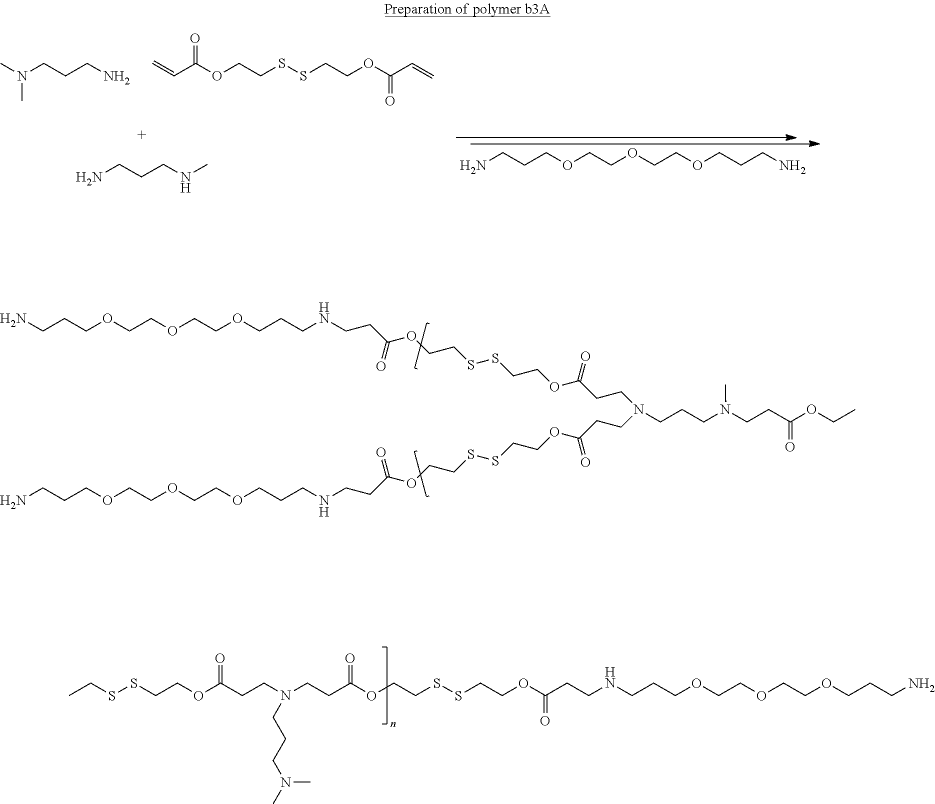

Amine monomer 3, acrylate monomer A, and branched dendrimer were added to a round bottom flask in a molar ratio of 0.4:1:0.2. Thereafter, dimethylformamide (N,N-dimethylformamide, DMF) was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b3A was obtained. [symbol] used in the reaction scheme in the present specification is a notation that is well known to those of ordinary skill in the art, indicating that a multi-step reaction occurs and that intermediates are omitted.

### Manufacturing Example 222

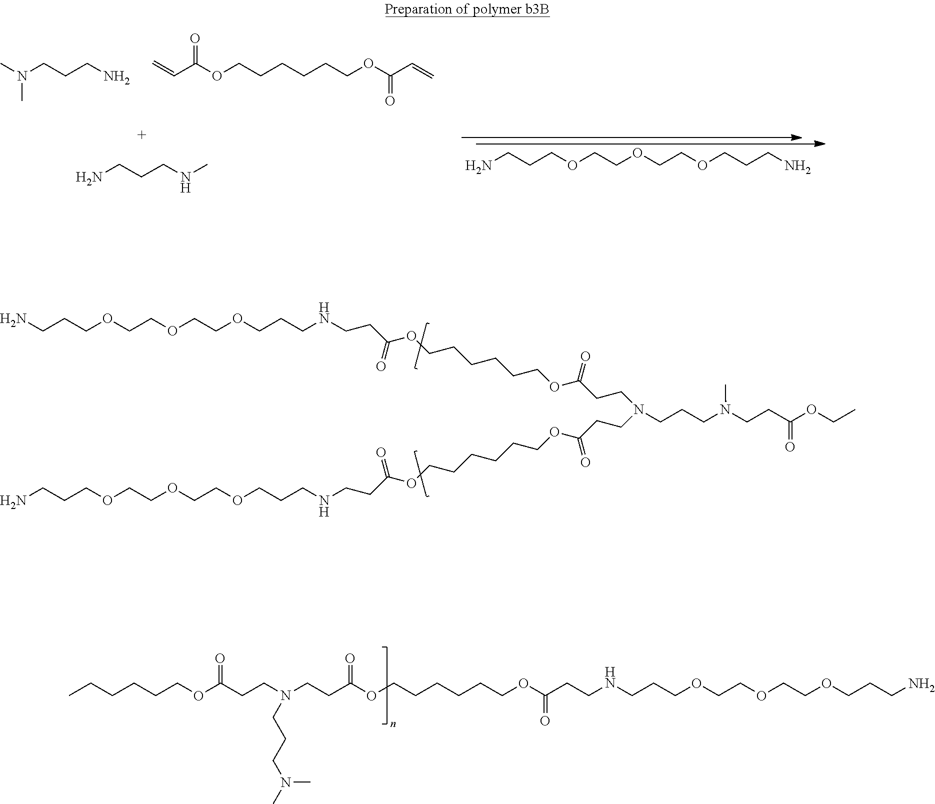

Amine monomer 3, acrylate monomer B, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b3B was obtained.

### Manufacturing Example 223

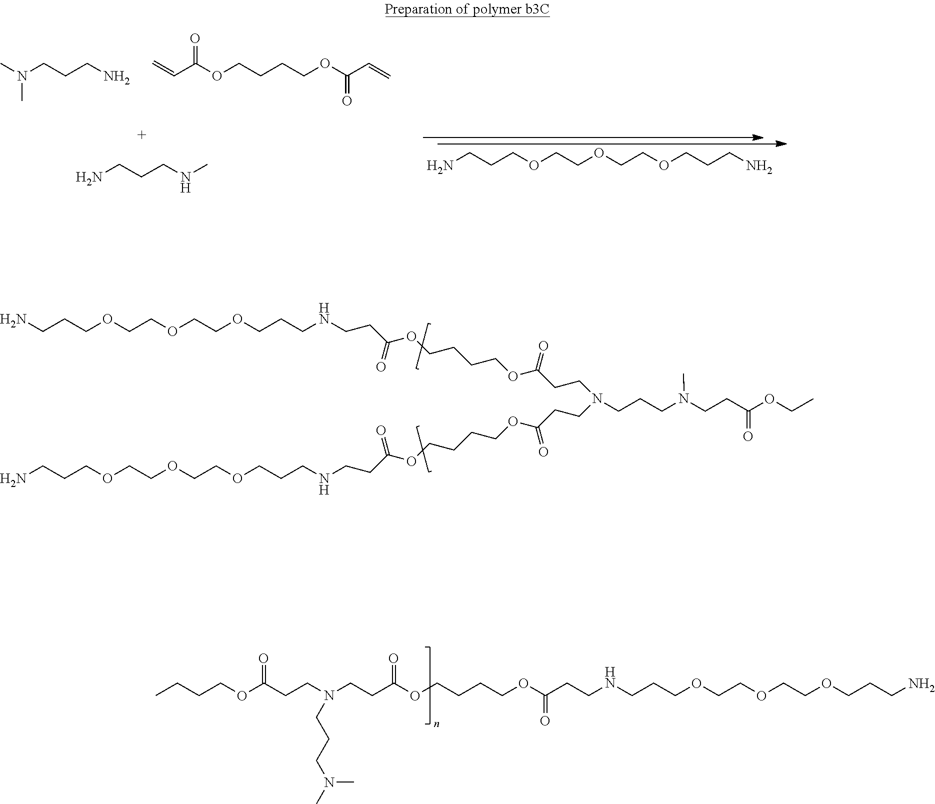

Amine monomer 3, acrylate monomer C, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b3C was obtained.

### Manufacturing Example 224

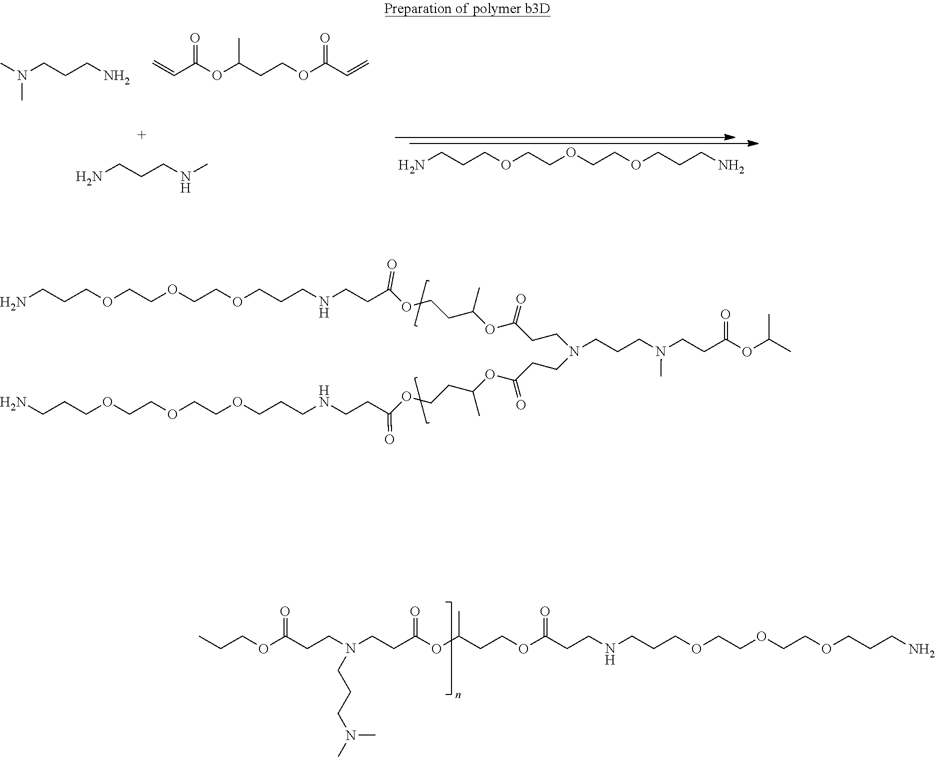

Amine monomer 3, acrylate monomer D, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b3D was obtained.

### Manufacturing Example 225

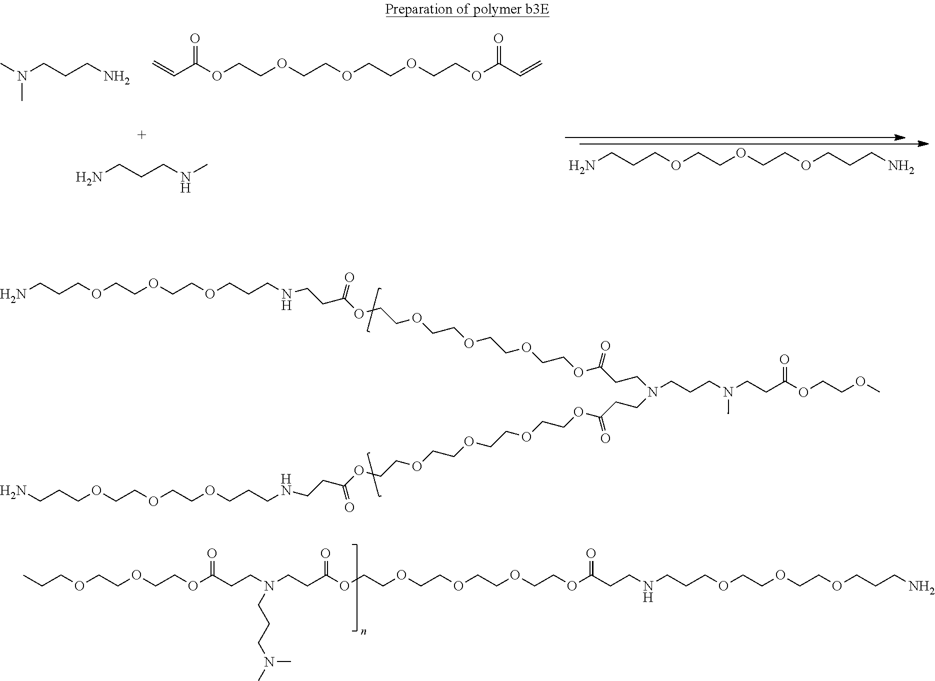

Amine monomer 3, acrylate monomer E, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b3E was obtained.

### Manufacturing Example 226

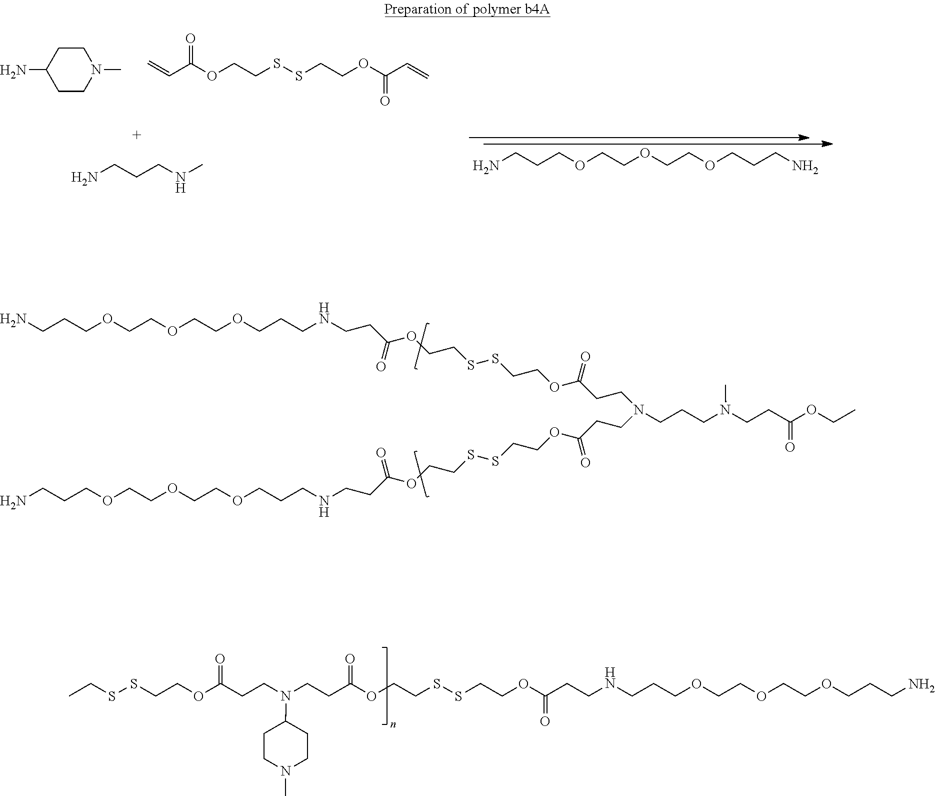

Amine monomer 4, acrylate monomer A, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b4A was obtained.

### Manufacturing Example 227

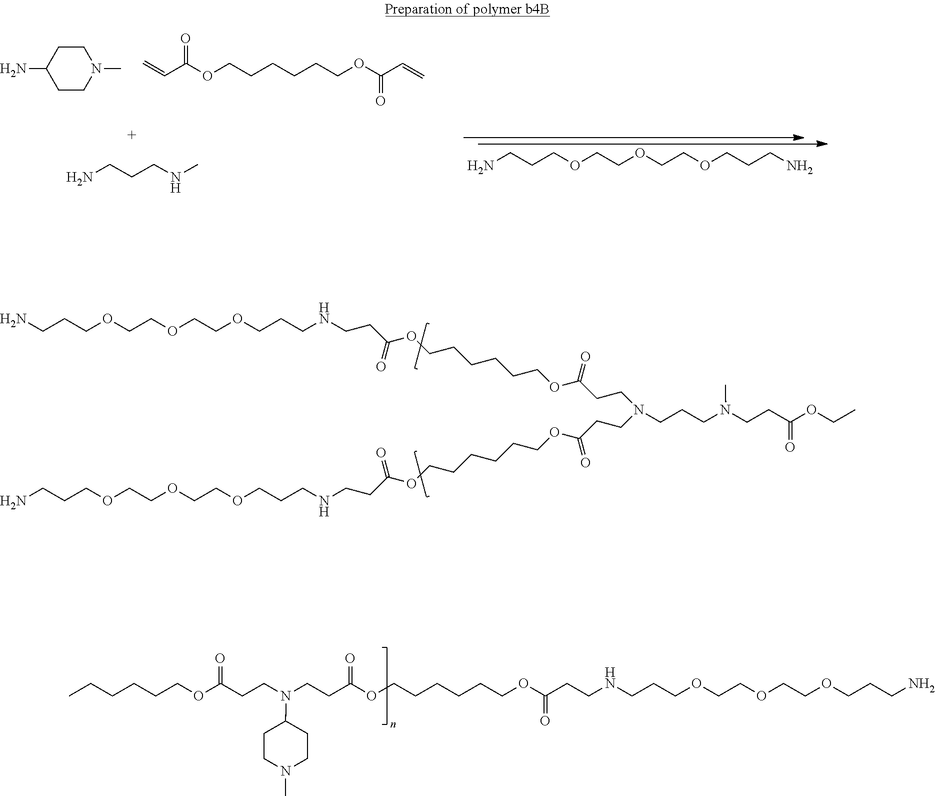

Amine monomer 4, acrylate monomer B, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b4B was obtained.

### Manufacturing Example 228

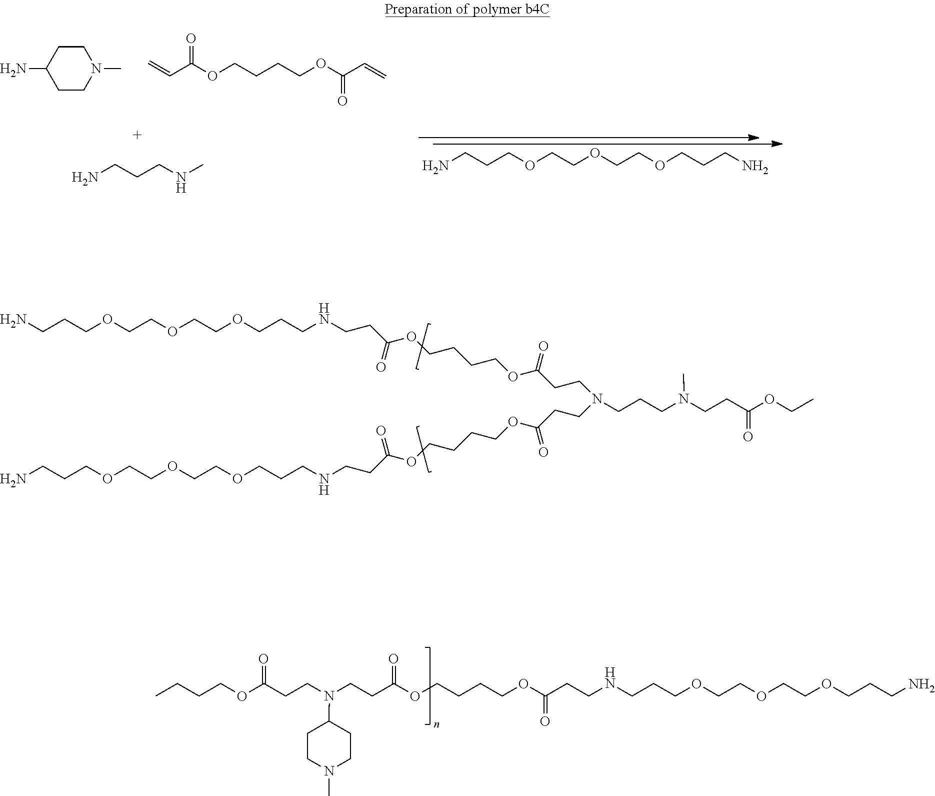

Amine monomer 4, acrylate monomer C, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b4C was obtained.

### Manufacturing Example 229

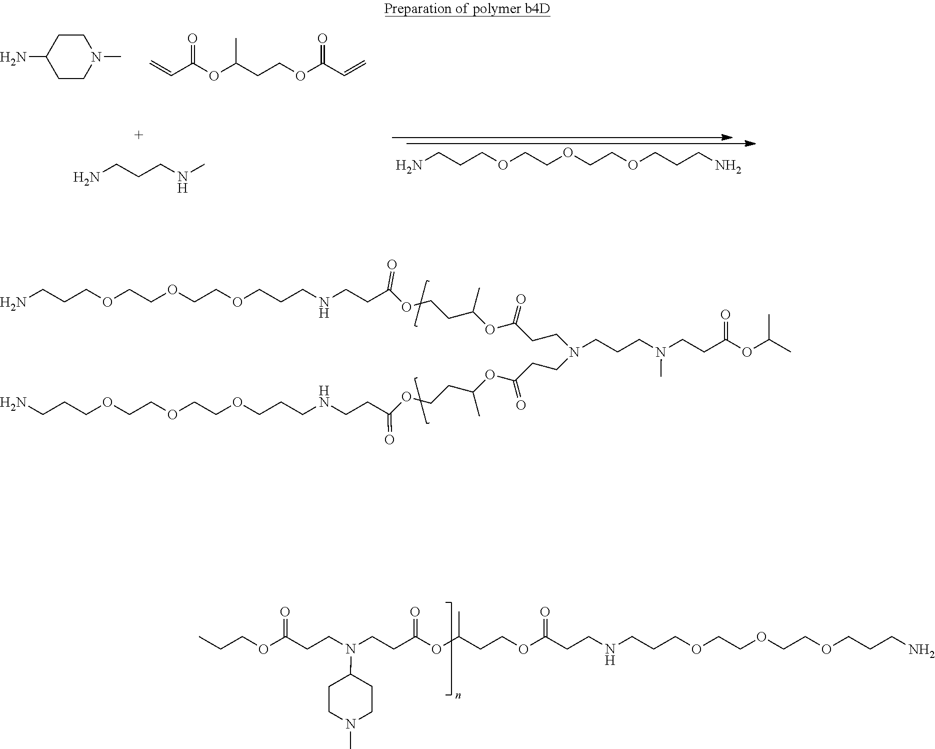

Amine monomer 4, acrylate monomer D, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b4D was obtained.

### Manufacturing Example 230

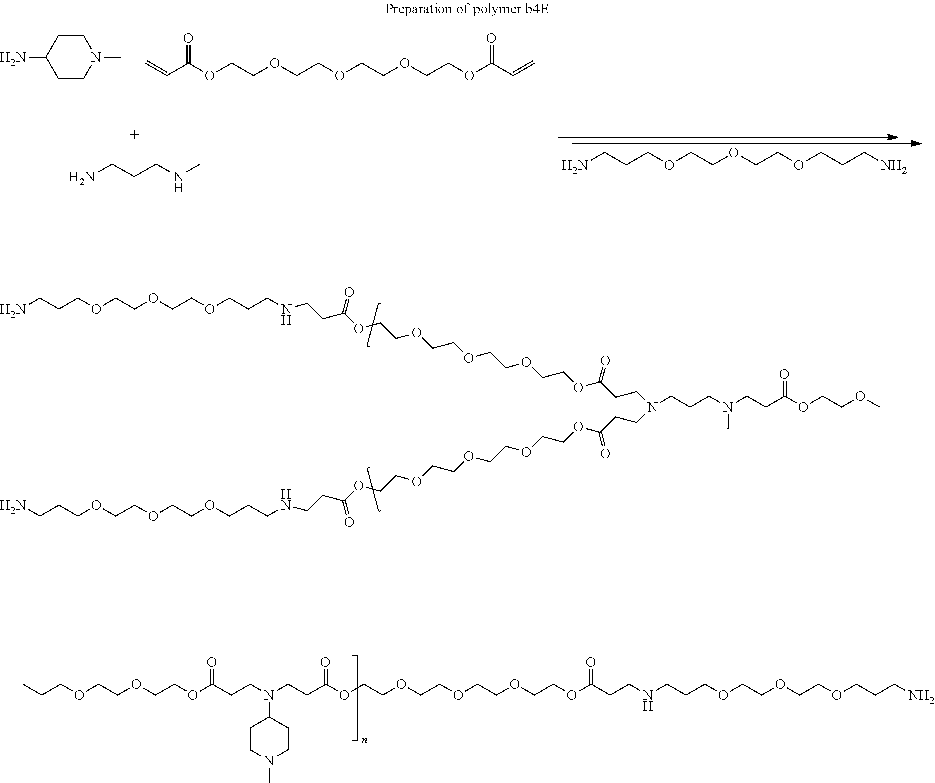

Amine monomer 4, acrylate monomer E, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b4E was obtained.

### Manufacturing Example 231

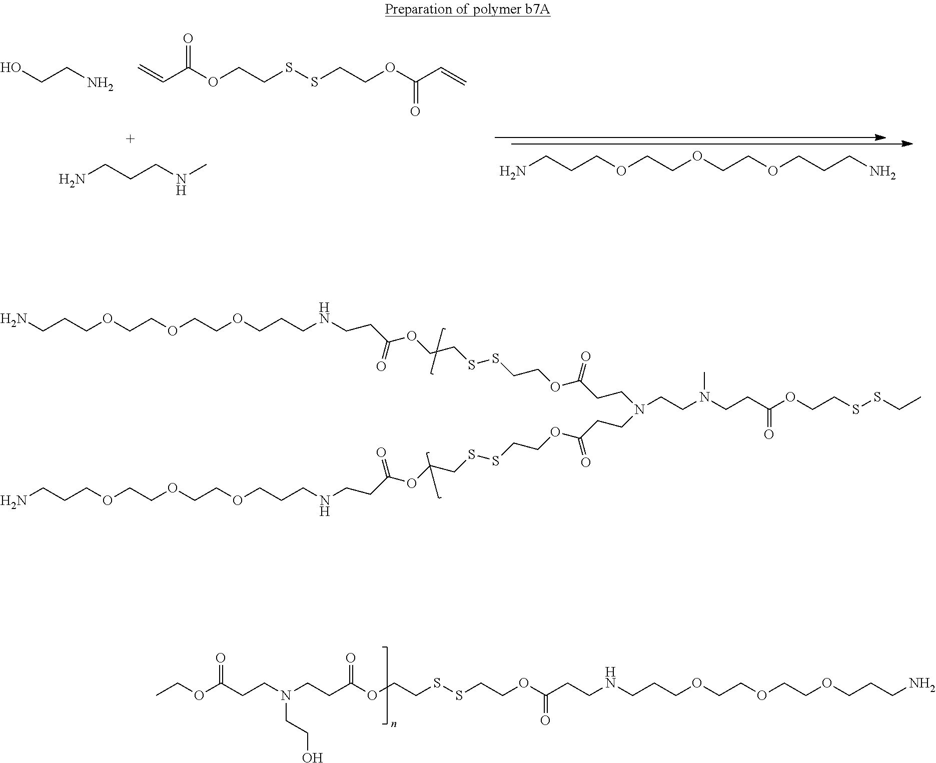

Amine monomer 7, acrylate monomer A, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b7A was obtained.

### Manufacturing Example 232

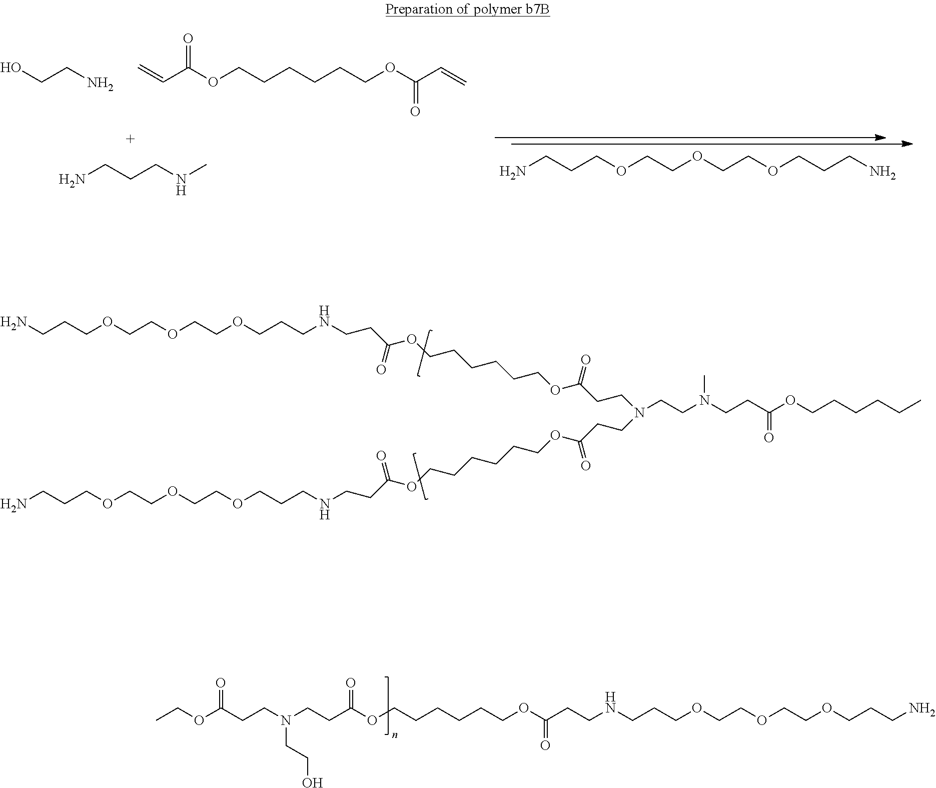

Amine monomer 7, acrylate monomer B, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b7B was obtained.

### Manufacturing Example 233

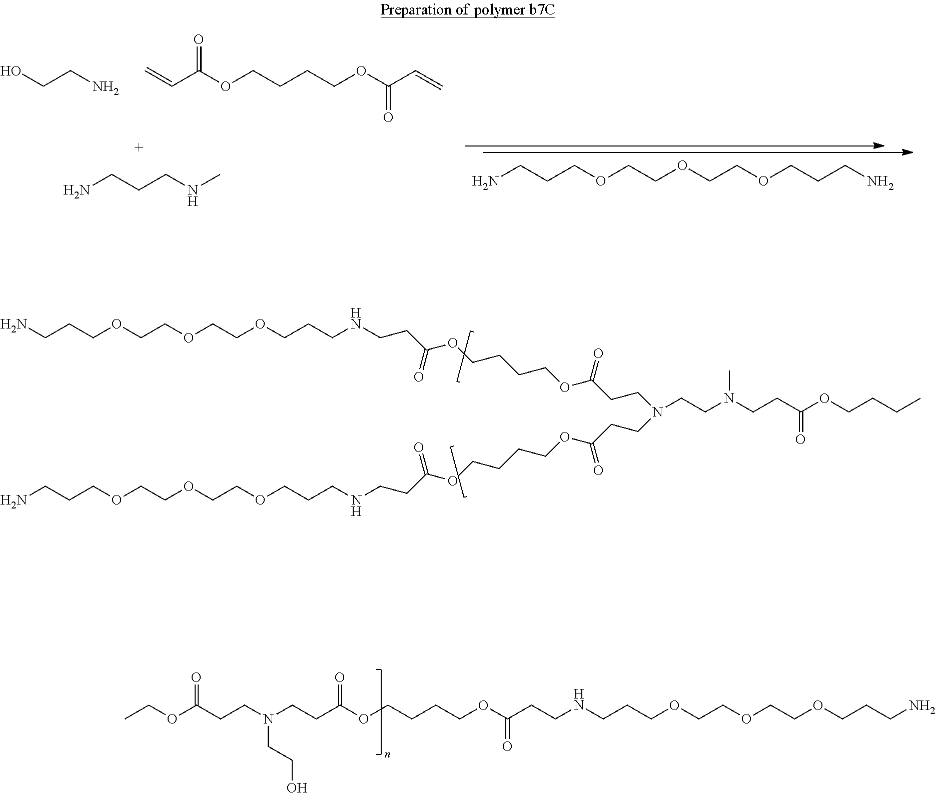

Amine monomer 7, acrylate monomer C, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b7C was obtained.

### Manufacturing Example 234

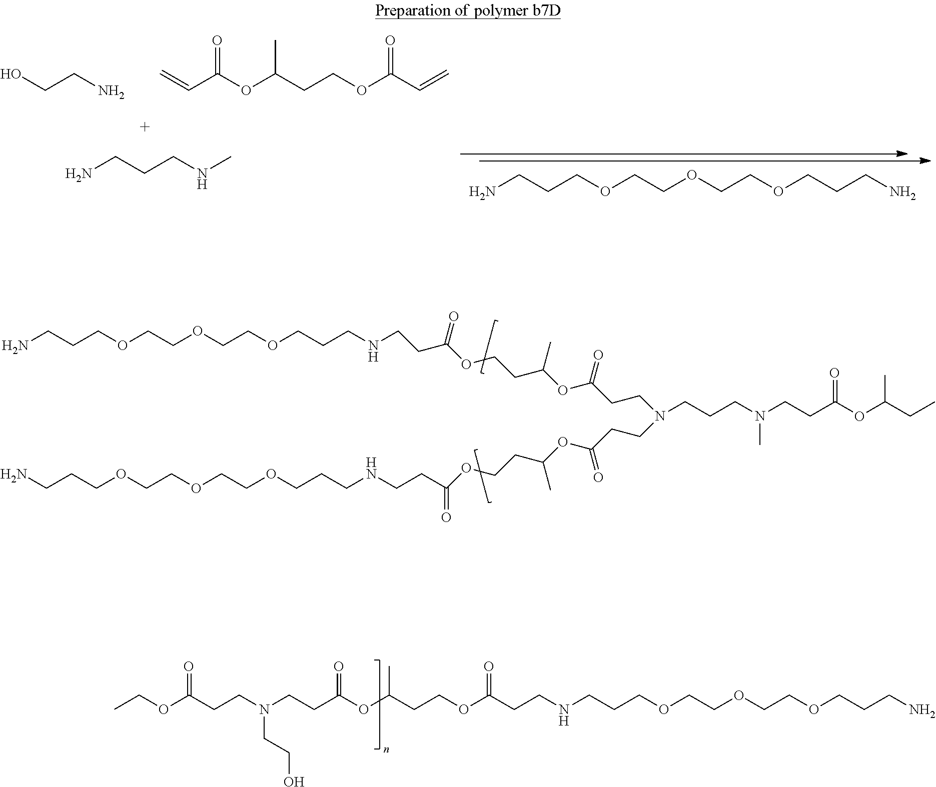

Amine monomer 7, acrylate monomer D, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b7D was obtained.

### Manufacturing Example 235

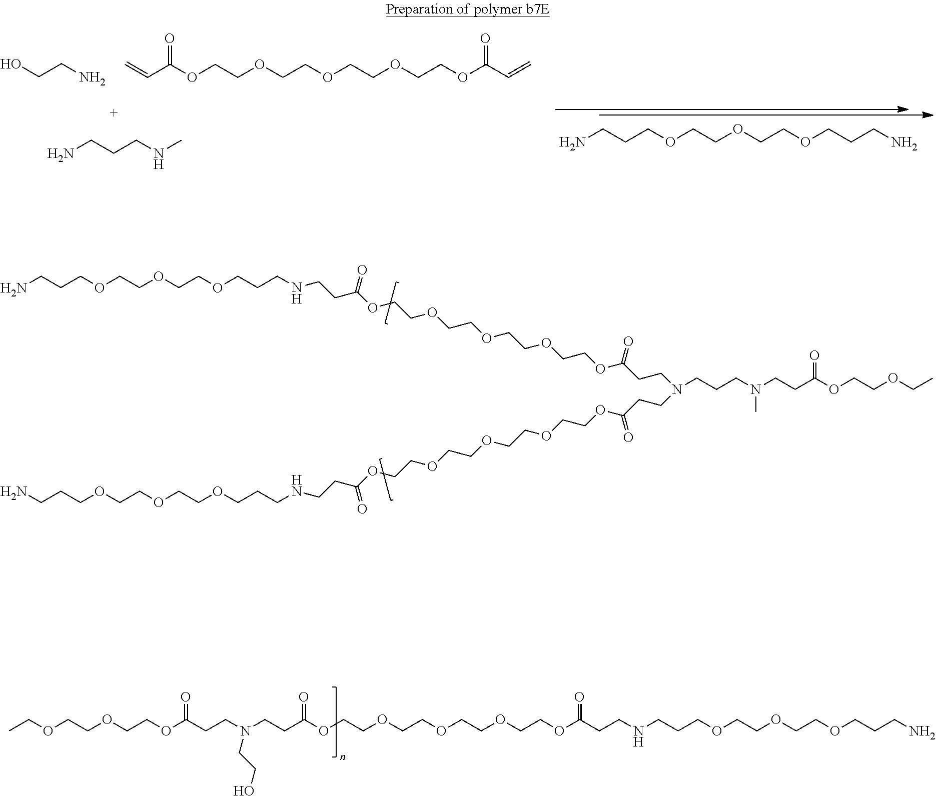

Amine monomer 7, acrylate monomer E, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b7E was obtained.

### Manufacturing Example 236

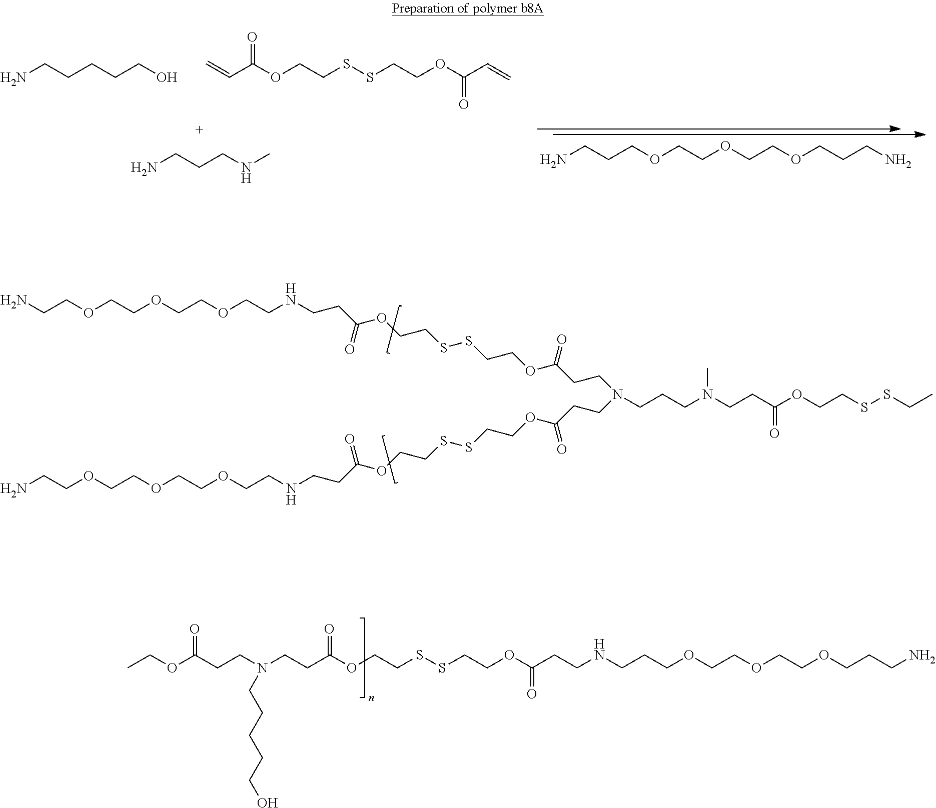

Amine monomer 8, acrylate monomer A, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b8A was obtained.

### Manufacturing Example 237

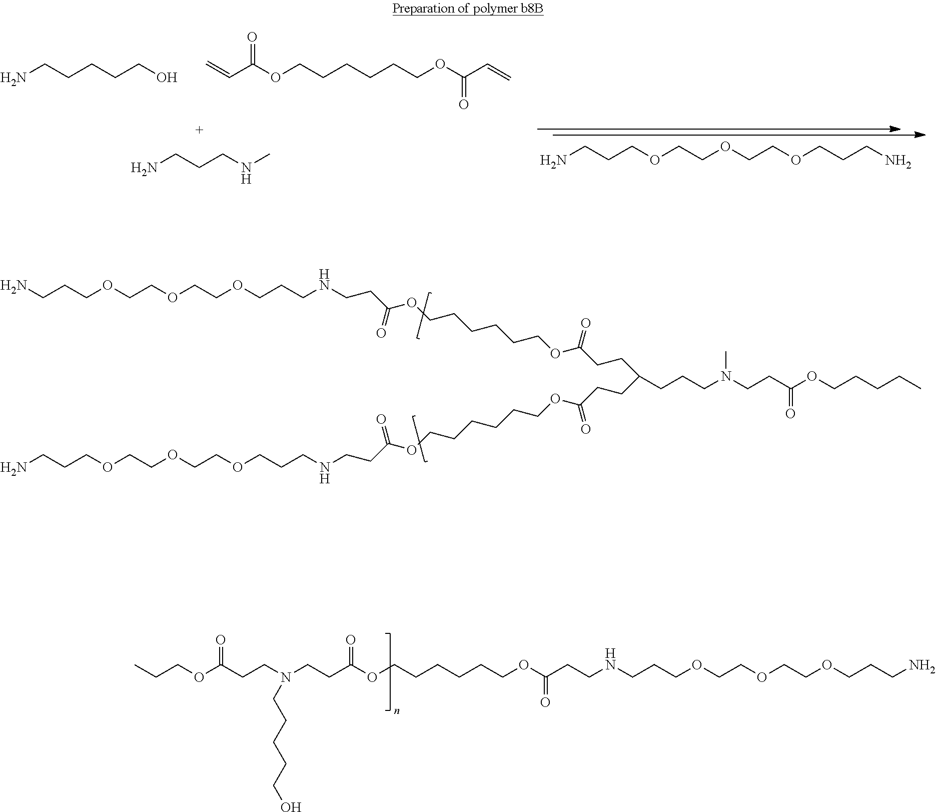

Amine monomer 8, acrylate monomer B, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b8B was obtained.

### Manufacturing Example 238

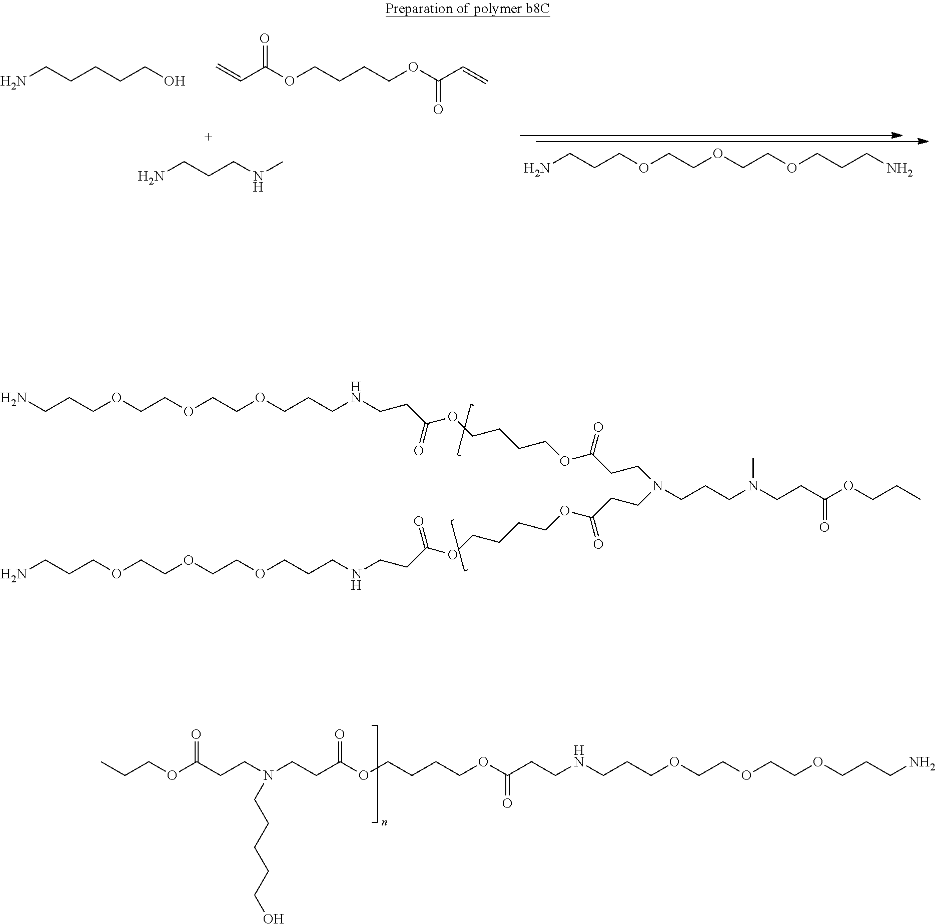

Amine monomer 8, acrylate monomer C, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b8C was obtained.

### Manufacturing Example 239

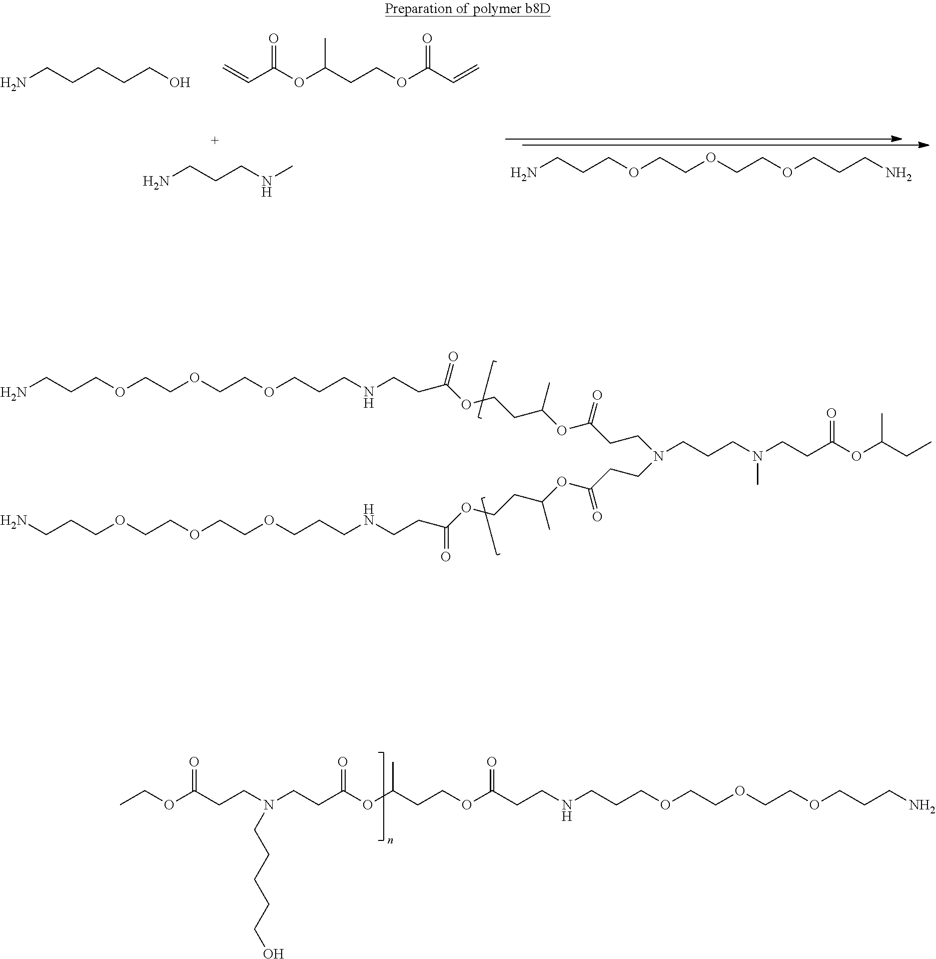

Amine monomer 8, acrylate monomer D, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b8D was obtained.

### Manufacturing Example 240

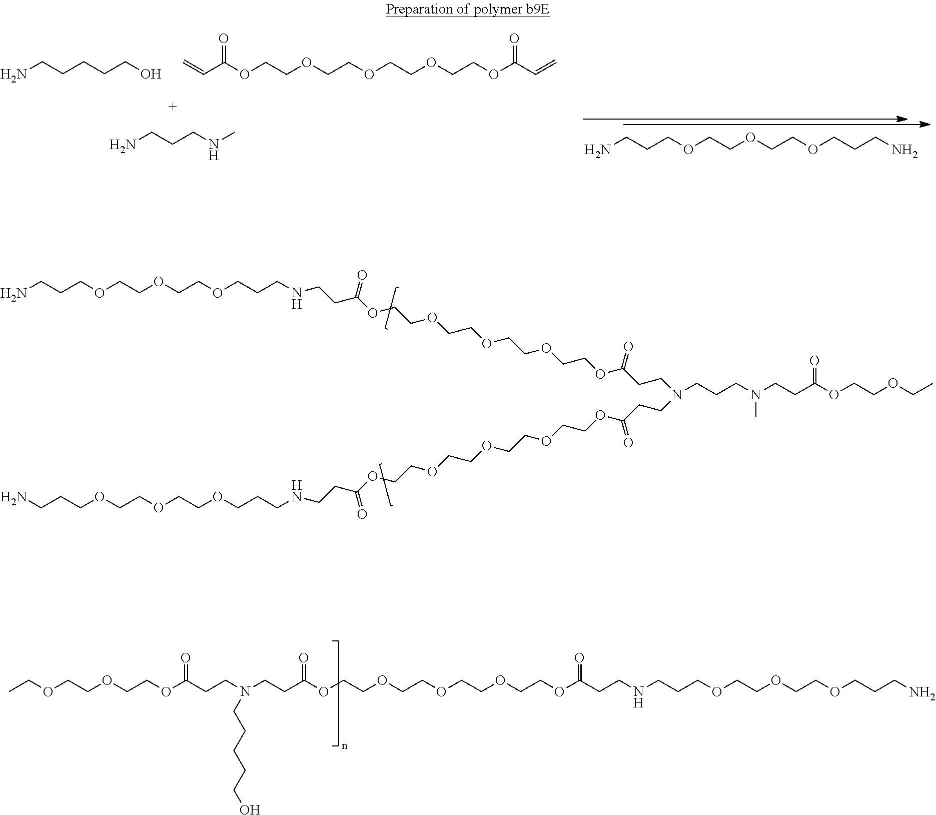

Amine monomer 8, acrylate monomer E, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b8E was obtained.

### Manufacturing Example 241

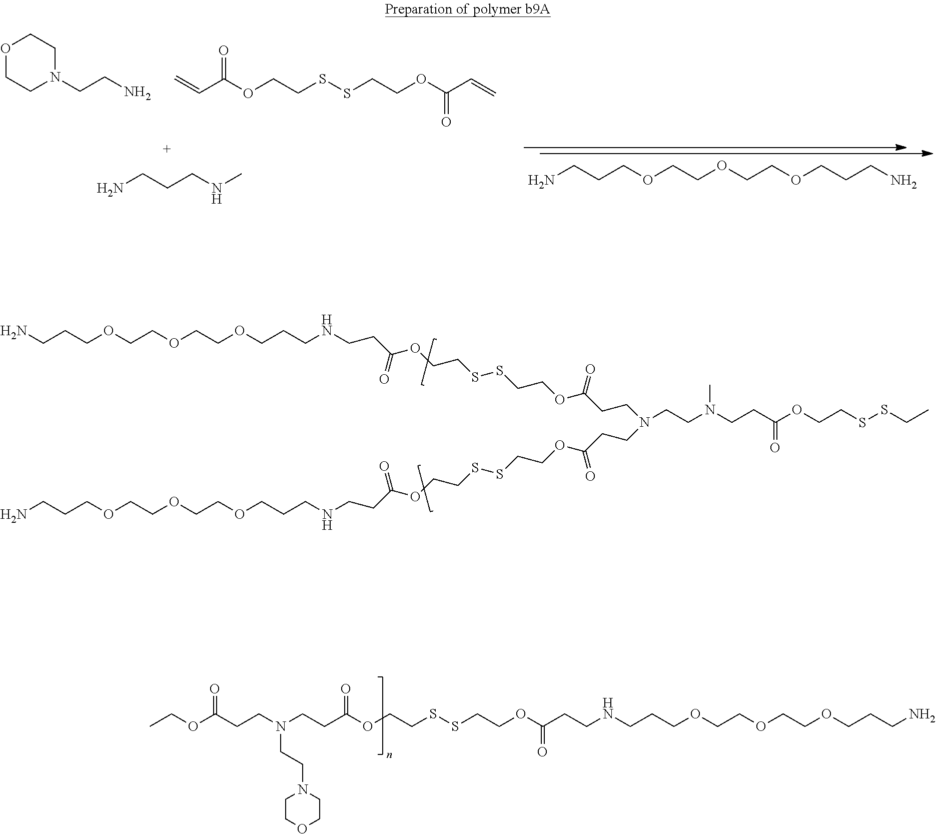

Amine monomer 9, acrylate monomer A, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b9A was obtained.

### Manufacturing Example 242

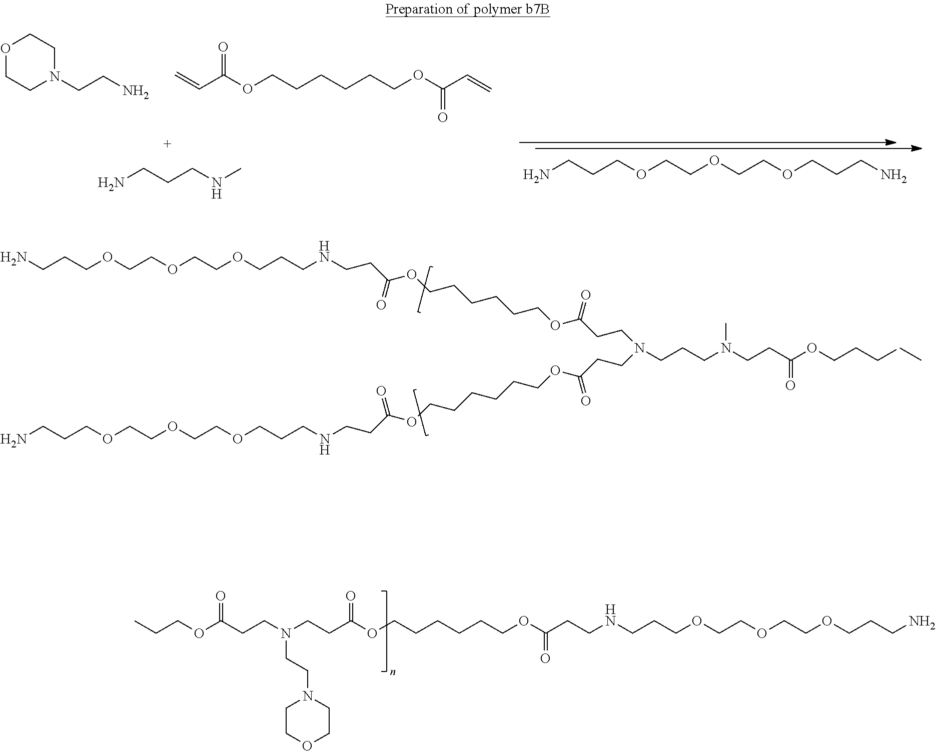

Amine monomer 9, acrylate monomer B, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b9B was obtained.

### Manufacturing Example 243

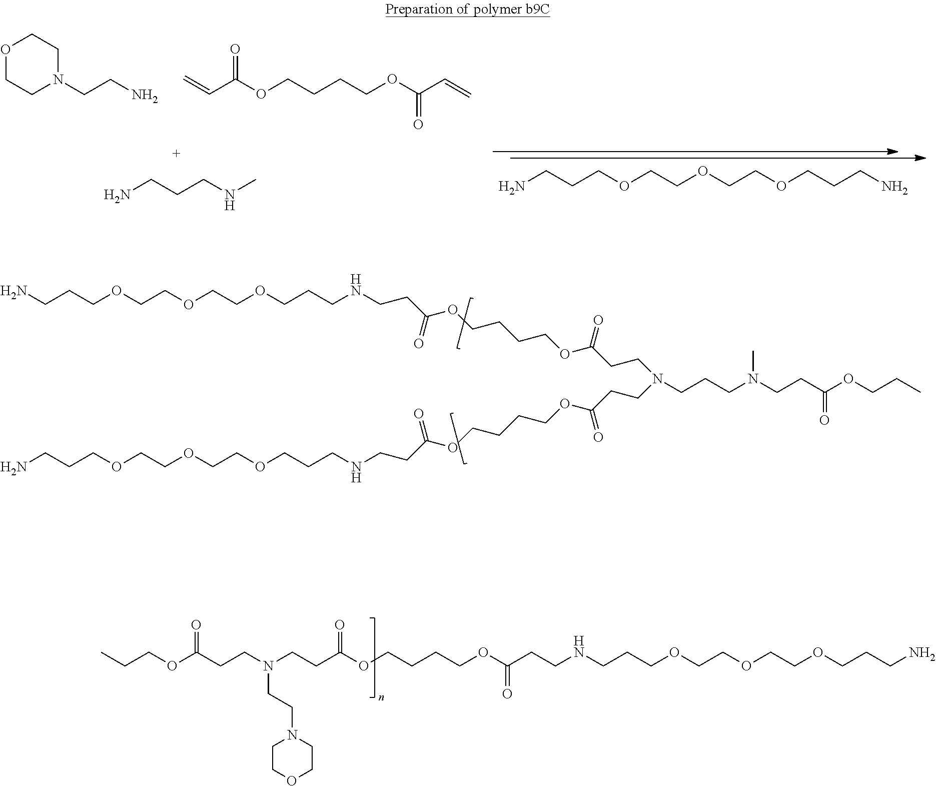

Amine monomer 9, acrylate monomer C, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b9C was obtained.

### Manufacturing Example 244

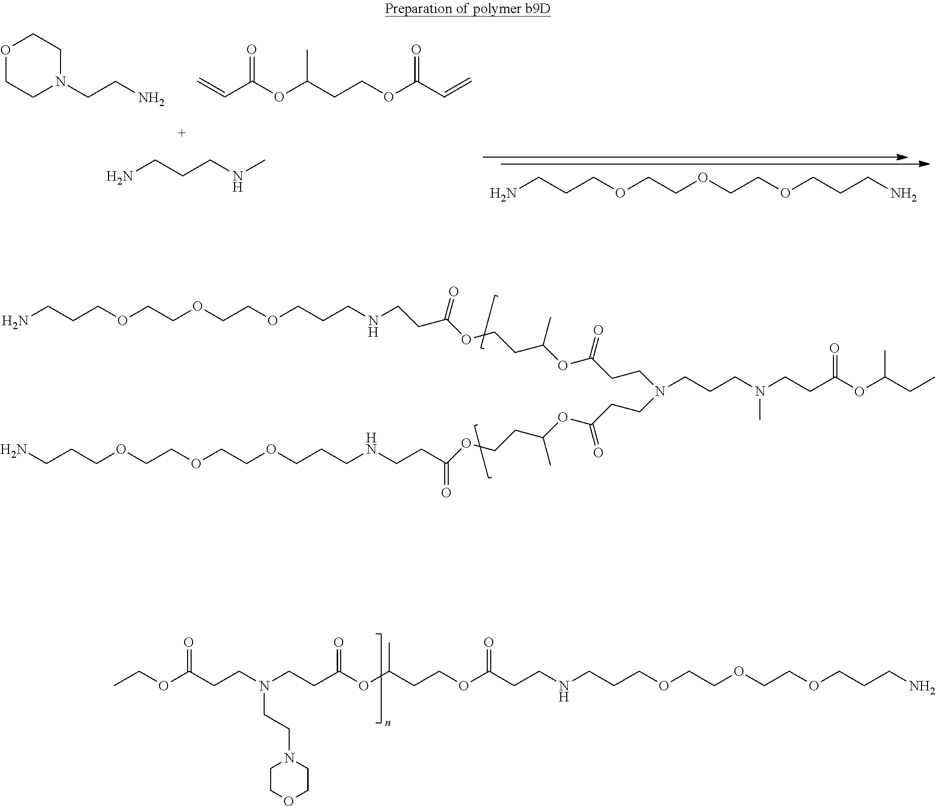

Amine monomer 9, acrylate monomer D, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b9D was obtained.

### Manufacturing Example 245

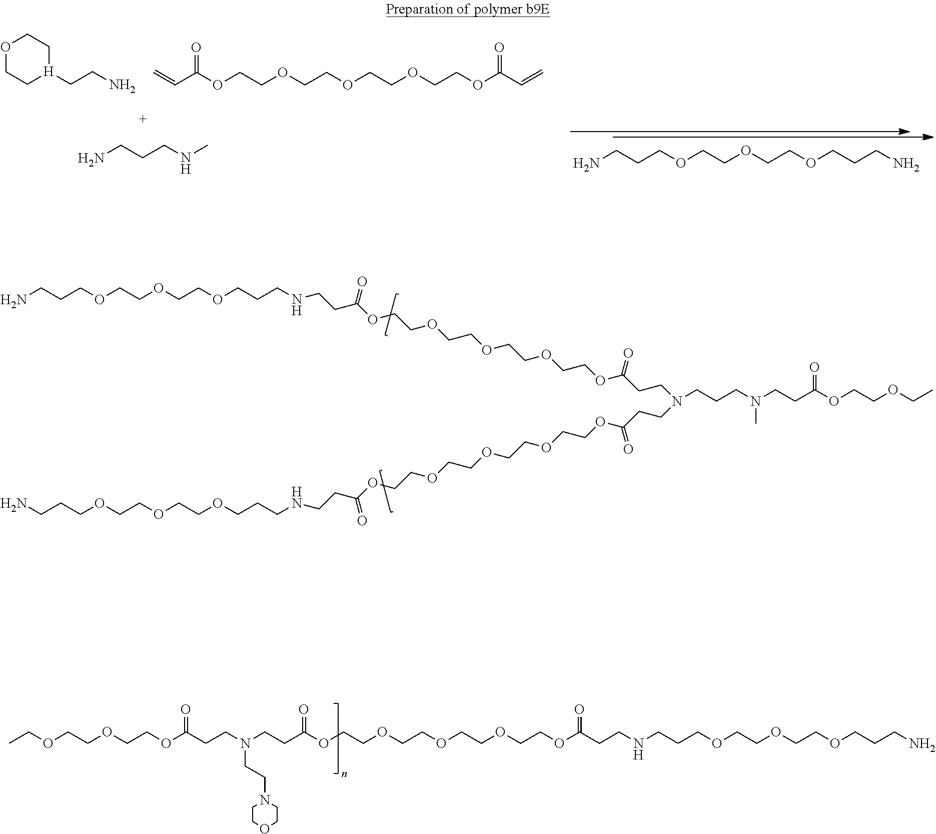

Amine monomer 9, acrylate monomer E, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b9E was obtained.

### Manufacturing Example 246

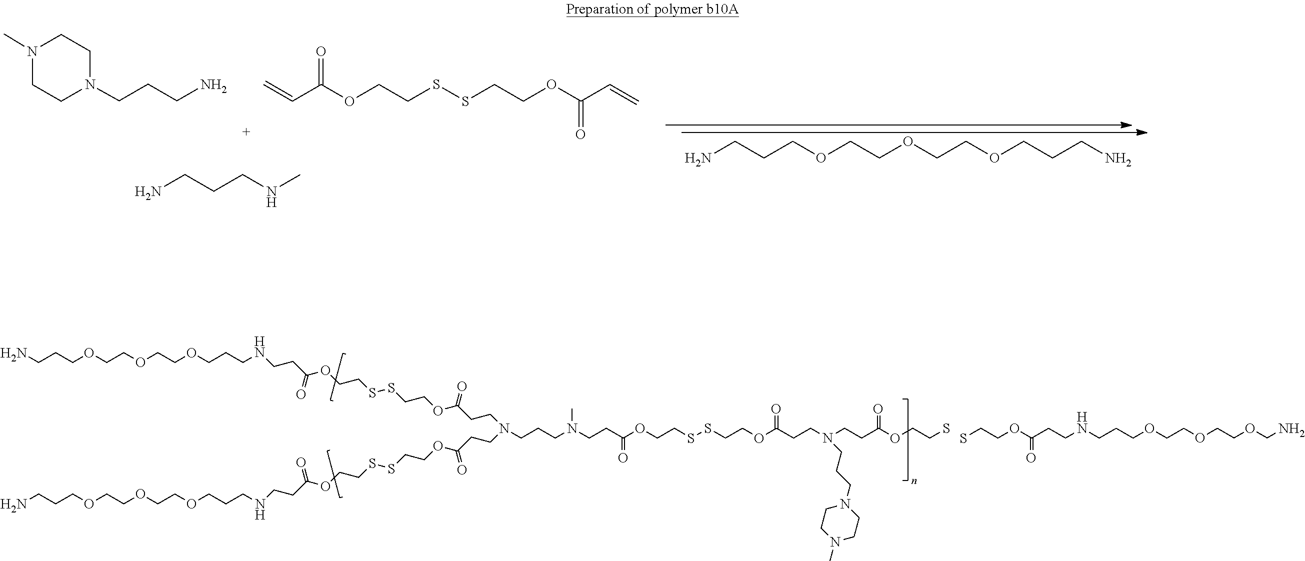

Amine monomer 10, acrylate monomer A, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b10A was obtained.

### Manufacturing Example 247

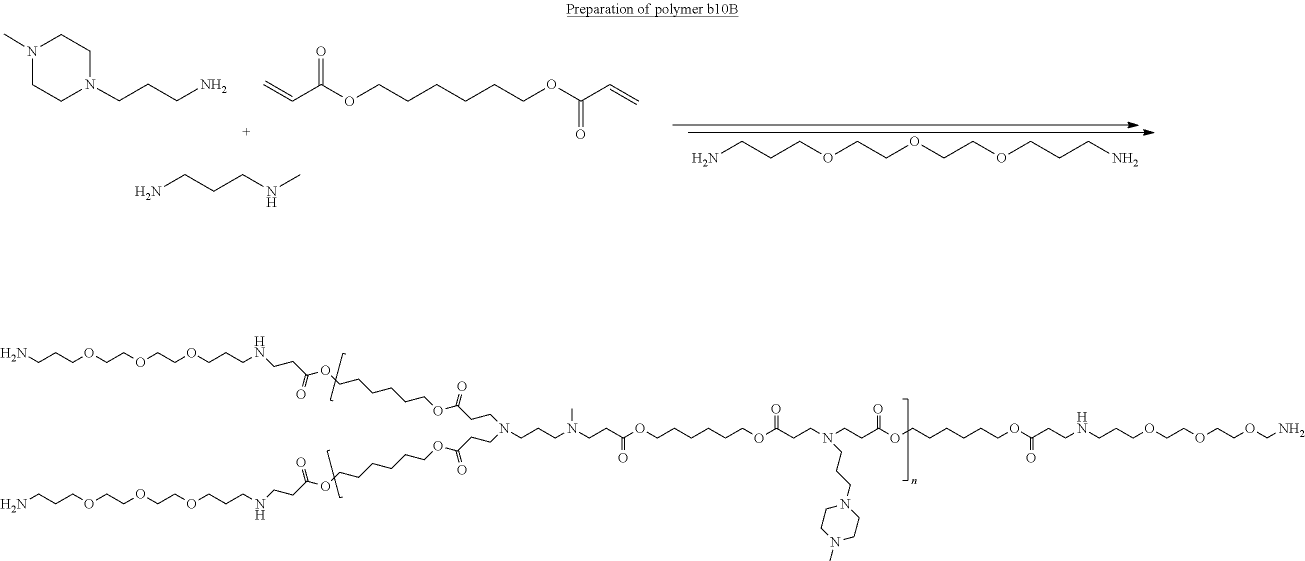

Amine monomer 10, acrylate monomer B, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b10B was obtained.

### Manufacturing Example 248

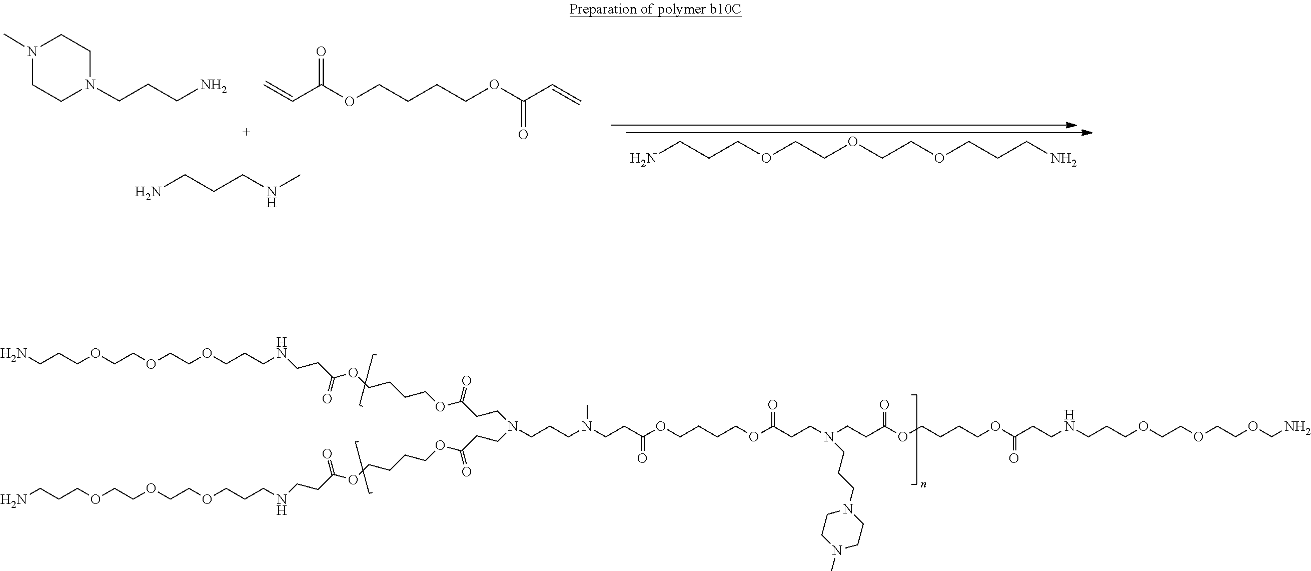

Amine monomer 10, acrylate monomer C, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b10C was obtained.

### Manufacturing Example 249

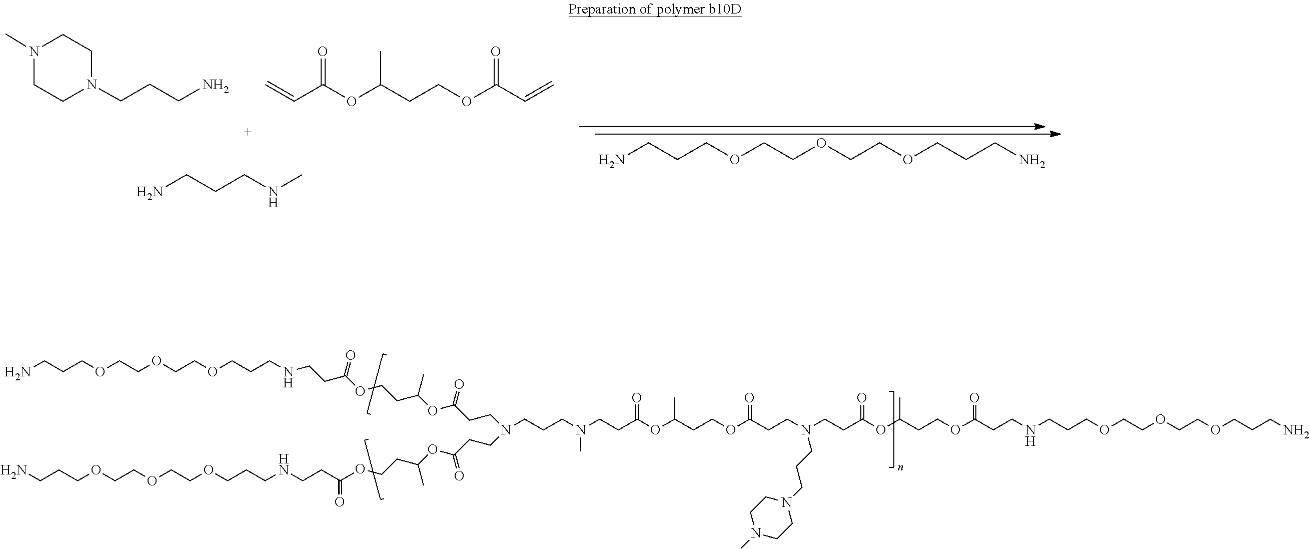

Amine monomer 10, acrylate monomer D, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b10D was obtained.

### Manufacturing Example 250

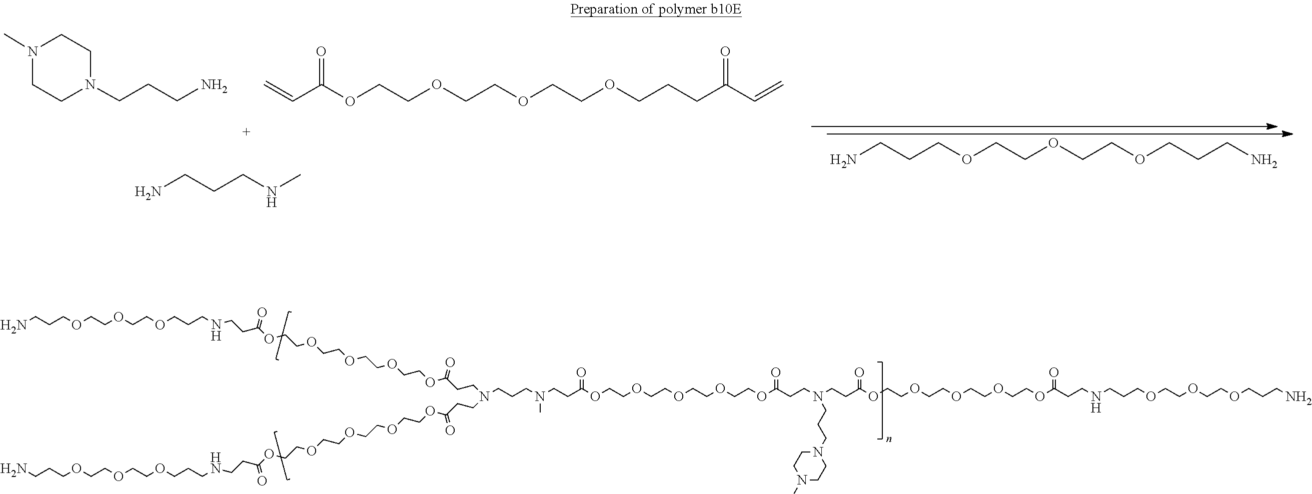

Amine monomer 10, acrylate monomer E, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b10E was obtained.

### Manufacturing Example 251

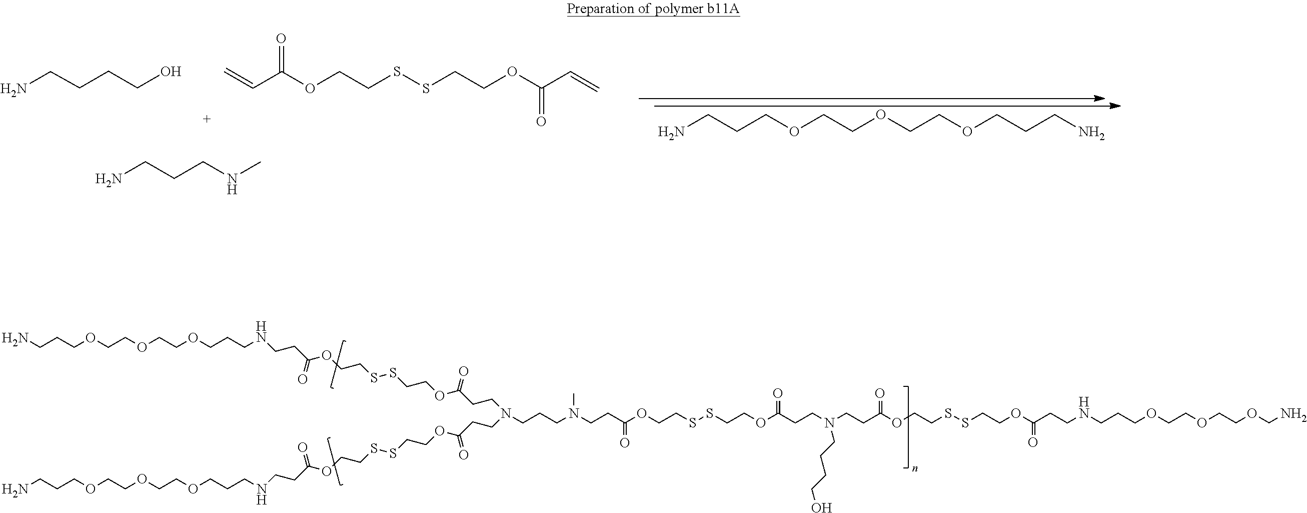

Amine monomer 11, acrylate monomer A, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b11A was obtained.

### Manufacturing Example 252

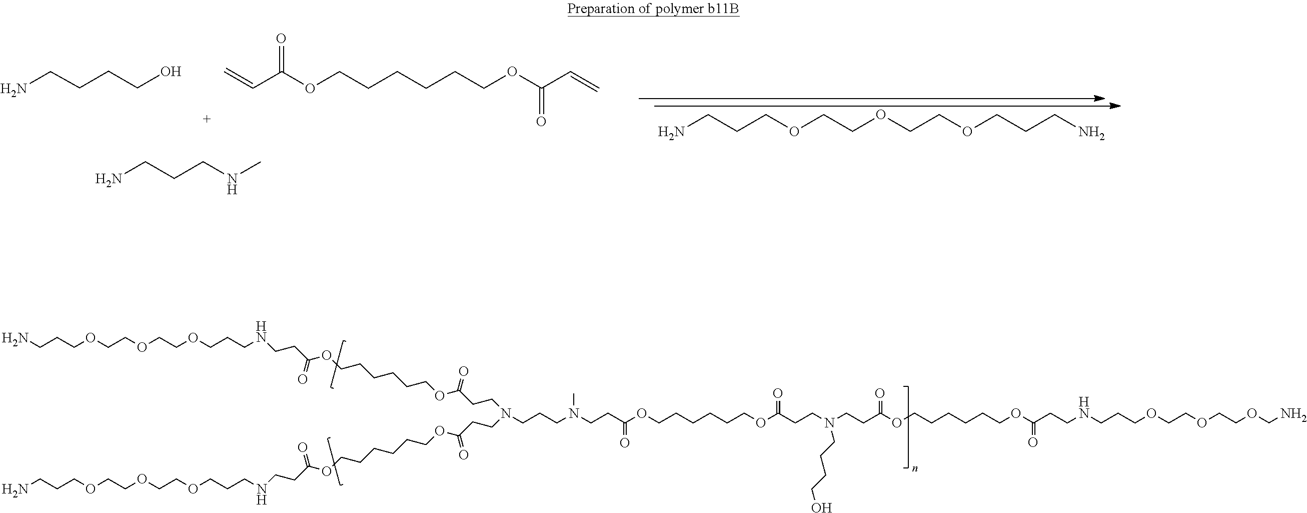

Amine monomer 11, acrylate monomer B, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b11B was obtained.

### Manufacturing Example 253

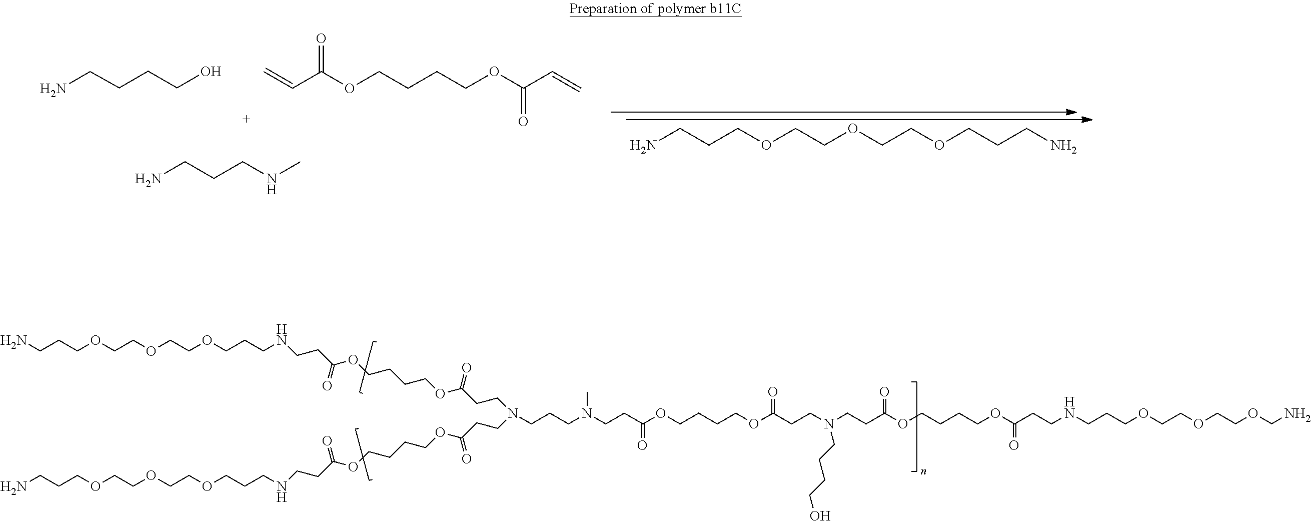

Amine monomer 11, acrylate monomer C, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b11C was obtained.

### Manufacturing Example 254

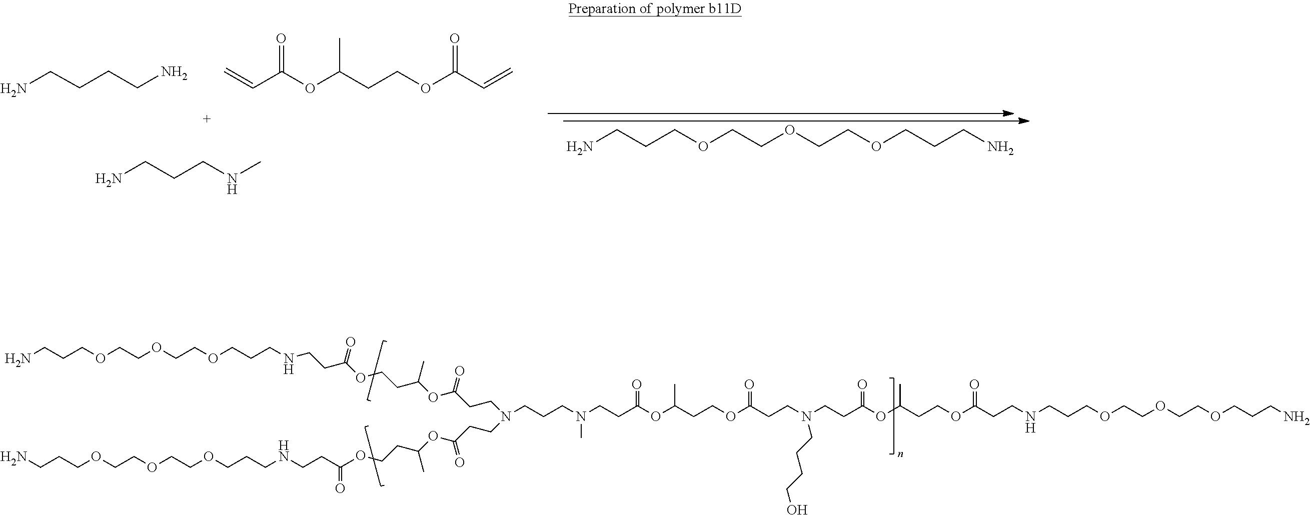

Amine monomer 11, acrylate monomer D, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b11D was obtained.

### Manufacturing Example 255

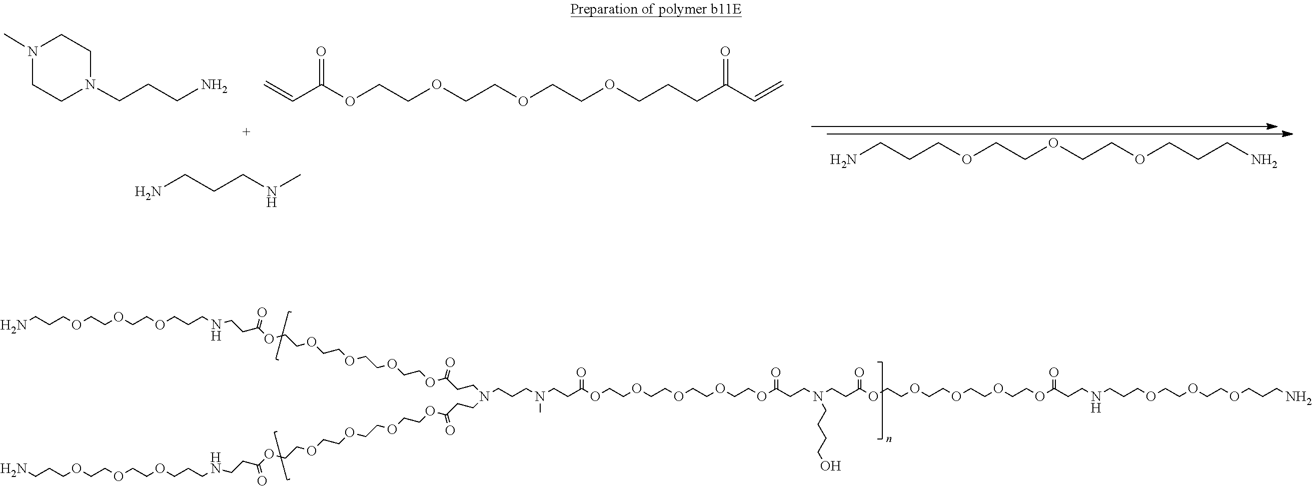

Amine monomer 11, acrylate monomer E, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b11E was obtained.

### Manufacturing Example 256

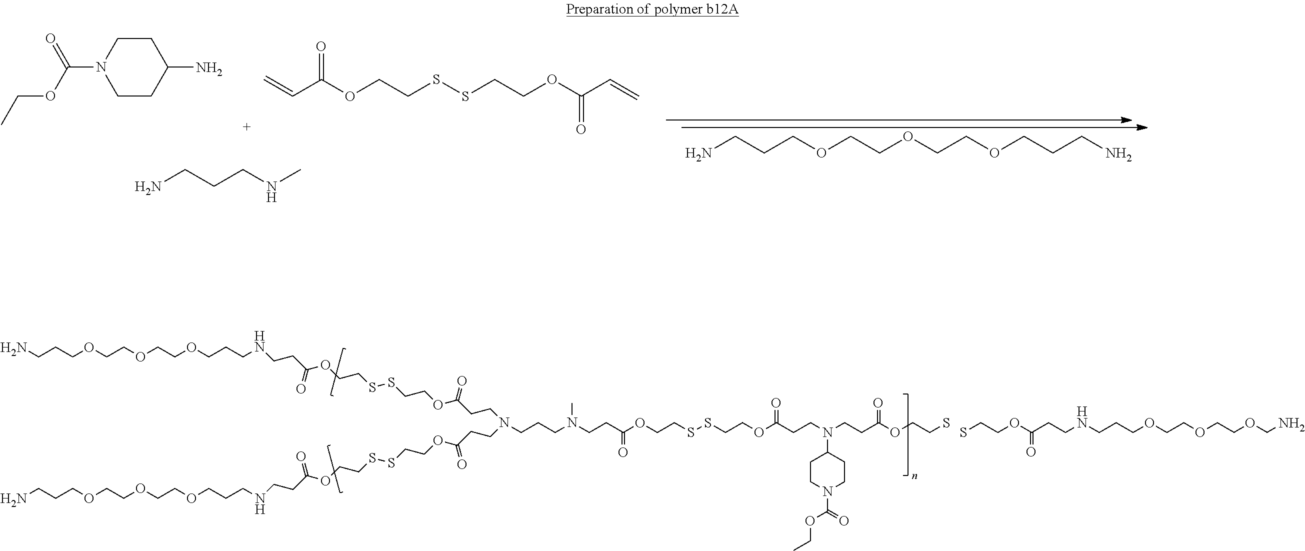

Amine monomer 12, acrylate monomer A, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b12A was obtained.

### Manufacturing Example 257

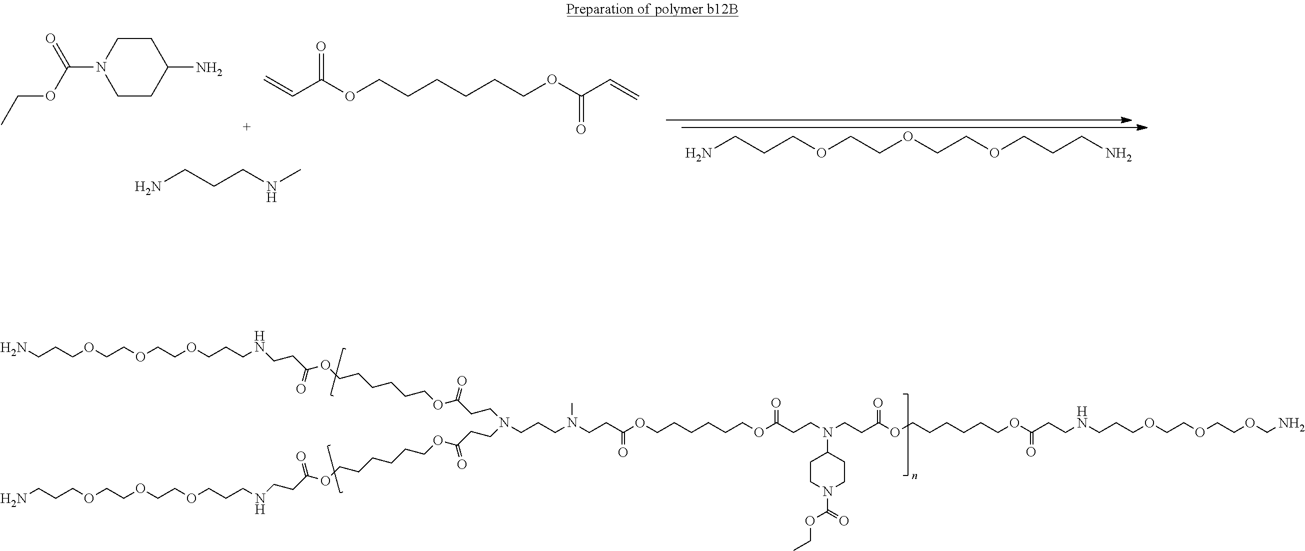

Amine monomer 12, acrylate monomer B, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b12B was obtained.

### Manufacturing Example 258

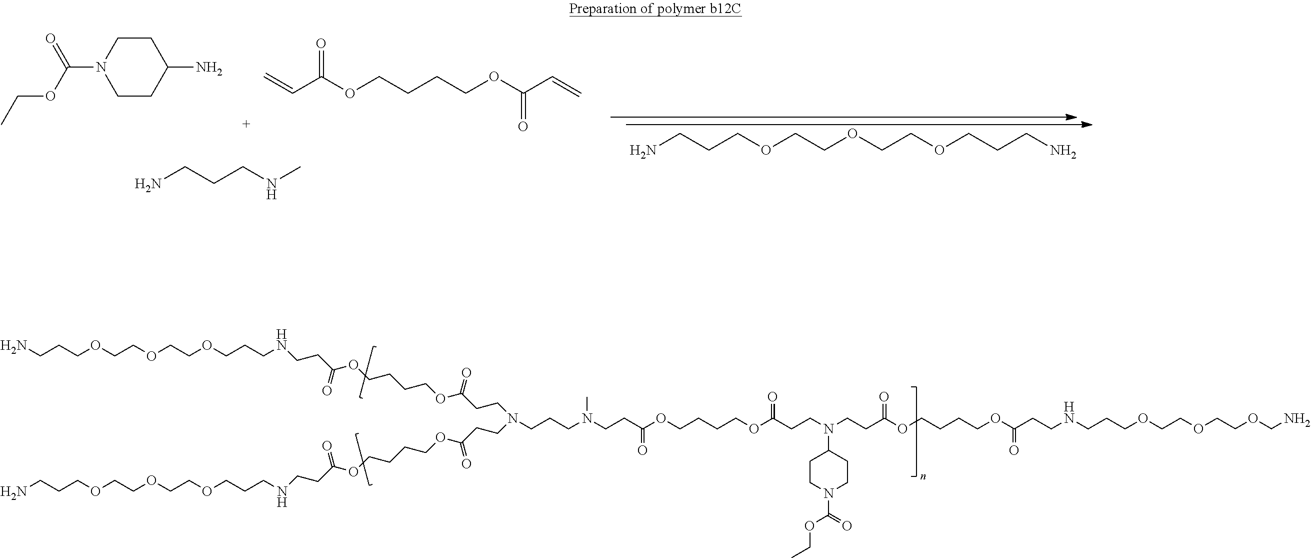

Amine monomer 12, acrylate monomer C, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b12C was obtained.

### Manufacturing Example 259

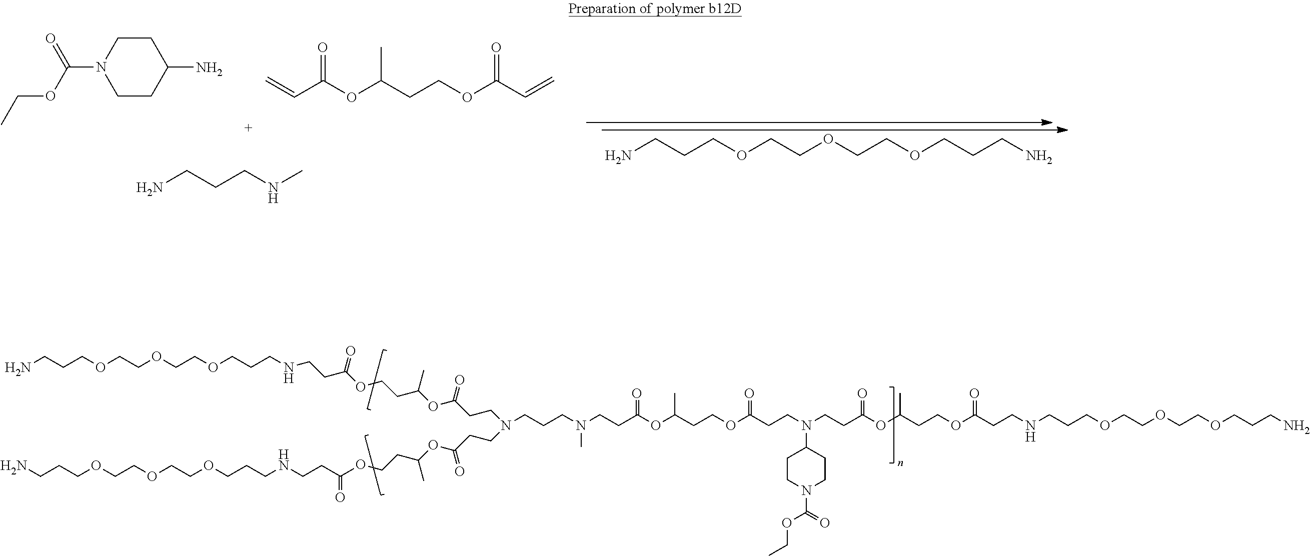

Amine monomer 12, acrylate monomer D, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b12D was obtained.

### Manufacturing Example 260

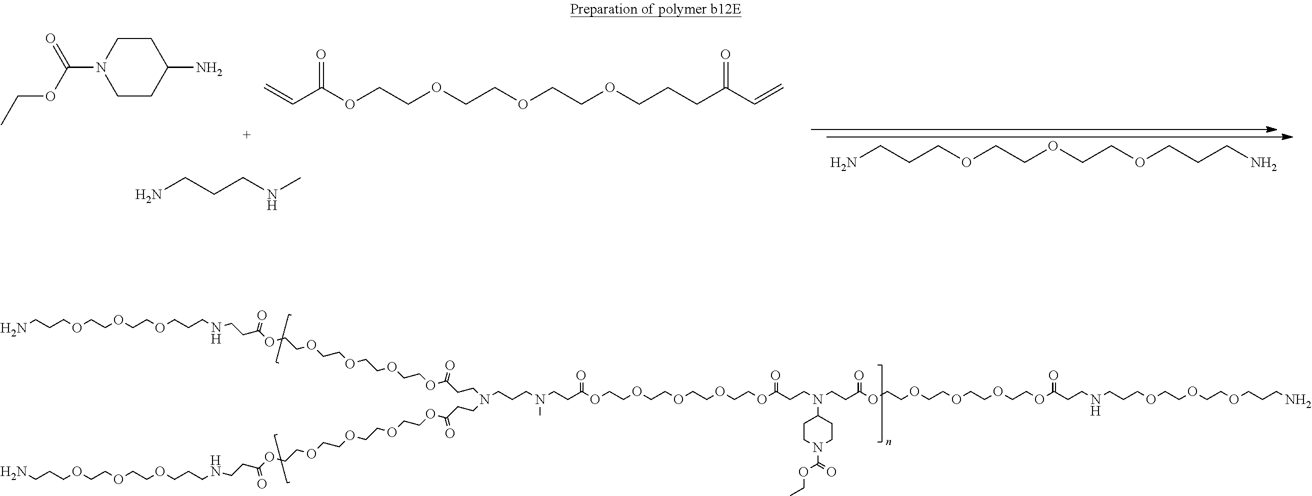

Amine monomer 12, acrylate monomer E, and branched dendrimer were added to a round bottom flask in a molar ratio of 0.4:1:0.2. Thereafter, DMF was added to the flask so that the concentration was 150 mg/ml, and the reaction was carried out at 90° C. for 48 hours. Thereafter, the mixture was cooled to room temperature, and then an end-capping agent was dissolved in DMF to a concentration of 150 mg/ml and added in a molar ratio of 2, and the reaction was carried out for 24 hours at room temperature, and then b12E was obtained.

### Example 1

### Preparation of Polymeric Nanoparticles (PNP)

mRNA was added to the polymer prepared by any one of Manufacturing Examples 1 to 260 above, and polymeric nanoparticles (PNP), which are nucleic acid delivery vehicles of the present invention, were prepared by the following method.

The polymer prepared by any one of Manufacturing Example 1 to 260 above was diluted in a pH 5.0, 25 mM sodium acetate buffer at a ratio for each polymer. Thereafter, 6 μl of the polymer solution and 6 μl of the mRNA solution having a concentration of 1 mg/ml were mixed and then stabilized at 4° C. for 30 minutes. Thereafter, the mixture was diluted with 38 μl of PBS to make a total volume of 50 μl, and used in the following test examples.

### Test Example 1

### Analysis of Synthesized Polymer

The polymers obtained by the methods of Manufacturing Example 1 to 260 above were analyzed by the following methods.

### 1.1 ¹H-NMR

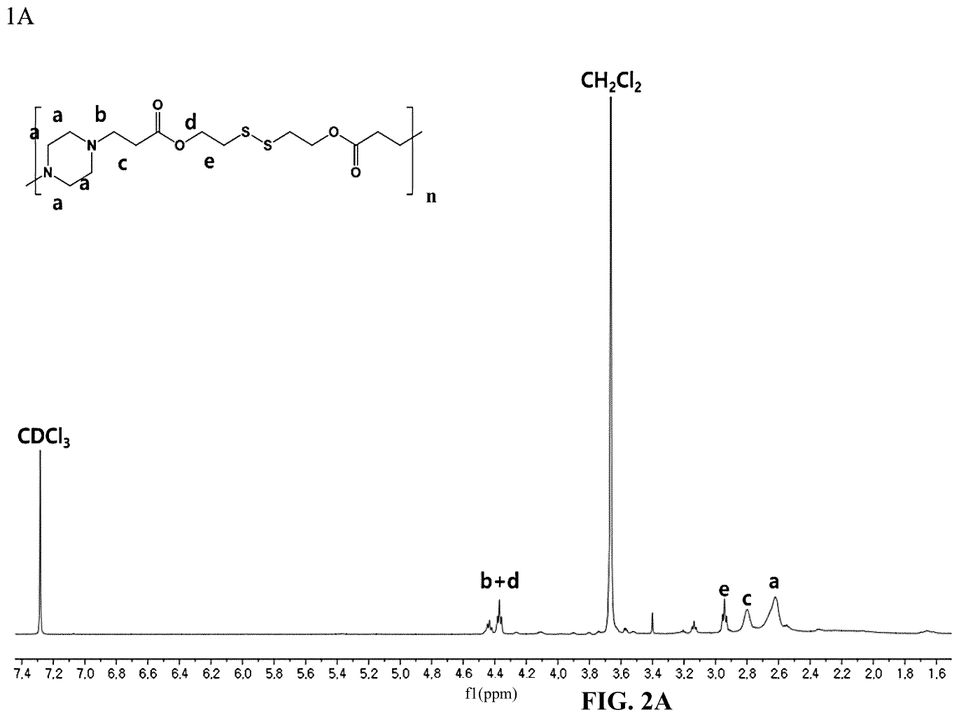

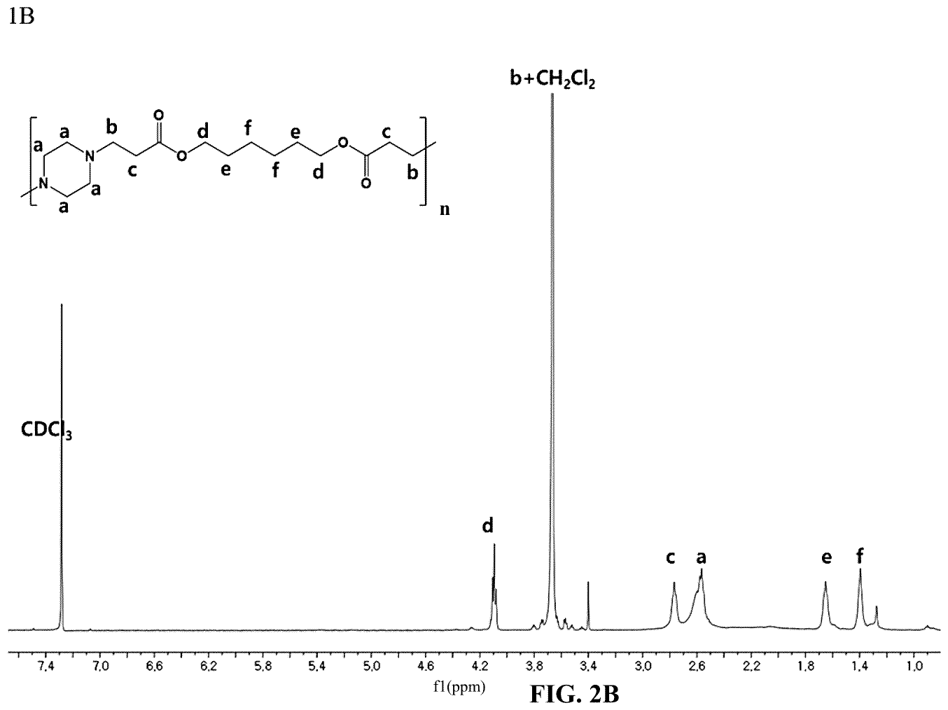

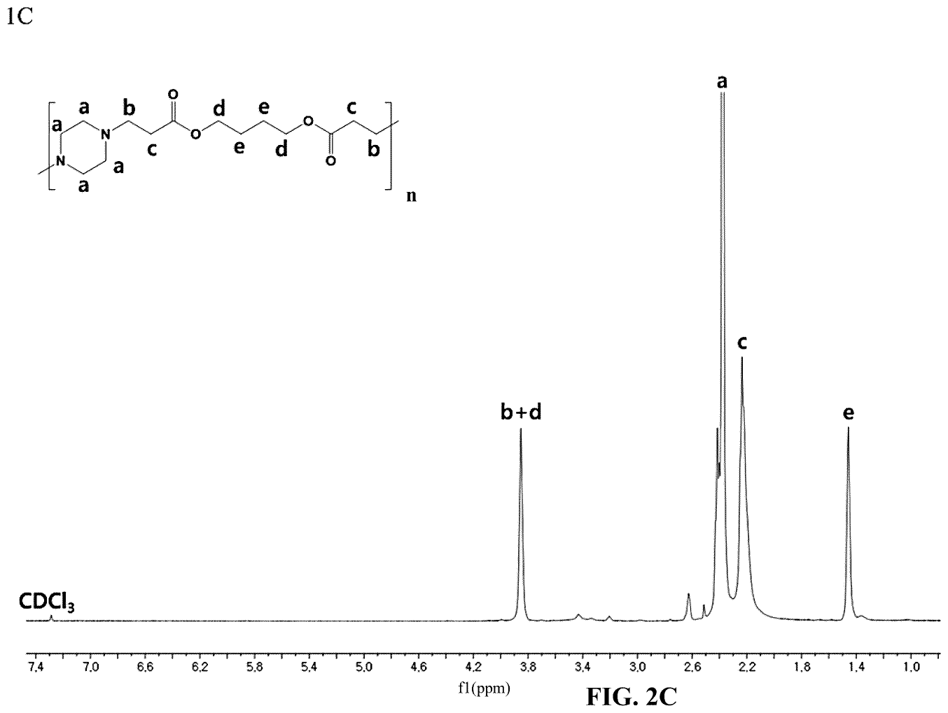

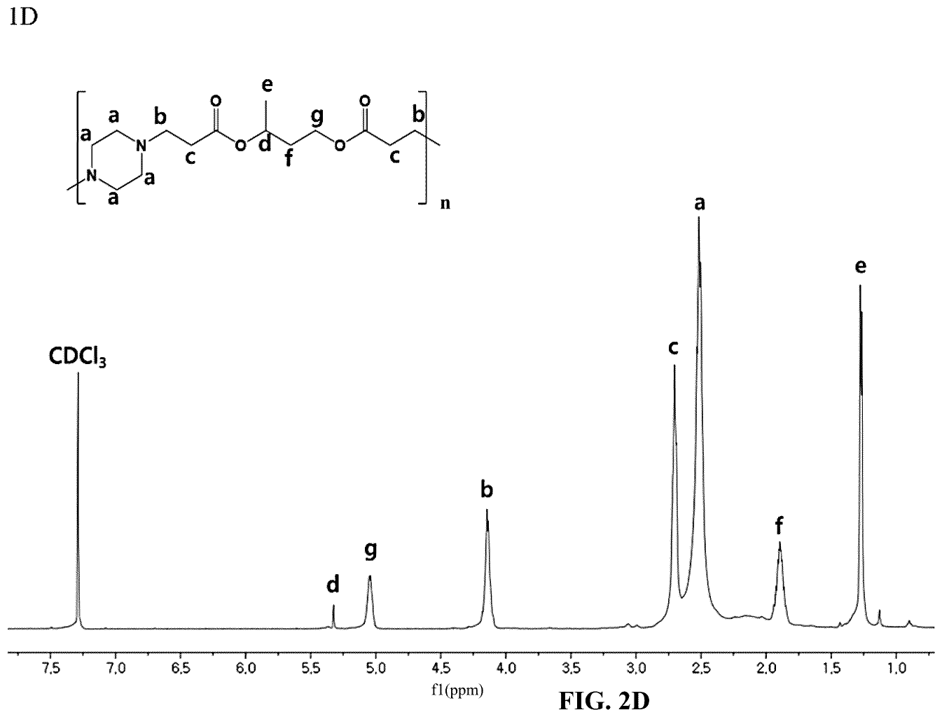

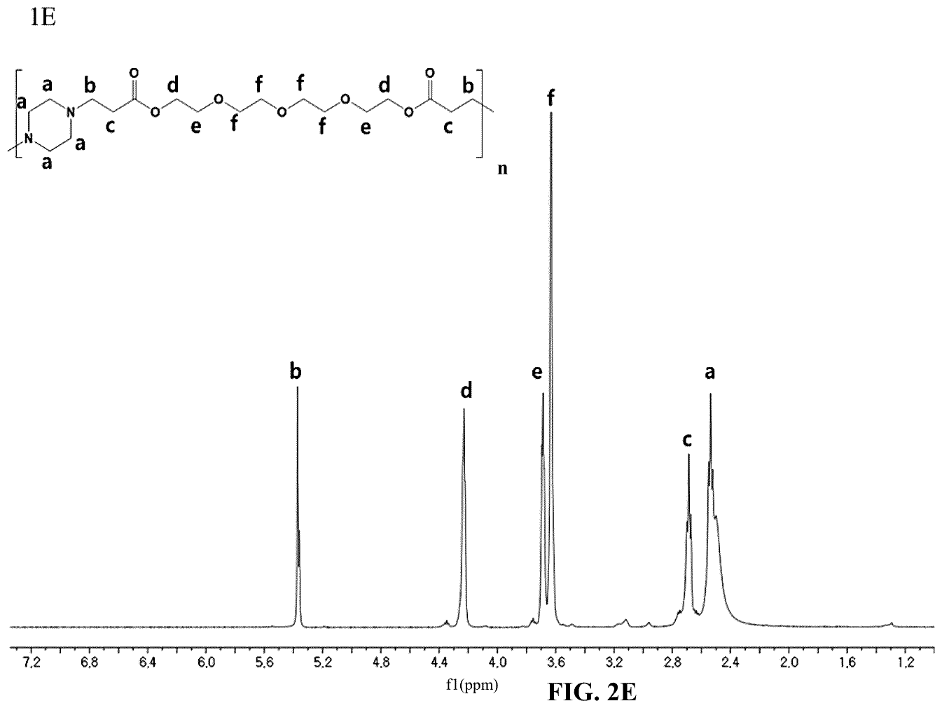

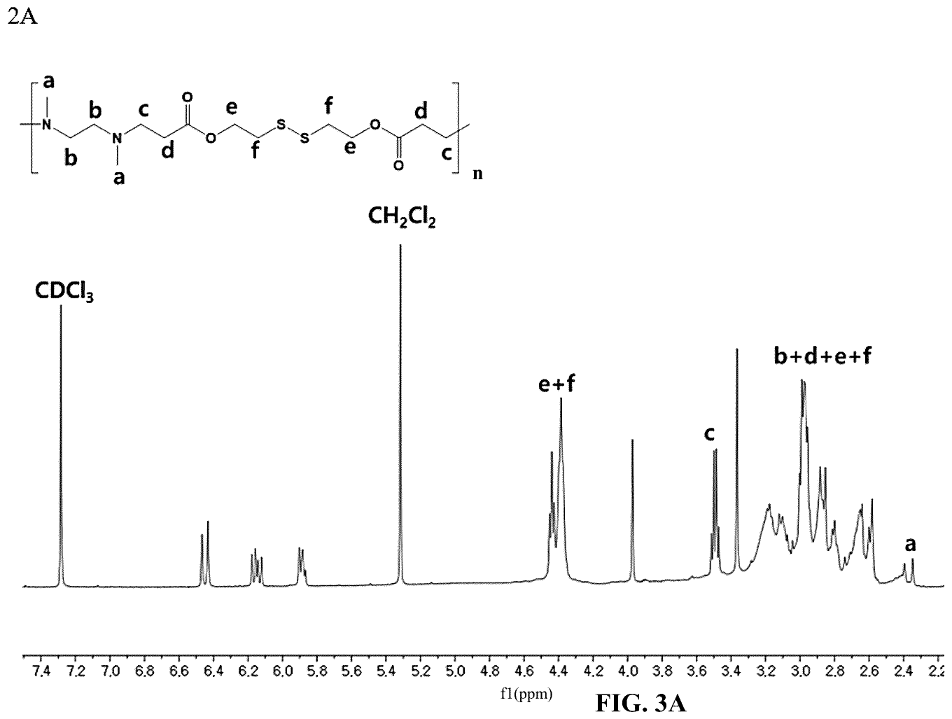

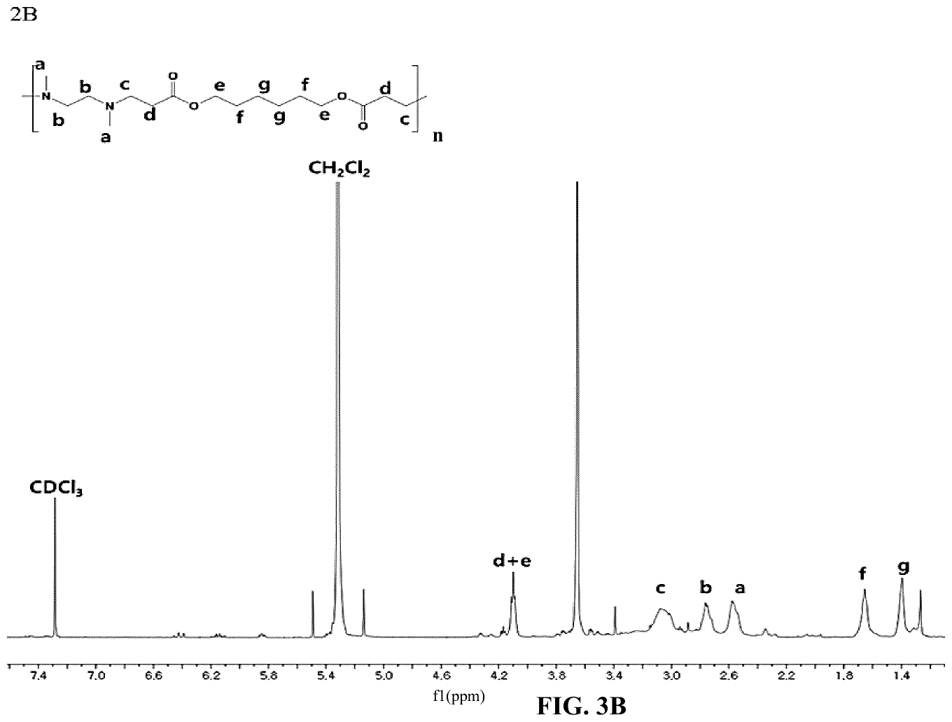

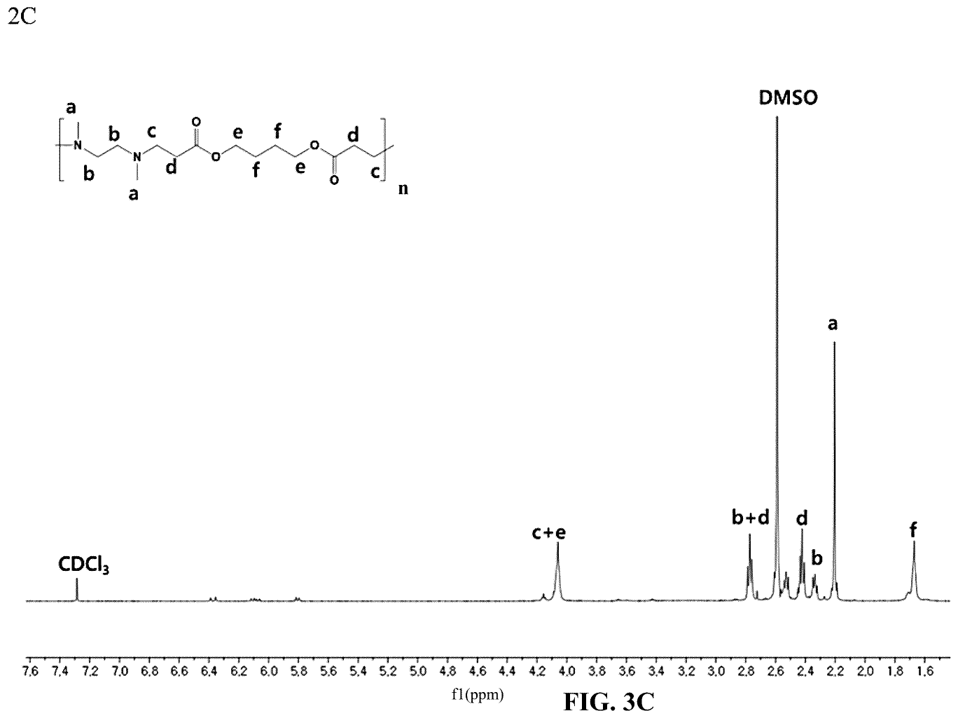

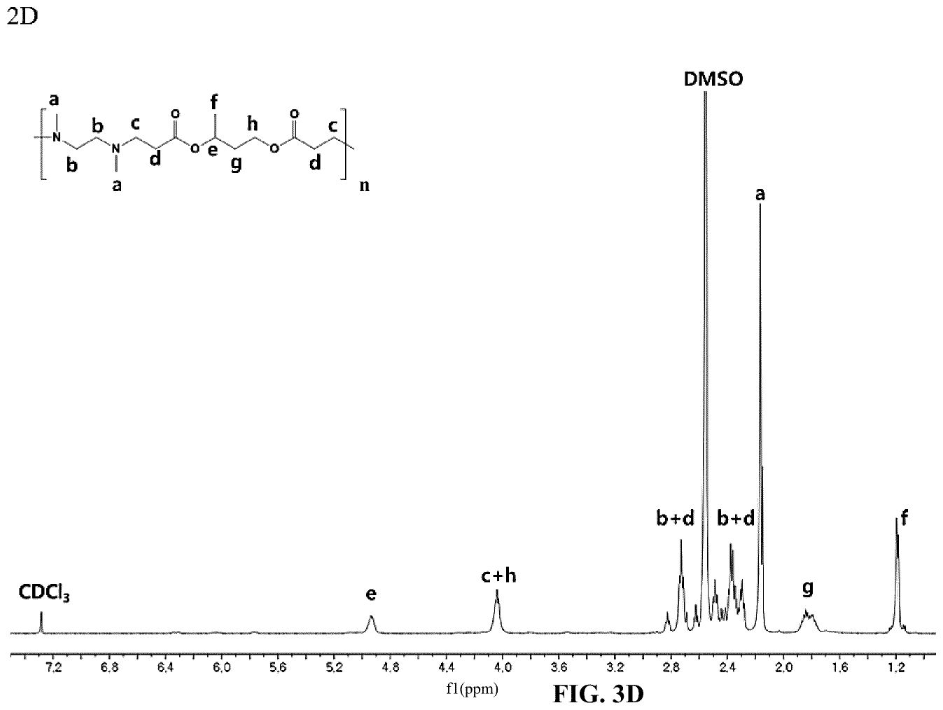

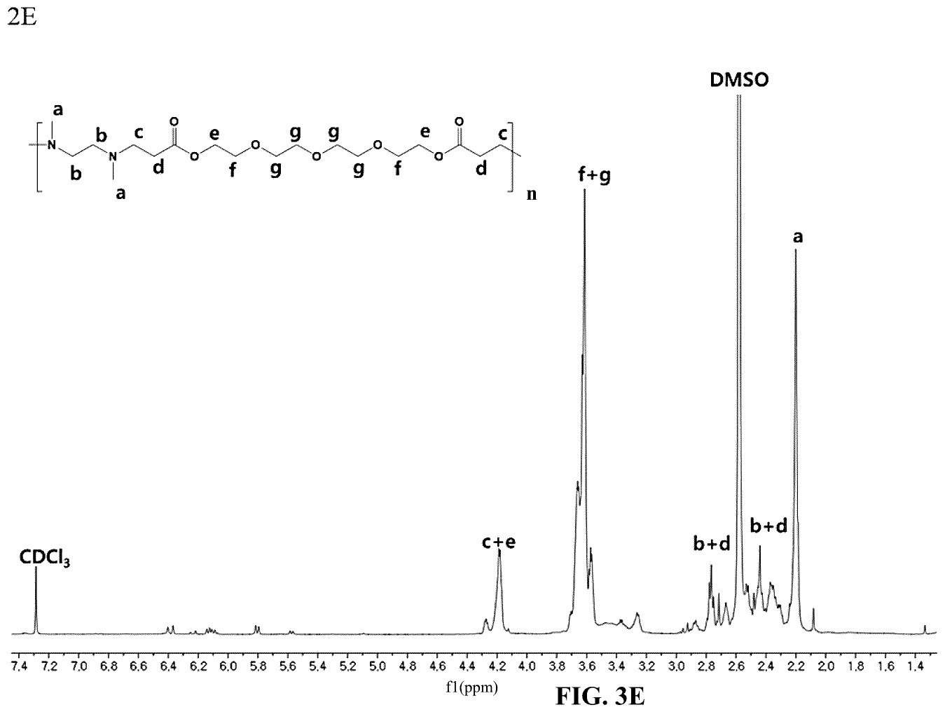

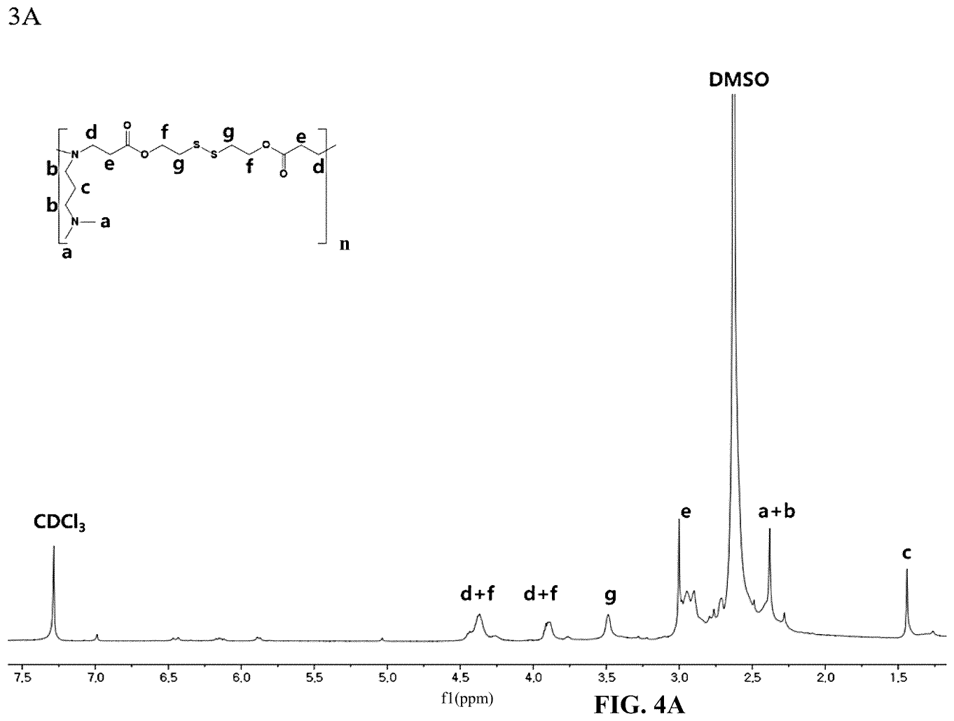

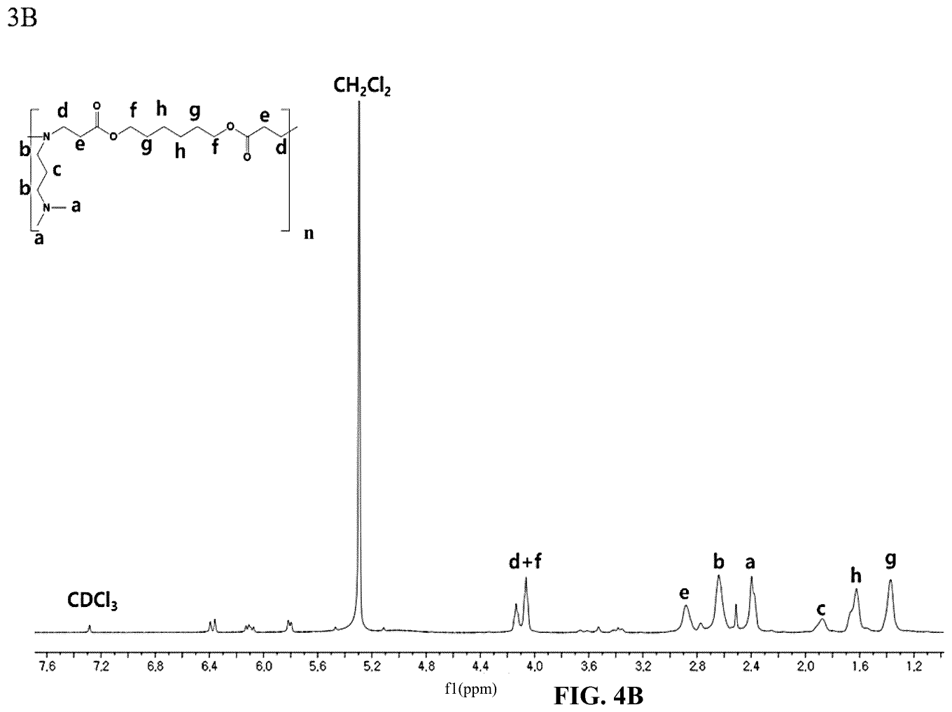

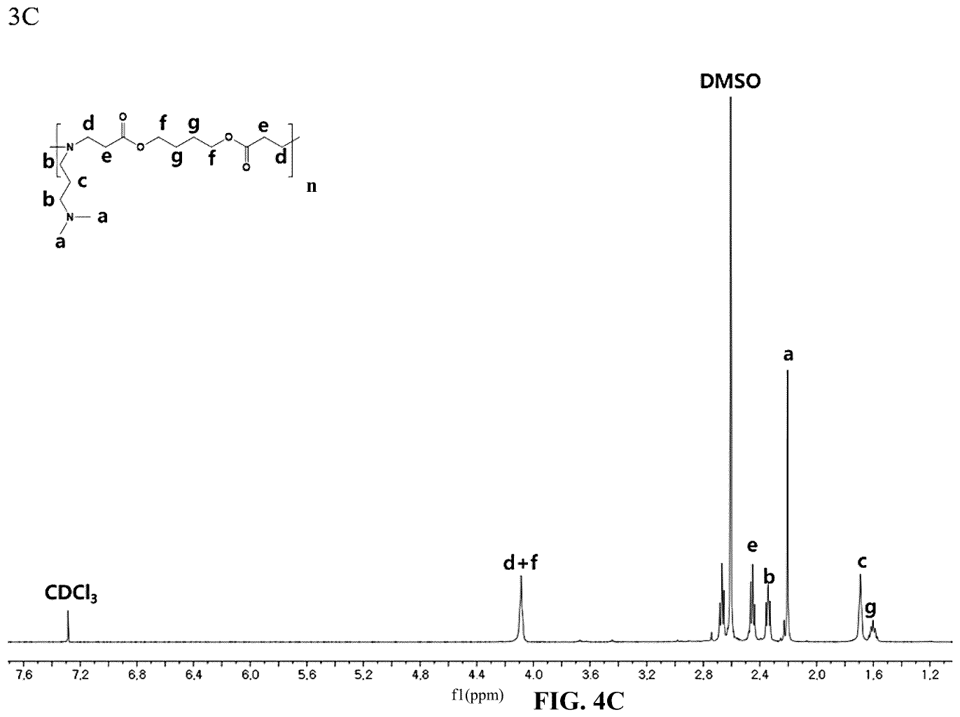

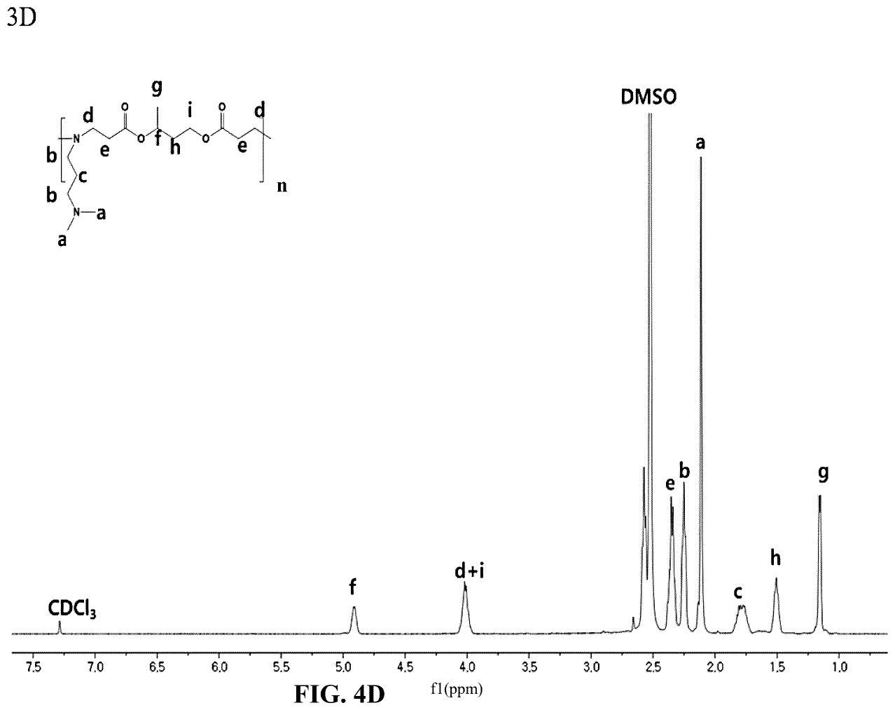

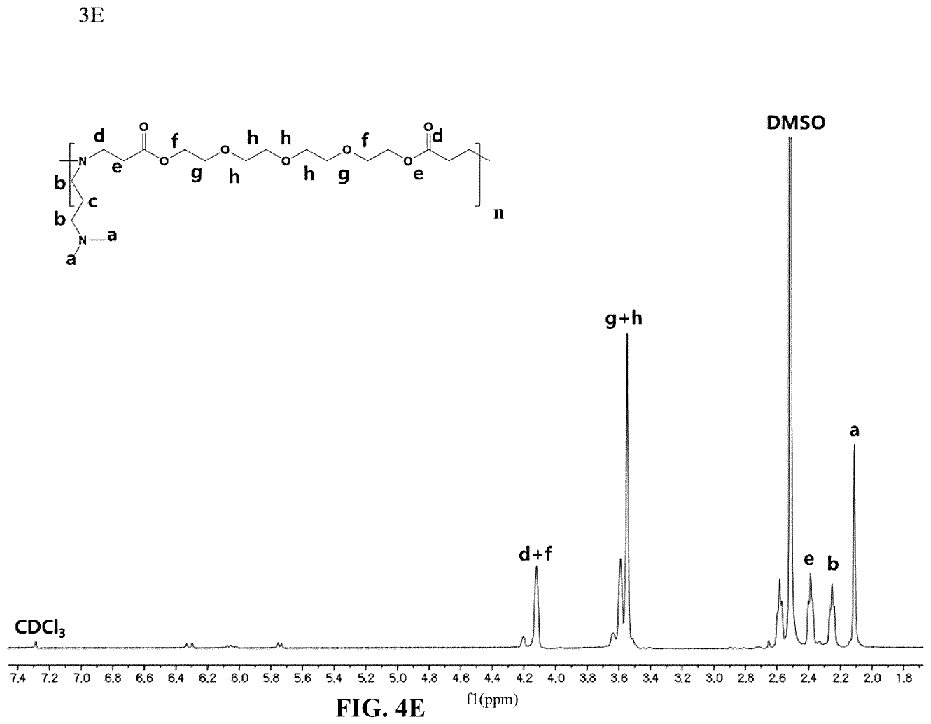

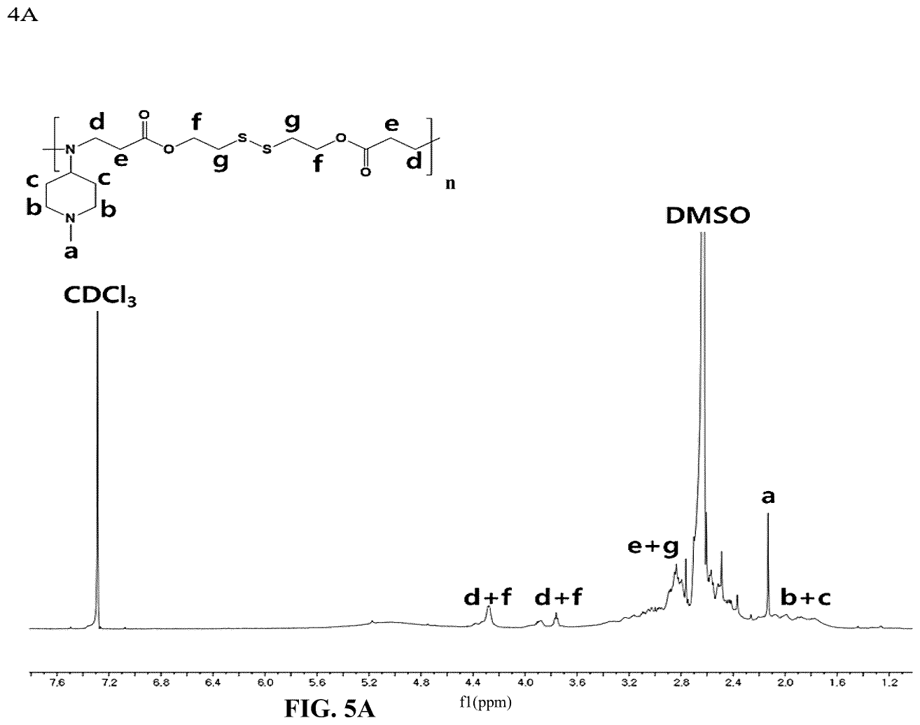

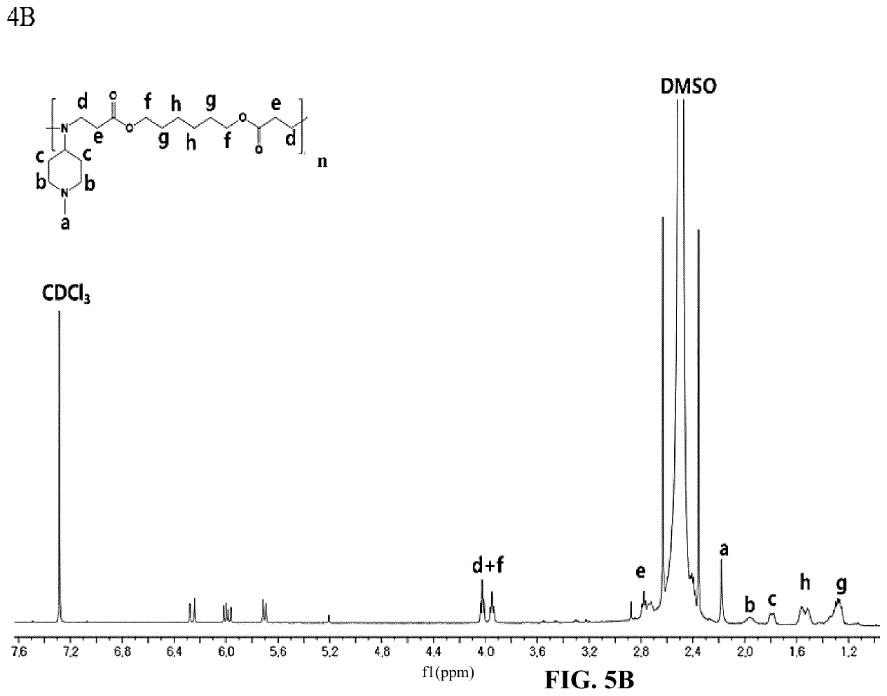

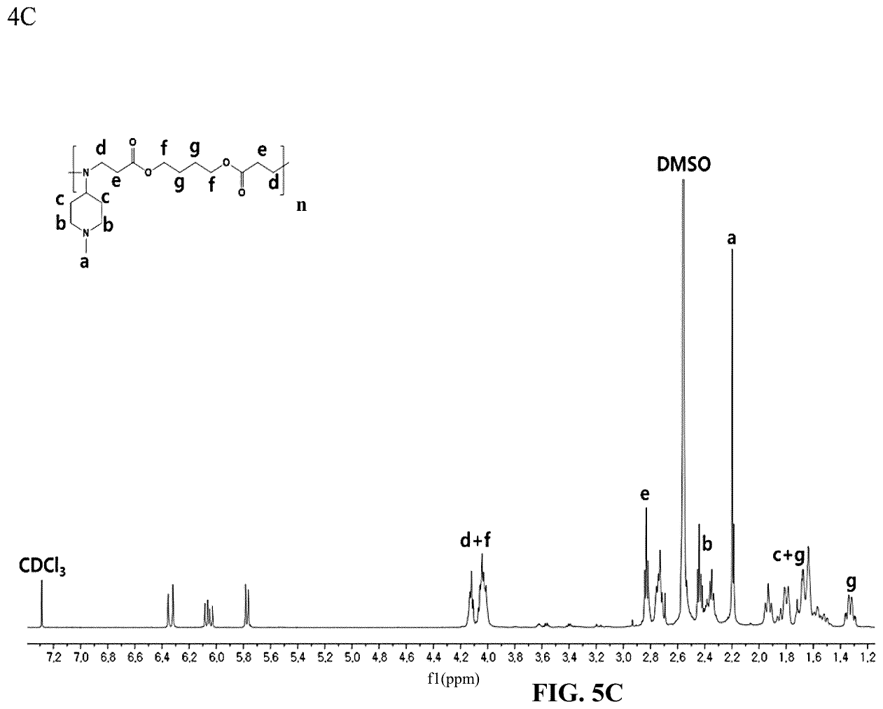

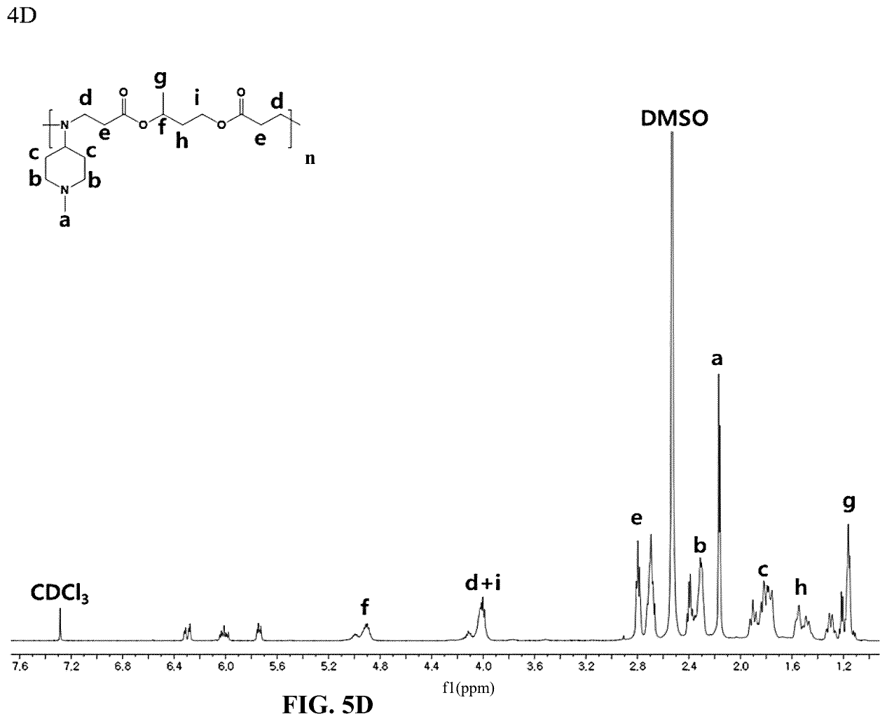

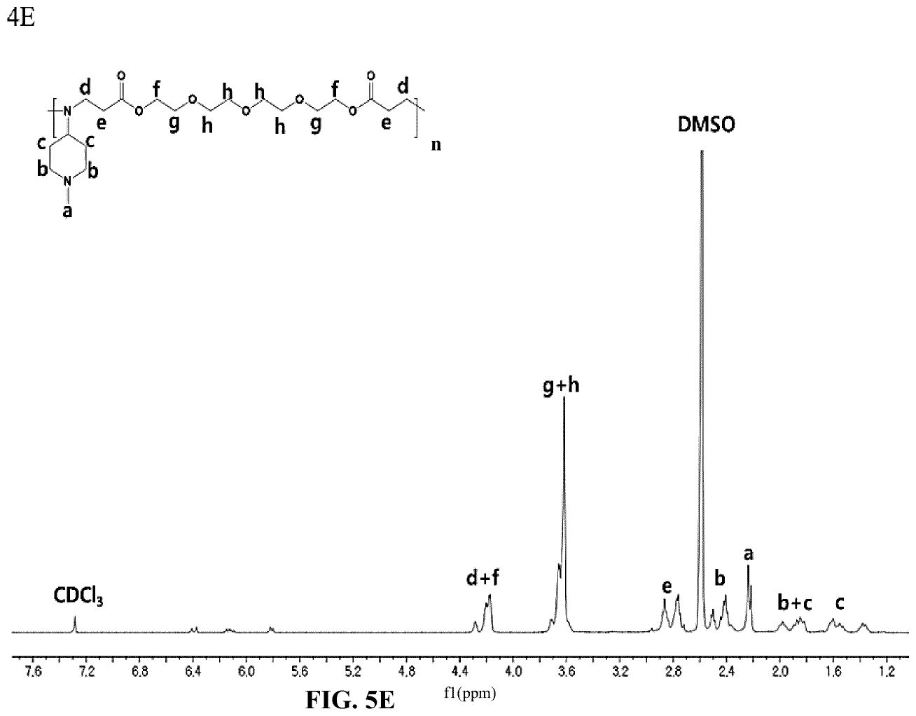

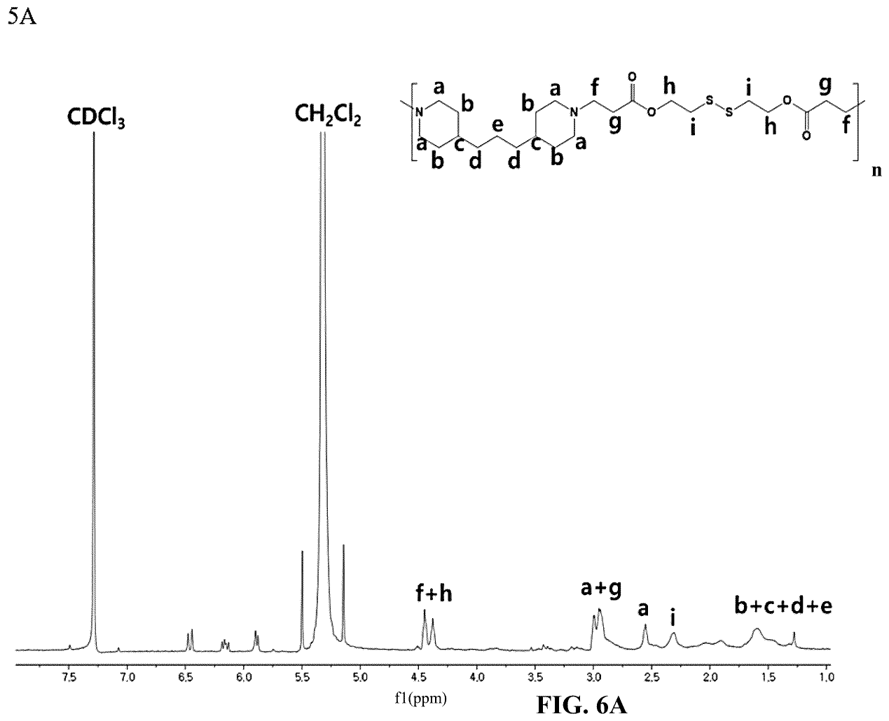

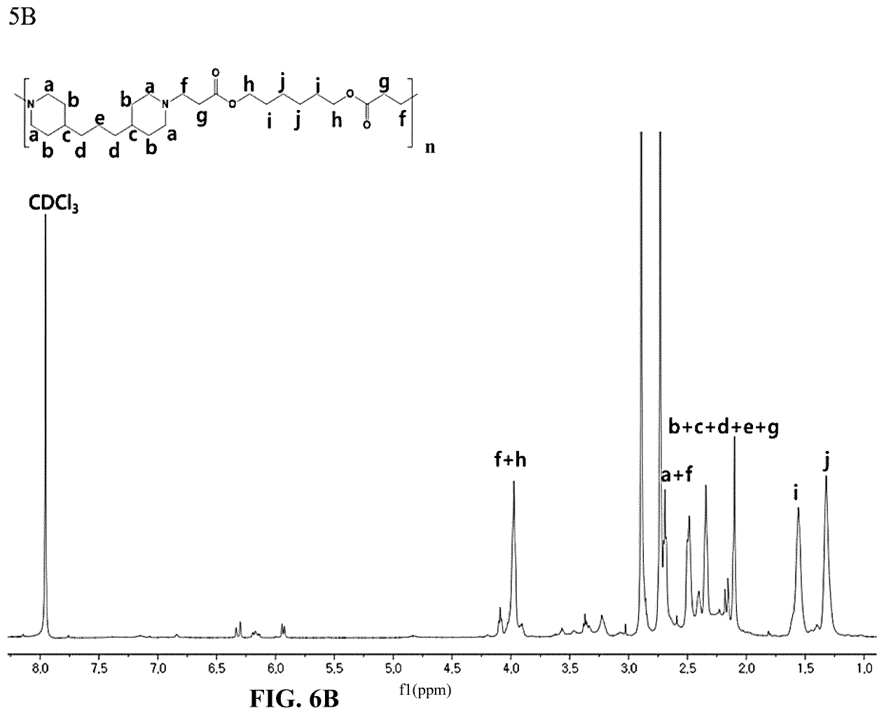

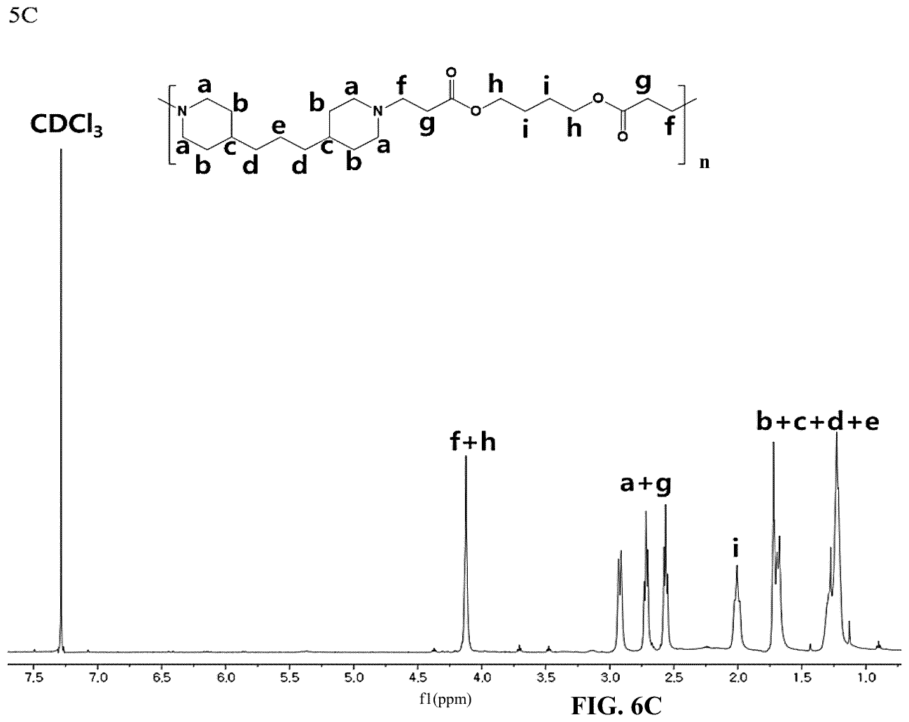

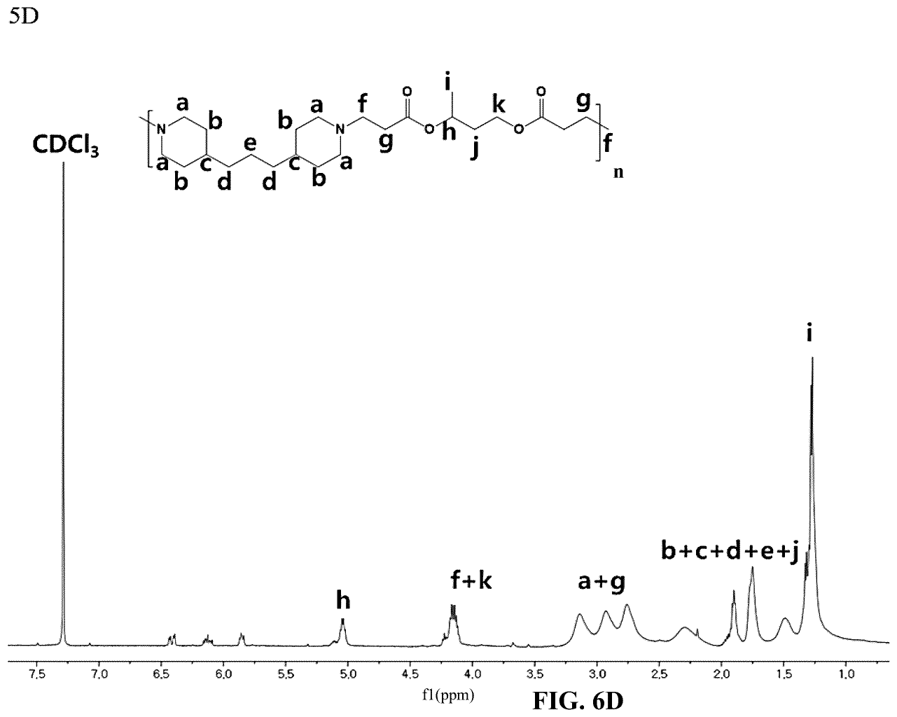

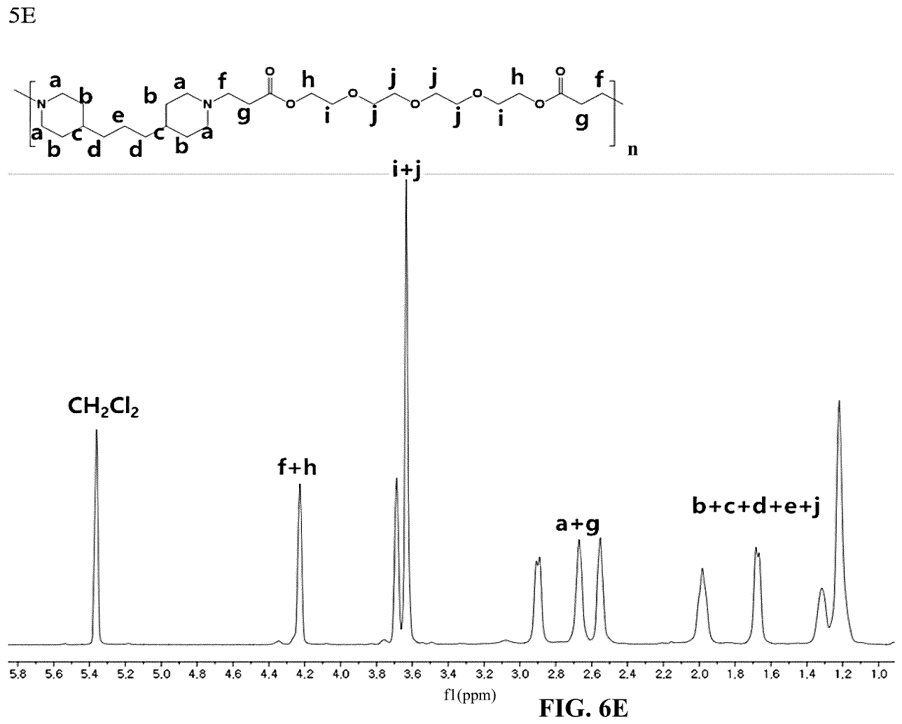

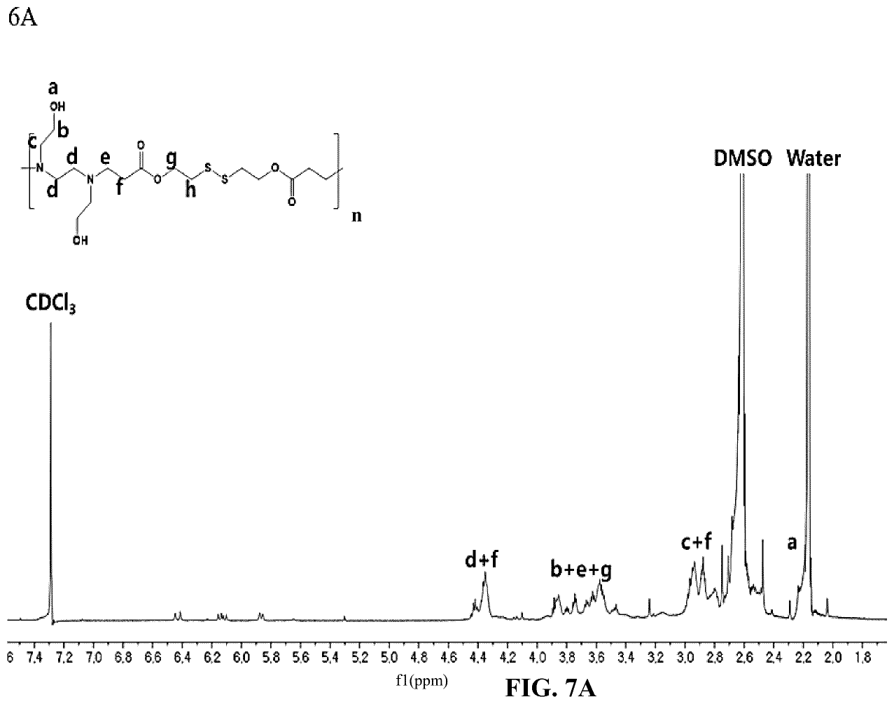

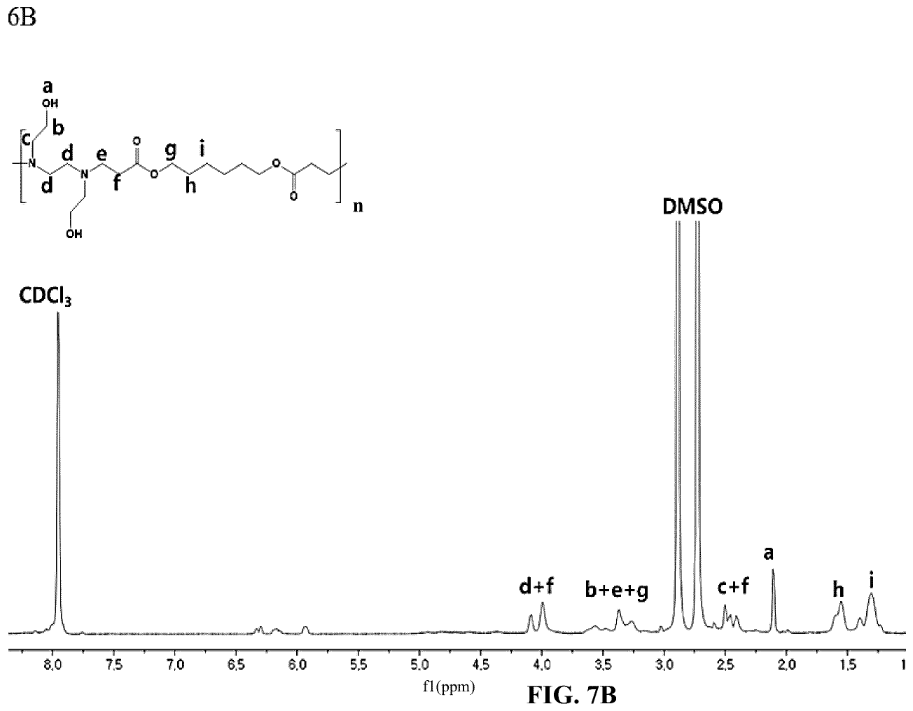

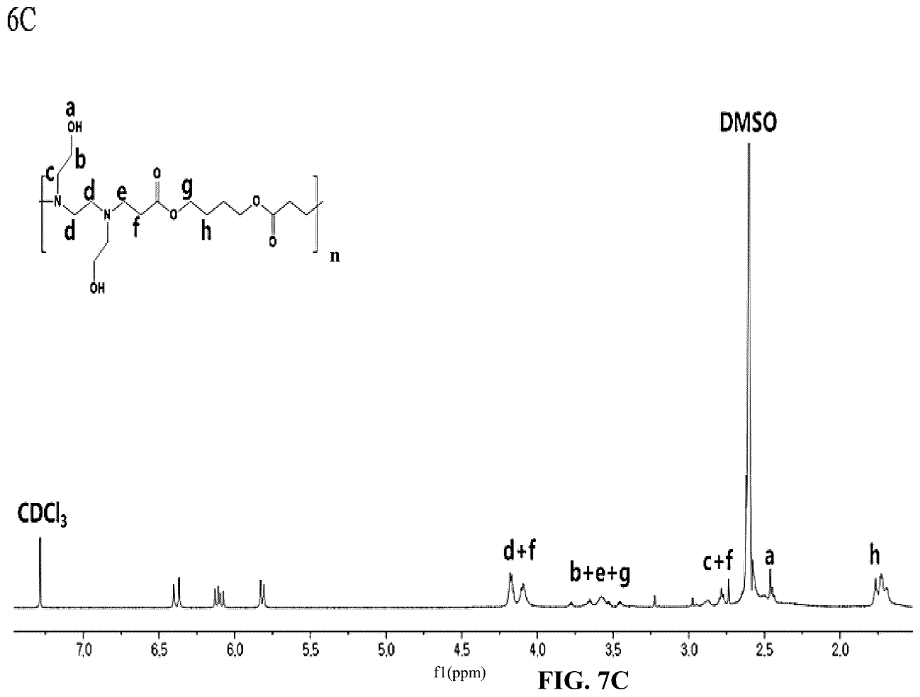

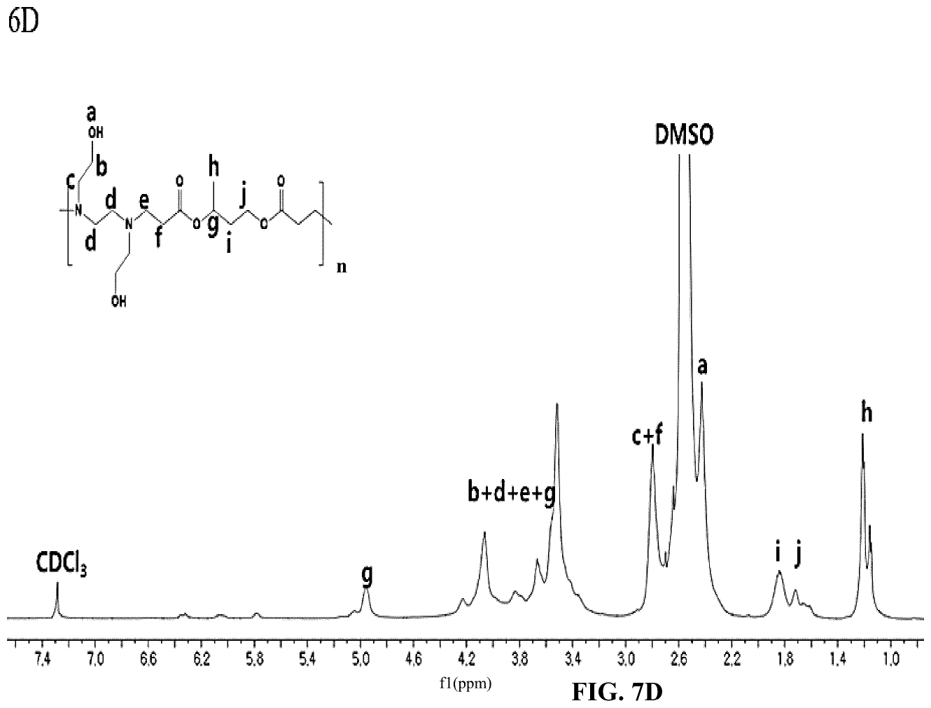

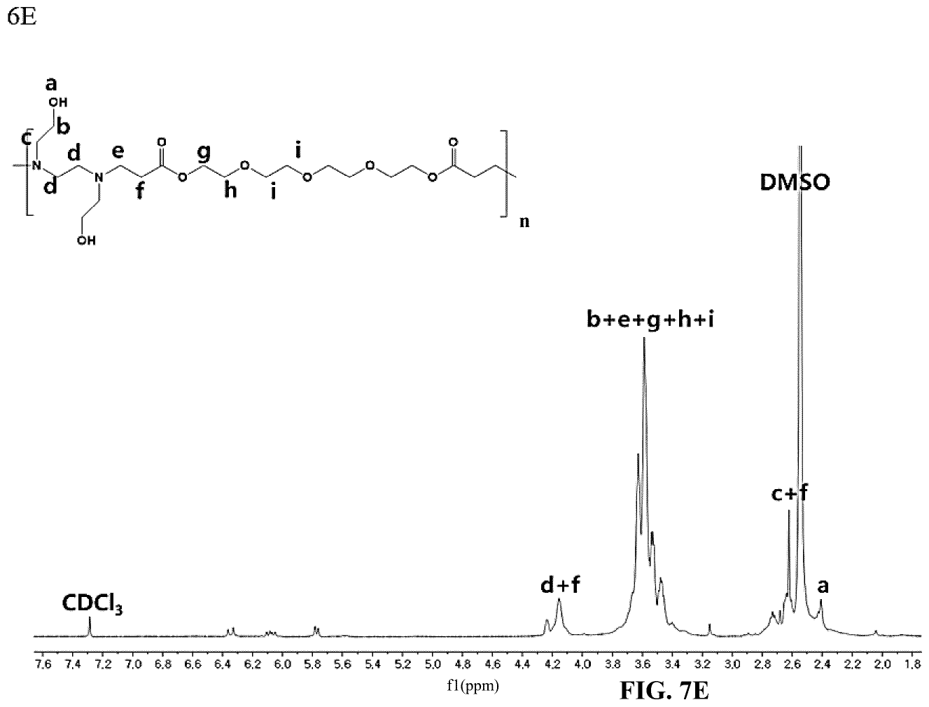

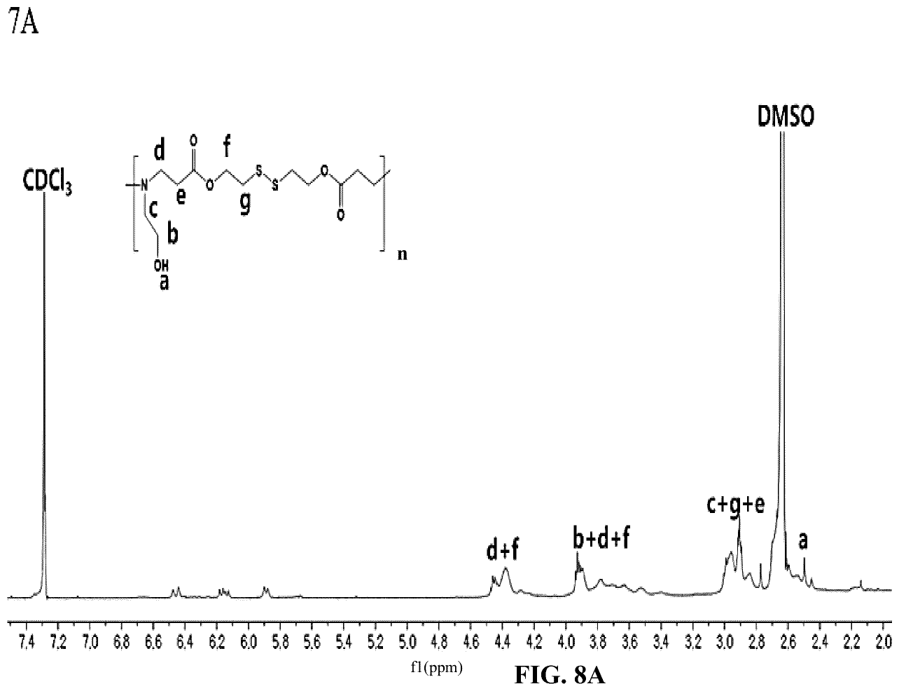

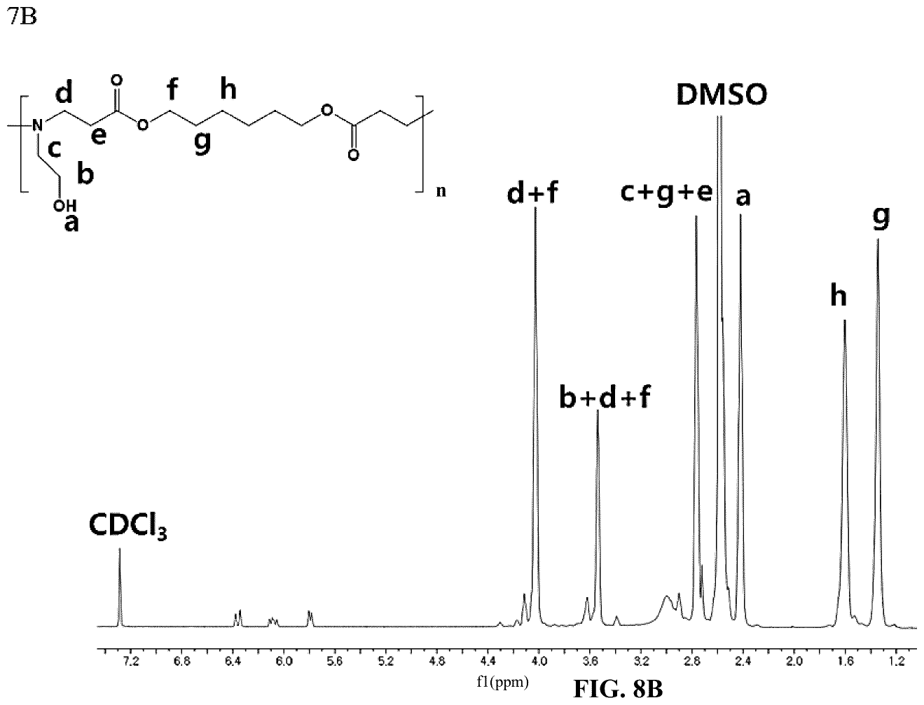

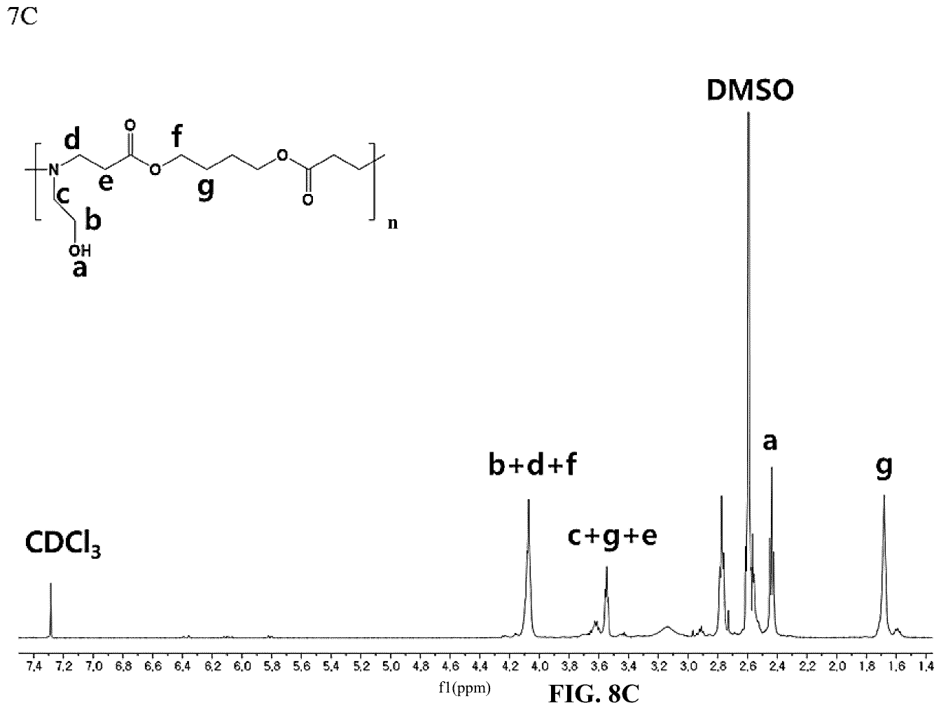

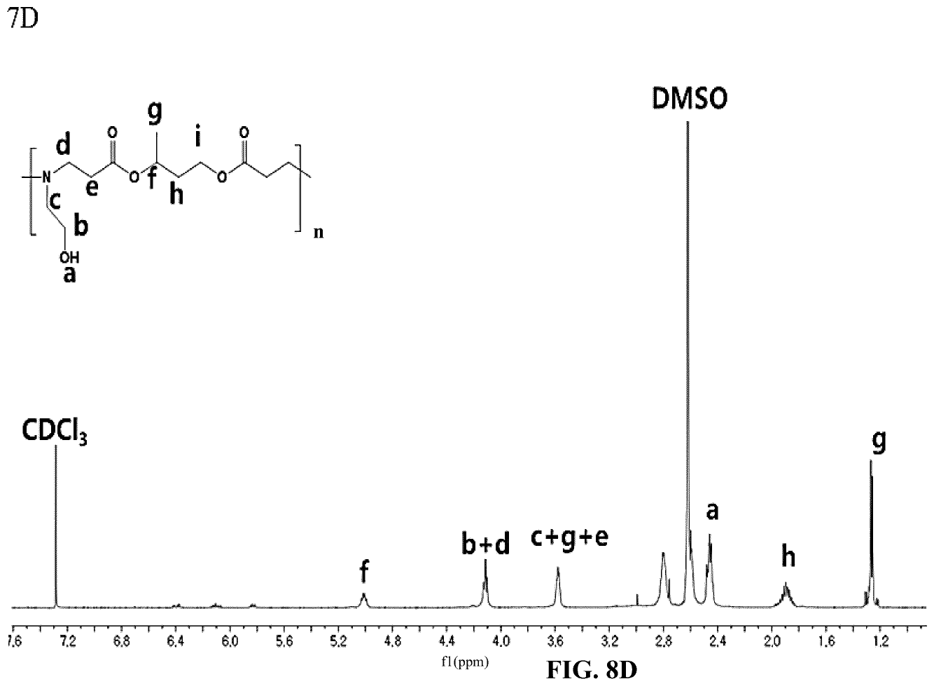

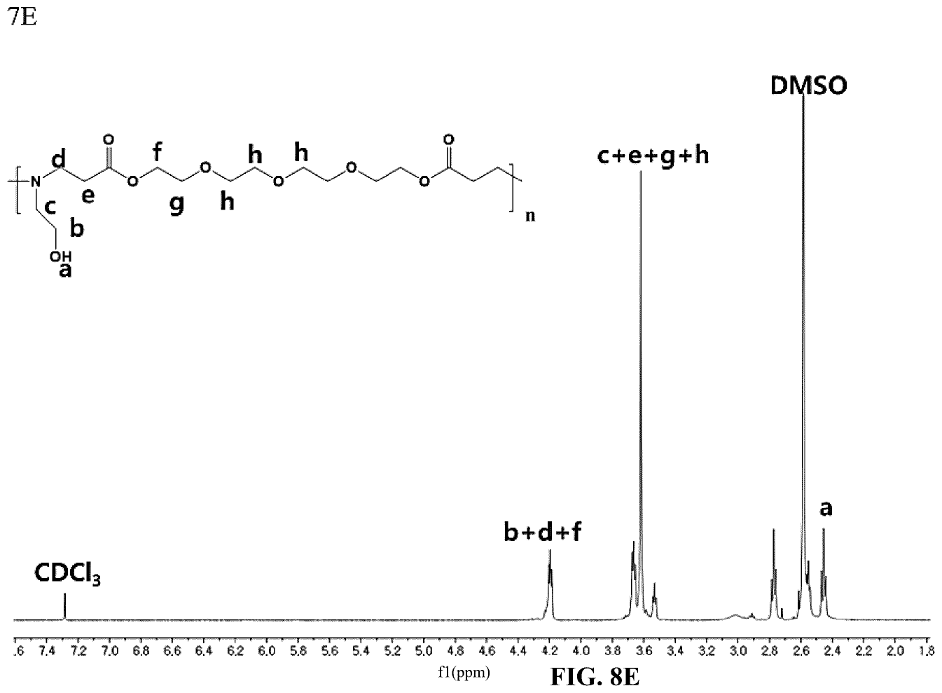

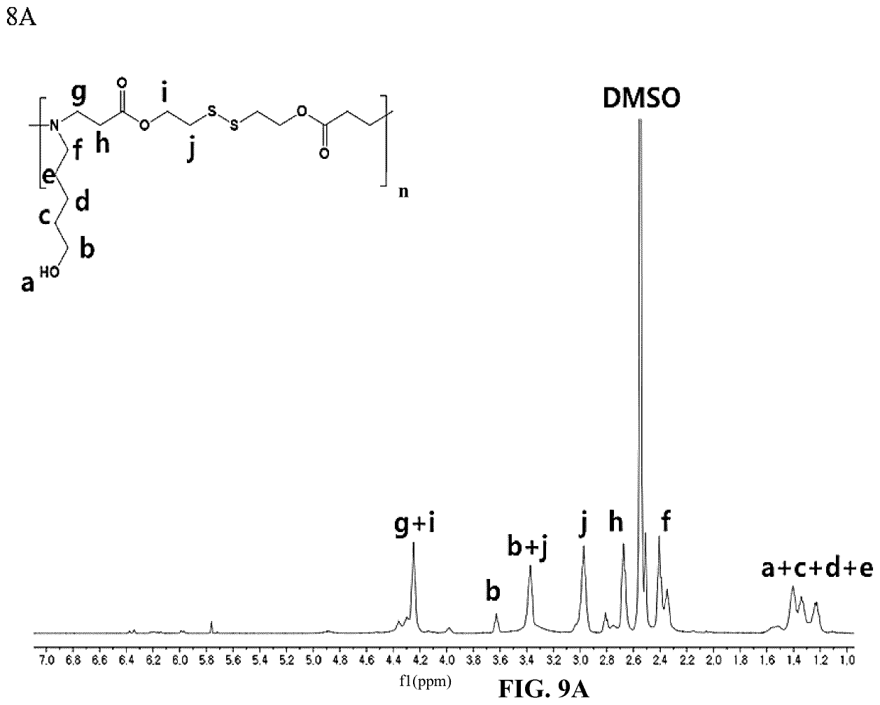

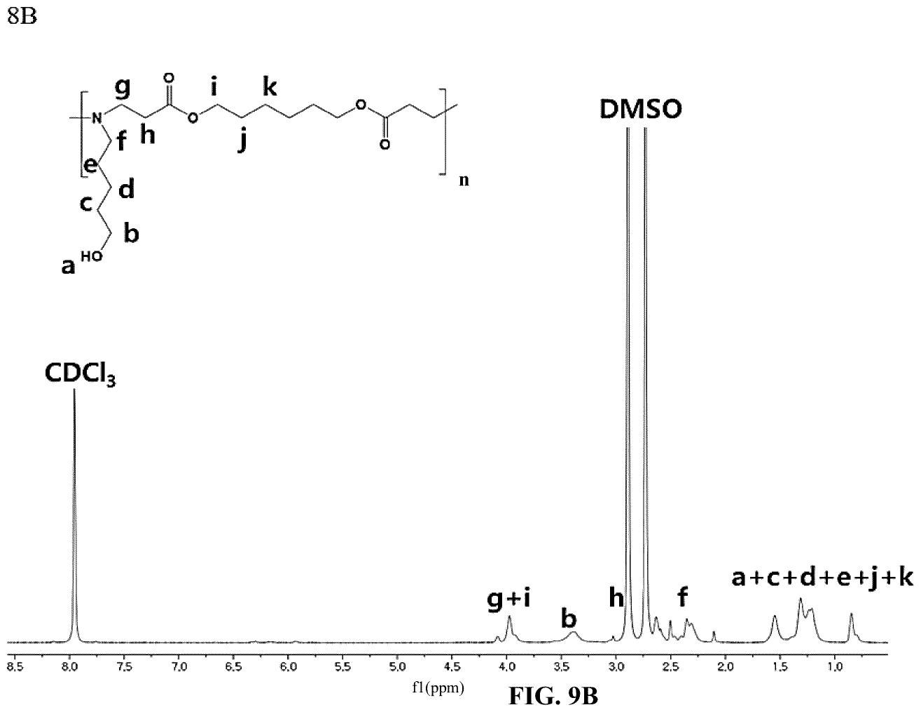

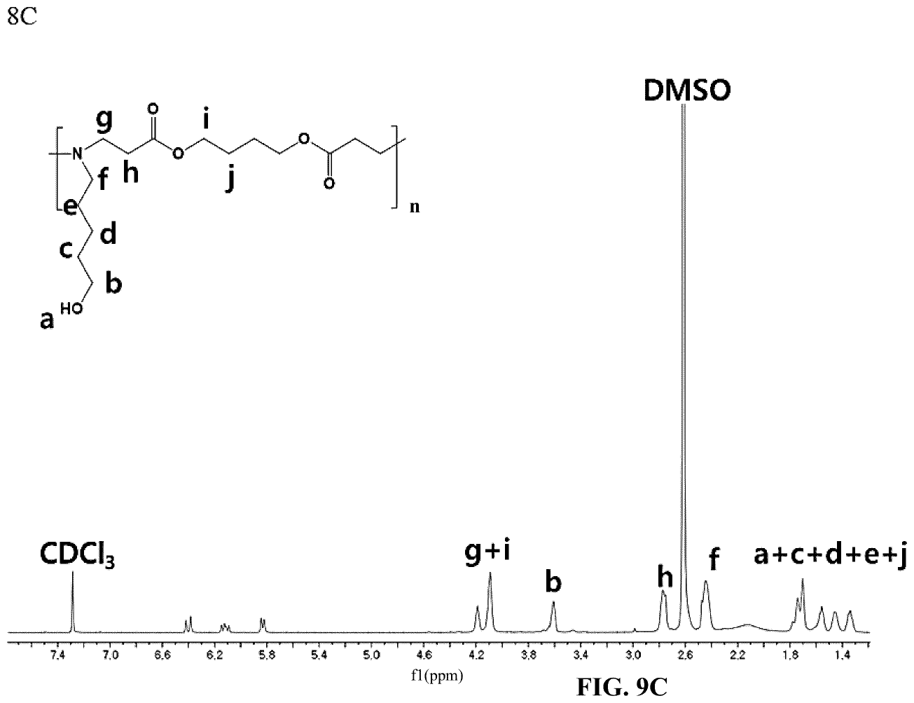

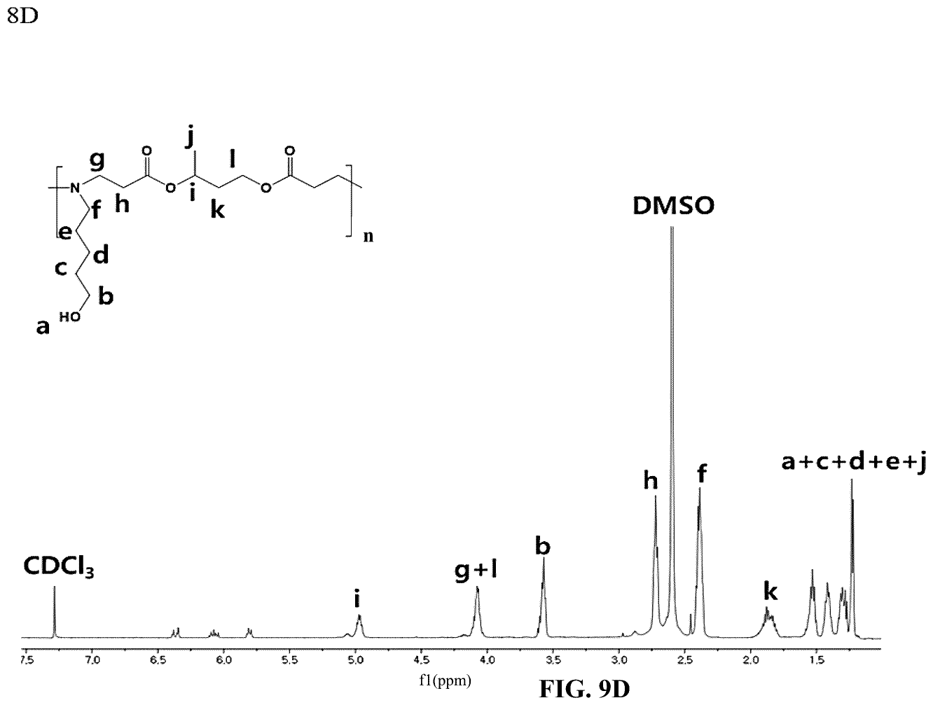

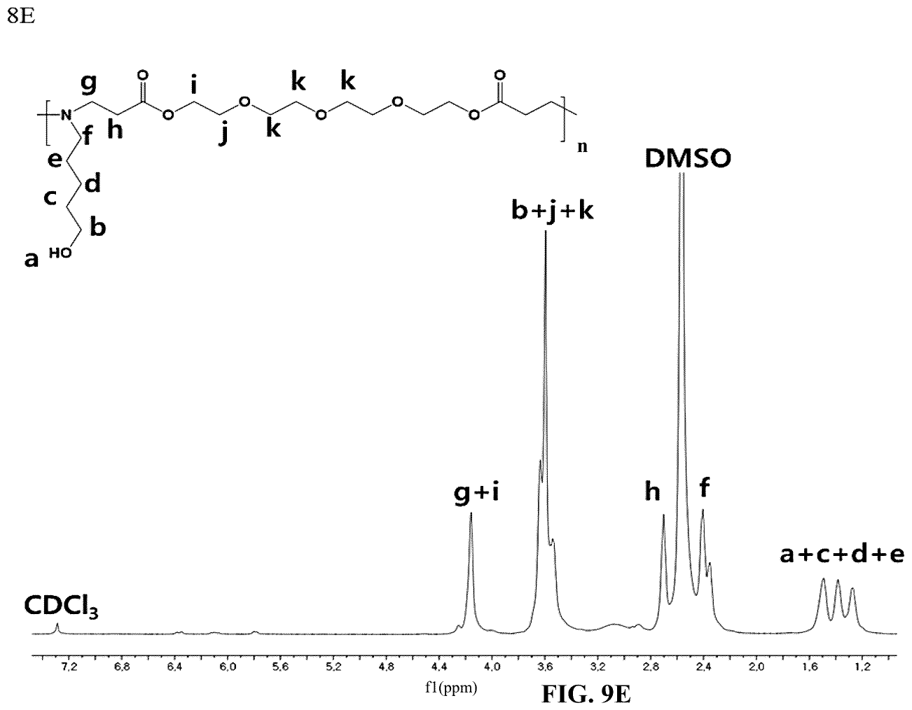

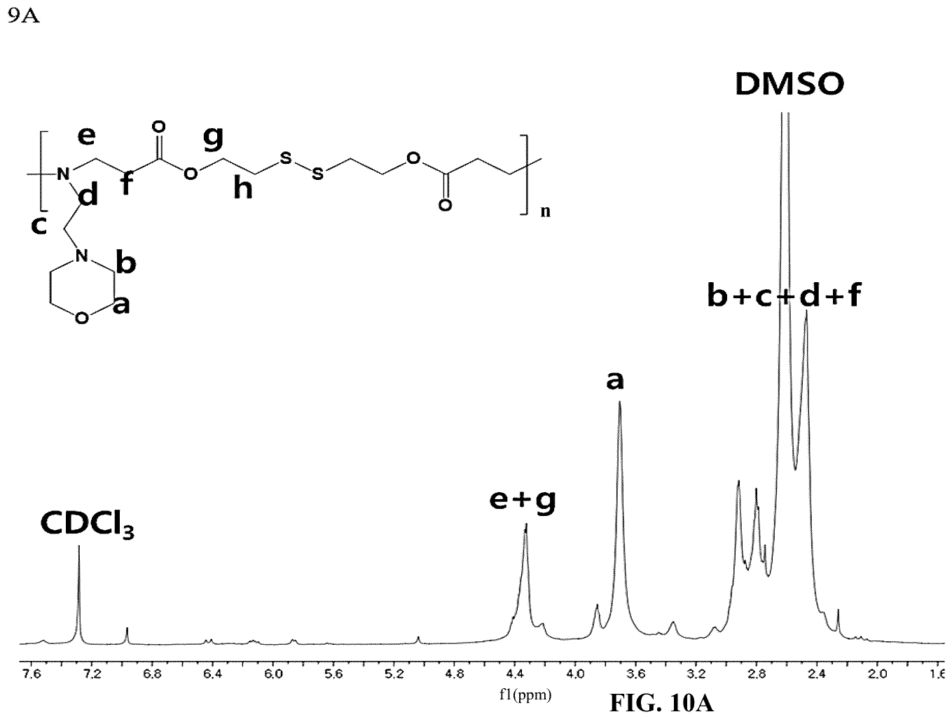

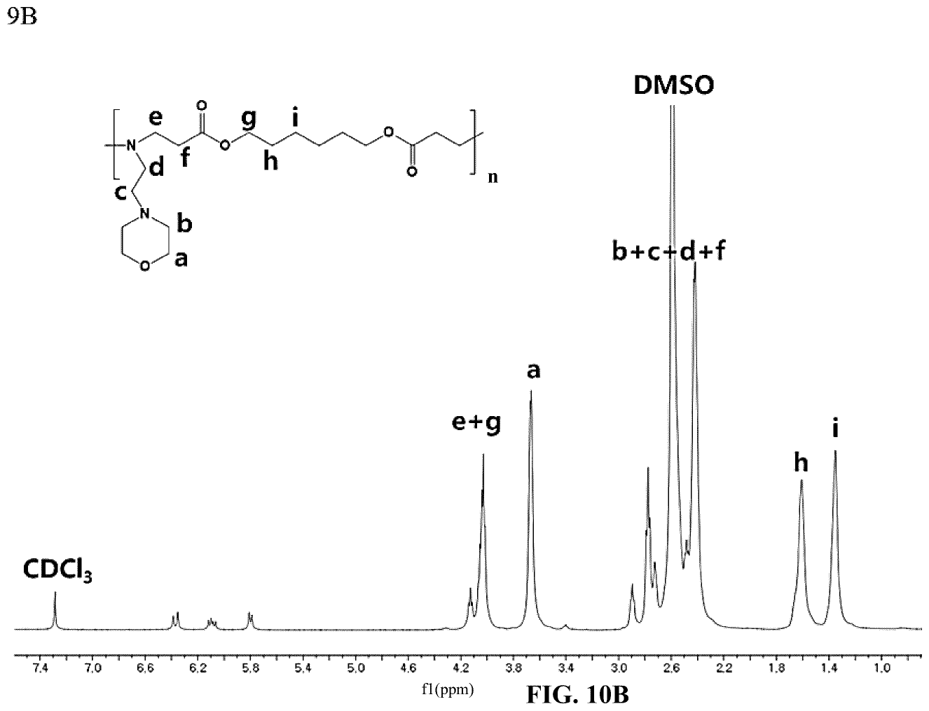

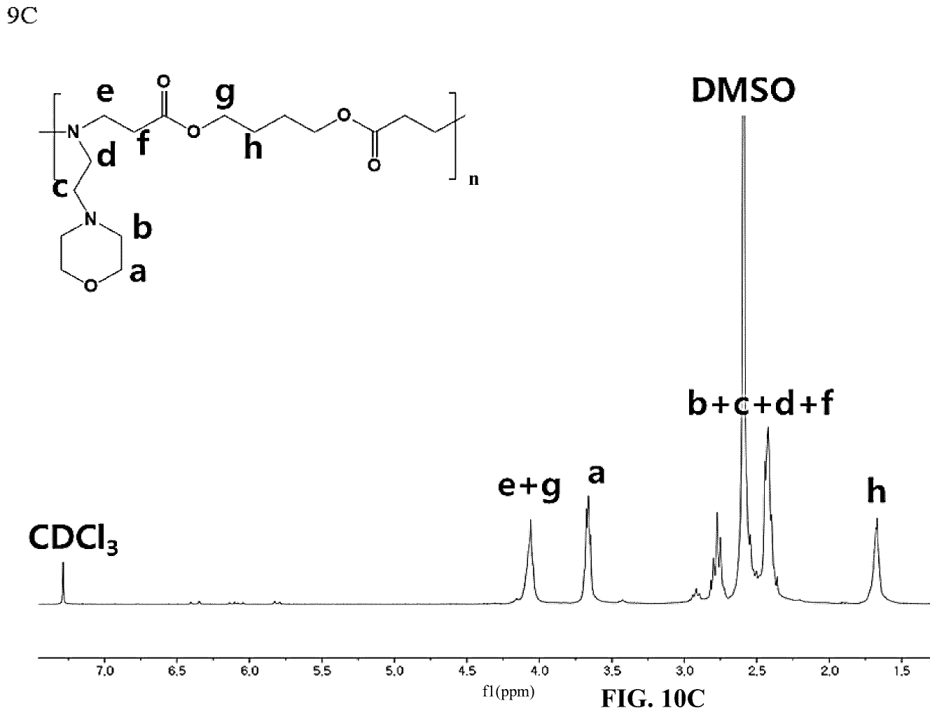

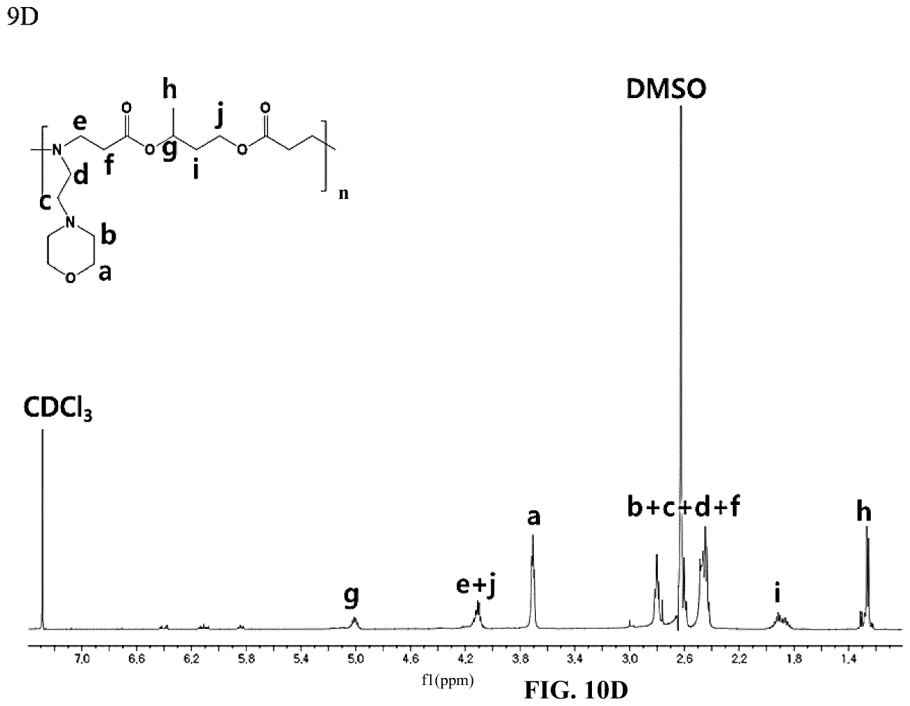

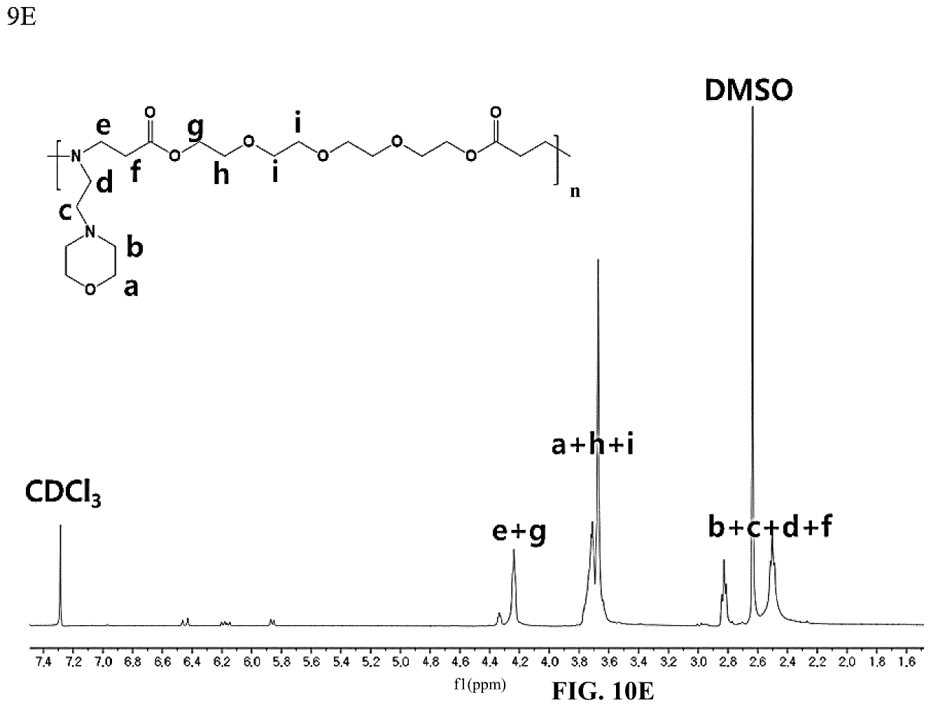

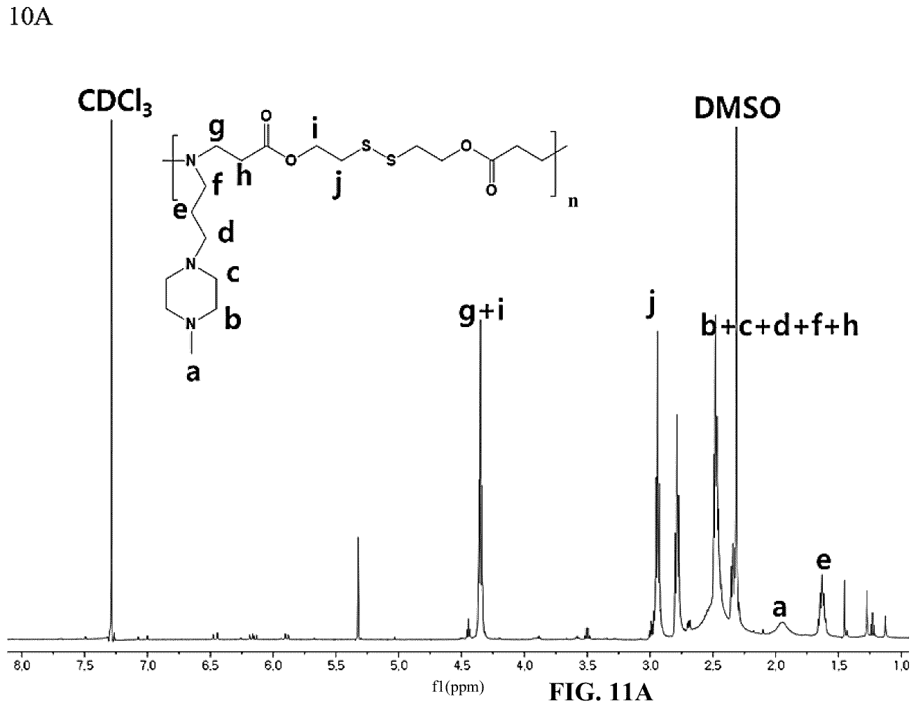

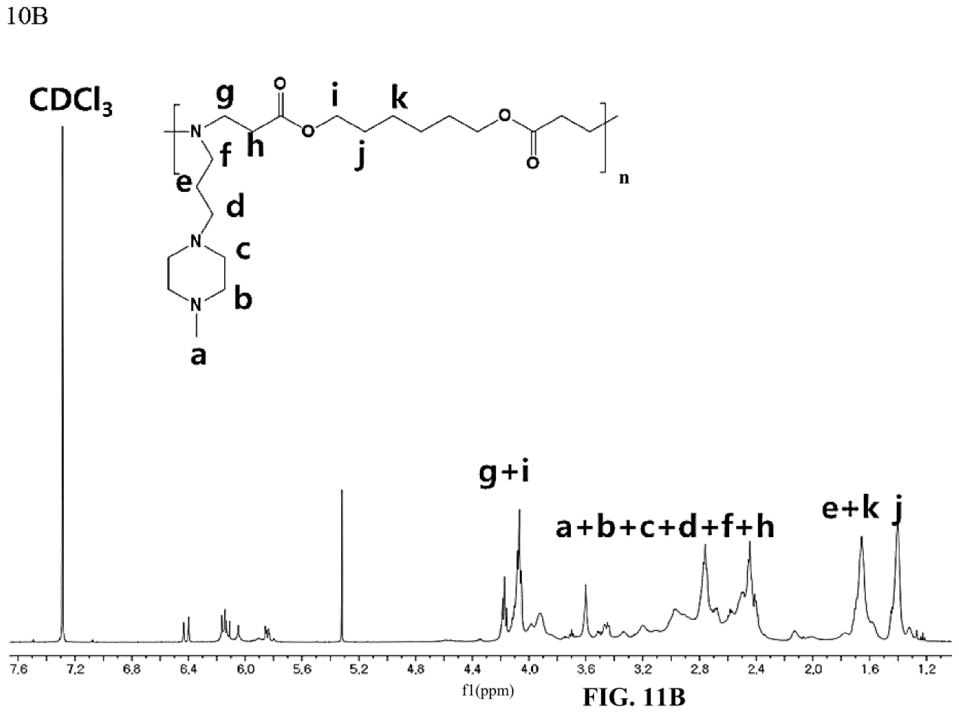

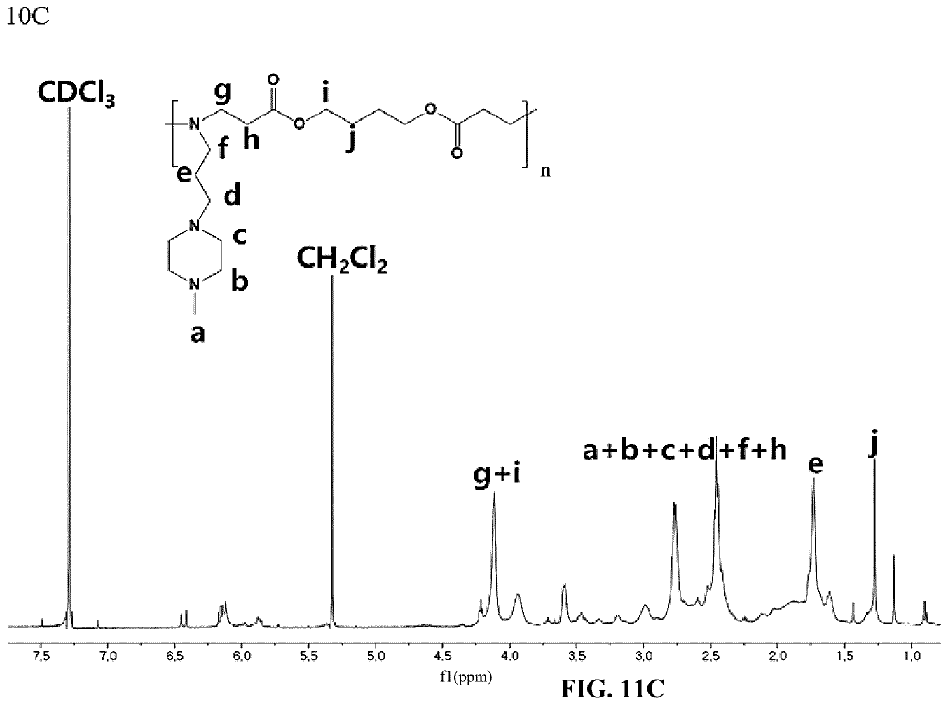

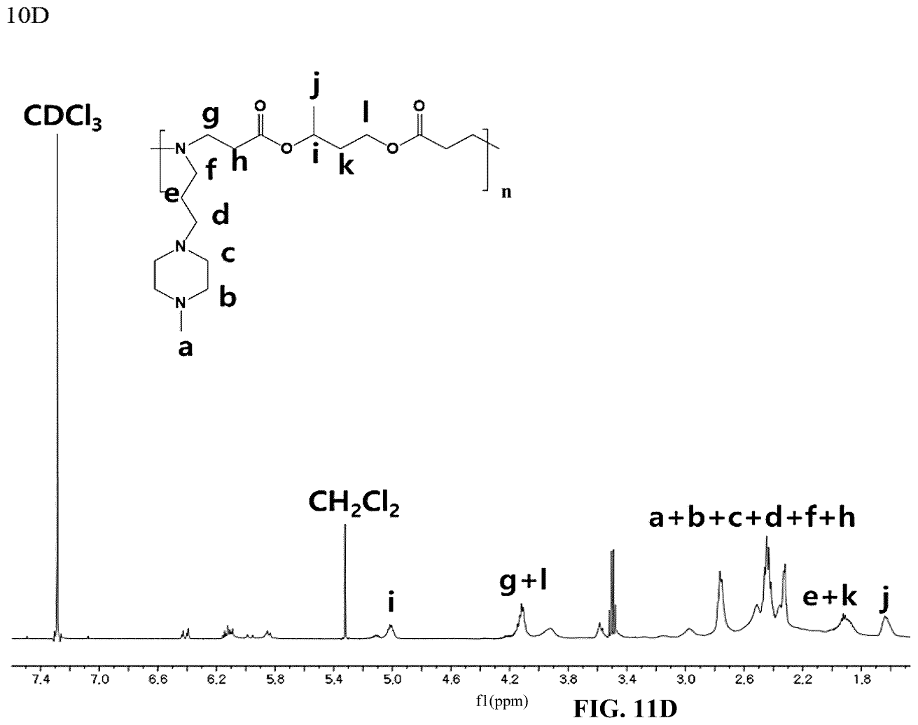

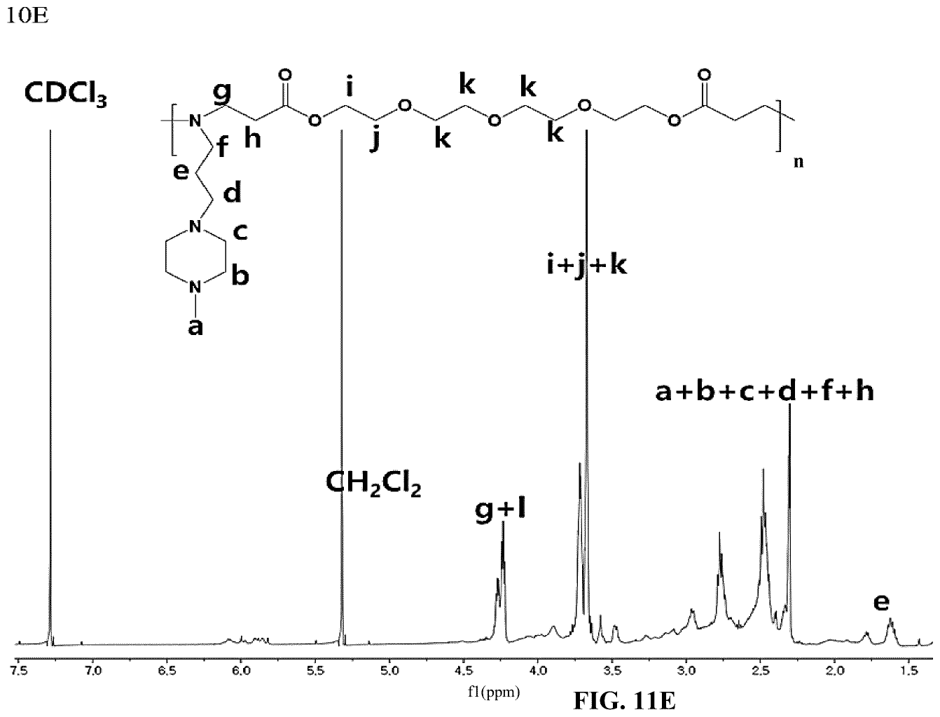

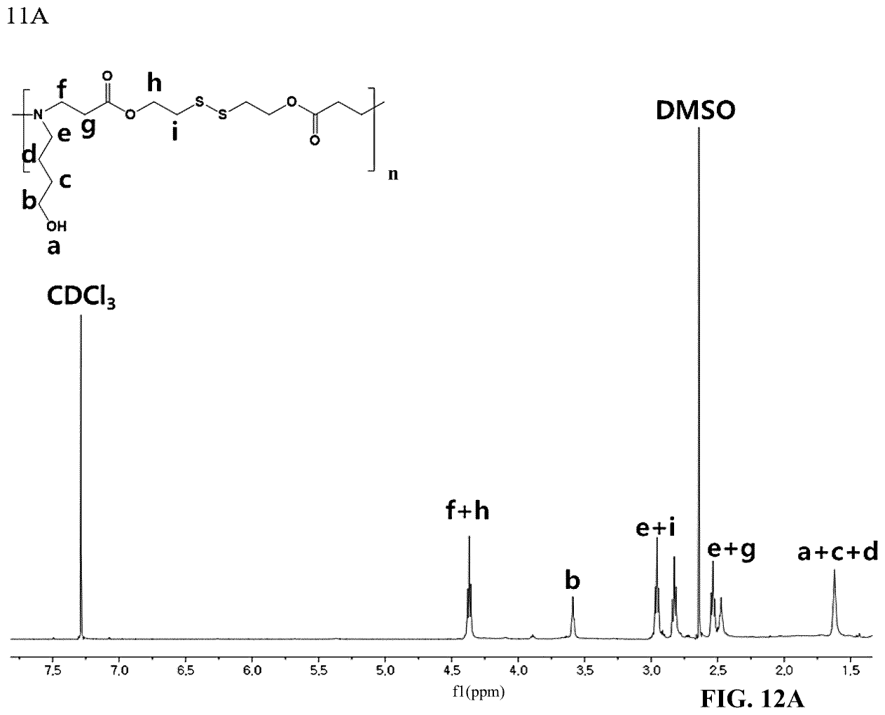

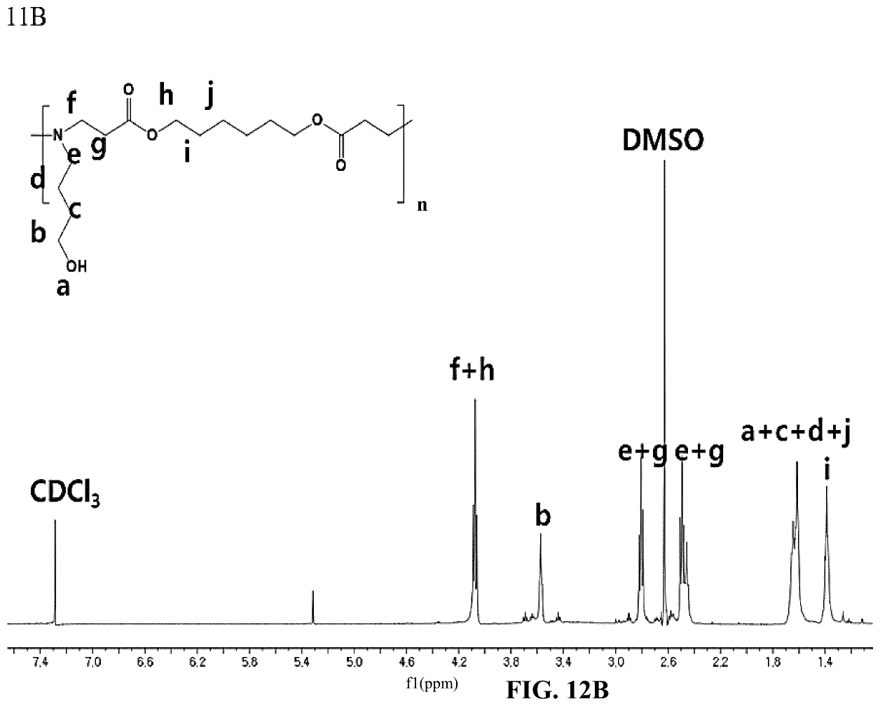

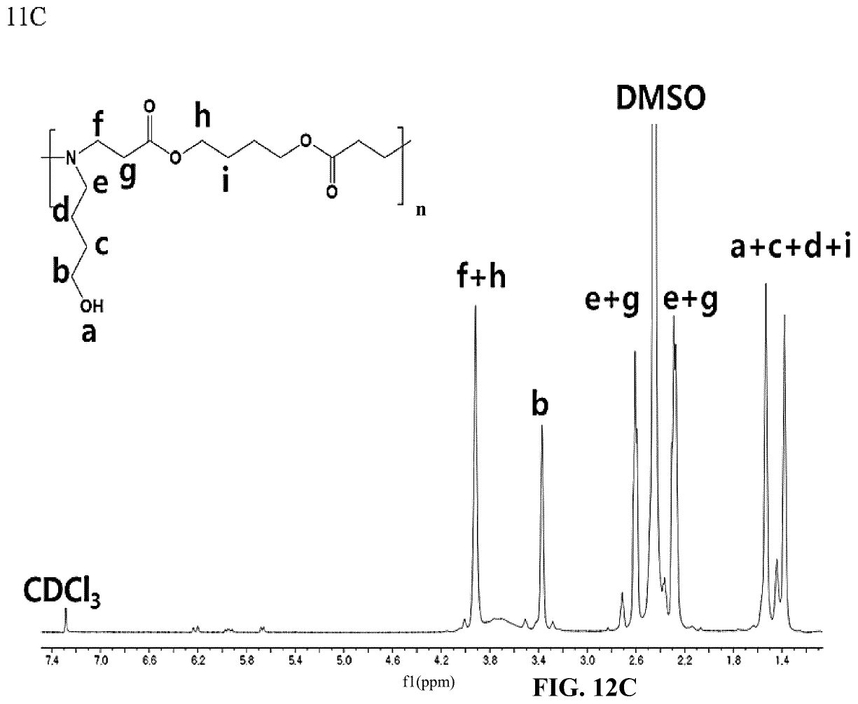

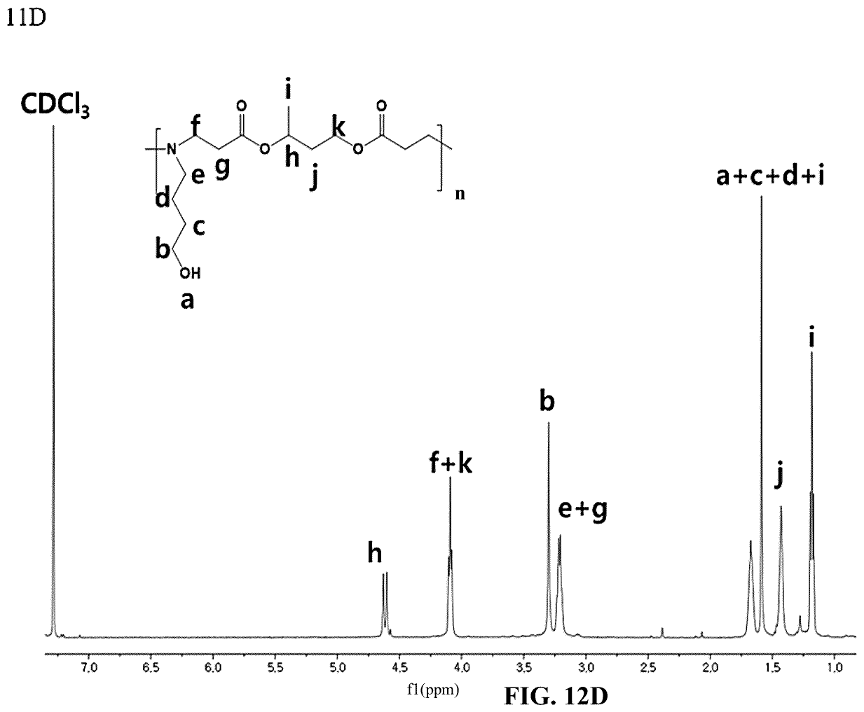

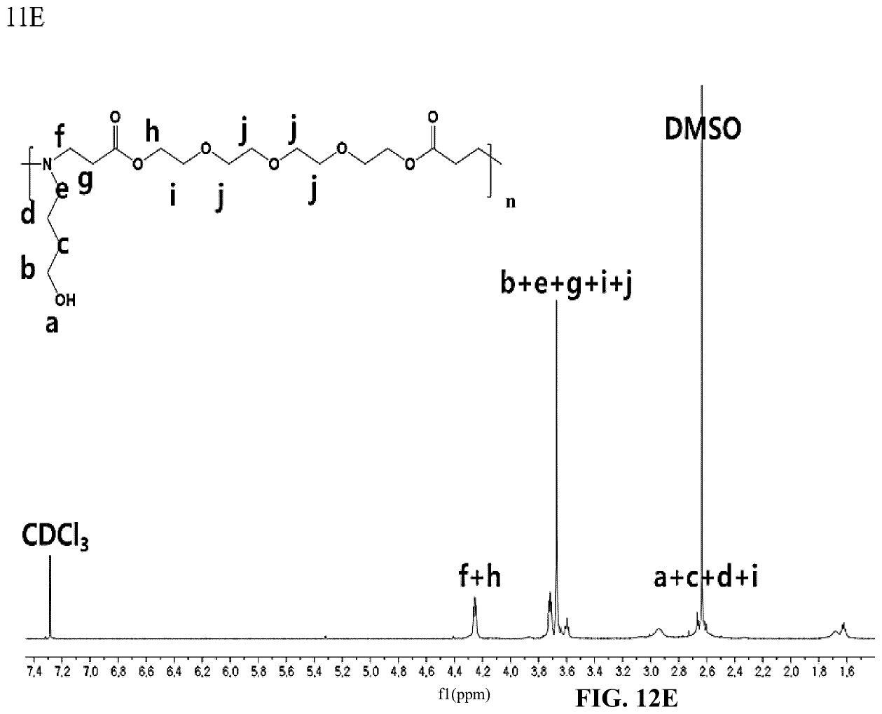

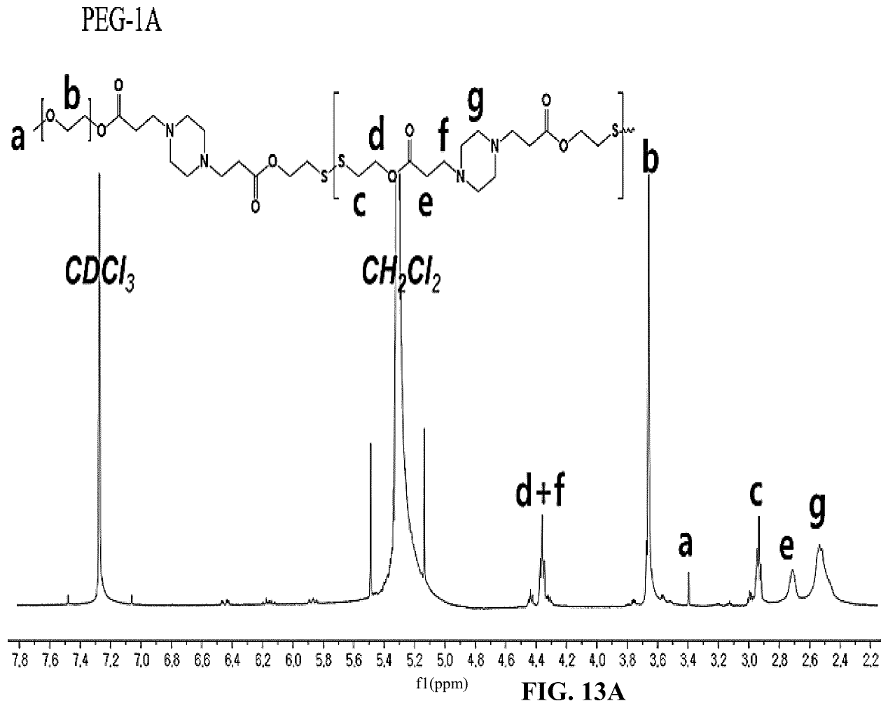

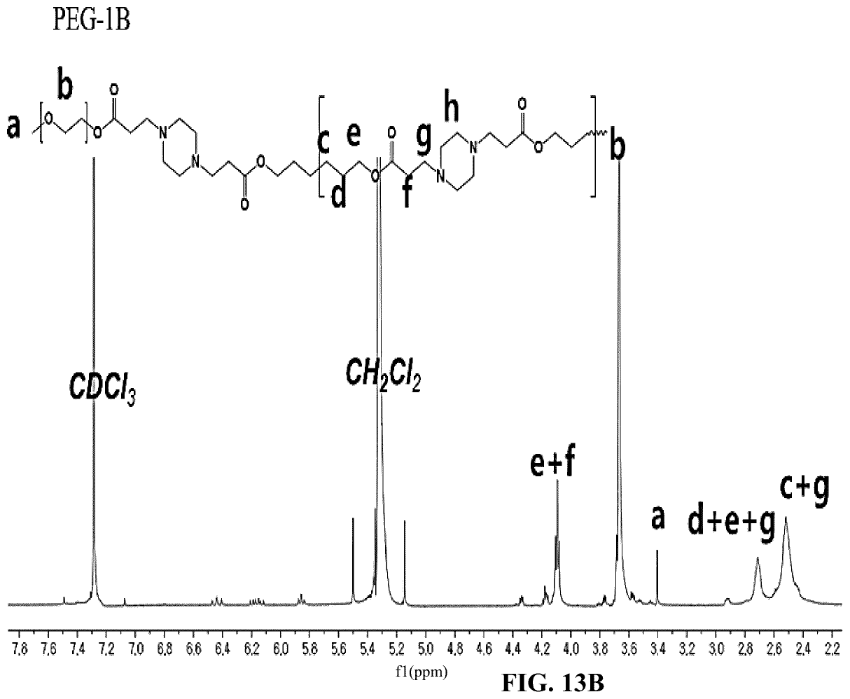

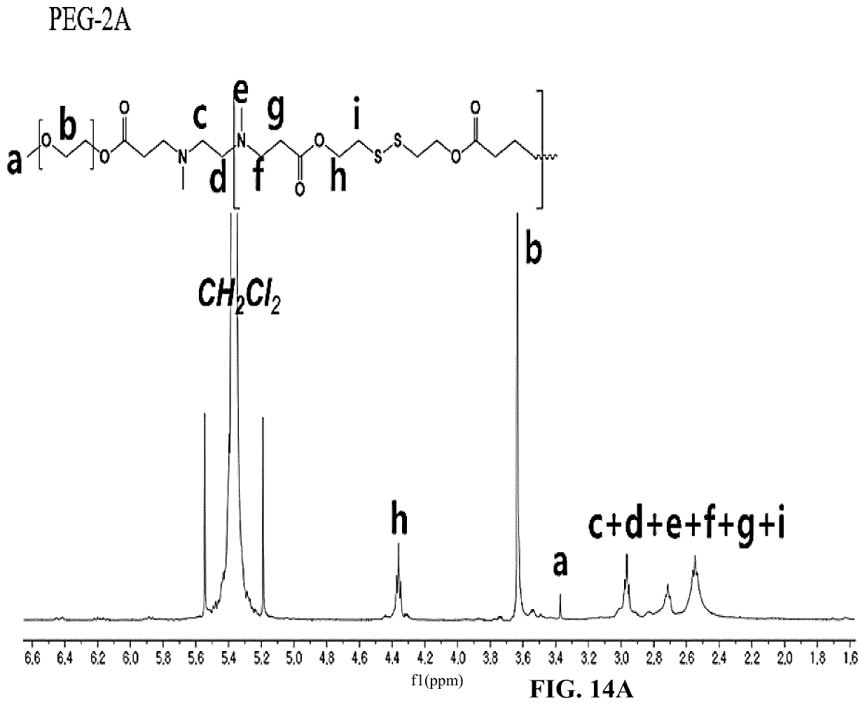

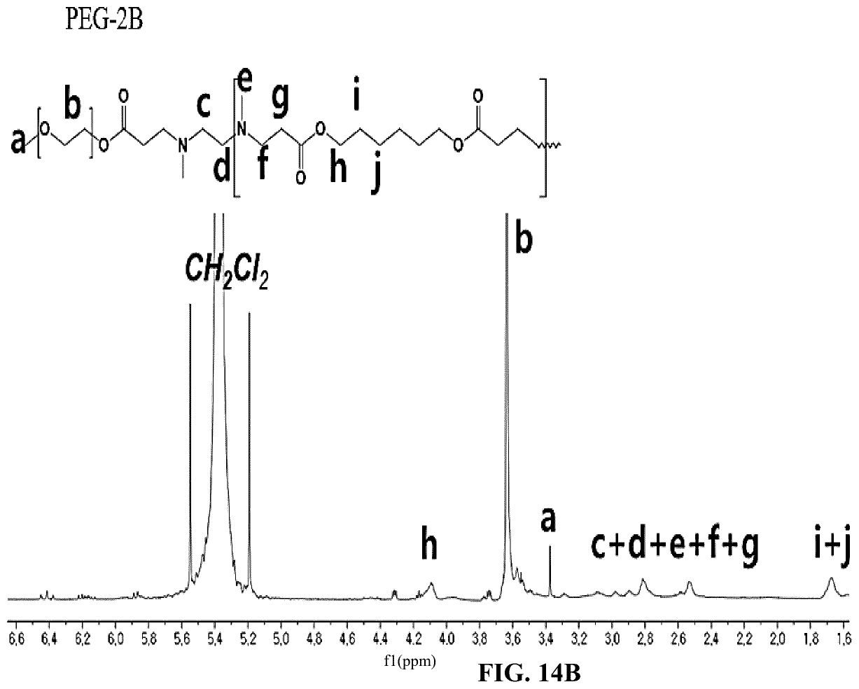

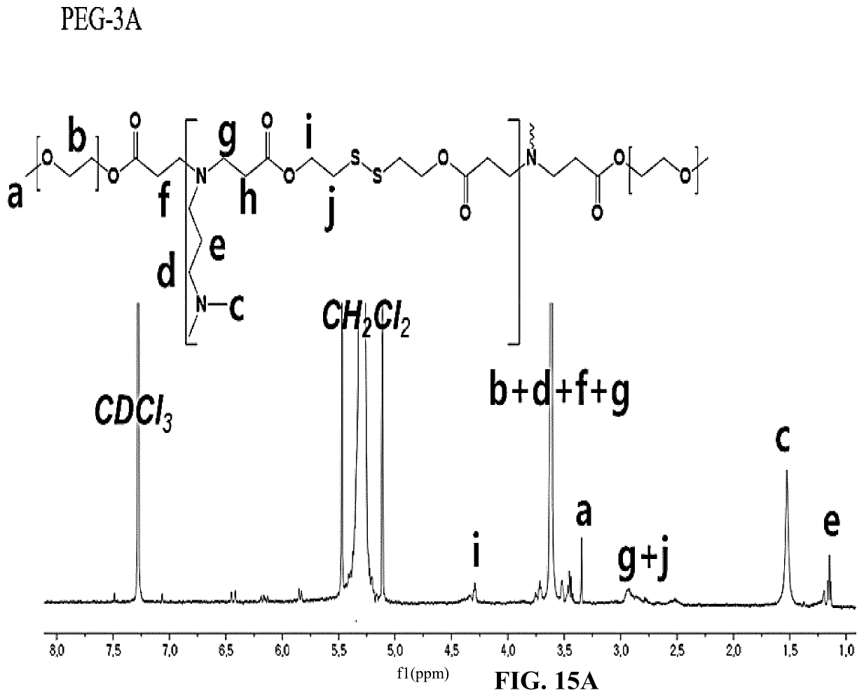

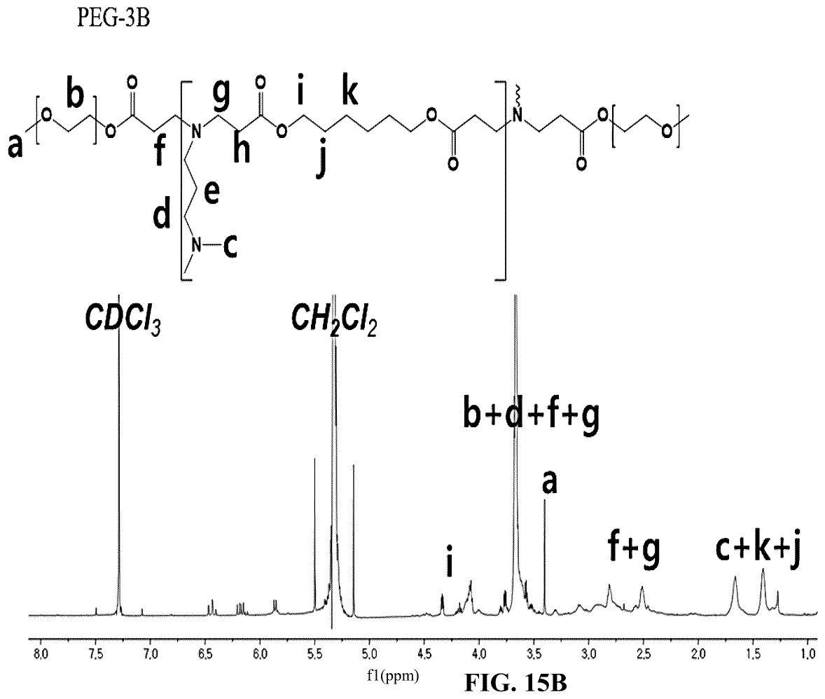

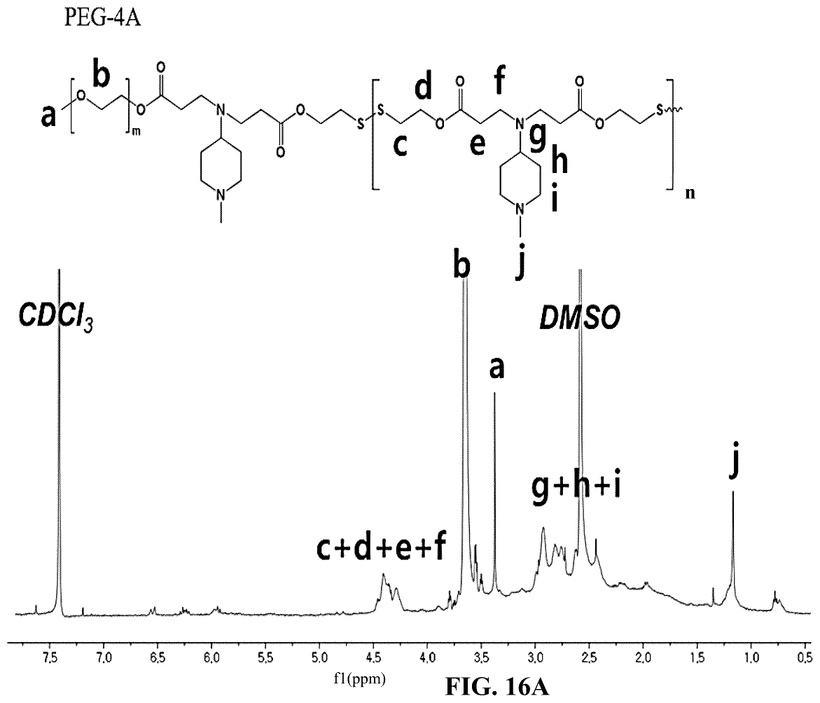

¹H-NMR analysis was performed on the synthesized polymer using deuterated chloroform (CDCl3) solvent, and the results are shown in FIGS. 2A to 16B. As shown in FIGS. 2A to 16B, it was confirmed that all polymers were prepared normally.

### 1.2 Hydrophobic Gel Permeable Chromatography

The solvent was removed from the synthesized polymer, and then analysis was performed using hydrophobic gel permeable chromatography (hydrophobic GPC) by dissolving the polymer in tetrahydrofuran (THF) solvent at a concentration of 1 to 3 mg/ml.

### Test Example 2

### Analysis of Polymeric Nanoparticle (PNP) Delivery Vehicle

The polymeric nanoparticle (PNP) prepared in the same manner as in Example 1 above was analyzed by the following method.

### 2.1 Agarose Gel Electrophoresis

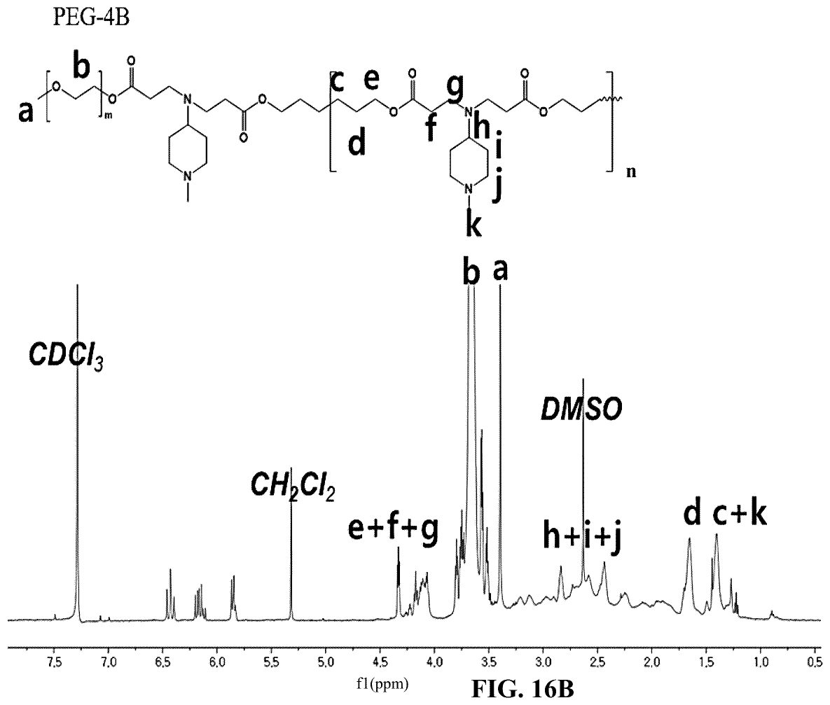

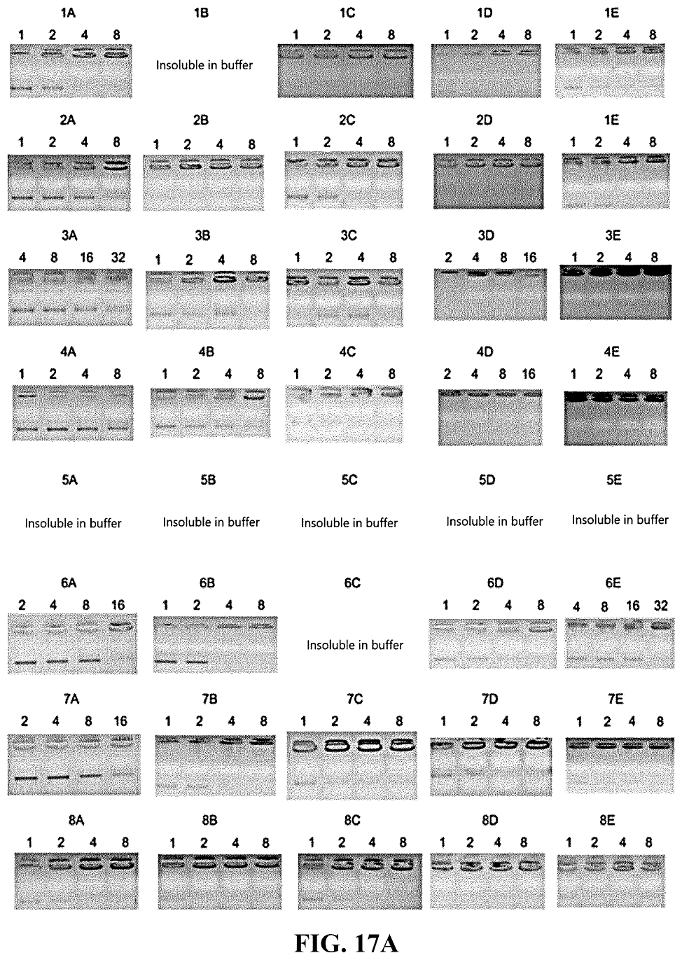

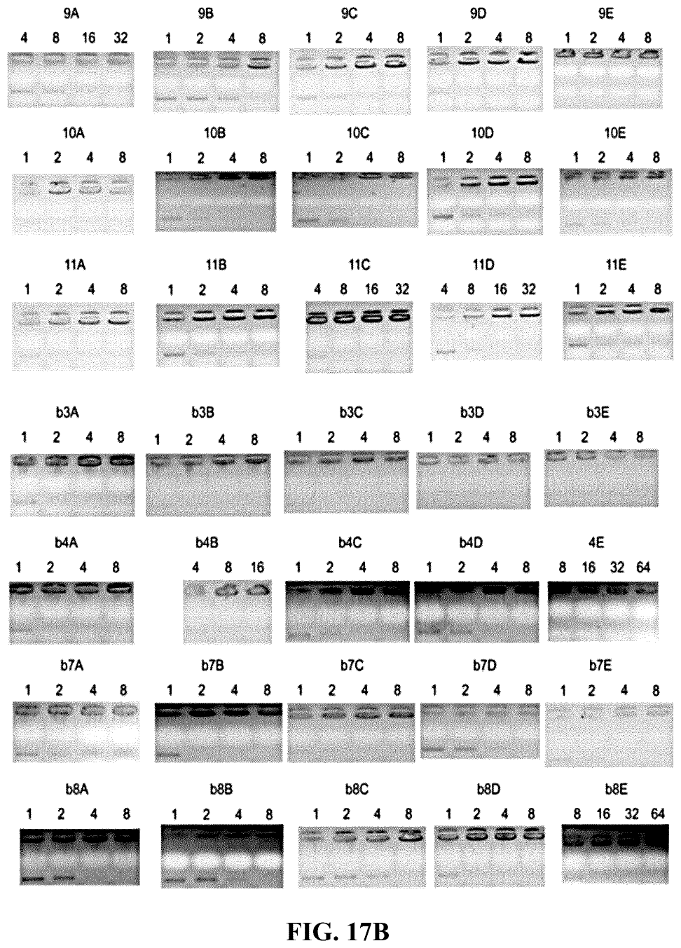

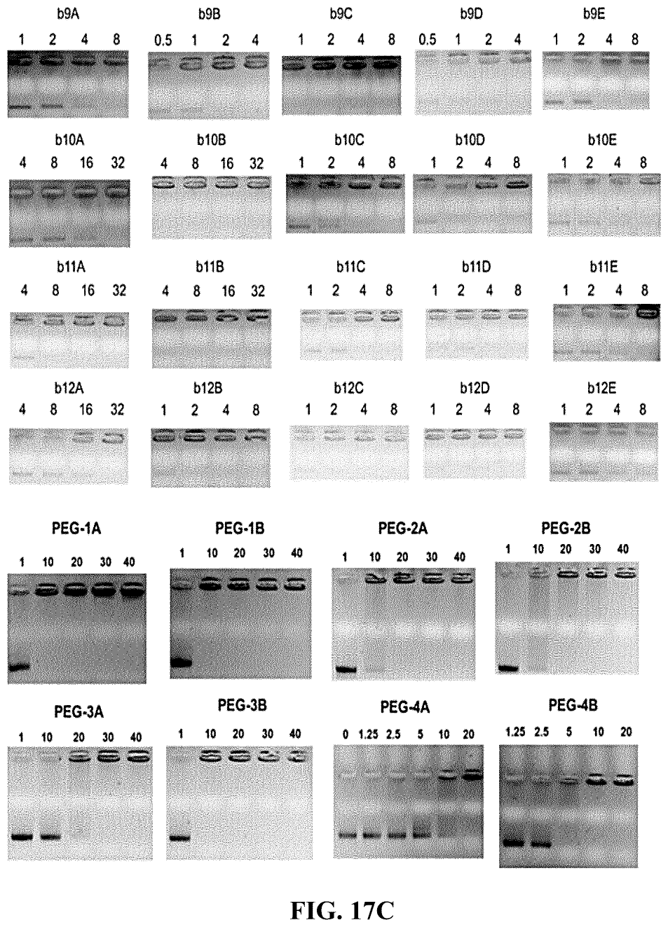

Agarose gel electrophoresis was performed on the polymers having pH sensitivity by adjusting the N/P ratio (the ratio of amine; N contained in the polymer and the ratio of phosphate; P contained in mRNA) of mRNA and polymer. The pH of the 25 mM NaOAc buffer was set to 5.0, and 5 μl of the solution in which the polymer was dissolved according to the N/P ratio was mixed with 5 μl of mRNA at a concentration of 200 ng/5 μl, and then gently vortexed. Thereafter, the mixture was stabilized at 4° C. for 30 minutes, loaded onto a 1 w/v % agarose gel containing GelRed, and electrophoresed at 100 V for 10 minutes. As a result of the experiment, as shown in FIGS. 17A to 17C, when a complex is formed at different N/P (Polymer/mRNA) ratios for each polymer, it can be confirmed that the mRNA band is not formed in the gel but is caught in the inlet. For example, the 6B polymer completely formed a complex starting from an N/P ratio of 4.

### 2.2 Zeta Potential and Dynamic Light Scattering Measurement

For the polymers having pH sensitivity, the N/P ratio for each polymer was adjusted, and 6 μl of the solution dissolved in a pH 5.0, 25 mM NaOAc buffer was mixed with 6 μl of luciferase mRNA (FLuc) of SEQ ID NO: 1 at a concentration of 1 mg/ml, and then gently vortexed. Thereafter, the mixture was stabilized at 4° C. for 30 minutes, and 38 μl of PBS was added to make a final volume of 50 μl.

The zeta potential and size of the polymer-mRNA complex were measured by diluting the complex solution described above 10 times in distilled water and using a zeta potential meter and a dynamic light scattering (DLS) device. As shown in FIG. 18, it was confirmed that the zeta potential changed depending on the pH. Through this, it was confirmed that the mRNA complex was formed due to a positive charge at low pH, and it was confirmed that it could have low toxicity and high mRNA delivery efficiency by changing to neutral in the pH in the body.

### Test Example 3

Confirmation of mRNA Expression In Vivo of Polymeric Nanoparticles (PNP)

The polymeric nanoparticles prepared according to Example 1 above were injected intramuscularly into animals to confirm the expression of mRNA.

3.1 Confirmation of In Vivo mRNA Expression

A representative polymer was synthesized according to the method described in the above manufacturing example, and polymeric nanoparticles were prepared according to Manufacturing Example 261, and mRNA expression level screening was performed. The method for forming the polymer-mRNA complex used for in vivo injection to confirm the mRNA expression is as follows. The weight ratio of each polymer having pH sensitivity and a micelle structure was adjusted, and 6 μl of the solution dissolved in a pH 5.0, 25 mM NaOAc buffer was mixed with 6 μl of FLuc of SEQ ID NO: 1 at a concentration of 1 mg/ml, and then gently vortexed. Thereafter, the mixture was stabilized at 4° C. for 30 minutes, and 38 μl of PBS was added to make a final volume of 50 μl. Regarding the preparation of lipid nanoparticles (LNPs) used as a comparative group, the following preparation method was used. The molar ratio of ionizable lipid (SM-102):DSPC:cholesterol:PEG-lipid was set to 50:10:38.5:1.5 and dissolved in ethanol. Thereafter, the mRNA diluted in a 25 mM, pH 5.0 sodium acetate buffer was mixed with the mixture dissolved in ethanol so that the volume ratio was 3:1 and the N/P ratio was 5.67. Thereafter, Centrifugation was performed at least three times at 1000 g for 5 minutes with PBS added using a 100 kDa Amicon. Finally, the mRNA was diluted with PBS so that the concentration was 6 ug/50 ul.

TABLE 4

| SEQ | |
|---|---|
| ID | |
| NO | RNA sequence |
| 1 | ATGGAGGACGCCAAGAACATCAAGAAGGGCCCCGCCCCCTTCTACCCCCTGGAGG |
| | ACGGCACCGCCGGCGAGCAGCTGCACAAGGCCATGAAGCGGTACGCCCTGGTGC |
| | CCGGCACCATCGCCTTCACCGACGCCCACATCGAGGTGGACATCACCTACGCCGA |
| | GTACTTCGAGATGAGCGTGCGGCTGGCCGAGGCCATGAAGCGGTACGGCCTGAAC |
| | ACCAACCACCGGATCGTGGTGTGCAGCGAGAACAGCCTGCAGTTCTTCATGCCCG |
| | TGCTGGGCGCCCTGTTCATCGGCGTGGCCGTGGCCCCCGCCAACGACATCTACAA |
| | CGAGCGGGAGCTGCTGAACAGCATGGGCATCAGCCAGCCCACCGTGGTGTTCGTG |
| | AGCAAGAAGGGCCTGCAGAAGATCCTGAACGTGCAGAAGAAGCTGCCCATCATC |
| | CAGAAGATCATCATCATGGACAGCAAGACCGACTACCAGGGCTTCCAGAGCATGT |
| | ACACCTTCGTGACCAGCCACCTGCCCCCCGGCTTCAACGAGTACGACTTCGTGCCC |
| | GAGAGCTTCGACCGGGACAAGACCATCGCCCTGATCATGAACAGCAGCGGCAGC |
| | ACCGGCCTGCCCAAGGGCGTGGCCCTGCCCCACCGGACCGCCTGCGTGCGGTTCA |
| | GCCACGCCCGGGACCCCATCTTCGGCAACCAGATCATCCCCGACACCGCCATCCT |
| | GAGCGTGGTGCCCTTCCACCACGGCTTCGGCATGTTCACCACCCTGGGCTACCTGA |
| | TCTGCGGCTTCCGGGTGGTGCTGATGTACCGGTTCGAGGAGGAGCTGTTCCTGCG |
| | GAGCCTGCAGGACTACAAGATCCAGAGCGCCCTGCTGGTGCCCACCCTGTTCAGC |
| | TTCTTCGCCAAGAGCACCCTGATCGACAAGTACGACCTGAGCAACCTGCACGAGA |
| | TCGCCAGCGGCGGCGCCCCCCTGAGCAAGGAGGTGGGCGAGGCCGTGGCCAAGC |
| | GGTTCCACCTGCCCGGCATCCGGCAGGGCTACGGCCTGACCGAGACCACCAGCGC |
| | CATCCTGATCACCCCCGAGGGCGACGACAAGCCCGGCGCCGTGGGCAAGGTGGT |
| | GCCCTTCTTCGAGGCCAAGGTGGTGGACCTGGACACCGGCAAGACCCTGGGCGTG |
| | AACCAGCGGGGCGAGCTGTGCGTGCGGGGCCCCATGATCATGAGCGGCTACGTG |
| | AACAACCCCGAGGCCACCAACGCCCTGATCGACAAGGACGGCTGGCTGCACAGC |
| | GGCGACATCGCCTACTGGGACGAGGACGAGCACTTCTTCATCGTGGACCGGCTGA |
| | AGAGCCTGATCAAGTACAAGGGCTACCAGGTGGCCCCCGCCGAGCTGGAGAGCA |
| | TCCTGCTGCAGCACCCCAACATCTTCGACGCCGGCGTGGCCGGCCTGCCCGACGA |
| | CGACGCCGGCGAGCTGCCCGCCGCCGTGGTGGTGCTGGAGCACGGCAAGACCAT |
| | GACCGAGAAGGAGATCGTGGACTACGTGGCCAGCCAGGTGACCACCGCCAAGAA |
| | GCTGCGGGGCGGCGTGGTGTTCGTGGACGAGGTGCCCAAGGGCCTGACCGGCAA |
| | GCTGGACGCCCGGAAGATCCGGGAGATCCTGATCAAGGCCAAGAAGGGCGGCAA |
| | GATCGCCGTGTGA |

In order to confirm in vivo mRNA expression using the composition above, luciferase mRNA (FLuc) of SEQ ID NO: 1 was injected into the gastrocnemius muscle of the mouse hind leg at a concentration of 6 μg/50 μl. Thereafter, mRNA expression was confirmed after 24 hours. 200 μl of luciferin was injected intraperitoneally into each individual at a concentration of 15 mg/ml. 10 minutes after luciferin injection, mRNA expression was confirmed with an exposure time of 15 seconds.

As a result, as shown in Table 5 below, when delivering mRNA using the polymers according to the present invention, it was confirmed that mRNA expression was equivalent or higher than that of free mRNA (in the present specification, free mRNA refers to mRNA without a delivery vehicle), and it was confirmed that among them, 22 polymers showed higher mRNA expression than lipid nanoparticle (LNP) delivery vehicles. In addition, experiments using representative branched polymers also showed a high mRNA expression rate compared to free mRNA. Therefore, it can be seen that the delivery vehicle of the present invention can be used as an mRNA delivery vehicle.

TABLE 5

| Manufacturing | | mRNA expression |
|---|---|---|
| Example | Item No. | efficiency |
| 1 | 1A | 3.61 |
| 3 | 1C | 9.56 |
| 4 | 1D | 6.39 |
| 5 | 1E | 5.39 |
| 6 | 2A | 2.02 |
| 9 | 2D | 8.37 |
| 10 | 2E | 27.79 |
| 11 | 3A | 6.58 |
| 13 | 3C | 17.81 |
| 14 | 3D | 1.07 |
| 15 | 3E | 10.19 |
| 16 | 4A | 1.76 |
| 17 | 4B | 23.72 |
| 18 | 4C | 58.63 |
| 19 | 4D | 38.72 |
| 20 | 4E | 16.00 |
| 21 | 5A | 2.24 |
| 27 | 6B | 41.22 |
| 29 | 6D | 21.58 |
| 30 | 6E | 67.60 |
| 31 | 7A | 36.43 |
| 32 | 7B | 37.83 |
| 33 | 7C | 11.28 |
| 34 | 7D | 62.72 |
| 35 | 7E | 6.29 |
| 36 | 8A | 29.36 |
| 37 | 8B | 78.62 |
| 38 | 8C | 333.17 |
| 39 | 8D | 50.90 |
| 40 | 8E | 32.18 |
| 41 | 9A | 2.12 |
| 42 | 9B | 27.33 |
| 43 | 9C | 92.24 |
| 44 | 9D | 160.44 |
| 45 | 9E | 18.38 |
| 50 | 10E | 2.54 |
| 51 | 11A | 36.90 |
| 53 | 11C | 6.14 |
| 55 | 11E | 2.43 |
| 222 | b3B | 13.47 |
| 223 | b3C | 1.45 |
| 225 | b3E | 1.24 |
| 227 | b4B | 658.79 |
| 228 | b4C | 100.52 |
| 229 | b4D | 115.61 |
| 235 | b7E | 18.36 |
| 238 | b8C | 218.05 |
| 239 | b8D | 24.59 |
| 242 | b9B | 45.64 |
| 243 | b9C | 3.80 |
| 244 | b9D | 105.24 |
| 247 | b10B | 72.51 |
| 248 | b10C | 18.37 |
| 249 | b10D | 19.68 |
| 250 | b10E | 182.42 |
| 252 | b11B | 867.09 |
| 253 | b11C | 67.04 |
| 255 | b12E | 10.02 |
| 256 | b12A | 98.05 |
| 258 | b12C | 80.08 |
| 256 | b12D | 10.26 |
| Free mRNA | comparative group | 1 |
| Lipid nanoparticle | comparative group | 12.27 |

### Test Example 4

Confirmation of In Vivo mRNA Expression of Polymeric Nanoparticles (PNP) Comprising PEG

The mRNA expression efficiency using polymeric nanoparticles (PNP) comprising PEG at both ends was confirmed.

The polymeric nanoparticles prepared according to Example 1 above were injected intramuscularly into animals to confirm the expression rate of mRNA.

Eight polymers (PEG-1A, PEG-1B, PEG-2A, PEG-2B, PEG-3A, PEG-3B, PEG-4A, PEG-4B) prepared in the same manner as in the above manufacturing examples was screened for the in vivo mRNA expression efficiency. The method for forming the polymer-mRNA complex used for in vivo injection to confirm the mRNA expression is as follows. The weight ratio of each polymer was adjusted, and 6 μl of the solution dissolved in a pH 5.0, 25 mM NaOAc buffer was mixed with 6 μl of luciferase mRNA (FLuc) of SEQ ID NO: 1 at a concentration of 1 mg/ml, and then gently vortexed. Thereafter, the mixture was stabilized at 4° C. for 30 minutes, and 38 μl of PBS was added to make a final volume of 50 μl.

In order to confirm in vivo mRNA expression using the composition above, it was injected into the gastrocnemius muscle of the mouse hind leg. Thereafter, mRNA expression was confirmed 24 hours after intramuscular injection. 200 μl of luciferin was injected intraperitoneally into each individual at a concentration of 15 mg/ml. 10 minutes after luciferin injection, mRNA expression was confirmed with an exposure time of 15 seconds.

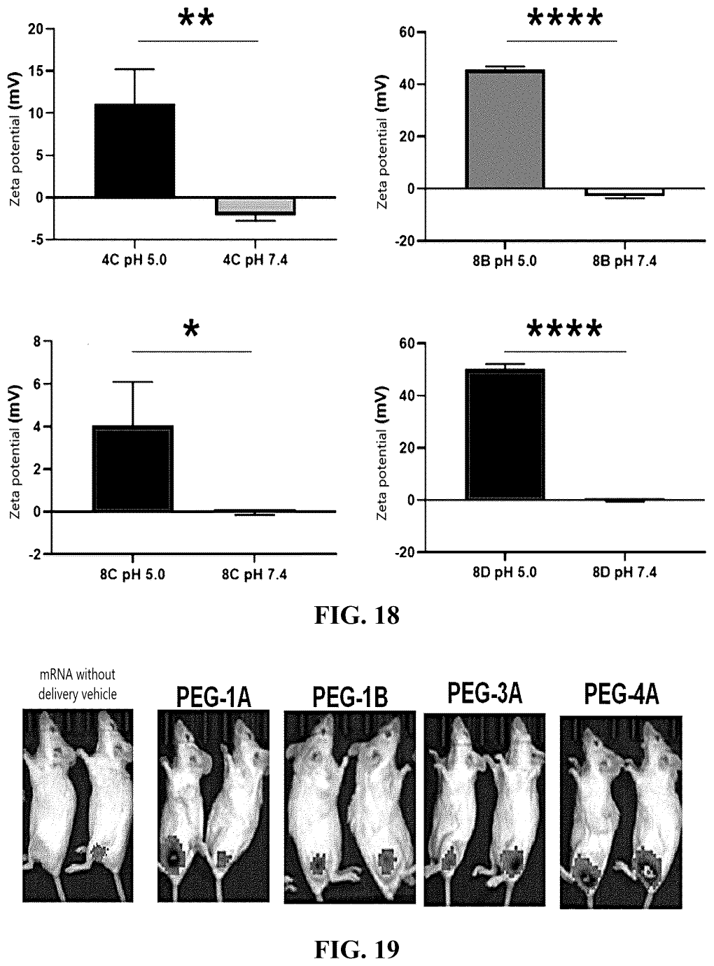

As a result, as shown in FIG. 19, PEG was included at both ends of the polymers prepared in Manufacturing Examples 111 to 220 above, and at this time, the mRNA expression of representative polymers was confirmed. As a result, it was confirmed that the expression rate was equal to or higher than that of free mRNA. In addition, it was confirmed that 1B prepared by Manufacturing Example 2 had poor solubility, but PEG-1B, which had PEG attached to both ends, had improved solubility and mRNA expression was significantly increased. Therefore, it can be seen that the delivery vehicle of the present invention can be effectively used as an mRNA delivery vehicle.

### Test Example 5

### Confirmation of In Vivo Expression Retention Time of Polymeric Nanoparticles (PNP) Comprising PEG

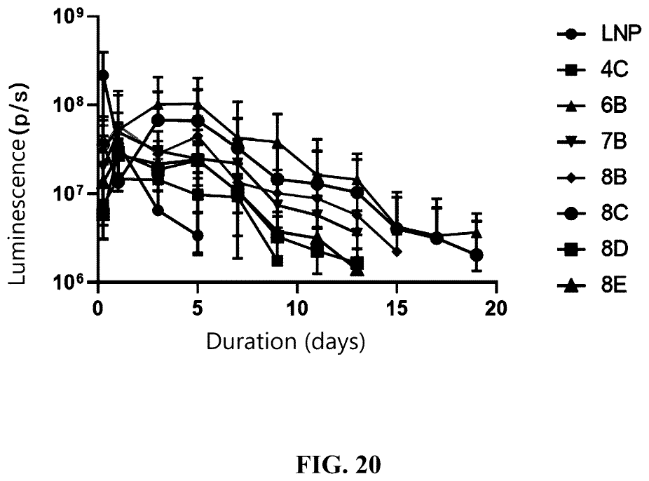

In order to confirm the expression retention time of mRNA, polymer-mRNA complexes were prepared using representative polymers of the manufacturing examples in the same manner as in Test Example 3, and then injected into the hind legs of mice, and measured for up to 20 days. The results are shown in FIG. 20.

As shown in FIG. 20, it was confirmed that in the case of lipid nanoparticle-mRNA complex (LNP-mRNA), expression reached its peak at 4 hours and then dropped below the baseline value at about 48 hours, whereas in the case of all polymeric nanoparticle-mRNA complexes used in the test, the expression time was maintained longer than that of lipid nanoparticle-mRNA complexes. In particular, it was confirmed that in the case of 6B and 8C polymer complexes, mRNA expression was maintained for more than 10 days, which means that the administration frequency can be drastically reduced compared to the existing method in the development of a gene therapeutic agent.

1. A polymer suitable for intracellular nucleic acid delivery, comprising a repeating unit of Formula 1 below: 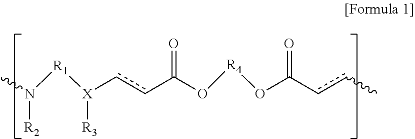
in the formula,
R1 is a bond or a C1-6 alkylene,
R2 and R3 are each independently (i) hydrogen, (ii) C1-6 alkyl which may be substituted with alkylamine or hydroxy, (iii) a 5- or 6-membered carbocycle or heterocycle, or (iv) C1-6 alkyl substituted with a 5- or 6-membered carbocycle or heterocycle,
said R2 and R3 may be linked together to form a C1-6 alkylene,
said 5- or 6-membered carbocycle or heterocycle may be substituted with C1-3 alkyl or C1-3 alkoxyacyl,
X is a nitrogen atom or a bond, and when X is a bond, said R3 is absent,
R4 is a linear or branched C1-15 alkylene, 1 to 5 carbon atoms of said alkylene may be replaced by oxygen or sulfur, and [symbol]
represents a single bond or a double bond.

2. A polymer suitable for intracellular nucleic acid delivery, comprising a repeating unit of Formula 2 below: 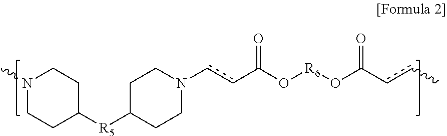
in the formula,
R5 is a C1-6 alkylene,
R6 is a linear or branched C1-15 alkylene, 1 to 5 carbon atoms of said alkylene may be replaced by oxygen or sulfur, and [symbol]
represents a single bond or a double bond.

3. The polymer according to claim 1, wherein the repeating unit of Formula 1 is formed by a reaction between any one of the monomers of Formulas 3 to 13 below and any one of the monomers of Formulas 14 to 23 below:

| Formula | Structure |
|---|---|
| 3 | 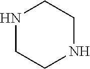 |
| 4 | 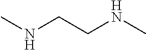 |
| 5 | 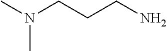 |
| 6 | 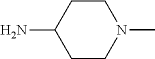 |
| 7 | 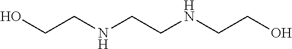 |
| 8 | 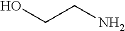 |
| 9 | 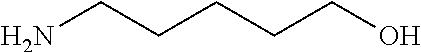 |
| 10 | 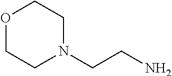 |
| 11 | 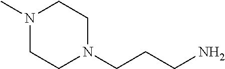 |
| 12 | 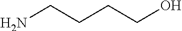 |
| 13 | 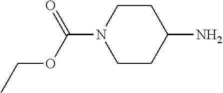 |
| 14 | 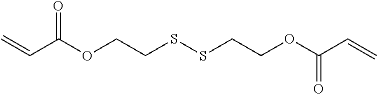 [SMILES: C=CC(=O)OCCSSCCOC(=O)C=C] |
| 15 | 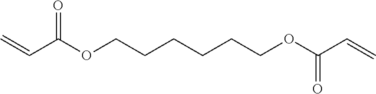 [SMILES: C=CC(=O)OCCCCCCOC(=O)C=C] |
| 16 | 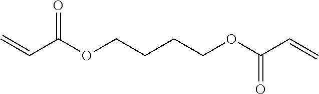 |
| 17 | 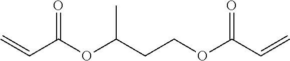 |
| 18 | 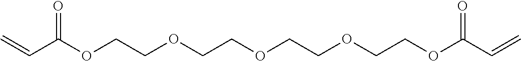 [SMILES: C=CC(=O)OCCOCCOCCOCCOC(=O)C=C] |
| 19 | 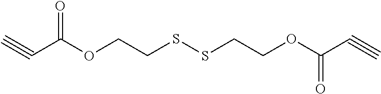 [SMILES: C#CC(=O)OCCSSCCOC(=O)C#C] |
| 20 | 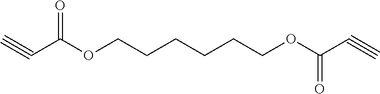 [SMILES: C#CC(=O)OCCCCCCOC(=O)C#C] |
| 21 | 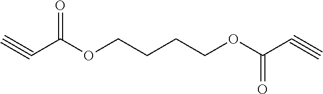 |
| 22 | 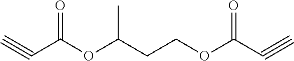 |
| 23 | 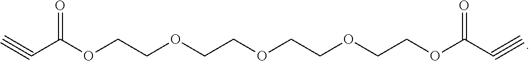 [SMILES: C#CC(=O)OCCOCCOCCOCCOC(=O)C#C] |

4. The polymer according to claim 2, wherein the repeating unit of Formula 2 is formed by a reaction between a monomer of Formula 24 below and any one of the monomers of Formulas 14 to 23 below:

| Formula | Structure |
|---|---|
| 14 | 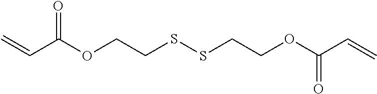 [SMILES: C=CC(=O)OCCSSCCOC(=O)C=C] |
| 15 | 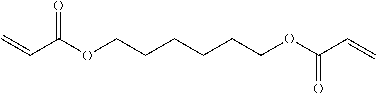 [SMILES: C=CC(=O)OCCCCCCOC(=O)C=C] |
| 16 | 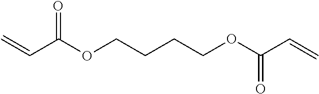 |
| 17 | 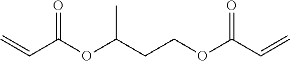 |
| 18 | 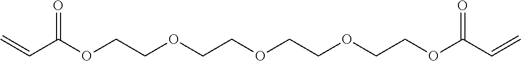 [SMILES: C=CC(=O)OCCOCCOCCOCCOC(=O)C=C] |
| 19 | 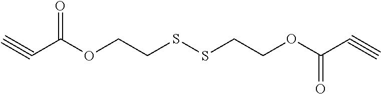 [SMILES: C#CC(=O)OCCSSCCOC(=O)C#C] |
| 20 | 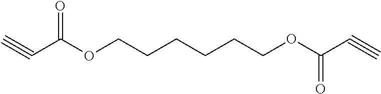 [SMILES: C#CC(=O)OCCCCCCOC(=O)C#C] |
| 21 | 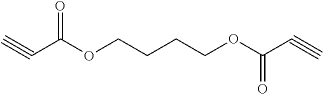 |
| 22 | 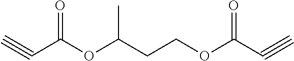 |
| 23 | 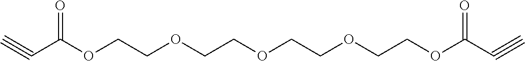 [SMILES: C#CC(=O)OCCOCCOCCOCCOC(=O)C#C] |
| 24 | 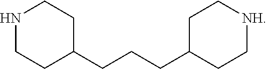 [SMILES: C(CC1CCNCC1)CC1CCNCC1] |

5. A polymer comprising a repeating unit selected from the group consisting of repeating units of the chemical structures set forth in the table below:
Chemical structural formula

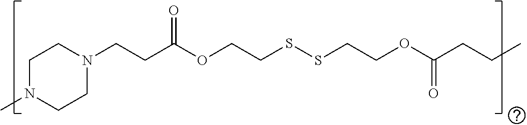

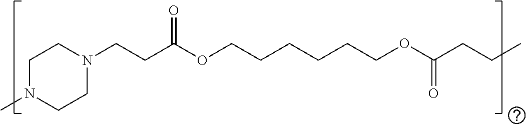

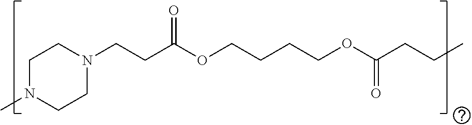

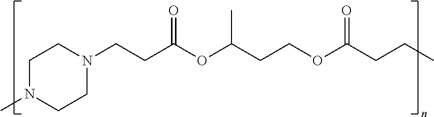

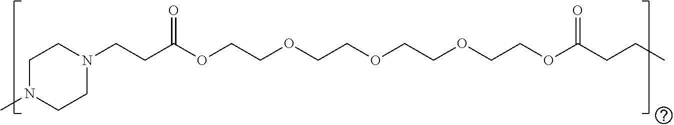

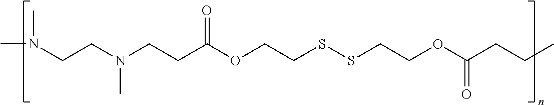

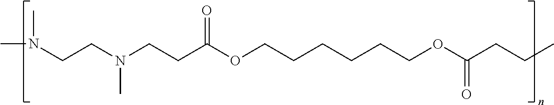

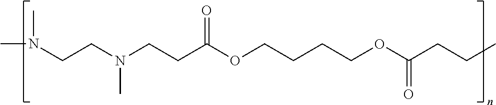

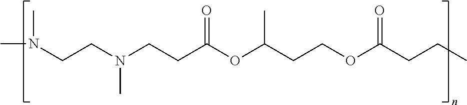

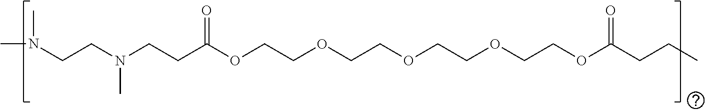

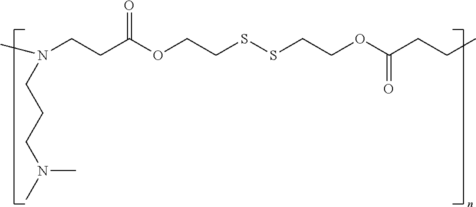

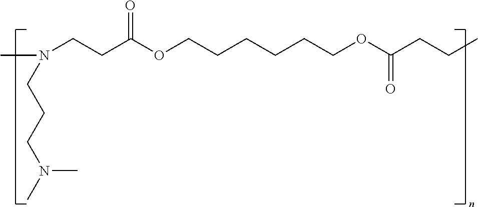

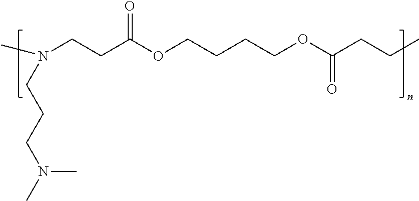

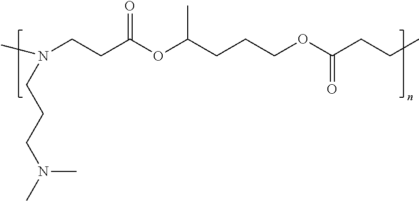

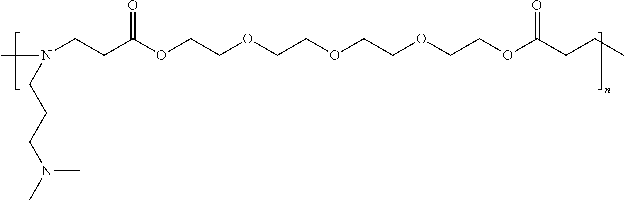

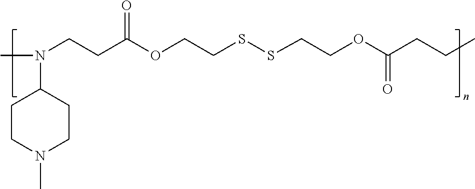

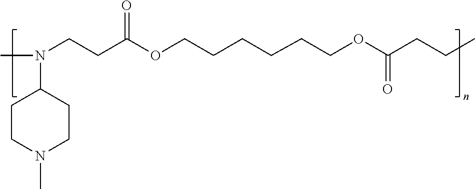

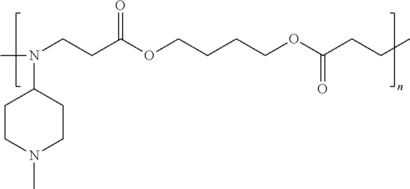

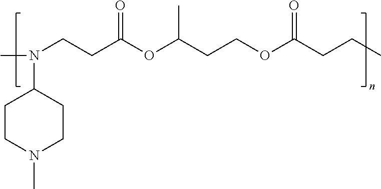

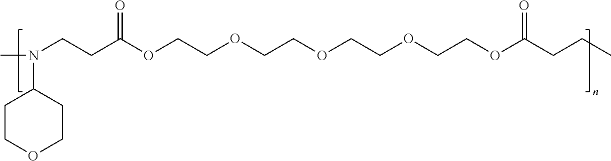

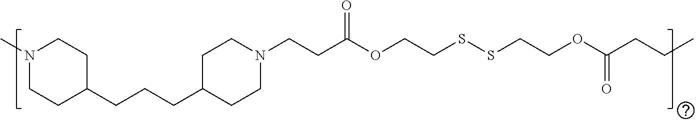

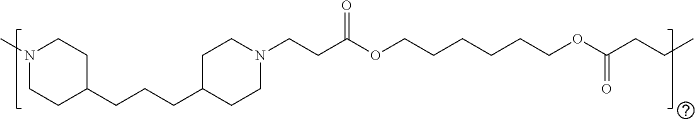

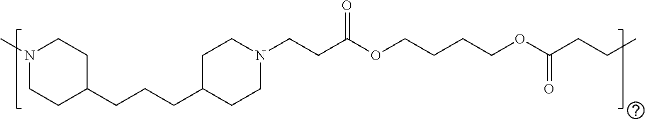

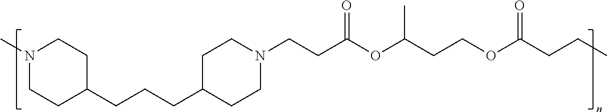

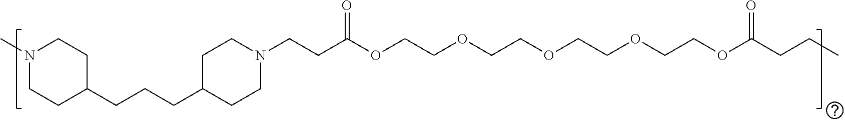

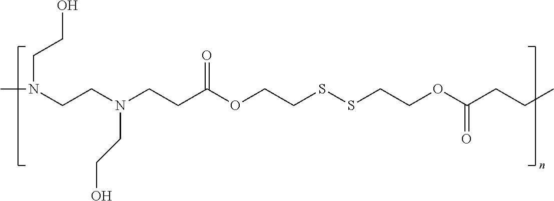

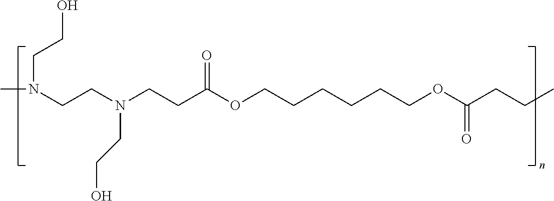

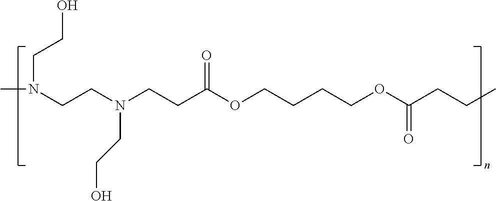

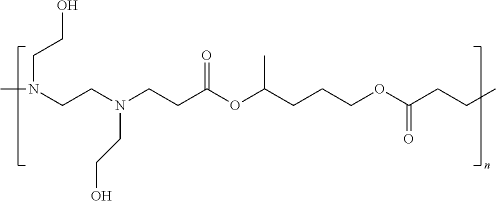

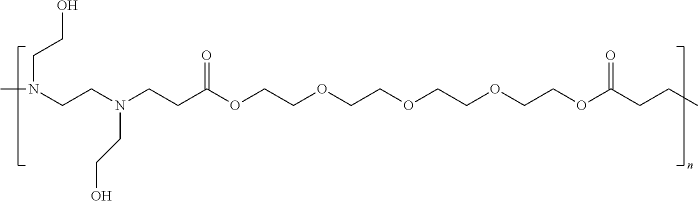

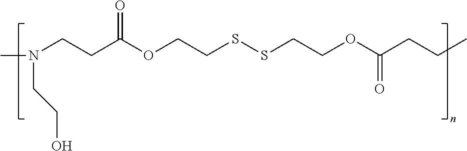

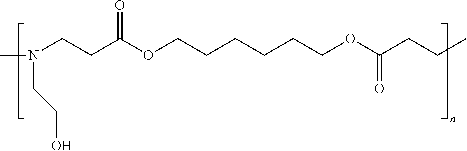

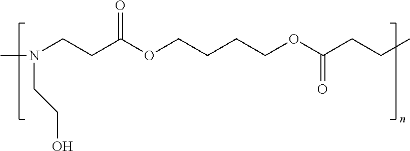

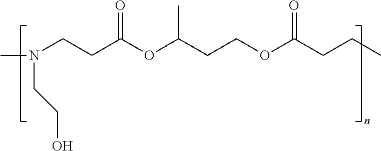

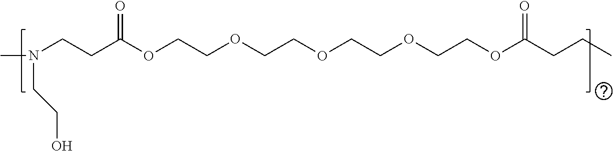

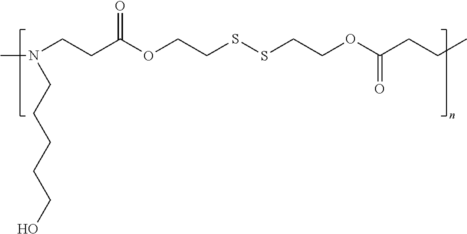

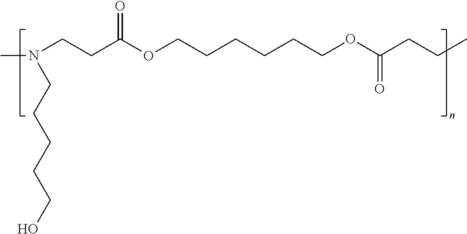

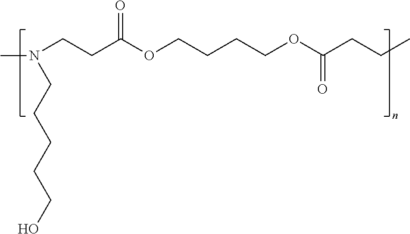

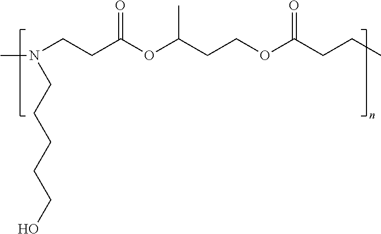

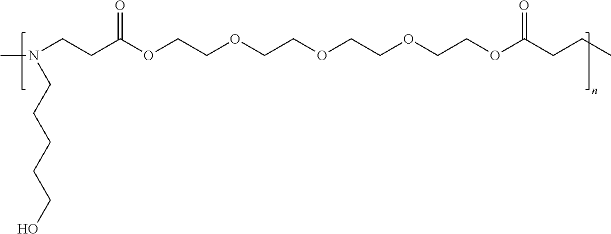

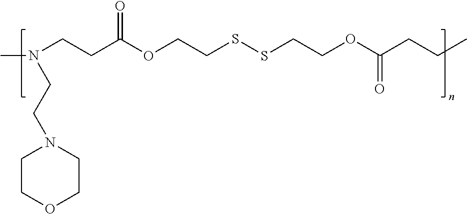

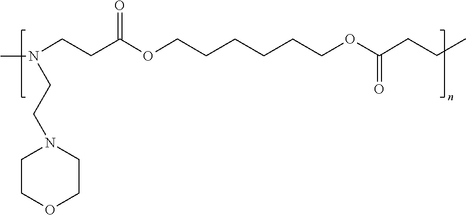

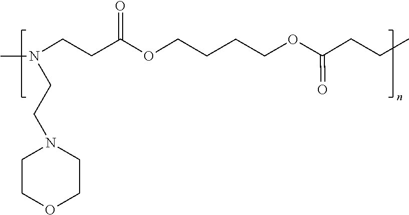

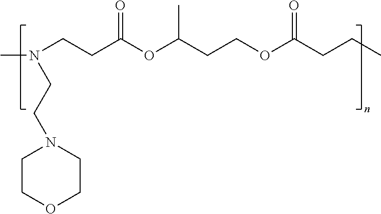

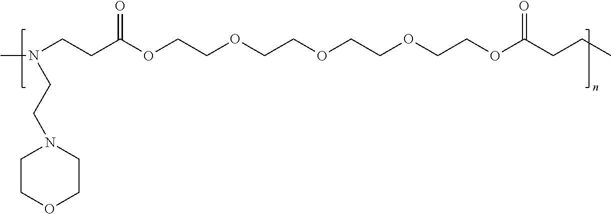

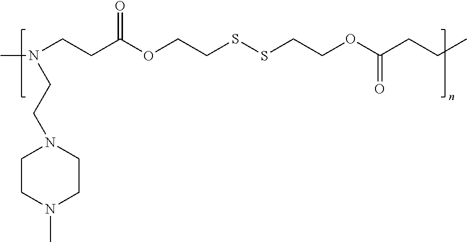

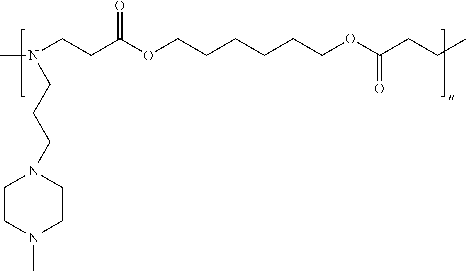

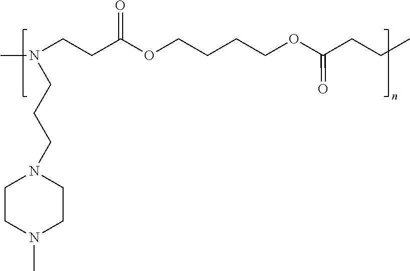

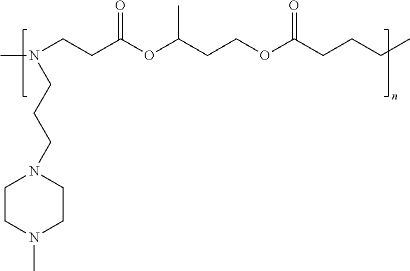

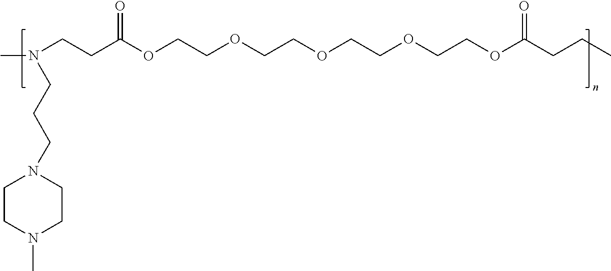

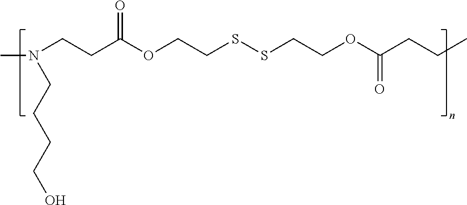

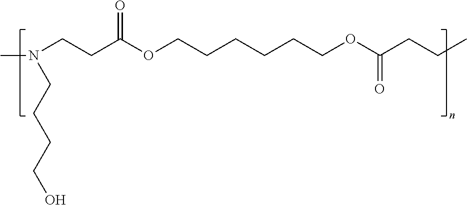

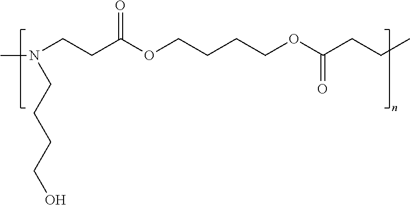

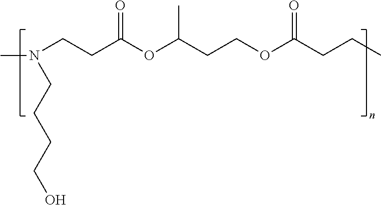

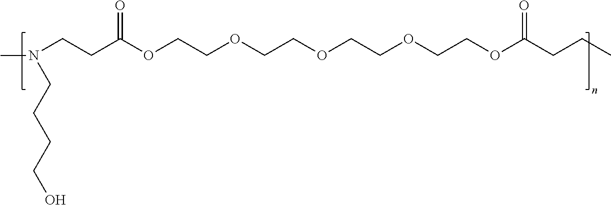

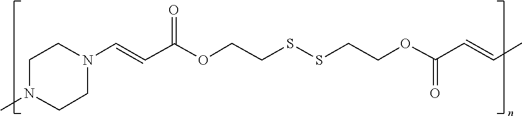

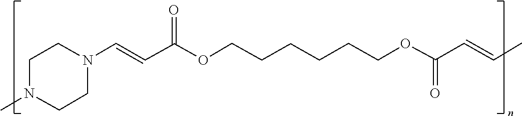

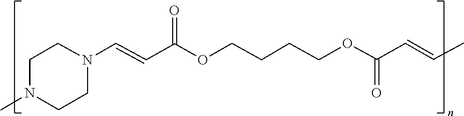

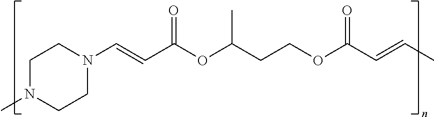

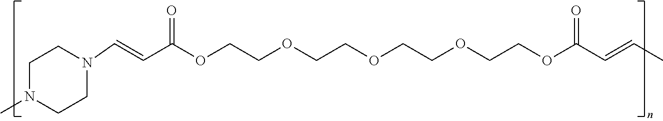

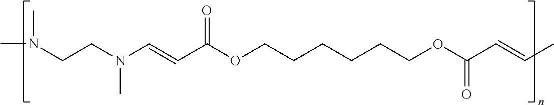

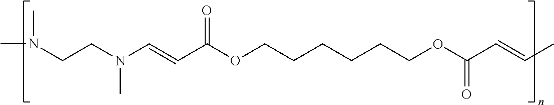

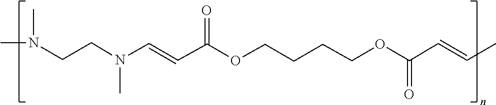

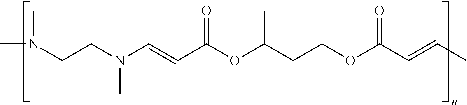

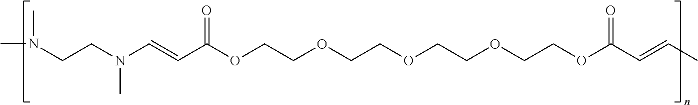

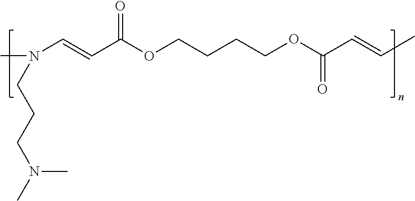

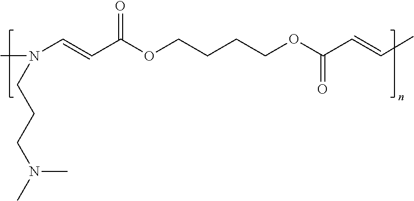

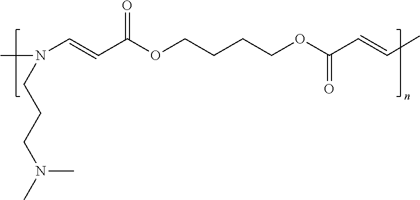

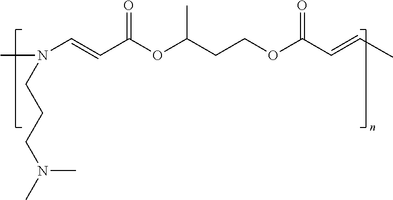

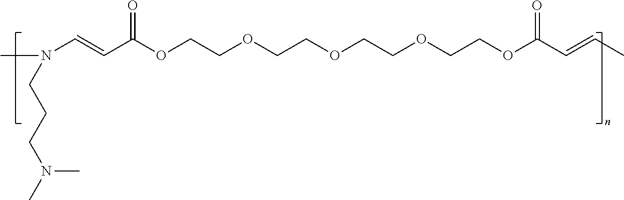

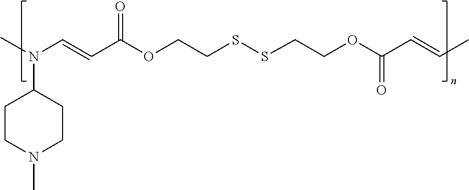

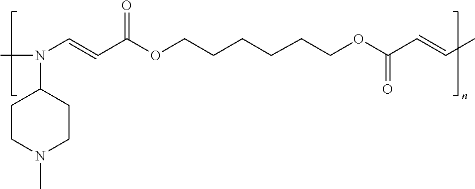

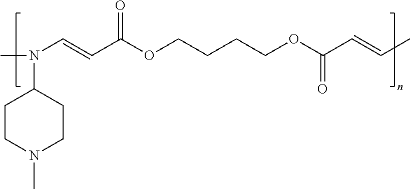

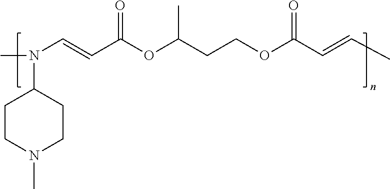

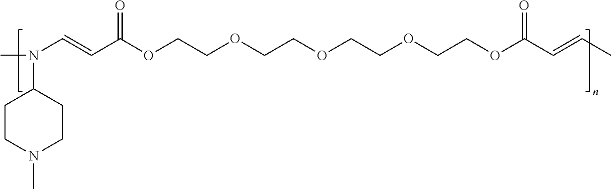

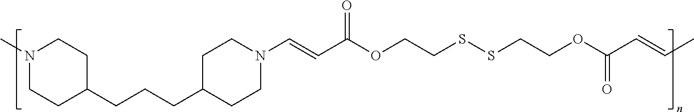

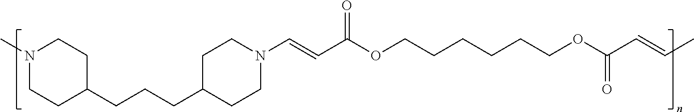

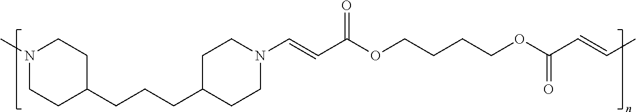

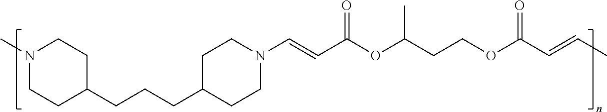

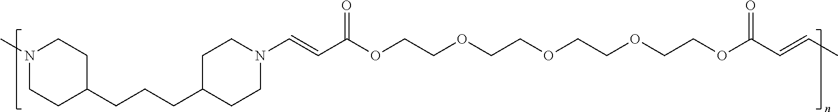

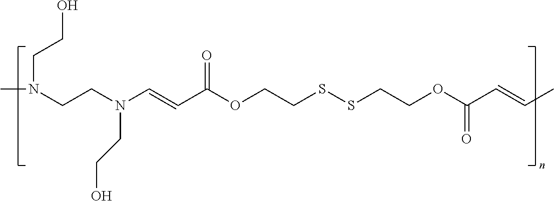

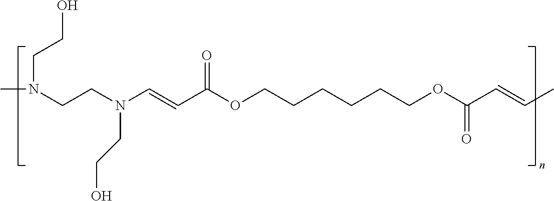

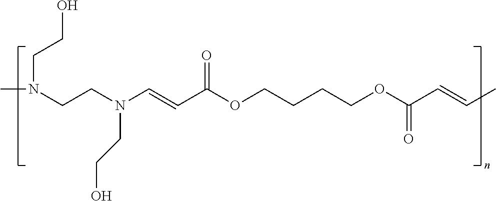

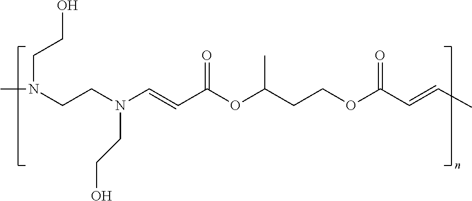

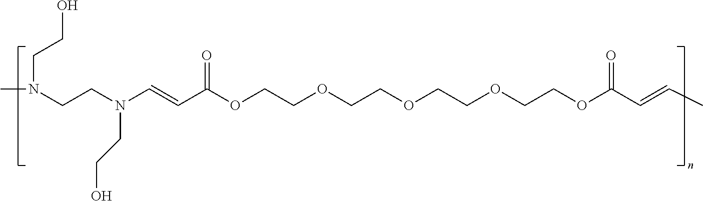

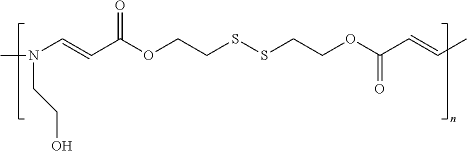

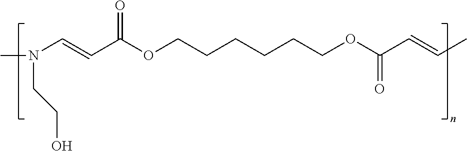

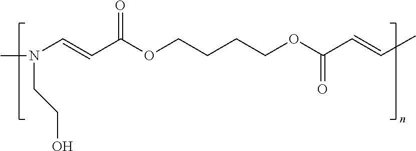

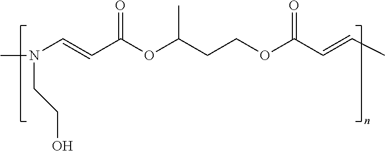

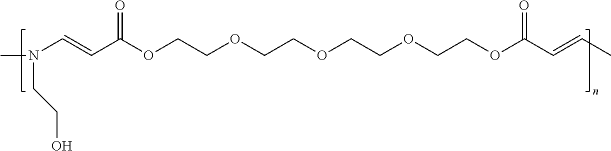

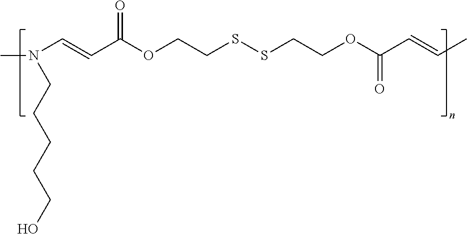

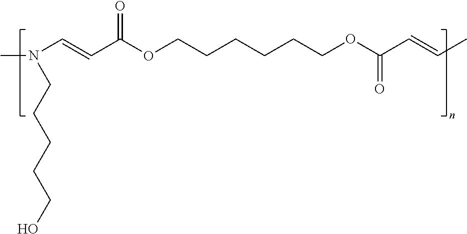

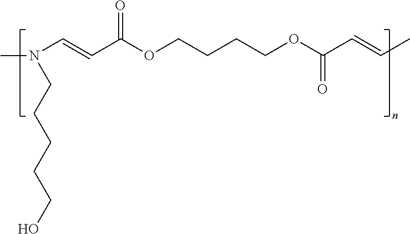

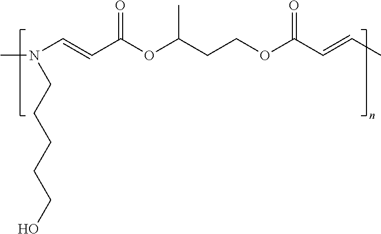

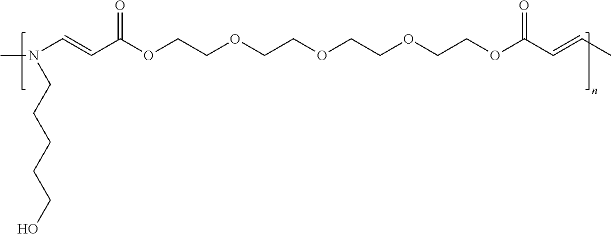

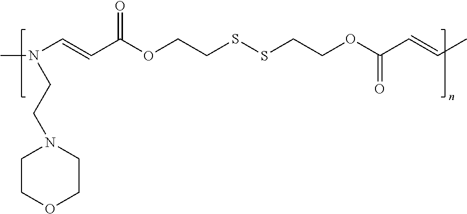

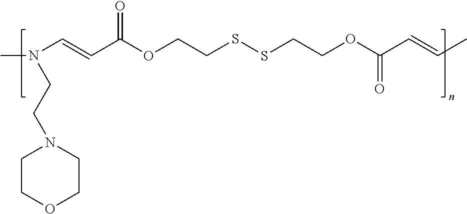

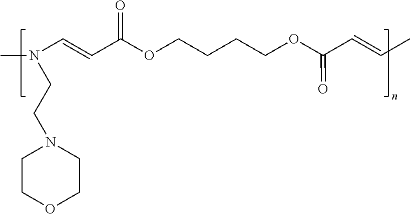

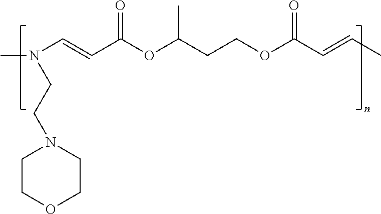

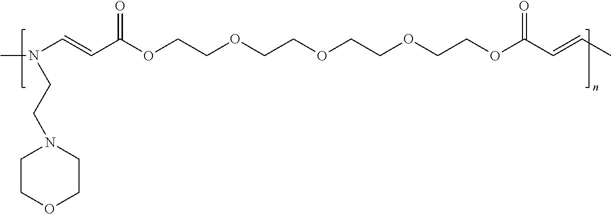

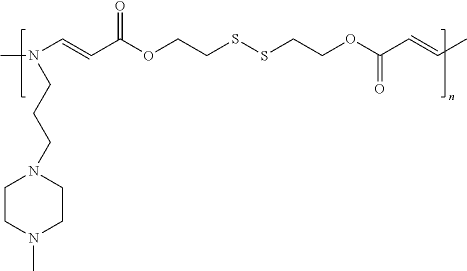

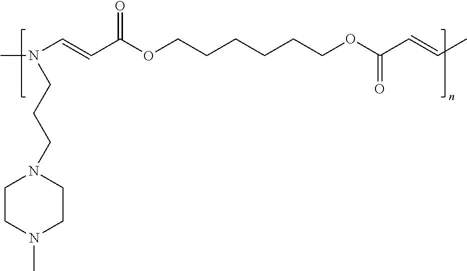

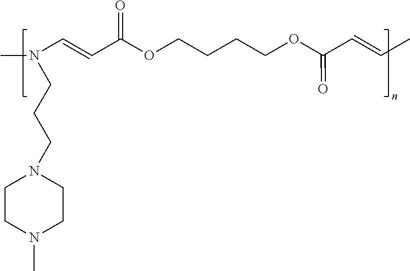

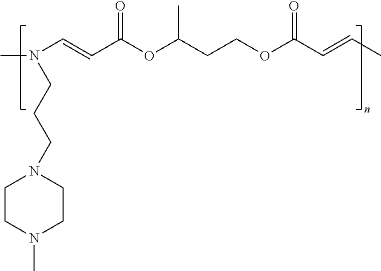

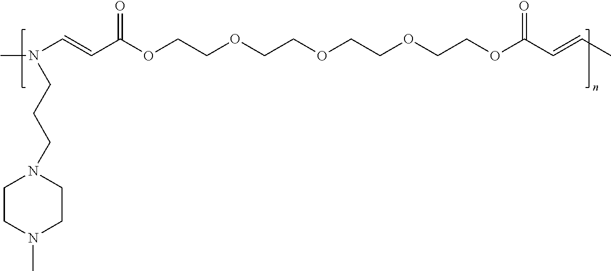

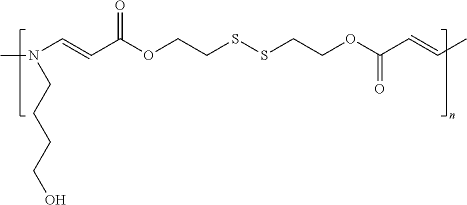

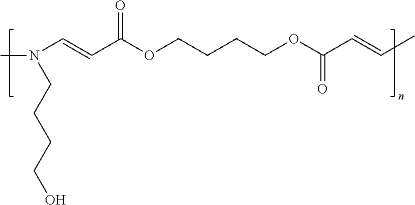

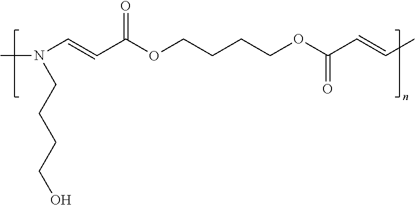

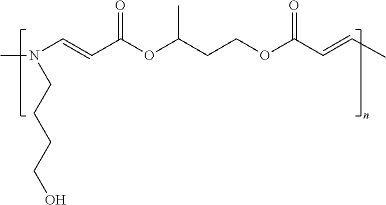

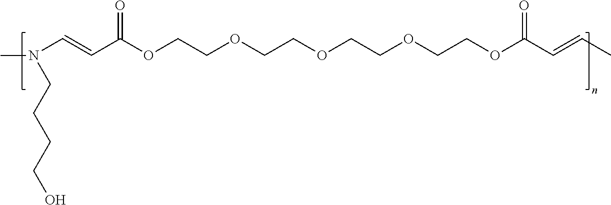

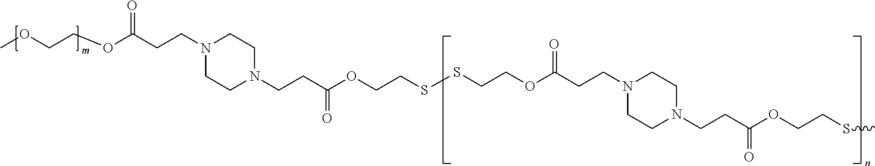

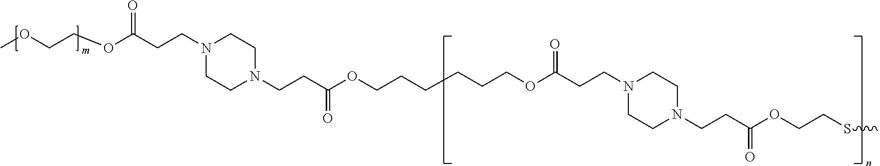

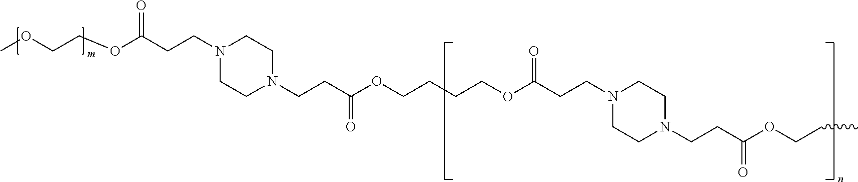

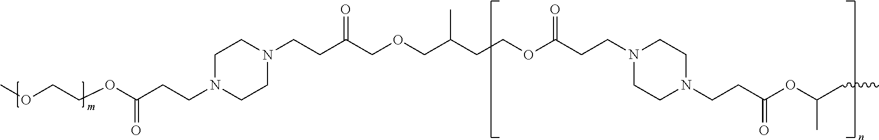

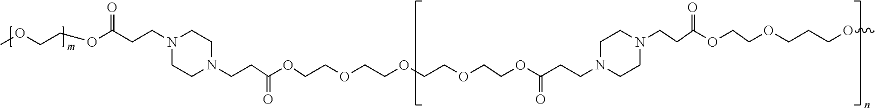

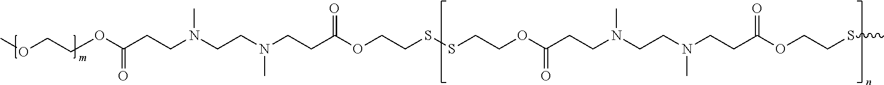

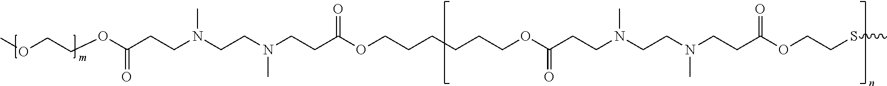

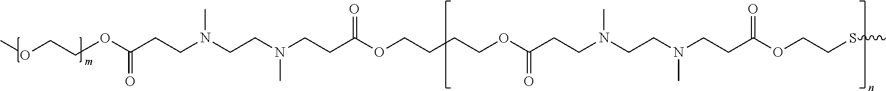

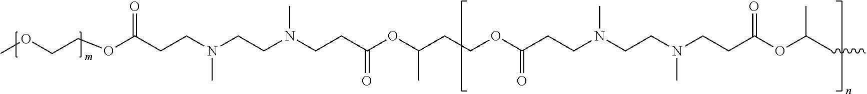

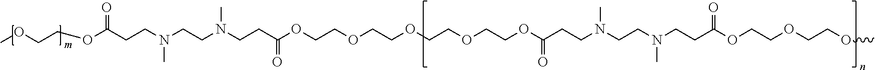

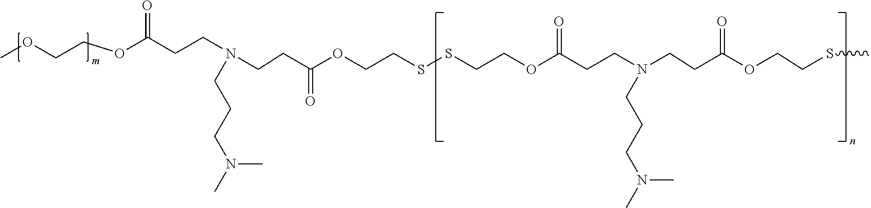

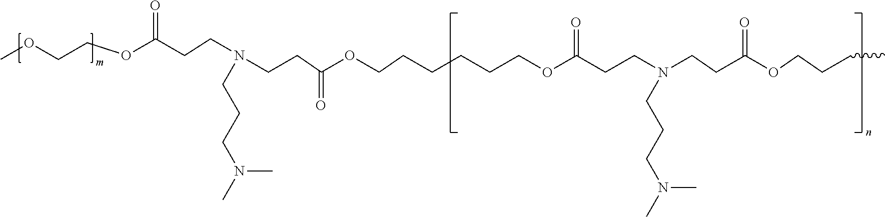

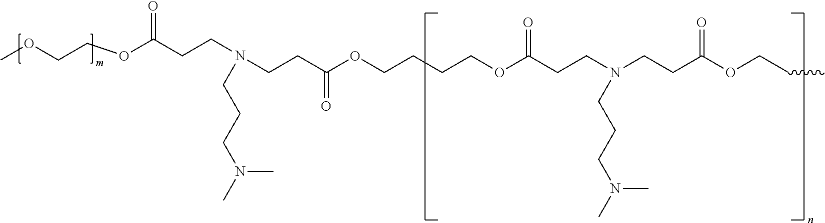

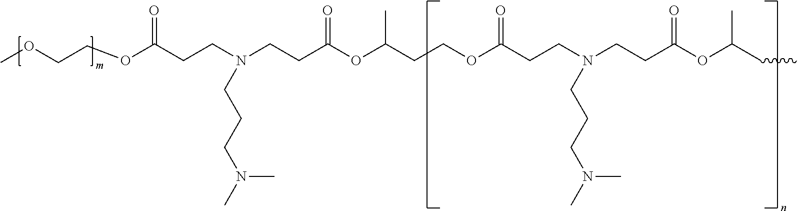

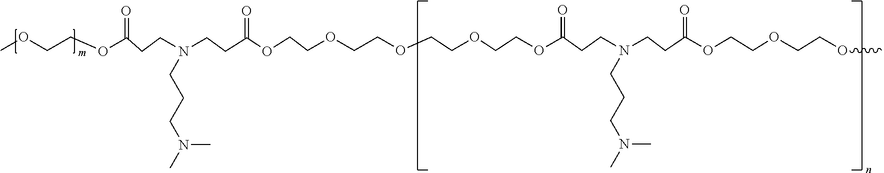

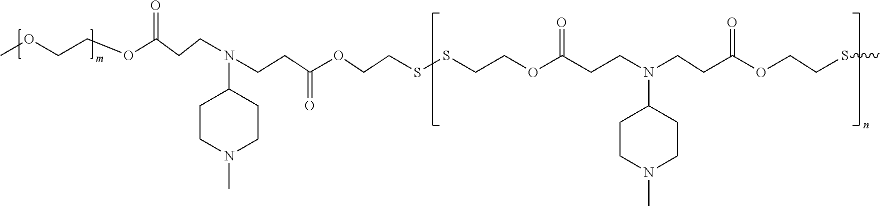

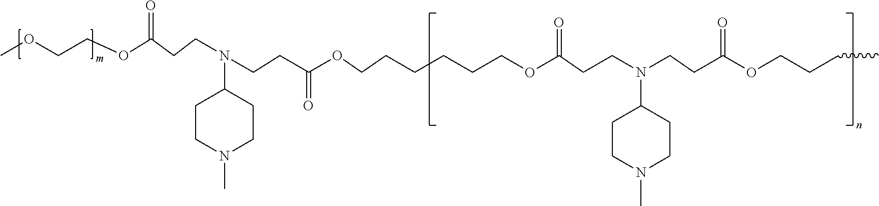

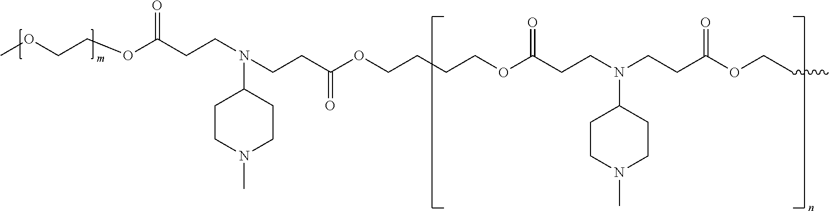

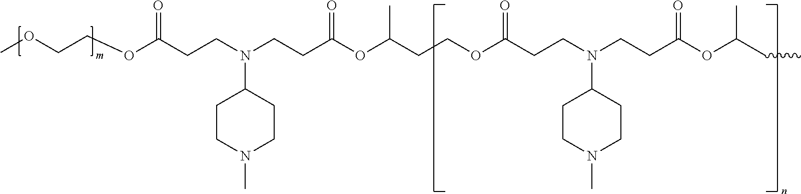

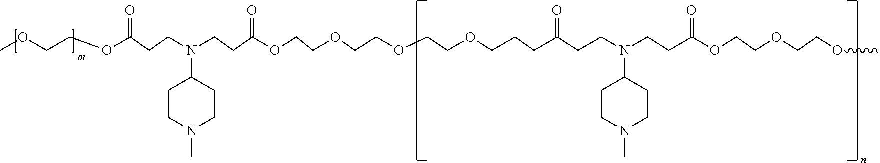

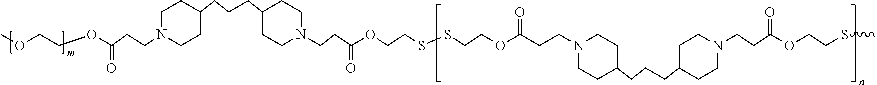

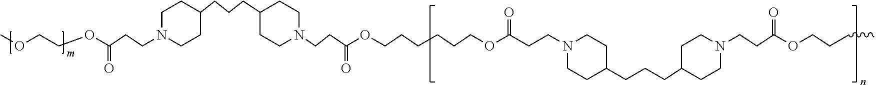

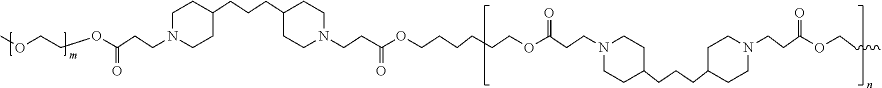

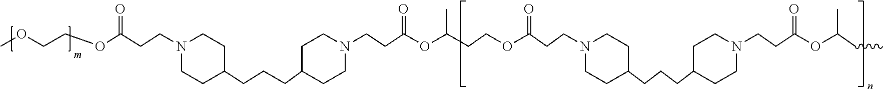

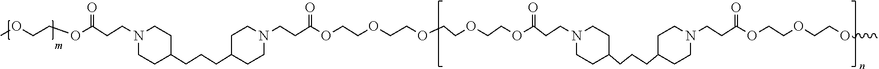

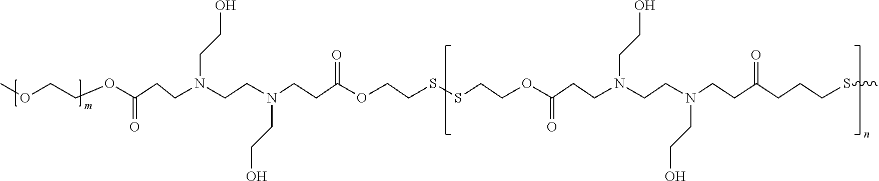

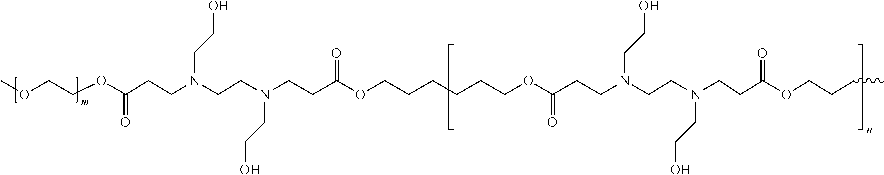

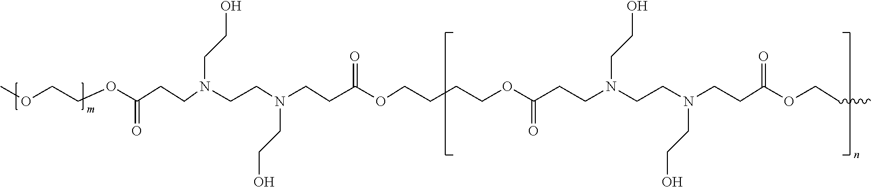

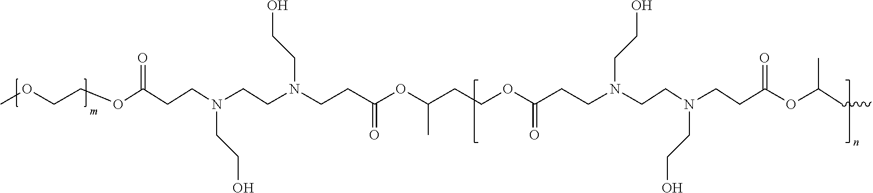

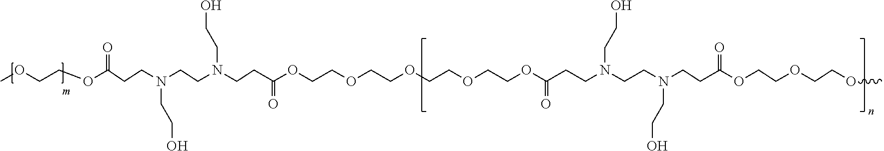

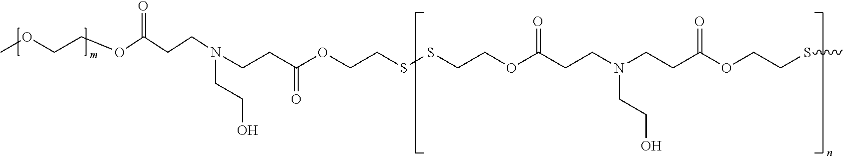

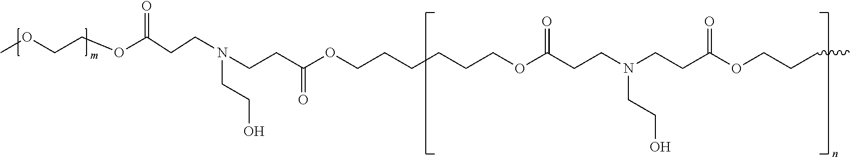

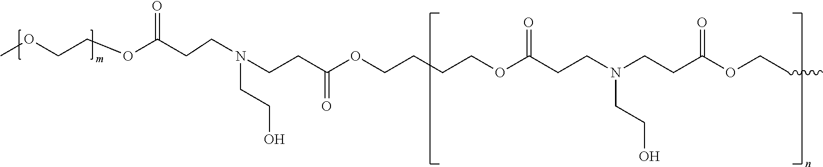

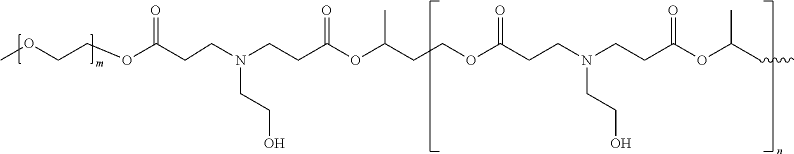

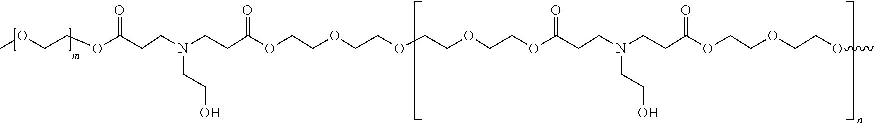

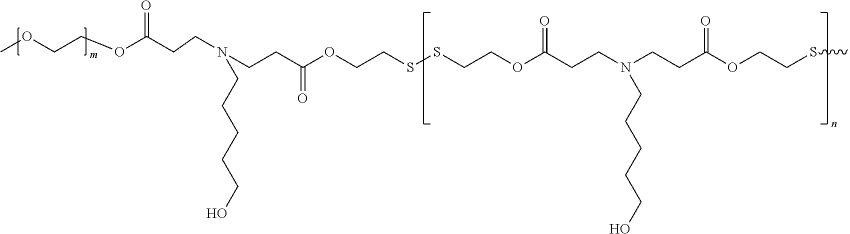

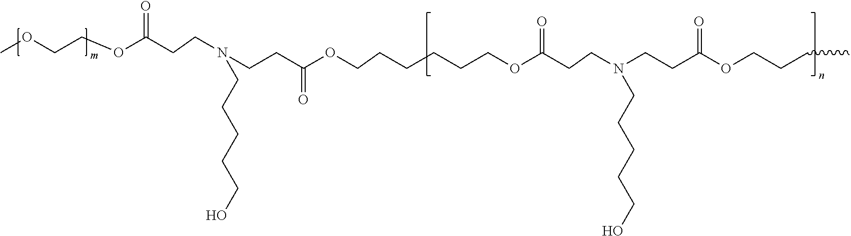

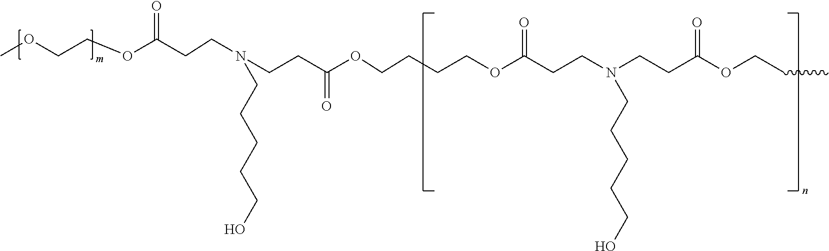

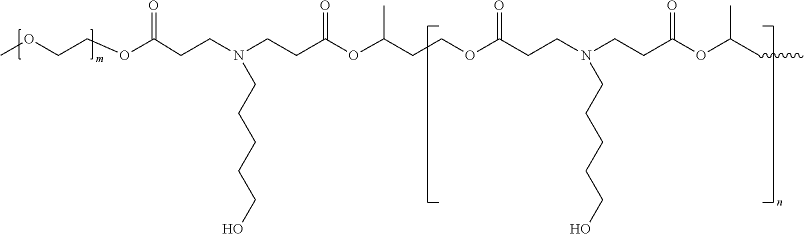

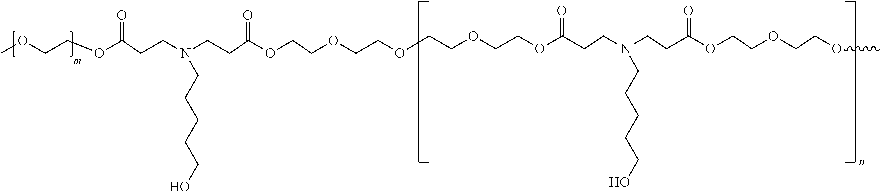

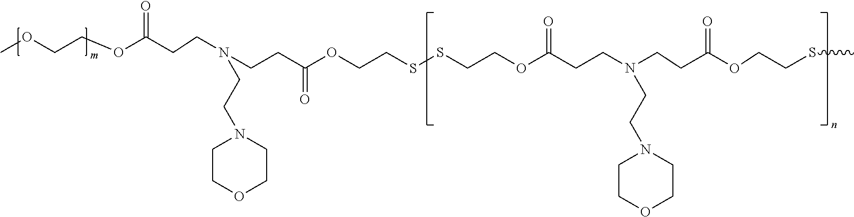

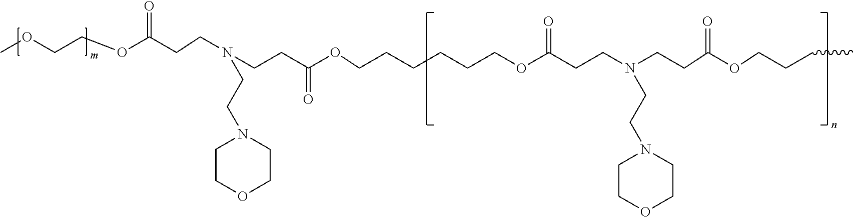

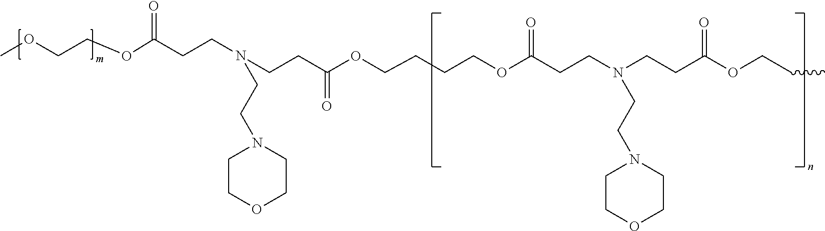

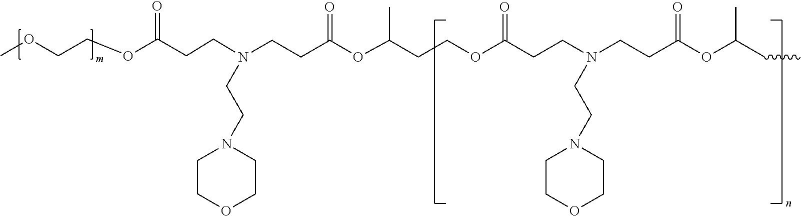

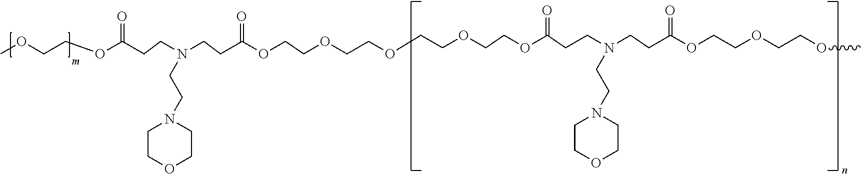

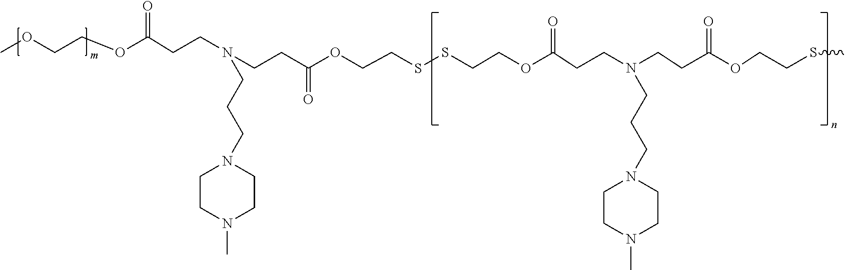

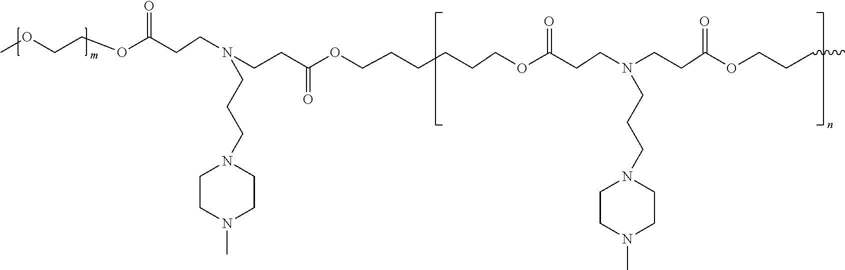

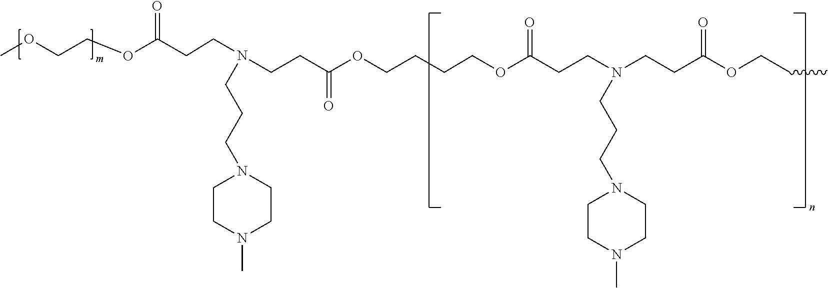

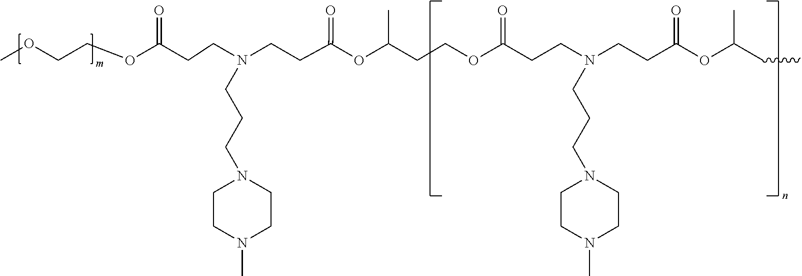

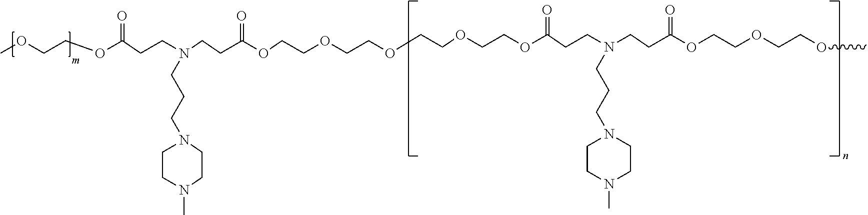

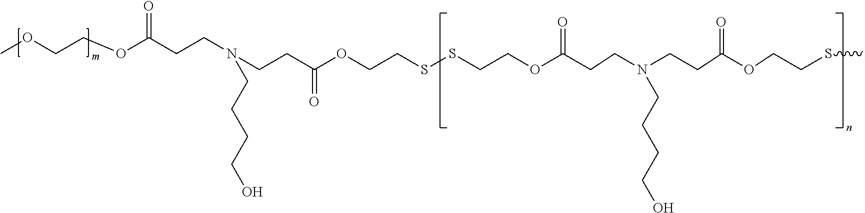

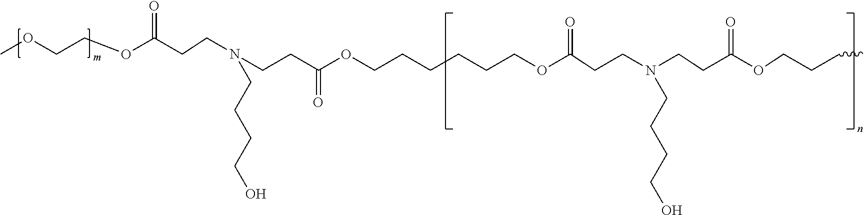

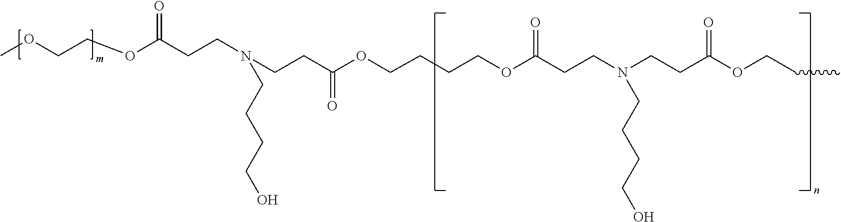

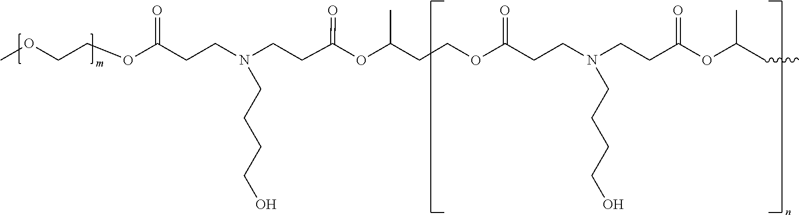

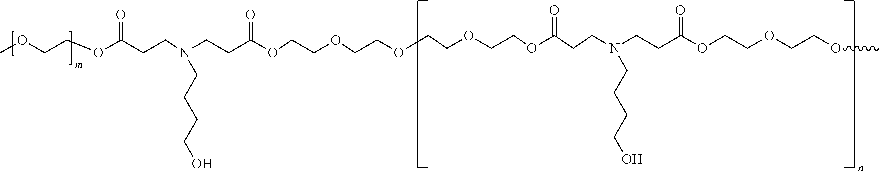

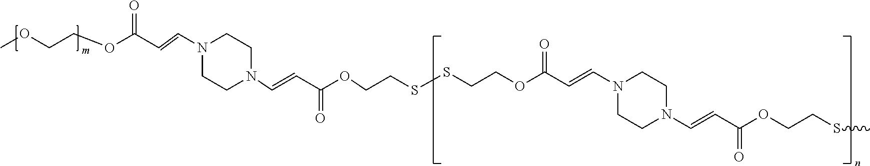

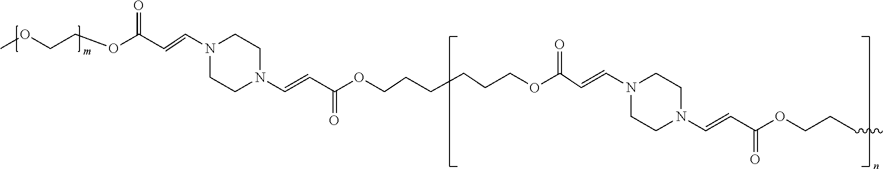

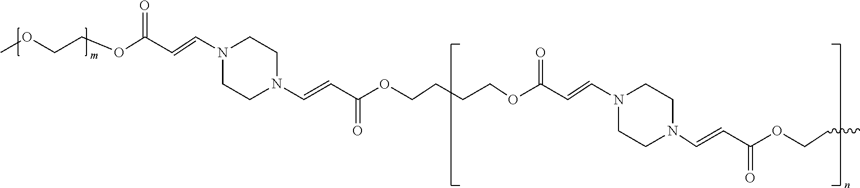

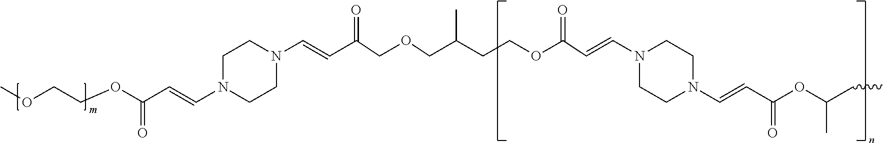

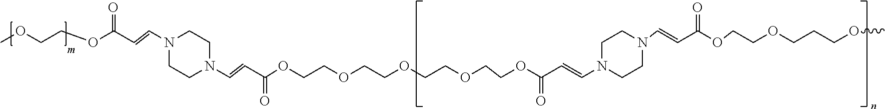

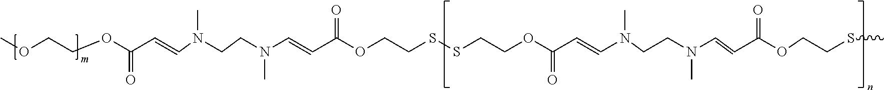

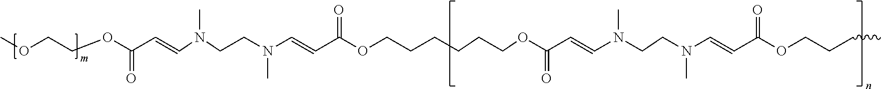

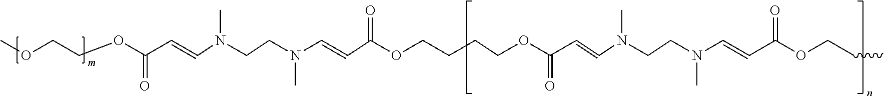

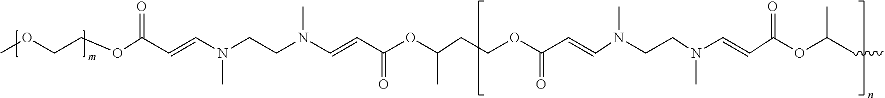

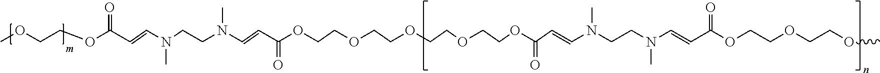

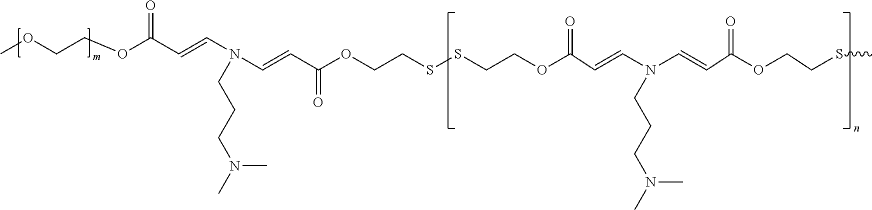

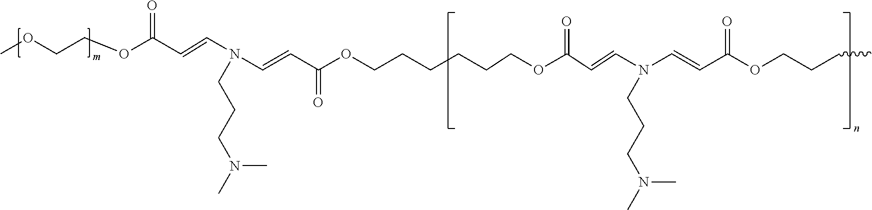

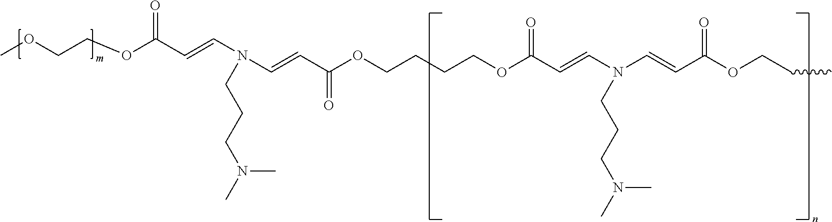

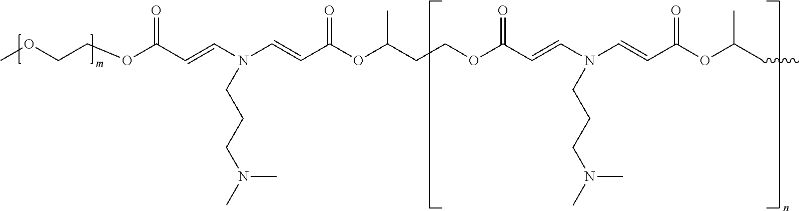

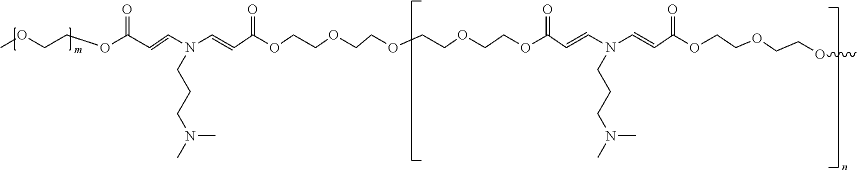

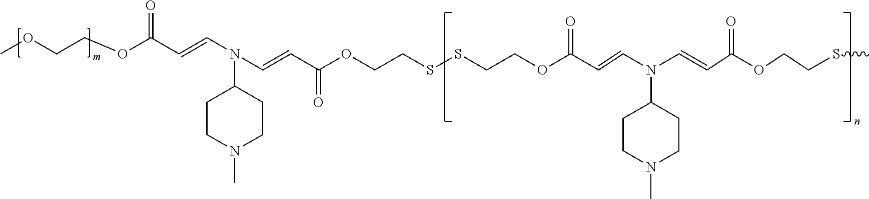

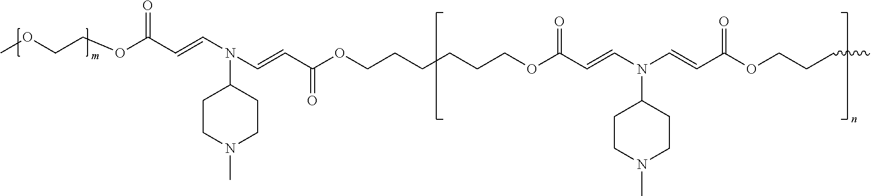

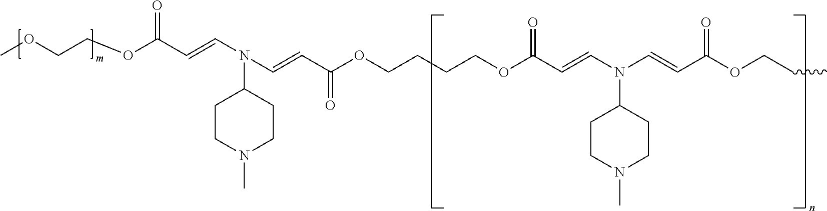

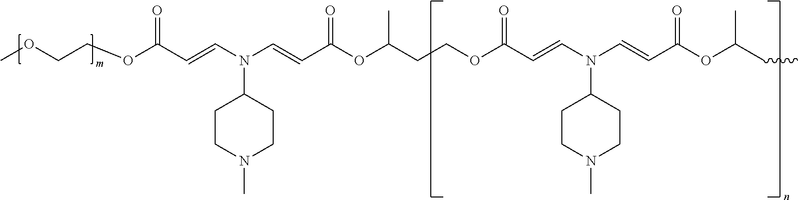

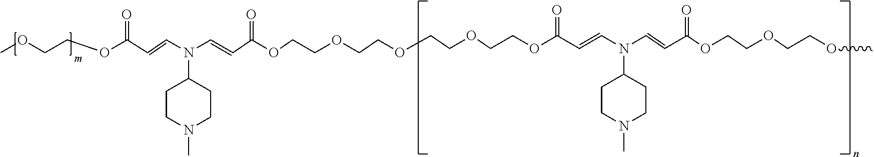

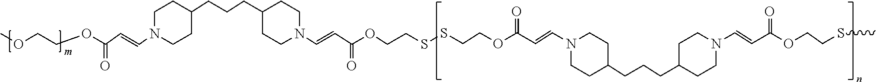

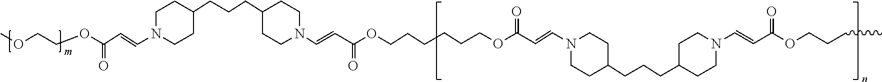

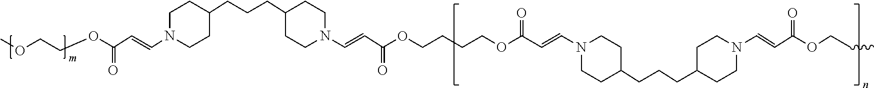

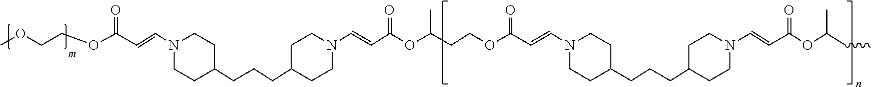

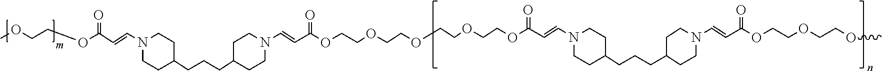

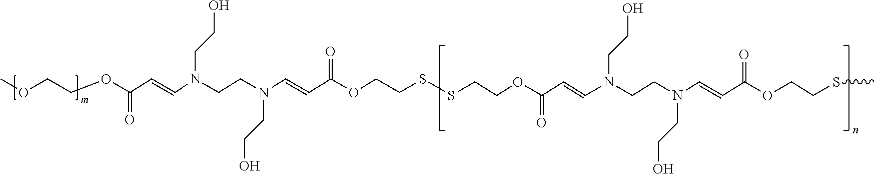

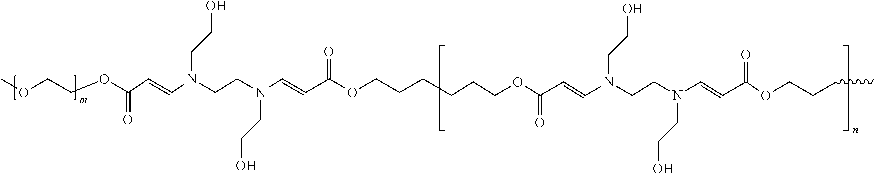

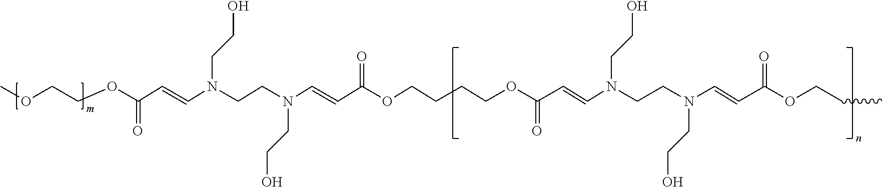

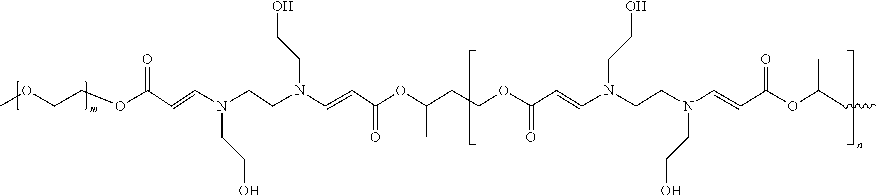

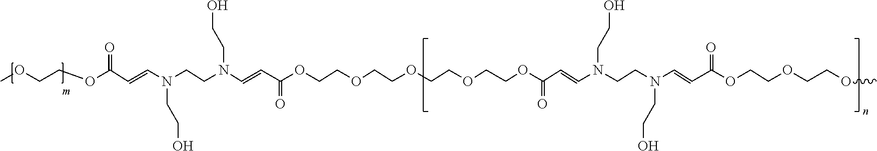

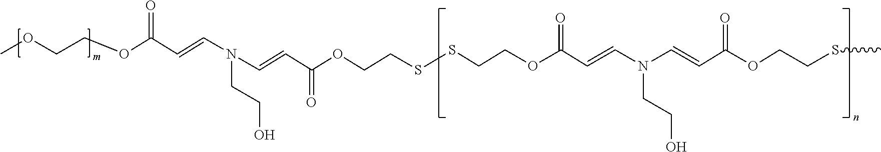

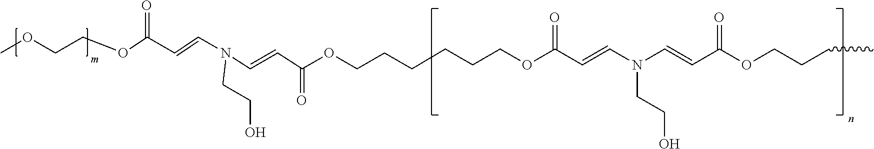

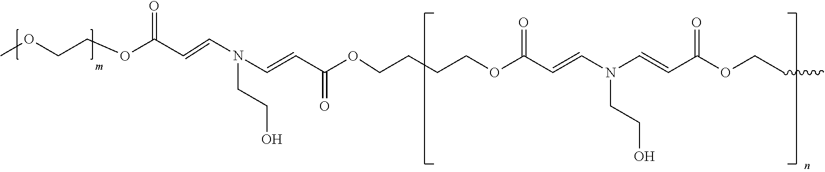

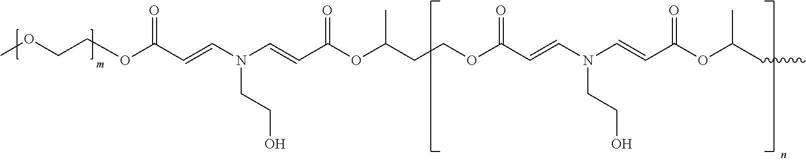

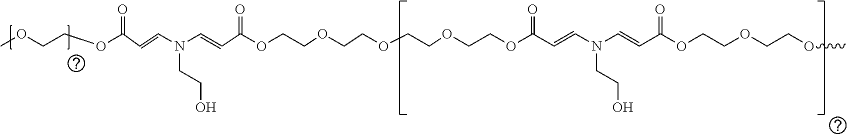

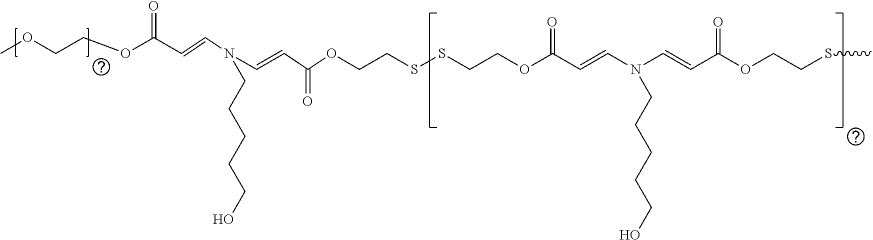

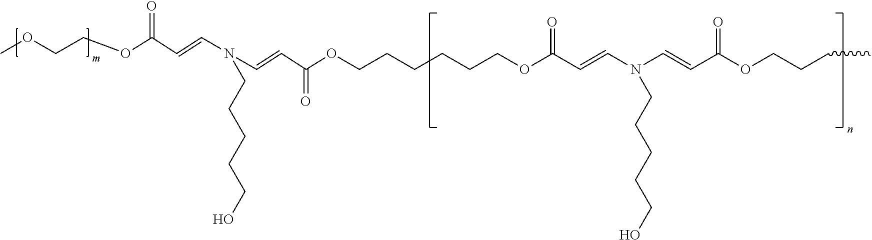

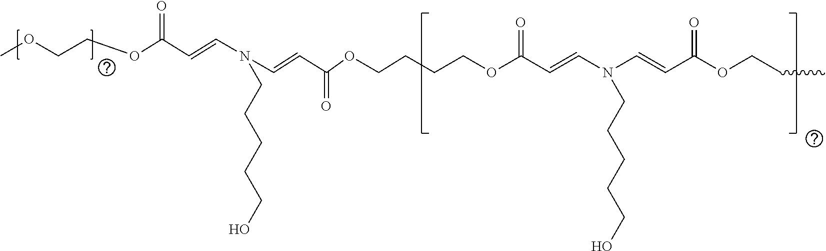

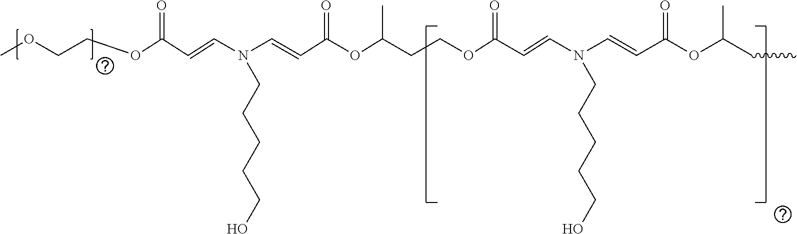

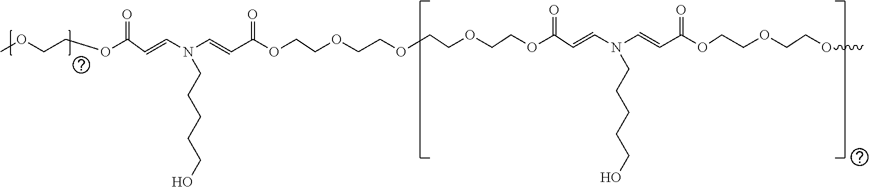

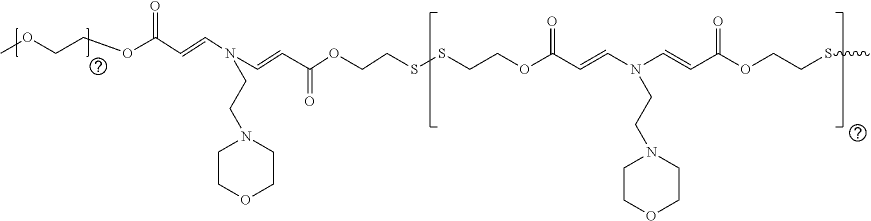

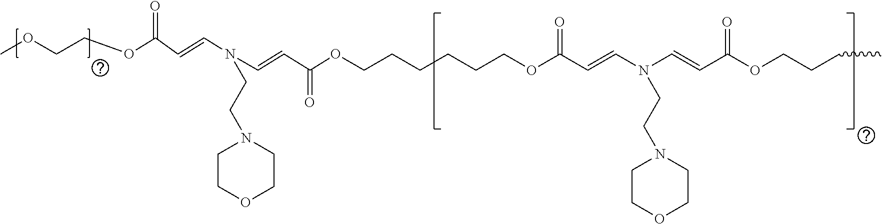

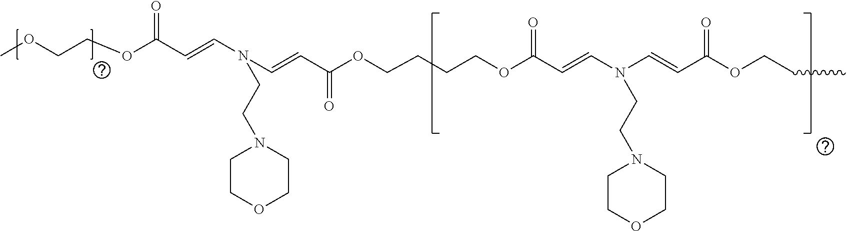

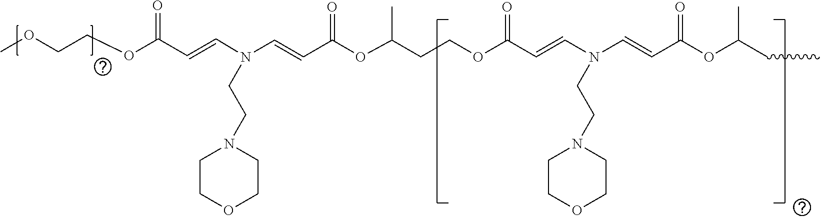

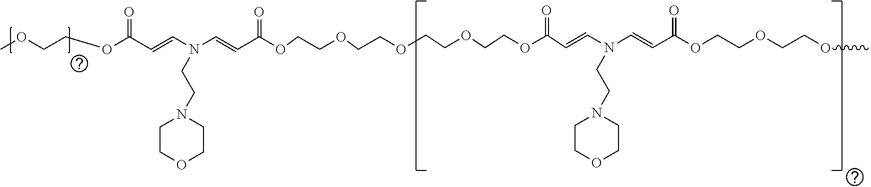

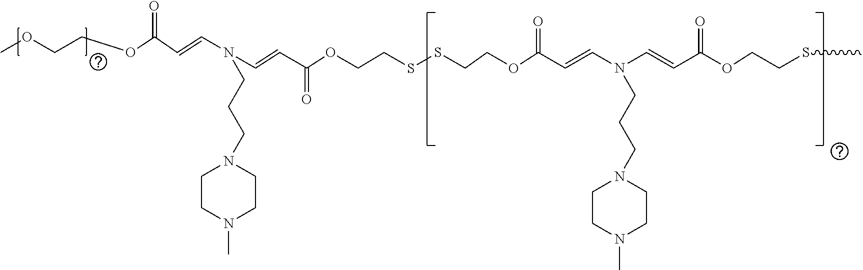

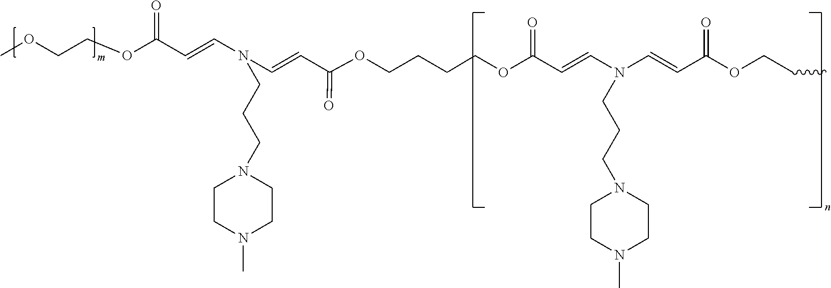

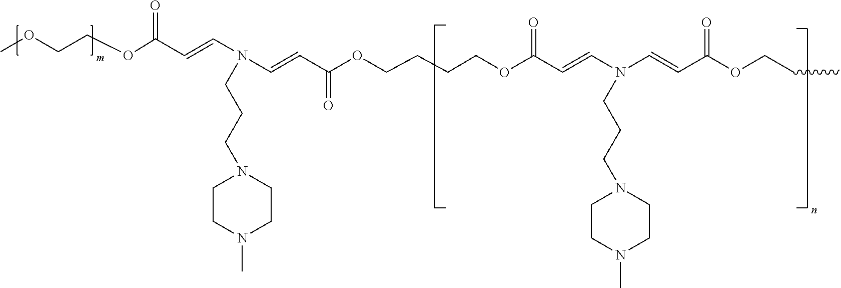

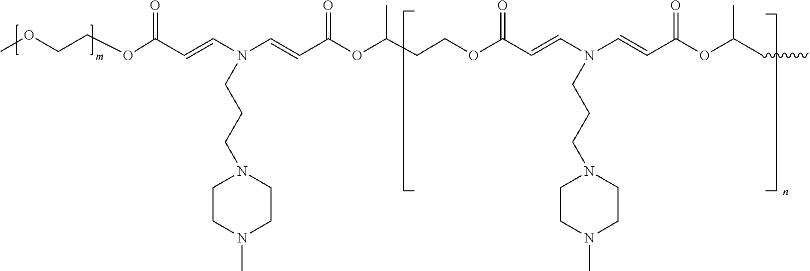

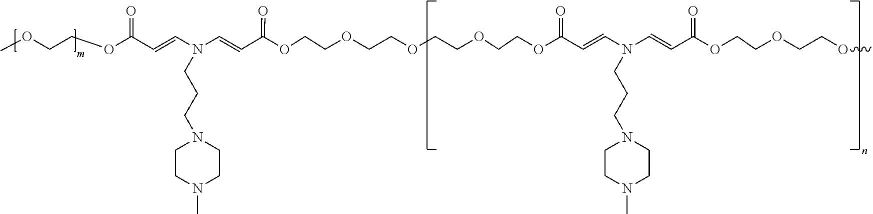

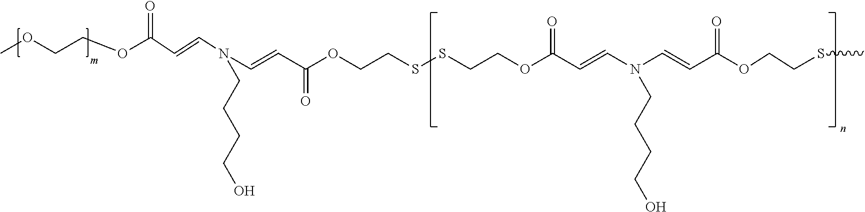

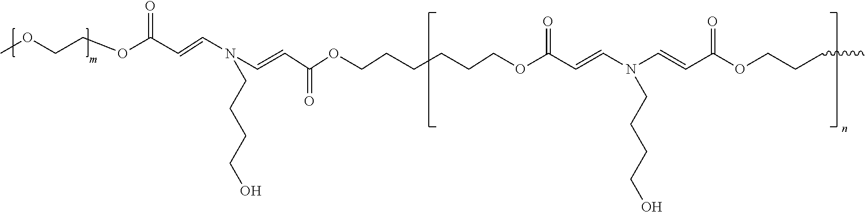

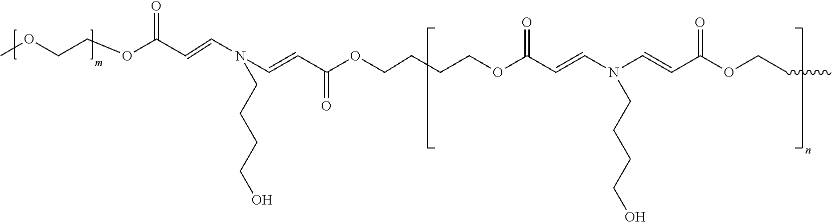

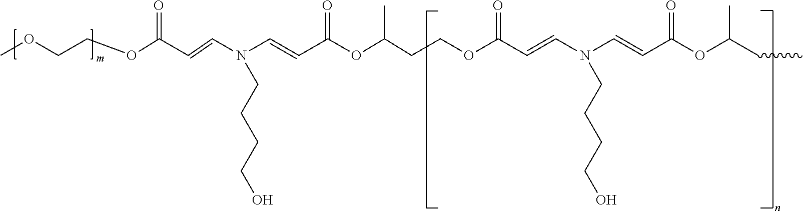

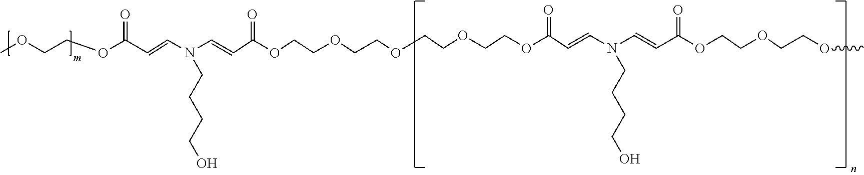

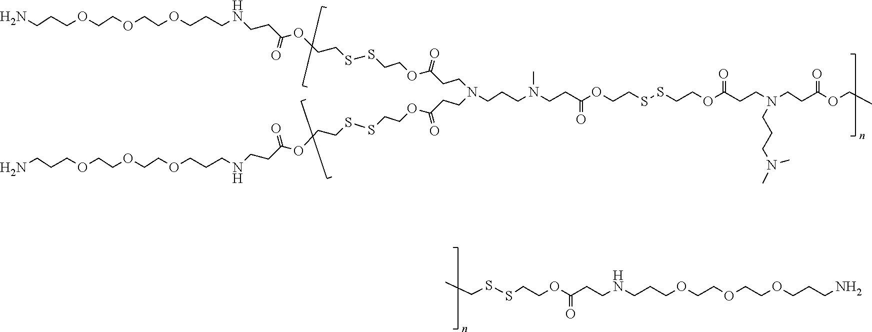

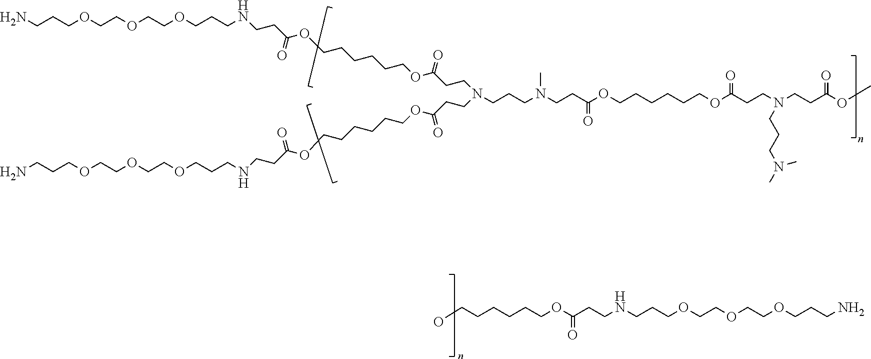

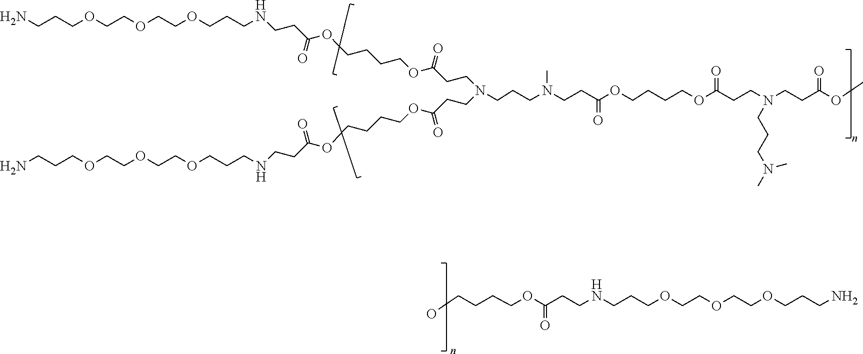

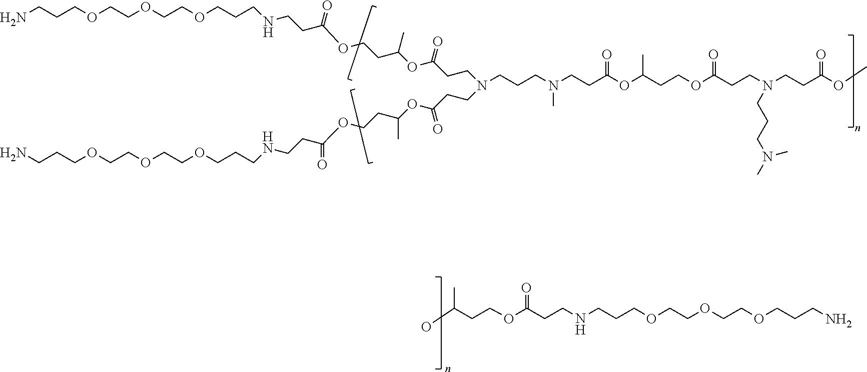

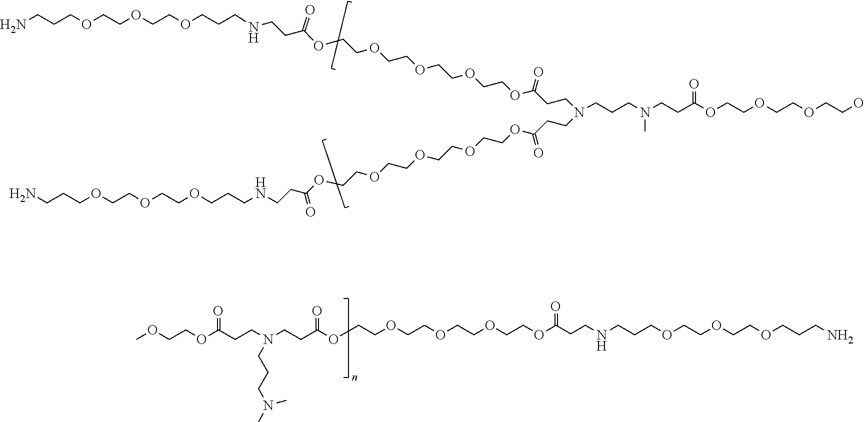

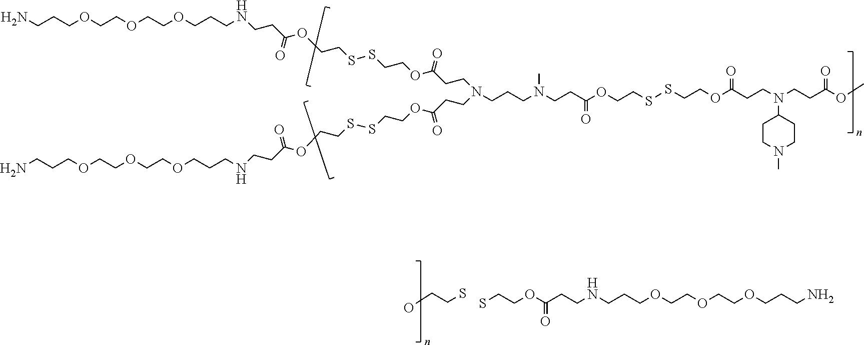

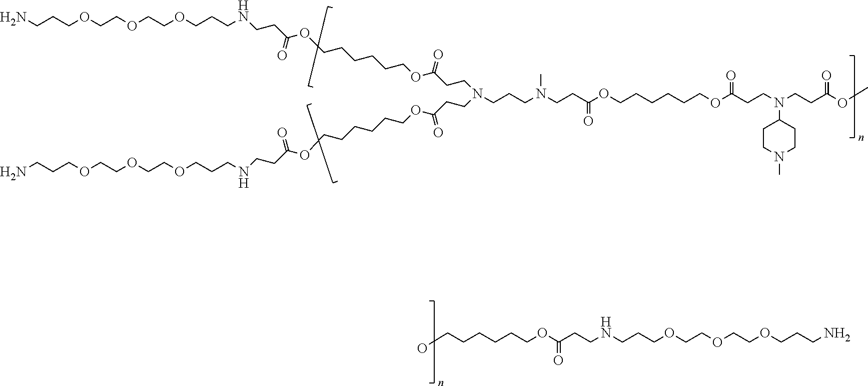

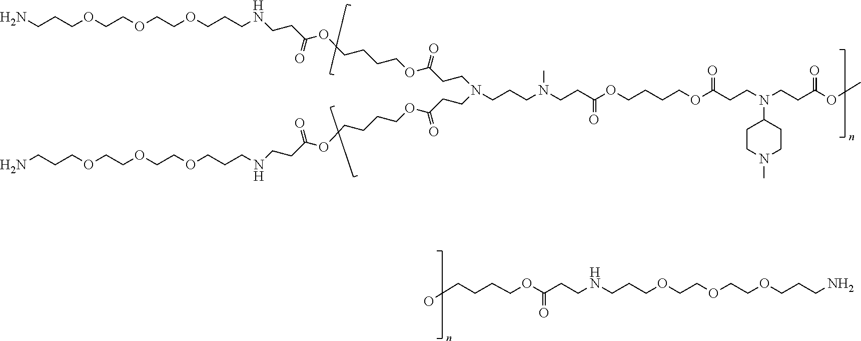

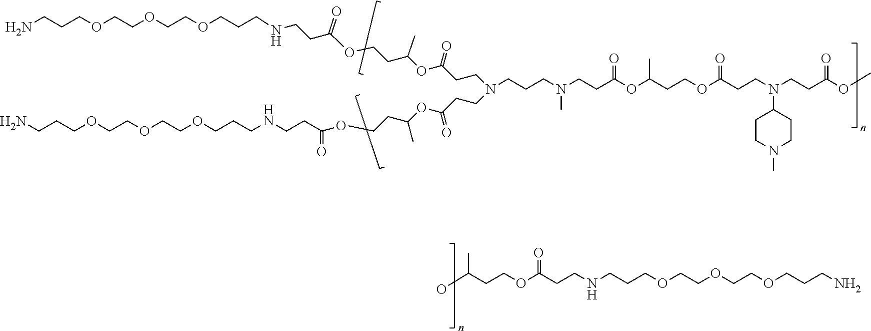

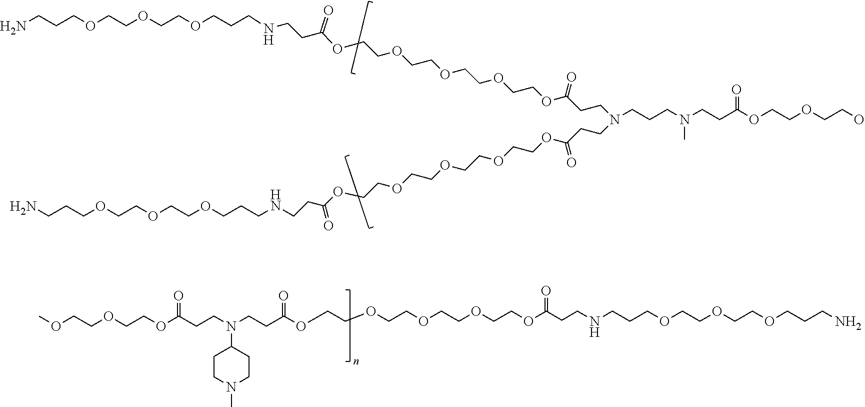

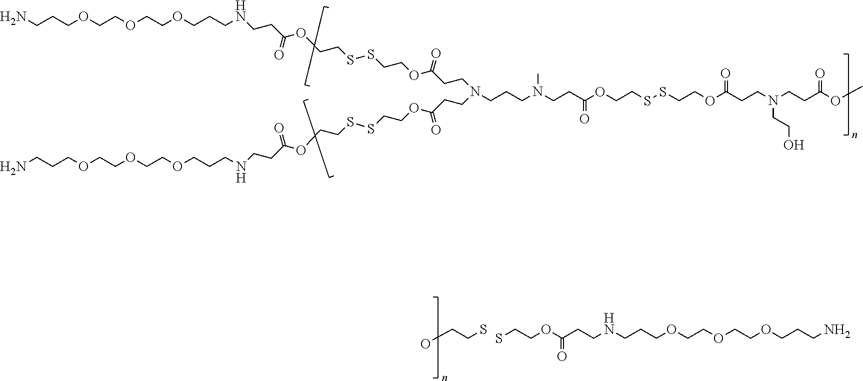

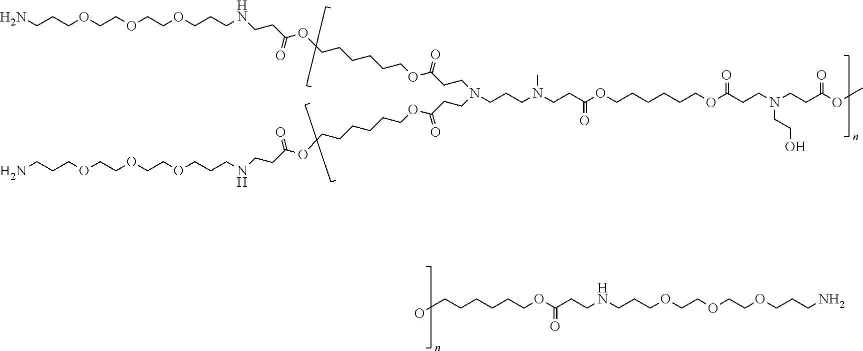

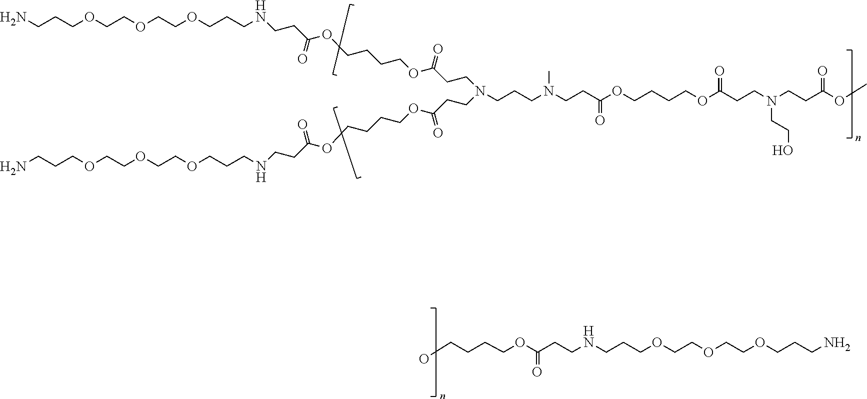

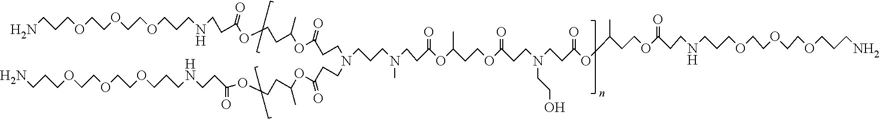

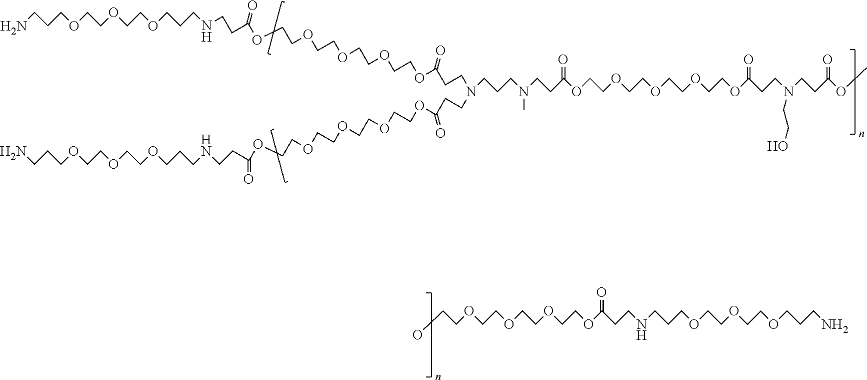

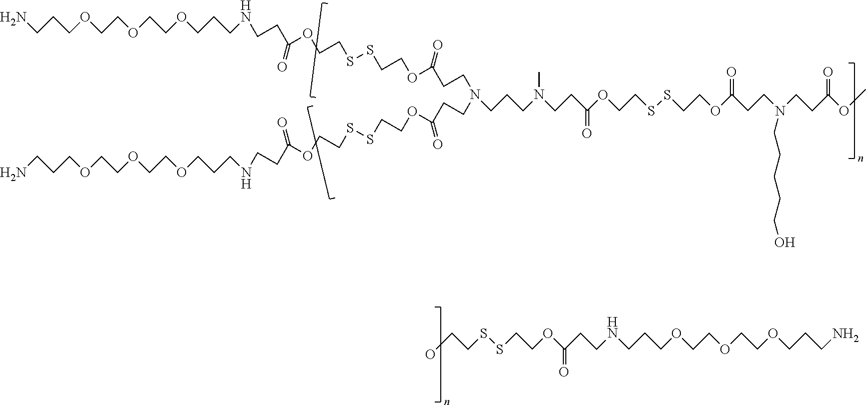

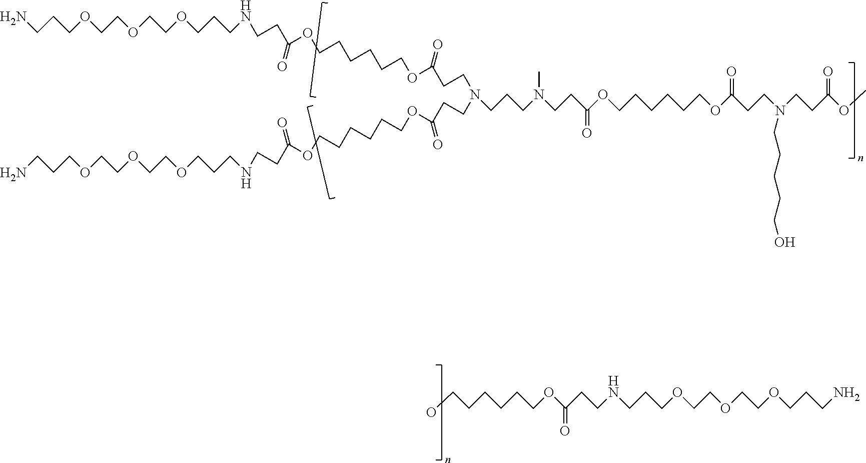

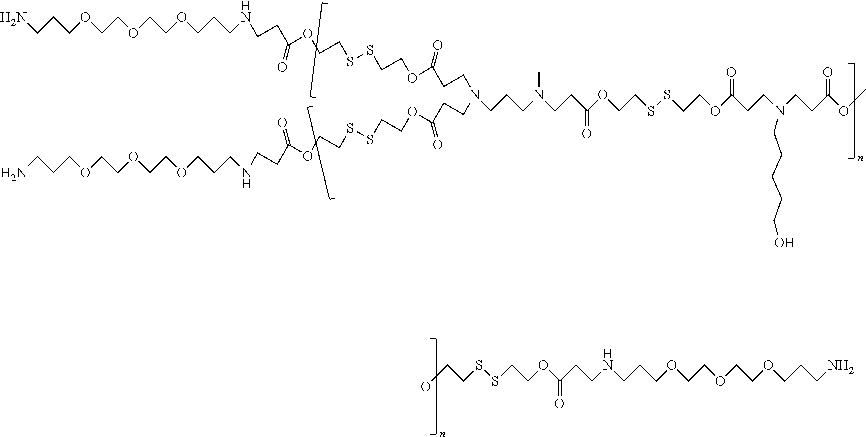

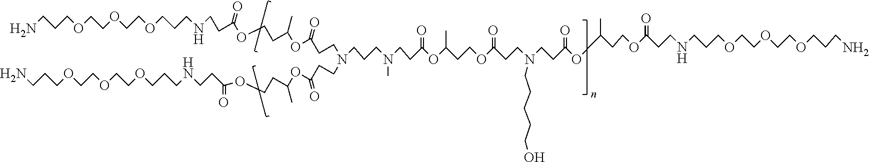

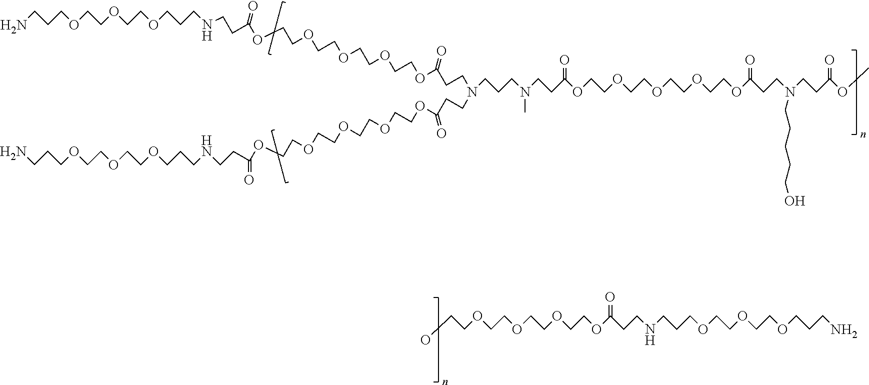

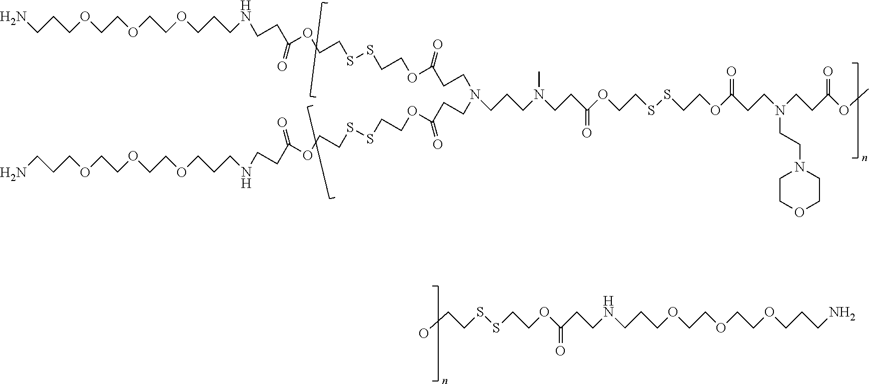

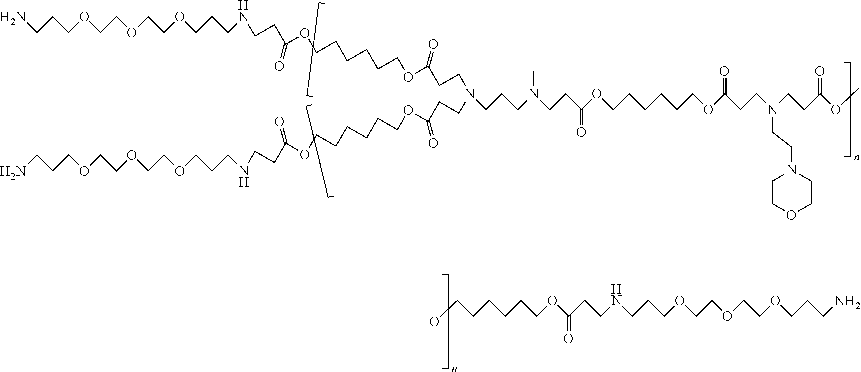

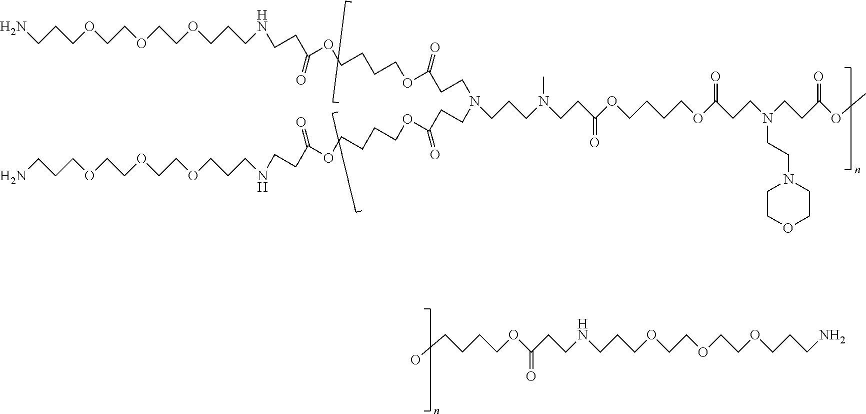

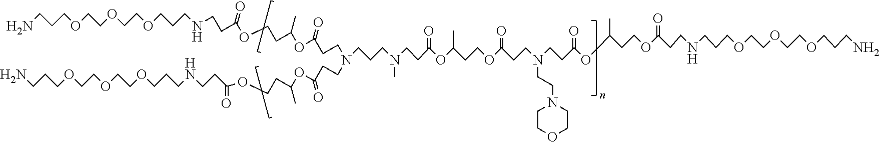

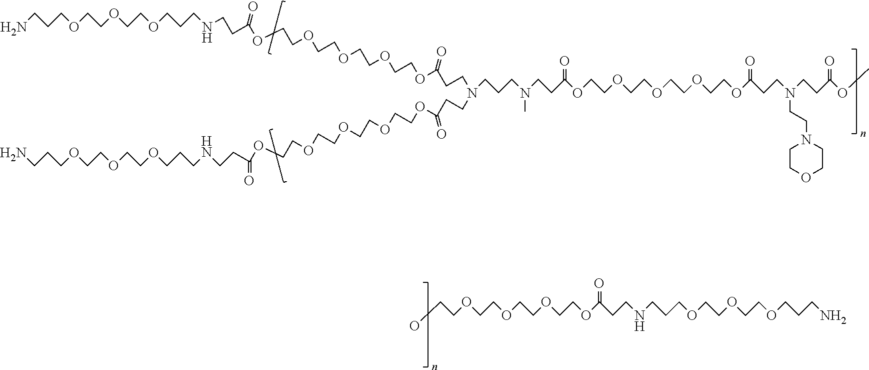

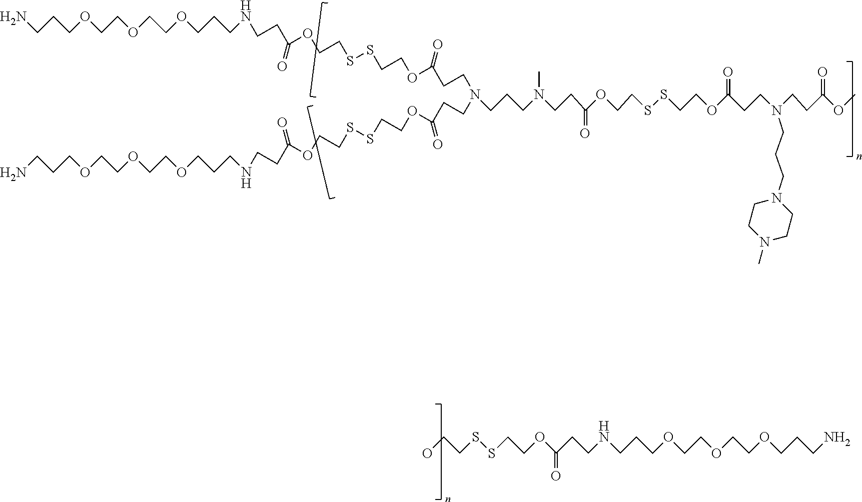

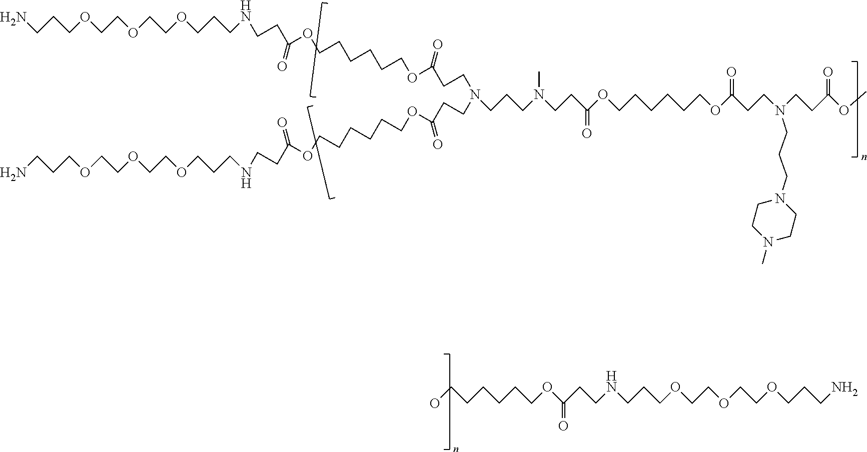

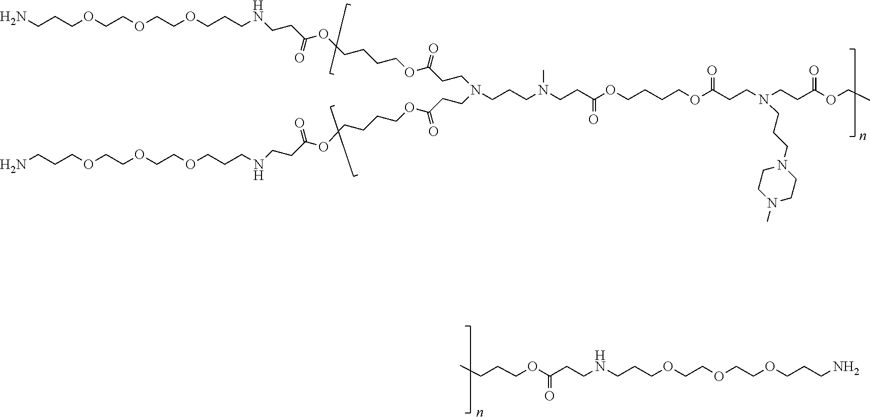

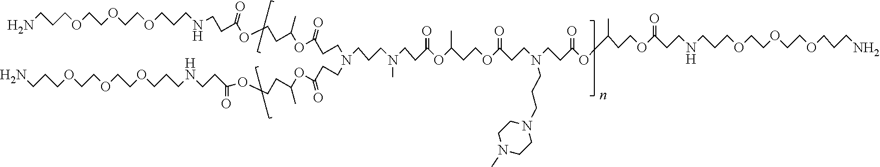

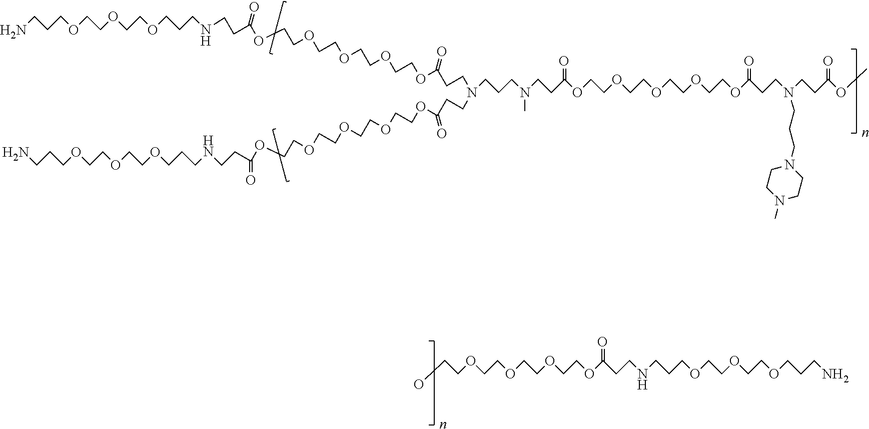

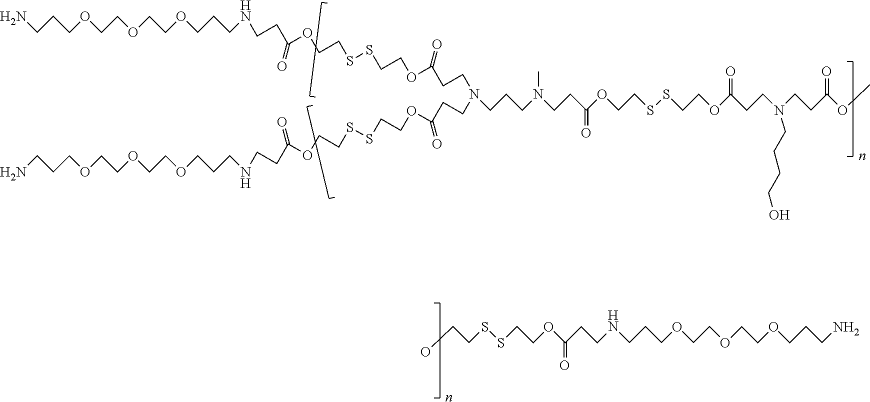

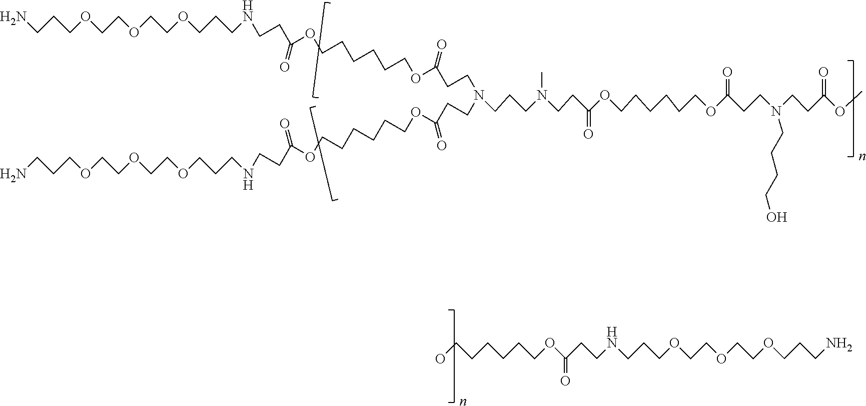

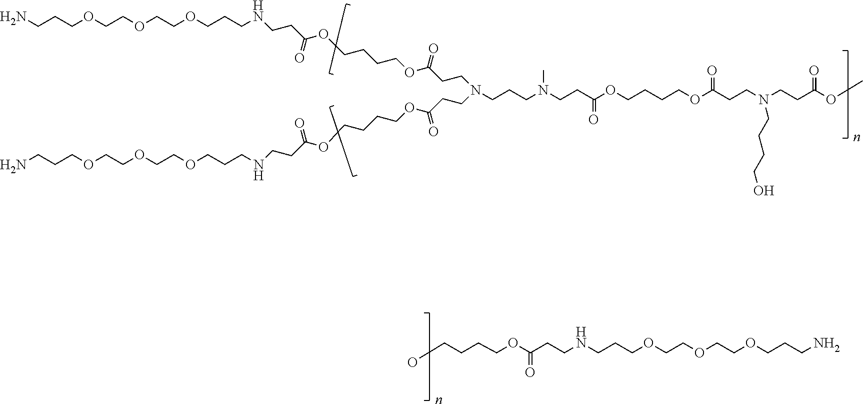

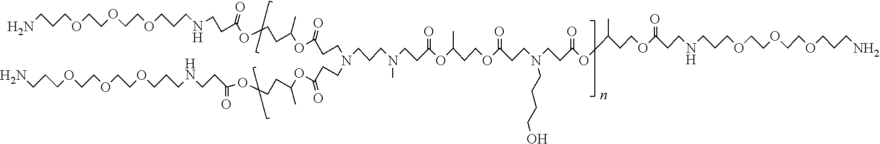

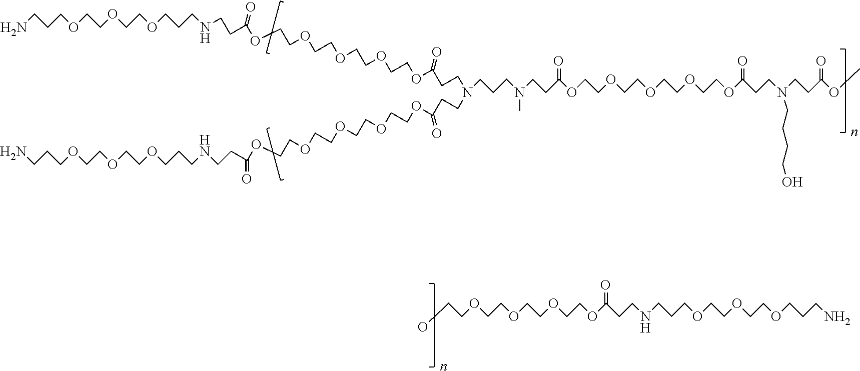

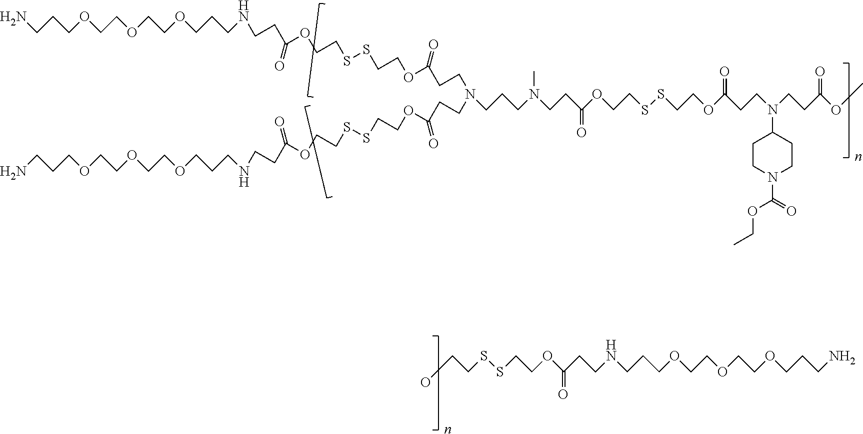

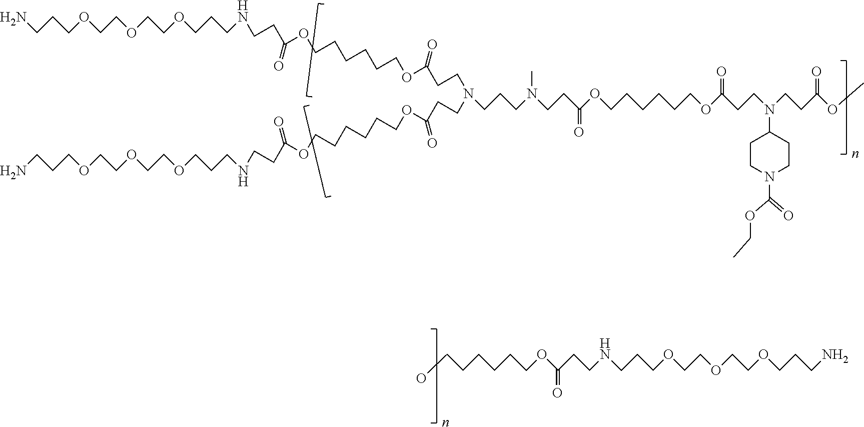

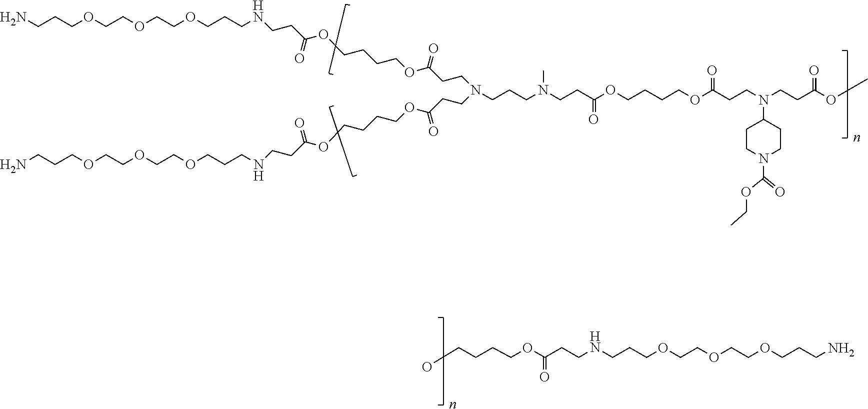

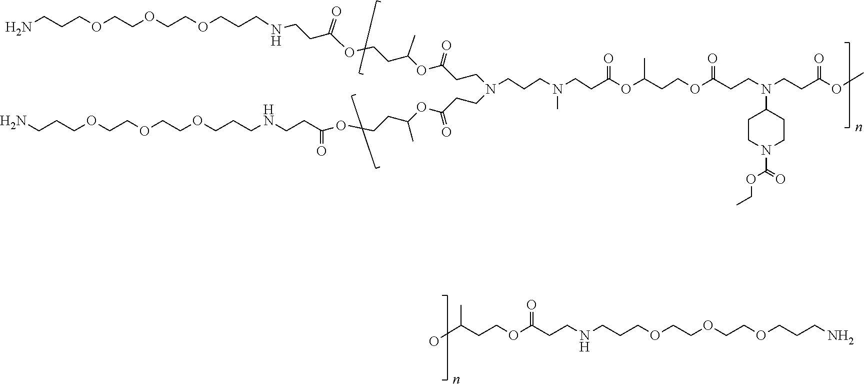

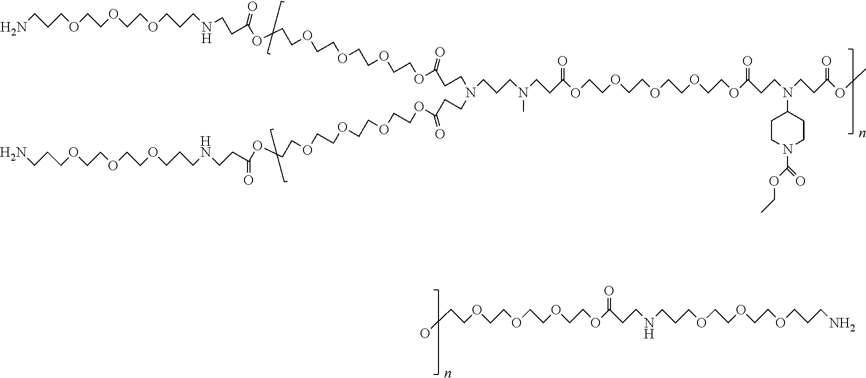

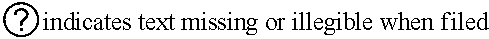

6. The polymer according to claim 1, wherein the polymer comprises a branched monomer of Formula 25 below:

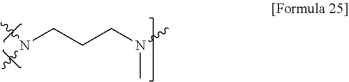

7. The polymer according to claim 1, wherein the polymer comprises a structure of Formula 26 below: 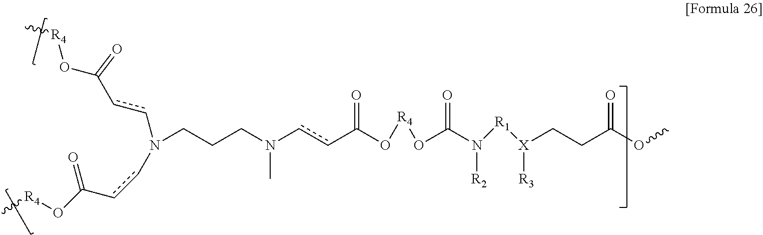
in the formula,
R1 is a bond or a C1-6 alkylene,
R2 and R3 are each independently (i) hydrogen, (ii) C1-6 alkyl which may be substituted with alkylamine or hydroxy, (iii) a 5- or 6-membered carbocycle or heterocycle, or (iv) C1-6 alkyl substituted with a 5- or 6-membered carbocycle or heterocycle,
said R2 and R3 may be linked together to form a C1-6 alkylene,
said 5- or 6-membered carbocycle or heterocycle may be substituted with C1-3 alkyl or C1-3 alkoxyacyl,
X is a nitrogen atom or a bond, and when X is a bond, said R3 is absent,
R4 is a linear or branched C1-15 alkylene, 1 to 5 carbon atoms of said alkylene may be replaced by oxygen or sulfur, and [symbol]
represents a single bond or a double bond.

8. The polymer according to claim 2, wherein the polymer comprises a structure of Formula 27 below: 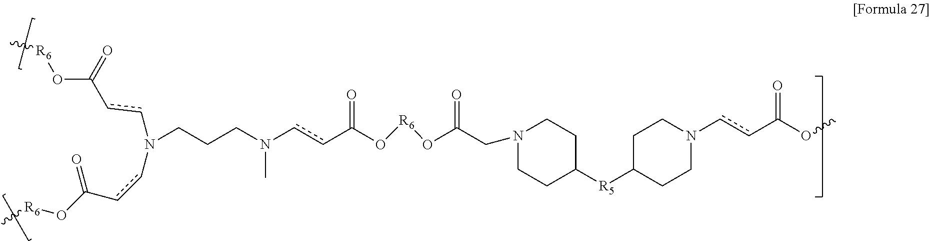
in the formula,
R5 is a C1-6 alkylene,
R6 is a linear or branched C1-15 alkylene, 1 to 5 carbon atoms of said alkylene may be replaced by oxygen or sulfur, and [symbol]
represents a single bond or a double bond.

9. The polymer according to claim 1, wherein one or both ends of the polymer are linked to a PEG-containing moiety comprising a structure of Formula 28 below: 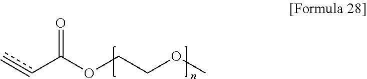
in the formula, [symbol]
represents a double bond or a triple bond, and
n is an integer in the range of 1 to 500.

10. A polymeric nanoparticle in which the polymer according to claim 1 and a nucleic acid molecule form a complex.

11. The polymeric nanoparticle according to claim 10, wherein the nucleic acid molecule is RNA.

12. The polymeric nanoparticle according to claim 10, wherein the nucleic acid molecule is DNA.

13. The polymeric nanoparticle according to claim 10, wherein the nucleic acid molecule is isolated in a pH-dependent manner and delivered into cells in the body.

14. The polymeric nanoparticle according to claim 11, wherein when delivered into the body, the expression of the nucleic acid molecule is maintained for a longer period of time compared to when the same nucleic acid molecule is delivered into the body in the form of a non-polymeric lipid nanoparticle.

15. A method for delivering a nucleic acid molecule into a cell using the polymeric nanoparticle according to claim 10.

16. The method according to claim 15, wherein the polymeric nanoparticle is delivered by parenteral, intramuscular, subcutaneous, or intravenous administration.

17. A pharmaceutical composition comprising the polymeric nanoparticle according to claim 10.

18. The pharmaceutical composition according to claim 17, wherein the pharmaceutical composition is a gene therapeutic agent.

19. The pharmaceutical composition according to claim 17, wherein the pharmaceutical composition is a vaccine.
